(19) United States
(12) Patent Application Publication
(10) Pub. No.: US 2006/0216707 A1
(43) Pub. Date: Sep. 28, 2006

(54) Nucleic acid array consisting of selective monocyte macrophage genes

(76) Inventors: Bruno Stuhlmuller, Berlin (DE); Thomas Haeupl, Erkner (DE); Olaf Kiesslich, Berlin (DE); Gerd-Rudiger Burmester, Berlin (DE)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/521,935

(22) PCT Filed: May 28, 2003
(86) PCT No.: PCT/DE03/01822

§ 371 Date: Jan. 24, 2005

(30) Foreign Application Priority Data
Jul. 24, 2002 (DE) 102 34 524.4

(51) Int. Cl. C12Q 1/68; C12M 1/34

(52) U.S. Cl. 435/6; 435/287.2

(57) ABSTRACT

The invention relates to an array comprising oligo- or poly-nucleotide probes, immobilized on a solid support. The array is characterized in that, sequences of a selection or all of the selective monocytic macrophagic genes given in Tables 1-6 are bonded on the surface. The array permits the diagnosis of rheumatoid arthritis and other chronic inflammatory diseases, a corresponding analysis of the efficacy of treatment and the monitoring of side-effects with the anti-tumor necrosis factor (TNF) therapy and thus permits the selection of the most effective therapy for each patient with rheumatoid arthritis. The invention further relates to a nucleic acid array for the prognosis and development of novel anti-TNF type medicaments and such medicaments with a mode of action in said regulatory circuit.

The present invention relates to an array consisting of oligo- or polynucleotide probes that are applied and immobilised on a solid substrate. The array is characterised in that sequences of a selection or all of the selective monocyte macrophage genes are fixed on the surface, which are enlisted in Tables 1 to 6. This nucleic-acid array permits the diagnosis of the rheumatoid arthritis, associated analysis of the efficiency of treatment and the monitoring of side effects occurring in the anti-tumour necrosis factor (TNF) therapy and hence the selection of the therapeutic approach that is most efficient for the respective patient suffering from rheumatoid arthritis. The present invention moreover relates to a nucleic-acid array for prognosis and for the development of novel pharmaceuticals with an anti-TNF orientation or of such pharmaceuticals that interfere with the control loop thereof.

The cells of the monocyte/macrophage system play a substantial role in the activation of and in maintaining inflammation cascades in the blood and in the tissue, e.g. in the course of rheumatoid arthritis, and also in other chronic inflammatory diseases as well as in auto-aggressive conditions. In the case of these diseases monocytes and macrophages are highly activated, display variations in the occupation of their surface molecules, enter into contact with other cells and secrete certain messenger substances such as those including TNF alpha, which contribute to a sustained inflammatory process. TNF alpha is a cytokine formed by monocytes/macrophages, lymphocytes and mastocytes, which takes an influence on inflammation, sepsis, lipid and protein metabolism, haematogenesis, angiogenesis, the healing of wounds and a defensive immune response and which produces also a cytolytic or cytostatic effect on tumour cells.

In the event of inflammatory diseases, monocytes/macrophages display a characteristic, pathologically varied gene expression pattern with distinct variations, compared against healthy probands. Bio-information methods known to those skilled in the art, e.g. the significance and cluster analysis, it is possible, inter alia, to identify or diagnose genes with a similar behaviour and highly or low-regulated genes from the hybridisation patterns of a nucleic-acid array.

With the ever-increasing availability of the high-throughput methods in the form of nucleic-acid arrays, with the exponentially growing information about the human genome and gene expression, as well as with the global networking of data bases storing structured biomedical information, the approach to and the view of chronic inflammatory and inflammatory-rheumatic conditions has been fundamentally changed. The molecular processes can be defined by application of the improved understanding of the molecular bases of cell-, tissue- and disease-specific gene expression whilst they contribute to enabling an early diagnosis and improved prognosis. On the other hand, micro-array technologies ensure the development of more efficient forms of therapy of the rheumatoid arthritis and other chronic inflammatory diseases, permitting a high-rate screening system.

Moreover, these multiple techniques allow for the accelerated development of pharmaceutical and biologically effective medicaments (biologicals) and also a more rapid assessment of the side effects of medicaments. For this reason, this method entails an economic profit, also in terms of national economics.

The micro-array technology constitutes a miniaturisation of analytic processes on the basis of DNA or RNA hybridisation in a high-throughput method. At the same time, it is possible to analyse thus many thousands of different DNA/DNA (DNA/RNA) interactions within a single test assay. mRNA expression profiles are determined by means of DNA arrays by hybridisation of labelled cRNA or cDNA samples. These technologies require a high degree of automation and standardisation, with the establishment and utilisation of appropriate sample and data bases (sequence information, oligo-nucleotides). The presently employed DNA arrays are distinguished by their substrate material (nylon membranes, glass surfaces, precious metal, vapour-deposited glass surfaces, synthetic materials), by the length or by the production of the DNA sequences immobilised on the substrate and by the labelling technique applied for a sample to be fixed. By way of analogy to the methods of DNA hybridisation in the Southern/Dot-blot test, it is possible to fix DNA sequences on a filter in punctiform shape and in a systematic succession, by spotting by means of a pressure head, by piezo printing methods (ink-jet technology) or by photolithographic methods (chemical direct synthesis on the substrate material). The DNA may be a cDNA, a PCR product or a synthetically produced oligo-nucleotide. Each of these sequences so applied is hence assigned to a specific site in a known array. The RNA may be decontaminated from a clinical sample or also a sample for pharmaceutical examination and, after transcription by reverse transcription, may be hybridised with the complementary nucleic-acid strands present on the array, which are applied in a high genome-encompassing number or also in a pre-selected number. The sample is labelled by means of integrated radio-active nucleotides, via biotin/streptavidin interactions, via amplifications of the digoxigenin enzyme or also via direct or indirect integrated fluorescence dyes. The information is read from the intensity of radioactivity or from fluorescence on a specific site of the substrate material and hence permits the conclusion of the particular relative quantity of the specifically linked DNA or RNA sequence, which had been present in the sample so labelled.

The activation and deactivation of genes constitutes the basis of all biologic processes and furnishes moreover an extremely sensitive response to varied outside conditions. The extraction of RNA form a biologic sample, the action of labelled cDNA or RNA upon a nucleic-acid array (hybridisation) and its analysis allow for the derivation of a great number of parts of information, with a minimum of time, about the condition of the cells in the biologic sample under varied conditions. The technology based on the hybridisation of nucleic acids entails the advantage of an extremely high specificity, sensitivity and comparatively easy high-speed feasibility.

When genes are activated or deactivated in monocytes/macrophages in a non-physiologic manner, this may be the cause of inflammatory diseases or a measurable sign of such diseases. In an ideal case, the therapy with medicaments producing an anti-TNF effect should normalise the pathologically varied gene expression in the respective cells to the level of that found in healthy patients.

From the examination of the gene expression profiles one could expect that a novel molecular characterisation of the rheumatoid arthritis and other chronic inflammatory diseases becomes possible and hence a classification by sub-groups can be established by patho-physiological peculiar characteristics. With the anti-inflammatory anti-TNF therapies, one can therefore expect prognostic forecasts about the level of aggressiveness in the future development. This would take an early influence on the selection and intensity of the drug-based therapy with the medicaments so far known and used as therapeutic in the case of chronic inflammations, and also with biologically effective TNF blockers. On the other hand, this leads to further starting points for scheduling the form of therapy in view of the potential side effects caused by the action of these medicaments, whilst it is possible to estimate the effects of the side effects at an early stage.

On account of therapies with an anti-TNF orientation in cases of rheumatoid arthritis and other chronic inflammatory or auto-aggressive diseases, firstly a potential genesis of neoplastic changes up to the formation of tumours is under discussion, and secondly, the anti-TNF therapy reduces the defensive immune response so that infections occur more often in treated patients, inter alia tuberculosis.

When nucleic-acid array systems are used it is possible to check the expression of tumour-relevant genes in the course of anti-TNF treatment and hence early indications of potential neoplastic changes can be obtained so that it becomes possible to counteract an incipient tumour formation at an early stage and the anti-TNF therapy can be appropriately adapted or, if necessary, stopped.

The invention is based on the problem of providing means for monitoring the efficiency and also the side effects of the anti-TNF therapy and for permitting also the fine diagnosis of an inflammatory disease and hence the selection of that form of therapy, which is most efficient for the respective patient. Another problem underlying the present invention consists in the follow-up monitoring of the efficiency and the side effects of novel pharmaceuticals with anti-TNF orientation within the scope of clinical studies. In accordance with the present invention, a new array is provided that consists of oligo- or poly-nucleotide probes that are applied and immobilised on a solid substrate. Compared against genome-wide DNA chips so far known, the advantage of the present invention resists in cost savings in the production of the nucleic-acid array because it contains predominantly only genes that are of interest for a solution to the problem of the invention, which minimises the expenditure in terms of data analysis and evaluation and reduces the costs thereof.

In accordance with the present invention, the problem is solved by a nucleic-acid array including sequences of a selection or of all of the selective monocyte macrophage genes mentioned in Tables 1 to 6, which are applied on the surface of the array. With reference to the name of the gene or the sequence, respectively, or the accession number it is possible to determine the sequence from data bases accessible to the general public, preferably GeneBank or EMBL. The sequences of the nucleic acids present on the array may consist of genes whose level of expression is changed by a therapy involving an Anti-TNF effect.

If necessary, further genes may be present on the inventive nucleic-acid array, expediently those which are known to be expressed in each cell and to constitute part of the basic genotype of the cell. The genes coding for these nucleic acids are usually referred to as house-keeping genes and are used for standardisation of the signals obtained. The array may contain the aforementioned sequences in the form of DNA, complementary RNA or chemically modified nucleic acids, preferably PNS (protein nucleic acid).

The genes or gene sequences may be selected genes of rheumatoid arthritis or other chronic inflammatory diseases, which are relevant for the disease or the side effects, selected preferably from the monocyte/macrophage cell system. If applicable, alleles, derivatives and/or splicing variants of the genes or gene fragment sequences or oligomer sequences may be present on the surface of the array. The congruence of the sequences on the array with the corresponding sequences in Tables 1 to 6 should be at least 80% in the protein-coding mRNA segments.

The substrate onto which the nucleic acids are applied may be any substrate whatsoever which is usually employed for RNA or DNA arrays. The methods for the application and immobilisation of the nucleic acids are part of prior art and are common to those skilled in the art. For coupling to the aforementioned sequences, the substrate may be coated with reactive groups, metal compounds or alloys. The genes or gene sequences may be applied, for instance, by spotting techniques, immobilising techniques or by techniques of in-situ synthesis of oligomers or in an enantiomorphic manner in the form of RNA.

The inventive array may be used, for instance, for measuring the monocyte/macrophage activation or the inflammatory activity in the blood or cell tissue in the case of inflammatory diseases, preferably rheumatoid arthritis. The array may be employed, for example, for the early diagnosis of the aforementioned diseases in genetically predisposed patients even before clinical symptoms become manifest. Another field of application is the fine diagnosis, preferably the subdivision of patients into sub-groups requiring each a different therapy or different medication. The array may furthermore be used to monitor the therapy, to follow up side effects, to establish a prognosis and to identify new pharmaceutical targets in the case of the aforementioned diseases.

To this end, blood or tissue is sampled on the patients under examination, from which RNA is isolated by the known standard techniques and prepared, if necessary, as total RNA or poly A+−RNA for further use. By application of reverse transcriptase it is possible to transcribe the RNA into cDNA and to label it with a marker, e.g. a fluorescent dye, a radioactive nuclide or an enzyme such as alkaline phosphatase. In addition, the RNA may be used in labelled or unlabelled condition for hybridisation of the nucleic-acid array. After hybridisation of the array with the nucleic-acid samples and after subsequent washing steps, the bonding of the sample to the sequences present on the array may be examined with any appropriate method. In the case of a fluorescent labelling, these methods are optical techniques whilst in the case of radioactive labelling of the samples auto radiography would be employed and in the case of enzyme labelling enzymatic analytical techniques are applied, e.g. the conversion of a colourless substrate into a multi-colour product.

An inverse analysis of total or mRNA with the sequences indicated in Tables 1 to 6, which is fixed to a solid phase, is equally possible. To this end, blood- or tissue-specific RNA molecules from up to 500 patients are fixed to the RNA micro arrays. The qualitative/quantitative analysis of the transcript quantity of relevant genes is then carried out with the selected genes, gene segments or oligomers described in Tables 1 to 6. The RNA samples are spotted on coupling substrates and are composed of total RNA or messenger RNA. The RNA serves as target for the highly significantly expressed genes derived from DNA micro arrays according to Tables 1 to 6, which are used as labelled probes for hybridisation. The coupling of biotinylated RNA or messenger RNA on glass substrates (slides) coated with streptavidin is proposed. After the RNA has been labelled with biotin derivatives the RNA is applied by spotting on glass or synthetic slides coated with poly-L-lysine or with streptavidin, with subsequent drying. RNA degradation is prevented in this manner. An alternative is the covalent coupling of RNA by bonding to reactive substrate materials, in which case preferably UV irradiation is used for catalysing. Additionally, a multiple simultaneous labelling of different genes, gene units or oligomers with various labelling species such as radio activity, fluorescein, digoxigenin and enzymatic markers is expedient.

Parallel different labelling of the probes with different fluorescent dyes is possible. As an alternative, enzymatic or even radioactive probe labelling should be mentioned. For a quantification and quality check, labelled house-keeping genes (alpha, beta, gamma actin, GAPDH, etc.) are employed. The analysis is preferably carried out in this case in parallel and simultaneously with a maximum of 50 gene probes per test set-up.

In addition to the simplification of the biometric analysis by coupling RNA species to substrate materials, this system allows for a rapid diagnosis and offers a complex high-speed diagnosis, prognosis and therapy control on an individual basis for the patients. In particular in the case of pharmacologic development strategies, the system allows for a rapid performance at a high throughput.

The following examples and illustrates only serve for explanation and do not restrict the scope of the invention in any way whatsoever.

### 1. Isolation of Monocytes

In the method employed here, the selection of selective highly pure monocytes of peripheral blood was applied in order to enable a finding (1) on the specificity of the disease, (2) on the application of the anti-TNF-alpha therapeutic as “biological”, (3) in relation to a comparison against a healthy proband, and eventually also (4) for assessment of gene-diagnostic potentials relevant in relation to anti-TNF-alpha. For this test, the peripheral haemo-leucocytes from peripheral blood were enriched by Fikoll gradient density centrifugation. This fraction, which constitutes an individually different composition of monocytes (5-12%), CD4+ T-cells (85-92%), CD8+ T-cells (5-10%), NK cells (2-5%), basophilic and neutrophile granulocytes, was subjected to further purification steps for the extraction of specific monocyte fractions. Both, negative selections operating on removal of all other cell fractions via magnetic beads anti-body interactions, and positive selections by CD14+labelling via magnetic beads, or even FACS cell grading techniques were applied. In both methods, monocyte cell purity levels of roughly 96% were achieved.

### 2. RNA Extraction

The pure monocyte fractions were received in RNA lysis buffer whereupon the RNA was purified by means of a commercially available RNA purification kit (Qiagen). The RNA was transcribed into cDNA by application of established cDNA transcription methods by reverse transcription and then subjected to a further step of linear amplification by application of the “Eberwine protocol” for the extraction of aRNA (amplified RNA). The quantity and the quality of RNA, cDNA and aRNA were verified each by gel electrophoresis, photometric analysis and measurements carried out with the Bioanalyzer 2100 (Agilent company).

### 3. Affymetrix Chip Hybridisation

Specific oligo-nucleotides derived directly from data base sequences were used as DNA samples for expression analyses in the system available from the Affymetrix company. These samples are hybridised on the array with targets from fluorescence-labelled, reversely transcribed samples in the form of cDNA or with linearly amplified samples in the form of aRNA.

Hybridisation of the genome-wide Affymetrix array (U-133A) and subsequent processing are carried out by machine under standard conditions in compliance with the instructions by the producer, Affymetrix, using a special hybridisation and washing device with the specific buffers. After hybridisation, gene expression patterns are produced via the ratio of fluorescence intensity levels at a defined wavelength. Such high-throughput expression analyses permit comparisons of the expression quantities of genes in healthy and diseased persons at the same time or comparisons of the gene expression before and after drug addition for risk assessment (pharmaceutical/toxicity genomics), for fine diagnosis and for assessment of the complexity of diseases.

### 4. Evaluation of Data

Here, aRNA samples from peripheral haemo-monocytes (1) of healthy blood donors, (2) of chronic active patients suffering from rheumatoid arthritis before treatment, and (3) after treatment with TNF-alpha antibodies were used. The success of the treatment was assessed by unambiguous laboratory clinical parameters and by the criteria of the internationally applicable parameter examinations (ACR criteria) to be applied on a clinical level. The objective and the purpose of this examination of groups of three were the establishment of characteristic gene expressions in the following group definitions:
1. A gene-regulatory specificity of a disease in active rheumatoid arthritis, without treatment, compared against the gene expression of healthy probands.
2. The characterisation of a gene-regulatory specific interpretation of the anti-TNF-alpha treatment and an assessment of the treatment by comparison against the gene expression of the active disease without treatment and by comparison against the gene expression of the healthy probands.
3. Ensured assessment of side effects by the anti-TNF medicament “biological”. Here, the specific gene expression of the patients suffering from rheumatoid arthritis, who are treated with anti-TNF-alpha, was compared against the gene expression of the same patients without treatment and the gene expression in healthy blood donors.

The individual gene expressions were processed and measured within the genome-wide human Affymetrix array (U-133A) in the appertaining Affymetrix hybridisation/washing and reading system. The evaluation is carried out in 4 steps:
1. The determination of the significant genes detected in the expression analysis, e.g. by the “fold-change method” or by SAM (Significance Analysis of Microarrays).
2. The separation of the significant genes into different sub-populations on the basis of the examination of the expression characteristics of these genes by means of cluster analysis carried out by techniques such as “hierarchical clustering”!, “self-organizing maps” or “k-means clustering”.
3. The evaluation of the behaviour of the significant genes within the clusters with integration of the clinical information (rheumatoid arthritis (RA), anti-TNF therapy) and on the basis of the empirical values furnished by specialists.
4. The classification of the involved genes by biologic pathways.

General Characteristics of the Significant Genes within the Clusters:

[image]

The gene expression behaviour of a healthy normal donor (NS) and of an active patient suffering from rheumatoid arthritis (RA) before and after anti-TNF-alpha therapy was compared by means of a cluster analysis. The results are represented in Illustrations 1 and 2.

[image]

The illustrations show the gene expressions by the cluster analysis (n=6 cluster). The number of the genes involved is indicated in brackets. The result of the cluster analysis furnishes a confidence interval in addition to the mean gene expression behaviour of all the genes contained in a cluster.

The clusters displayed the following characteristics:
CLUSTER-1: Compared against a healthy proband, the disease-specific gene expression is reduced; in this case, the anti-TNF treatment does not create a gene-regulatory effect.
CLUSTER-2: Side effects: Represented by the effect of the medication in anti-TNF-alpha treatment, there is a reduced expression of the associated genes in the treated patient.
CLUSTER-3: Compared against a healthy proband, the disease-specific gene expression is stronger. The anti-TNF-alpha treatment creates a positive effect.
CLUSTER-4: Compared against a healthy proband, the disease-specific gene expression is increased. The anti-TNF treatment creates a positive effect.
CLUSTER-5: Side effects: Represented by the effect of the medication in anti-TNF-alpha treatment, there is an increased expression of the associated genes in the treated patient.
CLUSTER-6: Compared against a healthy proband, the disease-specific gene expression is increased. The anti-TNF-alpha treatment does not create a gene-regulatory effect in this case.

Tables 1 to 6 show the genes contained in the clusters described above, together with the Affymetrix designation (left) and their defined GeneBank Accession Number, inclusive of a description.

TABLE 1
Genes from Cluster Analysis 1

| Affymetrix | |
|---|---|
| internal | |
| designation | Description of the sequence in the GeneBank data base |
| 211654_x_at | gb: M17565.1 /DEF = Human MHC class II DQ-beta associated with DRw6, DQw1 protein, complete cds. /FEA = mRNA /GEN = HLA-DQB1 |
| | /DB_XREF = gi: 188188 /FL = gb: M17565.1 |
| 213831_at | Consensus includes gb: X00452.1 /DEF = Human mRNA for DC classII histocompatibility antigen alpha-chain. /FEA = mRNA |
| | /PROD = DC classII histocompatibility antigenalpha-chain /DB_XREF = gi: 32265 /UG = Hs.198253 major histocompatibility complex, |
| | class II, DQ alpha 1 |
| 212203_x_at | Consensus includes gb: BF338947 /FEA = EST /DB_XREF = gi: 11285367 /DB_XREF = est: 602036012F1 /CLONE = IMAGE: 4184090 |
| | /UG = Hs.182241 interferon induced transmembrane protein 3 (1-8U) |
| 209480_at | gb: M16276.1 /DEF = Human HHC class II HLA-DR2-Dw12 mRNA DQw1-beta, complete cds. /FEA = mRNA /GEN = HLA-DRB2 |
| | /DB_XREF = gi: 188397 /UG = Hs.73931 major histocompatibility complex, class II, DQ beta 1 /FL = gb: M60028.1 gb: M17564.1 |
| | gb: M81140.1 gb: M81141.1 gb: M16276.1 gb: NM_002123.1 |
| 218345_at | gb: NM_018487.1 /DEF = Homo sapiens hepatocellular carcinoma-associated antigen 112 (HCA112), mRNA. /FEA = mRNA |
| | /GEN = HCA112 /PROD = hepatocellular carcinoma-associated antigen 112 /DB_XREF = gi: 8923717 /UG = Hs.12126 hepatocellular |
| | carcinoma-associated antigen 112 /FL = gb: AF258340.1 gb: NM_018487.1 |
| 221491_x_at | Consensus includes gb: AA807056 /FEA = EST /DB_XREF = gi: 2876632 /DB_XREF = est: oc35c12.s1 /CLONE = IMAGE: 1351702 |
| | /UG = Hs.279930 major histocompatibility complex, class II, DR beta 3 /FL = gb: M27635.1 gb: NM_022555.1 gb: M26038.1 |
| | gb: U95989.1 gb: U95819.1 gb: U66825.1 |
| 211734_s_at | gb: BC005912.1 /DEF = Homo sapiens, Fc fragment of IgE, high affinity I, receptor for; alpha polypeptide, clone MGC: 14507, |
| | mRNA, complete cds. /FEA = mRNA /PROD = Fc fragment of IgE, high affinity I, receptorfor; alpha polypeptide /DB_XREF = gi: 13543505 |
| | /FL = gb: BC005912.1 |
| 201330_at | gb: NM_002887.1 /DEF = Homo sapiens arginyl-tRNA synthetase (RARS), mRNA. /FEA = mRNA /GEN = RARS /PROD = arginyl-tRNA synthetase |
| | /DB_XREF = gi: 4506428 /UG = Hs.180832 arginyl-tRNA synthetase /FL = gb: BC000528.1 gb: NM_002887.1 |
| 201114_x_at | gb: NM_002792.1 /DEF = Homo sapiens proteasome (prosome, macropain) subunit, alpha type, 7 (PSMA7), mRNA. /FEA = mRNA |
| | /GEN = PSMA7 /PROD = proteasome (prosome, macropain) subunit, alphatype, 7 /DB_XREF = gi: 4506188 /UG = Hs.233952 proteasome |
| | (prosoma, macropain) subunit, alpha type, 7 /FL = gb: BC004427.1 gb: AF022815.1 gb: AF054185.1 gb: NM_002792.1 |
| 213418_at | Consensus includes gb: NM_002155.1 /DEF = Homo sapiens heat shock 70 kD protein 6 (HSP70B) (HSPA6), mRNA. /FEA = CDS |
| | /GEN = HSPA6 /PROD = heat shock 70 kD protein 6 (HSP70B) /DB_XREF = gi: 4504514 /UG = Hs.3268 heat shock 70 kD protein 6 (HSP70B) |
| | /FL = gb: NM_002155.1 |
| 208961_s_at | gb: AB017493.1 /DEF = Homo sapiens mRNA for DNA-binding zinc finger(GBF), complete cds. /FEA = mRNA |
| | /PROD = DNA-binding zinc finger(GBF) /DB_XREF = gi: 3582142 /UG = Hs.285313 core promoter element binding protein /FL = gb: BC000311.1 |
| | gb: BC004301.1 gb: AF001461.1 gb: AB017493.1 gb: NM_001300.2 |
| 217753_s_at | gb: NM_001029.1 /DEF = Homo sapiens ribosomal protein S26 (RPS26), mRNA. /FEA = mRNA /GEN = RPS26 /PROD = ribosomal protein S26 |
| | /DB_XREF = gi: 4506708 /UG = Hs.299465 ribosomal protein S26 /FL = gb: BC002604.1 gb: NM_001029.1 |
| 201403_s_at | gb: NM_004528.1 /DEF = Homo sapiens microsomal glutathione S-transferase 3 (MGST3), mRNA. /FEA = mRNA /GEN = MGST3 |
| | /PROD = microsomal glutathione S-transferase 3 /DB_XREF = gi: 4758713 /UG = Hs.111811 microsomal glutathione S-transferase 3 |
| | /FL = gb: BC000505.1 gb: BC003034.1 gb: AF026977.1 gb: NM_004528.1 |
| 203103_s_at | gb: NM_014502.1 /DEF = Homo sapiens nuclear matrix protein NMP200 related to splicing factor PRP19 (NMP200), mRNA. /FEA = mRNA |
| | /GEN = NMP200 /PROD = nuclear matrix protein NMP200 related tosplicing factor PRP19 /DB_XREF = gi: 7657380 /UG = Hs.173980 |
| | nuclear matrix protein NMP200 related to splicing factor PRP19 /FL = gb: NM_014502.1 |
| 221903_s_at | Consensus includes gb: BE046443 /FEA = EST /DB_XREF = gi: 8363496 /DB_XREF = est: hn47d10.x2 /CLONE = IMAGE: 3026803 /UG = Hs.18827 |
| | KIAA0849 protein |
| 217379_at | Consensus includes gb: AL121934 /DEF = Human DNA sequence from clone RP11-209A2 on chromosome 6. Contains an RPL10 |
| | (60S ribosomal protein L10) pseudogene, ESTs, STSs and GSSs /FEA = CDS /DB_XREF = gi: 9795199 /UG = Hs.272340 Human DNA sequence |
| | from clone RP11-209A2 on chromosome 6. Contains an RPL10 (60S ribosomal protein L10) pseudogene, ESTs, STSs end GSSs |
| 206120_at | gb: NM_001772.1 /DEF = Homo sapiens CD33 antigen (gp67) (CD33), mRNA. /FEA = mRNA /GEN = CD33 /PROD = CD33 antigen (gp67) |
| | /DB_XREF = gi: 4502654 /UG = Hs.83731 CD33 antigen (gp67) /FL = gb: M23197.1 gb: NM_001772.1 |
| 202737_s_at | gb: NM_012321.1 /DEF = Homo sapiens U6 snRNA-associated Sm-like protein (LSM4), mRNA. /FEA = mRNA /GEN = LSM4 /PROD = U6 snRNA-associated Sm- |
| | like protein /DB_XREF = gi: 6912485 /UG = Hs.76719 U6 snRNA-associated Sm-like protein /FL = gb: BC000387.1 gb: BC003652.1 gb: AF182290.1 |
| | gb: AF117235.1 gb: NM_012321.1 gb: AF251218.1 |
| 201416_at | Consensus includes gb: BG528420 /FEA = EST /DB_XREF = gi: 13519957 /DB_XREF = est: 602579853F1 /CLONE = IMAGE: 4719060 |
| | /UG = Hs.83484 SRY (sex determining region Y)-box 4 /FL = gb: NM_003107.1 |
| 214084_x_at | Consensus includes gb: AW072388 /FEA = EST /DB_XREF = gi: 6027386 /DB_XREF = est: xa07d05.x1 /CLONE = IMAGE: 2567625 /UG = Hs.1583 |
| | neutrophil cytosolic factor 1 (47 kD, chronic granulomatous disease, autosomal 1) |
| 204861_s_at | gb: NM_004536.1 /DEF = Homo sapiens baculoviral IAP repeat-containing 1 (BIRC1), mRNA. /FEA = mRNA /GEN = BIRC1 /PROD = baculoviral IAP repeat- |
| | containing 1 /DB_XREF = gi: 4758751 /UG = Hs.79019 baculoviral IAP repeat-containing 1 /FL = gb: U19251.1 gb: NM_004536.1 |
| 221666_s_at | gb: BC004470.1 /DEF = Homo sapiens, clone MGC: 10332, mRNA, complete cds. /FEA = mRNA /PROD = Unknown (protein for MGC: 10332) |
| | /DB_XREF = gi: 13325315 /UG = Hs.71869 apoptosis-associated speck-like protein containing a CARD /FL = gb: BC004470.1 |
| 218421_at | gb: NM_022766.1 /DEF = Homo sapiens hypothetical protein FLJ23239 (FLJ23239), mRNA. /FEA = mRNA /GEN = FLJ23239 /PROD = hypothetical protein |
| | FLJ23239 /DB_XREF = gi: 12232440 /UG = Hs.34516 hypothetical protein FLJ23239 /FL = gb: NM_022766.1 gb: BC004278.1 |
| 217794_at | gb: NM_018457.1 /DEF = Homo sapiens DKFZp564J157 protein (DKFZP564J157), mRNA. /FEA = mRNA /GEN = DKFZP564J157 |
| | /PROD = DKFZp564J157 protein /DB_XREF = gi: 8922156 /UG = Hs.63042 DKFZp564J157 protein /FL = gb: AF217517.1 gb: NM_018457.1 |
| 201558_at | gb: NM_003610.1 /DEF = Homo sapiens RAE1 (RNA export 1, S. pombe) homolog (RAE1), mRNA. /FEA = mRNA /GEN = RAE1 |
| | /PROD = RAE1 (RNA export 1, S. pombe) homolog /DB_XREF = gi: 4506398 /UG = Hs.196209 RAE1 (RNA export 1, S. pombe) homolog |
| | /FL = gb: U84720.1 gb: NM_003610.1 |
| 218055_s_at | gb: NM_018268.1 /DEF = Homo sapiens hypothetical protein FLJ10904 (FLJ10904), mRNA. /FEA = mRNA /GEN = FLJ10904 |
| | /PROD = hypothetical protein FLJ10904 /DB_XREF = gi: 8922759 /UG = Hs.16470 hypothetical protein FLJ10904 /FL = gb: NM_018268.1 |
| 202191_s_at | Consensus includes gb: BE439987 /FEA = EST /DB_XREF = gi: 9439470 /DB_XREF = est: HTM1-745F /UG = Hs.226133 growth arrest-specific 7 |
| | /FL = gb: AB007854.1 gb: NM_005890.1 |
| 205550_s_at | gb: NM_004899.1 /DEF = Homo sapiens brain and reproductive organ-expressed (TNFRSF1A modulator) (BRE), mRNA. /FEA = mRNA |
| | /GEN = BRE /PROD = brain and reproductive organ-expressed (TNFRSF1Amodulator) /DB_XREF = gi: 4757871 /UG = Hs.80426 brain and |
| | reproductive organ-expressed (TNFRSF1A modulator) /FL = gb: BC001251.1 gb: NM_004899.1 gb: L38616.1 |
| 202941_at | gb: NM_021074.1 /DEF = Homo sapiens NADH dehydrogenase (ubiquinone) flavoprotein 2 (24 kD) (NDUFV2), mRNA. /FEA = mRNA |
| | /GEN = NDUFV2 /PROD = NADH dehydrogenase (ubiquinone) flavoprotein 2(24 kD) /DB_XREF = gi: 10835024 /UG = Hs.51299 NADH dehydrogenase |
| | (ubiquinone) flavoprotein 2 (24 kD) /FL = gb: NM_021074.1 gb: BC001632.1 gb: M22538.1 |
| 217814_at | gb: NM_020198.1 /DEF = Homo sapiens GK001 protein (GK001), mRNA. /FEA = mRNA /GEN = GK001 /PROD = GK001 protein |
| | /DB_XREF = gi: 9910241 /UG = Hs.8207 GK001 protein /FL = gb: AF113221.1 gb: BC001300.1 gb: AF226054.1 gb: NM_020198.1 |
| 212051_at | Consensus includes gb: AA676803 /FEA = EST /DB_XREF = gi: 2657325 /DB_XREF = est: zj65b04.s1 /CLONE = IMAGE: 455119 |
| | /UG = Hs.13996 Homo sapiens cDNA: FLJ23260 fis, clone COL05804, highly similar to HSU90911 Human clone 23652 mRNA sequence |
| 212386_at | Consensus includes gb: BF592782 /FEA = EST /DB_XREF = gi: 11685106 /DB_XREF = est: 7j94d06.x1 /CLONE = IMAGE: 3442594 |
| | /UG = Hs.289068 Homo sapiens cDNA FLJ11918 fis, clone HEMBB1000272 |
| 218571_s_at | gb: NM_014169.1 /DEF = Homo sapiens HSPC134 protein (HSPC134), mRNA. /FEA = mRNA /GEN = HSPC134 /PROD = HSPC134 protein |
| | /DB_XREF = gi: 7661793 /UG = Hs.279761 HSPC134 protein /FL = gb: AF212243.1 gb: AF161483.1 gb: NM_014169.1 |
| 203462_x_at | gb: NM_003751.1 /DEF = Homo sapiens eukaryotic translation initiation factor 3, subunit 9 (eta, 116 kD) (EIF3S9), mRNA. |
| | /FEA = mRNA /GEN = EIF3S9 /PROD = eukaryotic translation initiation factor 3, subunit 9 (eta, 116 kD) /DB_XREF = gi: 4503526 |
| | /UG = Hs.57783 eukaryotic translation initiation factor 3, subunit 9 (eta, 116 kD) /FL = gb: U62583.1 gb: NM_003751.1 |
| 218642_s_at | gb: NM_024300.1 /DEF = Homo sapiens hypothetical protein MGC2217 (MGC2217), mRNA. /FEA = mRNA /GEN = MGC2217 |
| | /PROD = hypothetical protein MGC2217 /DB_XREF = gi: 13236525 /UG = Hs.323164 hypothetical protein MGC2217 /FL = gb: BC002546.1 |
| | gb: NM_024300.1 |
| 200024_at | gb: NM_001009.1 /DEF = Homo sapiens ribosomal protein S5 (RPS5), mRNA. /FEA = mRNA /GEN = RPS5 /PROD = ribosomal protein S5 |
| | /DB_XREF = gi: 4506728 /UG = Hs.76194 ribosomal protein S5 /FL = gb: NM_001009.1 gb: U14970.1 |
| 218101_s_at | gb: NM_004549.1 /DEF = Homo sapiens NADH dehydrogenase (ubiquinone) 1, subcomplex unknown, 2 (14.5 kD, B14.5b) (NDUFC2), |
| | mRNA. /FEA = mRNA /GEN = NDUFC2 /PROD = NADH dehydrogenase (ubiquinone) 1, subcomplexunknown, 2 (14.5 kD, B14.5b) |
| | /DB_XREF = gi: 4758783 /UG = Hs.193313 NADH dehydrogenase (ubiquinone) 1, subcomplex unknown, 2 (14.5 kD, B14.5b) |
| | /FL = gb: AF087659.1 gb: AF070652.1 gb: NM_004549.1 |
| 209329_x_at | gb: BC000587.1 /DEF = Homo sapiens, clone MGC: 2198, mRNA, complete cds. /FEA = mRNA /PROD = Unknown (protein for MGC: 2198) |
| | /DB_XREF = gi: 12653618 /UG = Hs.227152 mannan-binding lectin serine protease 1 (C4C2 activating component of Ra-reactive |
| | factor) /FL = gb: BC000587.1 |
| 208454_s_at | gb: NM_016134.1 /DEF = Homo sapiens aminopeptidase (LOC51670), mRNA. /FEA = CDS /GEN = LOC51670 /PROD = aminopeptidase |
| | /DB_XREF = gi: 7706386 /UG = Hs.278993 aminopeptidase /FL = gb: AF107834.1 gb: NM_016134.1 |
| 214351_x_at | Consensus includes gb: AA789278 /FEA = EST /DB_XREF = gi: 2849398 /DB_XREF = est: aj28b10.s1 /CLONE = 1391611 /UG = Hs.180842 |
| | ribosomal protein L13 |
| 207075_at | gb: NM_004895.1 /DEF = Homo sapiens chromosome 1 open reading frame 7 (C1ORF7), mRNA. /FEA = mRNA /GEN = C1ORF7 |
| | /PROD = chromosome 1 open reading frame 7 /DB_XREF = gi: 4757727 /UG = Hs.159483 chromosome 1 open reading frame 7 |
| | /FL = gb: AF054176.1 gb: NM_004895.1 |
| 201449_at | Consensus includes gb: AL567227 /FEA = EST /DB_XREF = gi: 12920378 /DB_XREF = est: AL567227 /CLONE = CS0DF027YA11 (3 prime) |
| | /UG = Hs.239489 TIA1 cytotoxic granule-associated RNA-binding protein /FL = gb: NM_022037.1 gb: M77142.1 |
| 213720_s_at | Consensus includes gb: AI831675 /FEA = EST /DB_XREF = gi: 5452346 /DB_XREF = est: wj50g03.x1 /CLONE = IMAGE: 2406292 /UG = Hs.78202 |
| | SWISNF related, matrix associated, actin dependent regulator of chromatin, subfamily a, member 4 |
| 201593_s_at | Consensus includes gb: AV716798 /FEA = EST /DB_XREF = gi: 10813950 /DB_XREF = est: AV716798 /CLONE = DCBAKB02 /UG = Hs.6375 |
| | uncharacterized hypothalamus protein HT010 /FL = gb: AF220184.1 gb: NM_018471.1 |
| 208095_s_at | gb: NM_001222.1 /DEF = Homo sapiens calciumcalmodulin-dependent protein kinase (CaM kinase) II gamma (CAMK2G), mRNA. |
| | /FEA = mRNA /GEN = CAMK2G /PROD = calciumcalmodulin-dependent protein kinase (CaMkinase) II gamma /DB_XREF = gi: 4502554 |
| | /UG = Hs.250857 calciumcalmodulin-dependent protein kinase (CaM kinase) II gamma /FL = gb: U81554.1 gb: NM_001222.1 |
| 201173_x_at | gb: NM_006600.1 /DEF = Homo sapiens nuclear distribution gene C (A. nidulans) homolog (NUDC), mRNA. /FEA = mRNA /GEN = NUDC |
| | /PROD = nuclear distribution gene C (A. nidulans) homolog /DB_XREF = gi: 5729952 /UG = Hs.263812 nuclear distribution gene C |
| | (A. nidulans) homolog /FL = gb: BC002399.1 gb: BC003132.1 gb: AB019408.1 gb: AF130736.1 gb: AF125465.1 gb: AF100760.1 gb: NM_006600.1 |
| 217839_at | gb: NM_006070.1 /DEF = Homo sapiens TRK-fused gene (TFG), mRNA. /FEA = mRNA /GEN = TFG /PROD = TRK-fused gene /DB_XREF = gi: 5174718 |
| | /UG = Hs.250897 TRK-fused gene /FL = gb: NM_006070.1 |
| 203229_s_at | gb: NM_003993.1 /DEF = Homo sapiens CDC-like kinase 2 (CLK2), transcript variant phclk2, mRNA. /FEA = mRNA /GEN = CLK2 |
| | /PROD = CDC-like kinase 2 isoform hclk2 /DB_XREF = gi: 4502882 /UG = Hs.73986 CDC-like kinase 2 /FL = gb: NM_003993.1 gb: L29218.1 |
| 203656_at | gb: NM_014845.1 /DEF = Homo sapiens KIAA0274 gene product (KIAA0274), mRNA. /FEA = mRNA /GEN = KIAA0274 /PROD = KIAA0274 gene |
| | product /DB_XREF = gi: 7662033 /UG = Hs.10037 KIAA0274 gene product /FL = gb: D87464.1 gb: NM_014845.1 |
| 218763_at | gb: NM_016930.1 /DEF = Homo sapiens syntaxin 18 (STX18), mRNA. /FEA = mRNA /GEN = STX18 /PROD = syntaxin 18 /DB_XREF = gi: 8394375 |
| | /UG = Hs.13406 syntaxin 18 /FL = gb: AB028741.1 gb: NM_016930.1 |
| 213846_at | Consensus includes gb: AA382702 /FEA = EST /DB_XREF = gi: 2035020 /DB_XREF = est: EST95939 /UG = Hs.3462 cytochrome c oxidase subunit VIIc |
| 201214_s_at | gb: NM_002712.1 /DEF = Homo sapiens protein phosphatase 1, regulatory subunit 7 (PPP1R7), mRNA. /FEA = mRNA /GEN = PPP1R7 |
| | /PROD = protein phosphatase 1, regulatory subunit 7 /DB_XREF = gi: 4506012 /UG = Hs.36587 protein phosphatase 1, regulatory |
| | subunit 7 /FL = gb: BC000910.1 gb: NM_002712.1 |
| 216505_x_at | Consensus includes gb: AL118502 /DEF = Human DNA sequence from clone RP11-371L19 on chromosome 20 Contains a novel gene, |
| | a gene similar to the gene for ribosomal protein S10, ESTs, STSs, GSSs and CpG islands /FEA = mRNA_3 /DB_XREF = gi: 8894621 |
| | /UG = Hs.284299 Human DNA sequence from clone RP11-371L19 on chromosome 20 Contains a noval gene, a gene similar to the gene |
| | for ribosomal protein S10, ESTs, STSs, GSSs and CpG islands |
| 203459_s_at | gb: NM_022575.1 /DEF = Homo sapiens vacuolar protein sorting 16 (yeast homolog) (VPS16), mRNA. /FEA = mRNA /GEN = VPS16 |
| | /PROD = vacuolar protein sorting 16 (yeast homolog) /DB_XREF = gi: 12007657 /UG = Hs.302441 vacuolar protein sorting 16 |
| | (yeast homolog) /FL = gb: AF308801.1 gb: NM_022575.1 |
| 202880_s_at | gb: NM_004762.1 /DEF = Homo sapiens pleckstrin homology, Sec7 and coiledcoil domains 1 (cytohesin 1) (PSCD1), transcript |
| | variant 1, mRNA. /FEA = mRNA /GEN = PSCD1 /PROD = cytohesin 1, isoform 1 /DB_XREF = gi: 4758963 /UG = Hs.1050 pleckstrin homology, |
| | Sec7 and coiledcoil domains 1 (cytohesin 1) /FL = gb: M85169.1 gb: NM_004762.1 gb: NM_017456.1 |
| 43511_s_at | Cluster Incl. AI201594: qc02h12.x1 Homo sapiens cDNA, 3 end /clone = IMAGE-1708487 /clone_end = 3 /gb = AI201594 |
| | /gi = 3754200 /UG = Hs.239333 /len = 591 |
| 203519_s_at | gb: NM_015542.1 /DEF = Homo sapiens regulator of nonsense transcripts 2; DKFZP434D222 protein (RENT2), mRNA. /FEA = mRNA |
| | /GEN = RENT2 /PROD = regulator of nonsense transcripts 2 /DB_XREF = gi: 11693131 /UG = Hs.3862 regulator of nonsense transcripts 2; |
| | DKFZP434D222 protein /FL = gb: AF301013.1 gb: NM_015542.1 gb: AY013249.1 gb: AF318574.1 |
| 206214_at | gb: NM_005084.1 /DEF = Homo sapiens phospholipase A2, group VII (platelet-activating factor acetylhydrolase, plasma) |
| | (PLA2G7), mRNA. /FEA = mRNA /GEN = PLA2G7 /PROD = phospholipase A2, group VII (platelet-activatingfactor acetylhydrolase, |
| | plasma) /DB_XREF = gi: 4826883 /UG = Hs.93304 phospholipase A2, group VII (platelet-activating factor acetylhydrolase, |
| | plasma) /FL = gb: U24577.1 gb: NM_005084.1 gb: U20157.1 |
| 209933_s_at | gb: AF020314.1 /DEF = Homo sapiens CMRF-35-H9 mRNA, complete cds. /FEA = mRNA /PROD = CMRF-35-H9 /DB_XREF = gi: 4103065 |
| | /UG = Hs.9688 leukocyte membrane antigen /FL = gb: AF020314.1 |
| 202105_at | gb: NM_001551.1 /DEF = Homo sapiens immunoglobulin (CD79A) binding protein 1 (IGBP1), mRNA. /FEA = mRNA /GEN = IGBP1 |
| | /PROD = immunoglobulin-binding protein 1 /DB_XREF = gi: 4557662 /UG = Hs.3631 immunoglobulin (CD79A) binding protein 1 |
| | /FL = gb: BC004137.1 gb: NM_001551.1 |
| 218138_at | gb: NM_018848.1 /DEF = Homo sapiens McKusick-Kaufman syndrome (MKKS), mRNA. /FEA = mRNA /GEN = MKKS /PROD = McKusick-Kaufman |
| | syndrome protein /DB_XREF = gi: 9055271 /UG = Hs.46743 McKusick-Kaufman syndrome /FL = gb: AF275813.1 gb: AF221992.1 |
| | gb: AF221993.1 gb: NM_018848.1 |
| 220864_s_at | gb: NM_015965.1 /DEF = Homo sapiens CGI-39 protein; cell death-regulatory protein GRIM19 (LOC51079), mRNA. /FEA = mRNA |
| | /GEN = LOC51079 /PROD = CGI-39 protein; cell death-regulatory proteinGRIM19 /DB_XREF = gi: 7705733 /UG = Hs.279574 CGI-39 protein; |
| | cell death-regulatory protein GRIM19 /FL = gb: AF132973.1 gb: AF155662.1 gb: NM_015965.1 |
| 213355_at | Consensus includes gb: AI989567 /FEA = EST /DB_XREF = gi: 5836448 /DB_XREF = est: ws34e03.x1 /CLONE = IMAGE: 2499100 /UG = Hs.34578 |
| | alpha2,3-sialyltransferase |
| 212587_s_at | Consensus includes gb: AI809341 /FEA = EST /DB_XREF = gi: 5395907 /DB_XREF = est: we96c09.x1 /CLONE = IMAGE: 2348944 /UG = Hs.170121 |
| | protein tyrosine phosphatase, receptor type, C |
| 205992_s_at | gb: NM_000585.1 /DEF = Homo sapiens interleukin 15 (IL15), mRNA. /FEA = mRNA /GEN = IL15 /PROD = interleukin 15 |
| | /DB_XREF = gi: 10835152 /UG = Hs.168132 interleukin 15 /FL = gb: NM_000585.1 gb: U14407.1 |
| 203262_s_at | gb: NM_004699.1 /DEF = Homo sapiens DNA segment on chromosome X (unique) 9928 expressed sequence (DXS9928E), mRNA. |
| | /FEA = mRNA /GEN = DXS9928E /PROD = XAP-5 protein /DB_XREF = gi: 4758219 /UG = Hs.54277 DNA segment on chromosome X (unique) 9928 |
| | expressed sequence /FL = gb: BC000028.1 gb: D83260.1 gb: AD001530.1 gb: NM_004699.1 |
| 213357_at | Consensus includes gb: AV701318 /FEA = EST /DB_XREF = gi: 10717648 /DB_XREF = est: AV701318 /CLONE = ADAAGD10 |
| | /UG = Hs.278626 ArgAbl-interacting protein ArgBP2 |
| 207628_s_at | gb: NM_017528.1 /DEF = Homo sapiens putative methyltransferase (HASJ4442), mRNA. /FEA = mRNA /GEN = HASJ4442 |
| | /PROD = putative methyltransferase /DB_XREF = gi: 8923713 /UG = Hs.155020 putative methyltransferase /FL = gb: NM_017528.1 |
| 201527_at | gb: NM_004231.1 /DEF = Homo sapiens ATPase, vacuolar, 14 kD (ATP6S14), mRNA. /FEA = mRNA /GEN = ATP6S14 /PROD = ATPase, vacuolar, |
| | 14 kD /DB_XREF = gi: 4757819 /UG = Hs.78089 ATPase, vacuolar, 14 kD /FL = gb: D49400.1 gb: NM_004231.1 |
| 202282_at | gb: NM_004493.1 /DEF = Homo sapiens hydroxyacyl-Coenzyme A dehydrogenase, type II (HADH2), mRNA. /FEA = mRNA /GEN = HADH2 |
| | /PROD = hydroxyacyl-Coenzyme A dehydrogenase, type II /DB_XREF = gi: 4758503 /UG = Hs.171280 hydroxyacyl-Coenzyme A dehydrogenase, |
| | type II /FL = gb: BC000372.1 gb: BC000829.1 gb: U73514.1 gb: U96132.1 gb: AF035555.1 gb: AF069134.1 gb: NM_004493.1 |
| 213735_s_at | Consensus includes gb: AI557312 /FEA = EST /DB_XREF = gi: 4489675 /DB_XREF = est: PT2.1_16_E11.r /UG = Hs.1342 cytochrome c oxidase |
| | subunit Vb |
| 212472_at | Consensus includes gb: BE965029 /FEA = EST /DB_XREF = gi: 11768976 /DB_XREF = est: 601658812R1 /CLONE = IMAGE: 3886131 /UG = Hs.198793 |
| | Homo sapiens cDNA: FLJ22463 fis, clone HRC10126 |
| 214805_at | Consensus includes gb: U79273.1 /DEF = Human clone 23933 mRNA sequence. /FEA = mRNA /DB_XREF = gi: 1710239 /UG = Hs.239483 Human clone |
| | 23933 mRNA sequence |
| 218084_x_at | gb: NM_014164.2 /DEF = Homo sapiens FXYD domain-containing ion transport regulator 5 (FXYD5), mRNA. /FEA = mRNA /GEN = FXYD5 |
| | /PROD = related to ion channel /DB_XREF = gi: 11612664 /UG = Hs.294135 FXYD domain-containing ion transport regulator 5 |
| | /FL = gb: NM_014164.2 gb: AF161462.1 |
| 218204_s_at | gb: NM_024513.1 /DEF = Homo sapiens FYVE and coiled-coil domain containing 1 (FYCO1), mRNA. /FEA = mRNA /GEN = FYCO1 |
| | /PROD = FYVE and coiled-coil domain containing 1 /DB_XREF = gi: 13470091 /UG = Hs.257267 FYVE and coiled-coil domain containing 1 |
| | /FL = gb: NM_024513.1 |
| 200823_x_at | gb: NM_000992.1 /DEF = Homo sapiens ribosomal protein L29 (RPL29), mRNA. /FEA = mRNA /GEN = RPL29 /PROD = ribosomal protein L29 |
| | /DB_XREF = gi: 4506628 /UG = Hs.183698 ribosomal protein L29 /FL = gb: U49083.1 gb: NM_000992.1 gb: U10248.1 |
| 208968_s_at | gb: BC002568.1 /DEF = Homo sapiens, hypothetical protein, clone MGC: 2478, mRNA, complete cds. /FEA = mRNA /PROD = hypothetical |
| | protein /DB_XREF = gi: 12803484 /UG = Hs.4900 hypothetical protein /FL = gb: AF248964.1 gb: BC002568.1 gb: AF116609.1 |
| 208887_at | gb: BC000733.1 /DEF = Homo sapiens, eukaryotic translation initiation factor 3, subunit 4 (delta, 44 kD), clone MGC: 2053, mRNA, |
| | complete cds. /FEA = mRNA /PROD = eukaryotic translation initiation factor 3, subunit 4 (delta, 44 kD) /DB_XREF = gi: 12653882 |
| | /UG = Hs.28081 eukaryotic translation initiation factor 3, subunit 4 (delta, 44 kD) /FL = gb: AF094850.1 gb: BC000733.1 gb: AF020833.1 |
| | gb: U96074.1 gb: NM_003755.1 |
| 214097_at | Consensus includes gb: AW024383 /FEA = EST /DB_XREF = gi: 5877913 /DB_XREF = est: wv03e06.x1 /CLONE = IMAGE: 2528482 /UG = Hs.1948 ribosomal |
| | protein S21 |
| 212191_x_at | Consensus includes gb: AW574664 /FEA = EST /DB_XREF = gi: 7246203 /DB_XREF = est: UI-HF-BL0-abw-d-10-0-UI.s1 /CLONE = IMAGE: 3057859 |
| | /UG = Hs.180842 ribosomal protein L13 |
| 217957_at | gb: NM_013242.1 /DEF = Homo sapiens similar to mouse Glt3 or D. malanogaster transcription factor IIB (AF093680), |
| | mRNA. /FEA = mRNA /GEN = AF093680 /PROD = similar to mouse Glt3 or D. malanogastertranscription factor IIB |
| | /DB_XREF = gi: 8392874 /UG = Hs.279818 similar to mouse Glt3 or D. malanogaster transcription factor IIB |
| | /FL = gb: BC005152.1 gb: AF093680.1 gb: NM_013242.1 |
| 201658_at | Consensus includes gb: AU151560 /FEA = EST /DB_XREF = gi: 11013081 /DB_XREF = est: AU151560 /CLONE = NT2RP2005555 /UG = Hs.242894 |
| | ADP-ribosylation factor-like 1 /FL = gb: NM_001177.2 gb: L28997.1 |
| 218123_at | gb: NM_017835.1 /DEF = Homo sapiens chromosome 21 open reading frame 59 (C21ORF59), mRNA. /FEA = mRNA /GEN = C21ORF59 |
| | /PROD = hypothetical protein FLJ20467 /DB_XREF = gi: 8923436 /UG = Hs.5811 chromosome 21 open reading frame 59 |
| | /FL = gb: NM_021254.1 gb: BC000709.1 gb: NM_017835.1 gb: AF282851.1 |
| 205241_at | gb: NM_005138.1. /DEF = Homo sapiens SCO (cytochrome oxidase deficient, yeast) homolog 2 (SCO2), nuclear gene |
| | encoding mitochondrial protein, mRNA. /FEA = mRNA /GEN = SCO2 /PROD = SCO (cytochrome oxidase deficient, yeast) |
| | homolog 2 /DB_XREF = gi: 4826991 /UG = Hs.278431 SCO (cytochrome oxidase deficient, yeast) homolog 2 /FL = gb: NM_005138.1 |
| 203740_at | gb: NM_005792.1 /DEF = Homo sapiens M-phase phosphoprotein 6 (MPHOSPH6), mRNA. /FEA = mRNA /GEN = MPHOSPH6 |
| | /PROD = M-phase phosphoprotein 6 /DB_XREF = gi: 5031918 /UG = Hs.152720 M-phase phosphoprotein 6 /FL = gb: BC005242.1 gb: NM_005792.1 |
| 221263_s_at | gb: NM_031287.1 /DEF = Homo sapiens hypothetical protein MGC3133 (MGC3133), mRNA. /FEA = mRNA /GEN = MGC3133 |
| | /PROD = hypothetical protein MGC3133 /DB_XREF = gi: 13775199 /FL = gb: NM_031287.1 |
| 218831_s_at | gb: NM_004107.1 /DEF = Homo sapiens Fc fragment of IgG, receptor, transporter, alpha (FCGRT), mRNA. |
| | /FEA = mRNA /GEN = FCGRT /PROD = Fc fragment of IgG, receptor, transporter, alpha /DB_XREF = gi: 4758345 |
| | /UG = Hs.111903 Fc fragment of IgG, receptor, transporter, alpha /FL = gb: NM_004107.1 gb: U12255.1 |
| 201400_at | gb: NM_002795.1 /DEF = Homo sapiens proteasome (prosome, macropain) subunit, beta type, 3 (PSMB3), mRNA. |
| | /FEA = mRNA /GEN = PSMB3 /PROD = proteasome (prosome, macropain) subunit, betatype, 3 /DB_XREF = gi: 4506196 |
| | /UG = Hs.82793 proteasome (prosome, macropain) subunit, beta type, 3 /FL = gb: NM_002795.1 gb: D26598.1 |
| 203136_at | gb: NM_006423.1 /DEF = Homo sapiens Rab acceptor 1 (prenylated) (RABAC1), mRNA. /FEA = mRNA /GEN = RABAC1 /PROD = Rab acceptor 1 |
| | (prenylated) /DB_XREF = gi: 5453959 /UG = Hs.11417 Rab acceptor 1 (prenylated) /FL = gb: NM_006423.1 gb: AF112202.1 |
| 205382_s_at | gb: NM_001928.1 /DEF = Homo sapiens D component of complement (adipsin) (DF), mRNA. /FEA = mRNA /GEN = DF |
| | /PROD = adipsincomplement factor D precursor /DB_XREF = gi: 4503308 /UG = Hs.155597 D component of complement (adipsin) |
| | /FL = gb: M84526.1 gb: NM_001928.1 |
| 208714_at | gb: AF092131.1 /DEF = Homo sapiens 51 kDa subunit of NADH dehydrogenase mRNA, complete cds. /FEA = mRNA |
| | /PROD = 51 kDa subunit of NADH dehydrogenase /DB_XREF = gi: 5138911 /UG = Hs.7744 NADH dehydrogenase (ubiquinone) |
| | flavoprotein 1 (51 kD) /FL = gb: AF053070.1 gb: AF092131.1 gb: NM_007103.1 |
| 209224_s_at | gb: BC003674.1 /DEF = Homo sapiens, NADH dehydrogenase (ubiquinone) 1 alpha subcomplex, 2 (8 kD, B8), |
| | clone MGC: 12315, mRNA, complete cds. /FEA = mRNA /PROD = NADH dehydrogenase (ubiquinone) 1 alphasubcomplex, 2 (8 kD, B8) |
| | /DB_XREF = gi: 13277539 /UG = Hs.163867 NADH dehydrogenase (ubiquinone) 1 alpha subcomplex, 2 (8 kD, B8) |
| | /FL = gb: BC003674.1 gb: AF047185.1 gb: NM_002488.1 gb: AF077029.1 |
| 210574_s_at | gb: AF241788.1 /DEF = Homo sapiens NPD011 (NPD011) mRNA, complete cds. /FEA = mRNA /GEN = NPD011 /PROD = NPD011 |
| | /DB_XREF = gi: 12005492 /UG = Hs.263812 nuclear distribution gene C (A. nidulans) homolog /FL = gb: AF241788.1 |
| 214259_s_at | Consensus includes gb: AI144075 /FEA = EST /DB_XREF = gi: 3665884 /DB_XREF = est: qi63e12.x1 /CLONE = IMAGE: 1861198 |
| | /UG = Hs.6980 aldo-keto reductase family 7, member A2 (aflatoxin aldehyde reductase) |
| 209234_at | Consensus includes gb: BF939474 /FEA = EST /DB_XREF = gi: 12356794 /DB_XREF = est: nac76d12.x1 /CLONE = IMAGE: 3440303 |
| | /UG = Hs.129908 KIAA0591 protein /FL = gb: BC001415.1 |
| 203654_s_at | gb: NM_004645.1 /DEF = Homo sapiens coilin (COIL), mRNA. /FEA = mRNA /GEN = COIL /PROD = coilin /DB_XREF = gi: 4758023 /UG = Hs.966 coilin |
| | /FL = gb: U06632.1 gb: NM_004645.1 |
| 201129_at | gb: NM_006276.2 /DEF = Homo sapiens splicing factor, arginineserine-rich 7 (35 kD) (SFRS7), mRNA. /FEA = mRNA /GEN = SFRS7 |
| | /PROD = splicing factor, arginineserine-rich 7 (35 kD) /DB_XREF = gi: 6857827 /UG = Hs.184167 splicing factor, |
| | arginineserine-rich 7 (35 kD) /FL = gb: BC000997.2 gb: L22253.1 gb: NM_006276.2 |
| 202451_at | gb: BC000365.1 /DEF = Homo sapiens, general transcription factor IIH, polypeptide 1 (62 kD subunit), clone MGC: 8323, mRNA, |
| | complete cds. /FEA = mRNA /PROD = general transcription factor IIH, polypeptide 1(62 kD subunit) /DB_XREF = gi: 12653194 |
| | /UG = Hs.89578 general transcription |
| | factor IIH, polypeptide 1 (62 kD subunit) /FL = gb: BC000365.1 gb: BC004452.1 gb: M95809.1 gb: NM_005316.1 |
| 218072_at | gb: NM_014186.1 /DEF = Homo sapiens HSPC166 protein (HSPC166), mRNA. /FEA = mRNA /GEN = HSPC166 /PROD = HSPC166 protein |
| | /DB_XREF = gi: 7661827 /UG = Hs.279836 HSPC166 protein /FL = gb: AL136688.1 gb: AF161515.1 gb: NM_014186.1 |
| 203063_at | gb: NM_014634.1 /DEF = Homo sapiens KIAA0015 gene product (KIAA0015), mRNA. /FEA = mRNA /GEN = KIAA0015 |
| | /PROD = KIAA0015 gene product /DB_XREF = gi: 7661861 /UG = Hs.278441 KIAA0015 gene product /FL = gb: D13640.1 gb: NM_014634.1 |
| 204037_at | Consensus includes gb: BF055366 /FEA = EST /DB_XREF = gi: 10809262 /DB_XREF = est: 7j78f10.x1 /CLONE = IMAGE: 3392587 |
| | /UG = Hs.75794 endothelial differentiation, lysophosphatidic acid G-protein-coupled receptor, 2 |
| | /FL = gb: U78192.1 gb: U80811.1 gb: NM_001401.1 |
| 218298_s_at | gb: NM_024952.1 /DEF = Homo sapiens hypothetical protein FLJ20950 (FLJ20950), mRNA. /FEA = mRNA /GEN = FLJ20950 |
| | /PROD = hypothetical protein FLJ20950 /DB_XREF = gi: 13376436 /UG = Hs.285673 hypothetical protein FLJ20950 /FL = gb: NM_024952.1 |
| 204158_s_at | gb: NM_006019.1 /DEF = Homo sapiens T-cell, immune regulator 1 (TCIRG1), mRNA. /FEA = mRNA /GEN = TCIRG1 /PROD = ATPase, H+ transporting, |
| | 116 kD /DB_XREF = gi: 5174620 /UG = Hs.46465 T-cell, immune regulator 1 /FL = gb: U45285.1 gb: NM_006019.1 |
| 208842_s_at | Consensus includes gb: W93787 /FEA = EST /DB_XREF = gi: 1422930 /DB_XREF = est: zd96c09.s1 /CLONE = IMAGE: 357328 /UG = Hs.6880 DKF2P434D156 |
| | protein /FL = gb: BC001408.1 |
| 203047_at | gb: NM_005990.1 /DEF = Homo sapiens serinethreonine kinase 10 (STK10), mRNA. /FEA = mRNA /GEN = STK10 /PROD = serinethreonine kinase 10 |
| | /DB_XREF = gi: 5174700 /UG = Hs.16134 serinethreonine kinase 10 /FL = gb: AB015718.1 gb: NM_005990.1 gb: AF119894.1 |
| 213408_s_at | Consensus includes gb: AK024034.1 /DEF = Homo sapiens cDNA FLJ13972 fis, clone Y79AA1001548, |
| | highly similar to PHOSPHATIDYLINOSITOL 4-KINASE ALPHA (EC 2.7.1.67). /FEA = mRNA /DB_XREF = gi: 10436297 |
| | /UG = Hs.171625 phosphatidylinositol 4-kinase, catalytic, alpha polypeptide |
| 209858_x_at | gb: BC002877.1 /DEF = Homo sapiens, Similar to hypothetical protein FLJ11585, clone MGC: 11258, mRNA, complete cds. /FEA = mRNA |
| | /PROD = Similar to hypothetical protein FLJ11585 /DB_XREF = gi: 12804048 /UG = Hs.315367 Homo sapiens, Similar to hypothetical protein |
| | FLJ11585, clone MGC: 11258, mRNA, complete cds /FL = gb: BC002877.1 |
| 35626_at | Cluster Incl. U30894: Human N-sulphoglucosamine sulphohydrolase mRNA, complete cds /cds = (12,1520) /gb = U30894 /gi = 1173542 |
| | /ug = Hs.31074 /len = 2657 |
| 201871_s_at | gb: NM_015853.1 /DEF = Homo sapiens ORF (LOC51035), mRNA. /FEA = mRNA /GEN = LOC51035 /PROD = unknown protein LOC51035 |
| | /DB_XREF = gi: 7705653 /UG = Hs.77868 ORF /FL = gb: BC000902.1. gb: M68864.1 gb: NM_015853.1 |
| 212204_at | Consensus includes gb: AL049944.1 /DEF = Homo sapiens mRNA; cDNA DKFZp564G2022 (from clone DKFZp564G2022); partial cds. |
| | /FEA = mRNA /GEN = DKFZp564G2022 /PROD = hypothetical protein /DB_XREF = gi: 4884189 /UG = Hs.16492 DKFZP564G2022 protein |
| 217802_s_at | gb: NM_022731.1 /DEF = Homo sapiens similar to rat nuclear ubiquitous casein kinase 2 (NUCKS), mRNA. /FEA = mRNA /GEN = NUCKS |
| | /PROD = similar to rat nuclear ubiquitous casein kinase2 /DB_XREF = gi: 12232386 |
| | /UG = Hs.118064 similar to rat nuclear ubiquitous casein kinase 2 /FL = gb: NM_022731.1 gb: BC000805.1 |
| 201960_s_at | gb: NM_015057.1 /DEF = Homo sapiens KIAA0916 protein (KIAA0916), mRNA. /FEA = mRNA /GEN = KIAA0916 /PROD = KIAA0916 protein |
| | /DB_XREF = gi: 7662379 /UG = Hs.151411 KIAA0916 protein /FL = gb: AF075587.1 gb: AF083244.1 gb: NM_015057.1 |
| 47608_at | Cluster Incl. AI697401: tq18h09.x1 Homo sapiens cDNA, 3 end /clone = IMAGE-2209217 /clone_end = 3 /gb = AI697401 /gi = 4985301 |
| | /ug = Hs.9741 /len = 639 |
| 210250_x_at | gb: AF067854.1 /DEF = Homo sapiens adenylosuccinate lyase (ADSL) mRNA, alternatively spliced, complete cds. |
| | /FEA = mRNA /GEN = ADSL /PROD = adenylosuccinate lyase /DB_XREF = gi: 3211983 /UG = Hs.75527 adenylosuccinate lyase /FL = gb: AF067854.1 |
| 218495_at | gb: NM_004182.1 /DEF = Homo sapiens ubiquitously-expressed transcript (UXT), mRNA. /FEA = mRNA /GEN = UXT |
| | /PROD = ubiquitously-expressed transcript /DB_XREF = gi: 4759297 /UG = Hs.172791 ubiquitously-expressed transcript |
| | /FL = gb: BC000720.1 gb: AF092737.1 gb: NM_004182.1 gb: AF083241.1 gb: AF083242.1 |
| 201106_at | gb: NM_002085.1 /DEF = Homo sapiens glutathione peroxidase 4 (phospholipid hydroperoxidase) (GPX4), mRNA. /FEA = mRNA /GEN = GPX4 |
| | /PROD = glutathione peroxidase 4 /DB_XREF = gi: 4504106 /UG = Hs.2706 glutathione peroxidase 4 (phospholipid hydroperoxidase) |
| | /FL = gb: NM_002085.1 |
| 203569_s_at | gb: NM_003611.1 /DEF = Homo sapiens chromosome X open reading frame 5 (CXORF5), mRNA. /FEA = mRNA /GEN = CXORF5 |
| | /PROD = chromosome X open reading frame 5 /DB_XREF = gi: 4503178 /UG = Hs.6483 oral-facial-digital syndrome 1 gene /FL = gb: NM_003611.1 |
| 201316_at | Consensus includes gb: AL523904 /FEA = EST /DB_XREF = gi: 12787397 /DB_XREF = est: AL523904 /CLONE = CS0DC003YB07 (3 prime) |
| | /UG = Hs.181309 proteasome (prosome, macropain) subunit, alpha type, 2 /FL = gb: NM_002787.1 |
| 218336_at | gb: NM_012394.2 /DEF = Homo sapiens prefoldin 2 (PFDN2), mRNA. /FEA = mRNA /GEN = PFDN2 /PROD = prefoldin 2 /DB_XREF = gi: 12408674 |
| | /UG = Hs.298229 prefoldin 2 /FL = gb: NM_012394.2 gb: AF165883.1 gb: AF117237.1 gb: AF151065.1 |
| 209370_s_at | Consensus includes gb: BE502377 /FEA = EST /DB_XREF = gi: 9704785 /DB_XREF = est: hy22g03.x1 /CLONE = IMAGE: 3198100 /UG = Hs.167679 |
| | SH3-domain binding protein 2 /FL = gb: AB000462.1 gb: AF000936.1 |
| 201036_s_at | gb: NM_005327.1 /DEF = Homo sapiens L-3-hydroxyacyl-Coenzyme A dehydrogenase, short chain (HADHSC), mRNA. /FEA = mRNA /GEN = HADHSC |
| | /PROD = L-3-hydroxyacyl-Coenzyme A dehydrogenase, shortchain /DB_XREF = gi: 4885386 /UG = Hs.8110 L-3-hydroxyacyl-Coenzyme A dehydrogenase, |
| | short chain /FL = gb: BC000306.1 gb: NM_005327.1 |
| 208776_at | Consensus includes gb: BF432873 /FEA = EST /DB_XREF = gi: 11445036 /DB_XREF = est: 7n28a02.x1 /CLONE = IMAGE: 3565730 /UG = Hs.90744 proteasome |
| | (prosome, macropain) 26S subunit, non-ATPase, 11 /FL = gb: BC000437.1 gb: BC004430.1 gb: AB003102.1 gb: AF001212.1 gb: NM_002815.1 |
| 214765_s_at | Consensus includes gb: AK024677.1 /DEF = Homo sapiens cDNA: FLJ21024 fis, clone CAE05651, highly similar to HUMPLT Human LTR mRNA. |
| | /FEA = mRNA /DB_XREF = gi: 10437016 /UG = Hs.264330 N-acylsphingosine amidohydrolase (acid ceramidase)-like |
| 204225_at | gb: NM_006037.2 /DEF = Homo sapiens histone deacetylase 4 (HDAC4), mRNA. /FEA = mRNA /GEN = HDAC4 /PROD = histone deacetylase 4 |
| | /DB_XREF = gi: 13259519 /UG = Hs.91400 histone deacetylase 4 /FL = gb: NM_006037.2 gb: AF132607.1 |
| 214170_x_at | Consensus includes gb: AA669797 /FEA = EST /DB_XREF = gi: 2631296 /DB_XREF = est: ag36c01.s1 /CLONE = IMAGE: 1118880 /UG = Hs.75653 |
| | fumarate hydratase |
| 218210_at | gb: NM_024619.1 /DEF = Homo sapiens hypothetical protein FLJ12171 (FLJ12171), mRNA. /FEA = mRNA /GEN = FLJ12171 /PROD = hypothetical |
| | protein FLJ12171 /DB_XREF = gi: 13375839 /UG = Hs.31431 hypothetical protein FLJ12171 /FL = gb: AL136631.1 gb: NM_024619.1 |
| 218652_s_at | gb: NM_017733.1 /DEF = Homo sapiens hypothetical protein FLJ20265 (FLJ20265), mRNA. /FEA = mRNA /GEN = FLJ20265 /PROD = hypothetical |
| | protein FLJ20265 /DB_XREF = gi: 8923239 /UG = Hs.7099 hypothetical protein FLJ20265 /FL = gb: BC001249.1 gb: BC000937.2 gb: NM_017733.1 |
| 201270_x_at | gb: NM_015332.1 /DEF = Homo sapiens KIAA1068 protein (KIAA1068), mRNA. /FEA = mRNA /GEN = KIAA1068 /PROD = KIAA1068 protein |
| | /DB_XREF = gi: 13357209 /UG = Hs.4770 KIAA1068 protein /FL = gb: BC003691.1 gb: NM_015332.1 |
| 203906_at | Consensus includes gb: AI652645 /FEA = EST /DB_XREF = gi: 4736624 /DB_XREF = est: wb30b07.x1 /CLONE = IMAGE: 2307157 /UG = Hs.4764 KIAA0763 gene |
| | product /FL = gb: AB018306.1 gb: NM_014869.1 |
| 221516_s_at | gb: BC002587.1 /DEF = Homo sapiens, hypothetical protein, clone MGC: 1067, mRNA, complete cds. /FEA = mRNA /PROD = hypothetical protein |
| | /DB_XREF = gi: 12803520 /UG = Hs.83869 hypothetical protein /FL = gb: BC002587.1 |
| 209492_x_at | gb: BC003679.1 /DEF = Homo sapiens, ATP synthase, H+ transporting, mitochondrial F0 complex, subunit e, clone MGC: 12532, mRNA, |
| | complete cds. /FEA = mRNA /PROD = ATP synthase, H+ transporting, mitochondrial F0complex, subunit e /DB_XREF = gi: 13277543 |
| | /UG = Hs.85539 ATP synthase, H+ transporting, mitochondrial F0 complex, subunit e /FL = gb: BC003679.1 |
| 212048_s_at | Consensus includes gb: AW245400 /FEA = EST /DB_XREF = gi: 6588393 /DB_XREF = est: 2822751.3prime /CLONE = IMAGE: 2822751 /UG = Hs.239307 |
| | tyrosyl-tRNA synthatase |
| 204214_s_at | gb: NM_006834.1 /DEF = Homo sapiens RAB32, member RAS oncogene family (RAB32), mRNA. /FEA = mRNA /GEN = RAB32 /PROD = RAB32, member RAS |
| | oncogene family /DB_XREF = gi: 5803132 /UG = Hs.32217 RAB32, member RAS oncogene family /FL = gb: U71127.1 gb: NM_006834.1 |
| 217796_s_at | gb: NM_017921.1 /DEF = Homo sapiens hypothetical protein FLJ20657 (FLJ20657), mRNA. /FEA = mRNA /GEN = FLJ20657 /PROD = hypothetical |
| | protein FLJ20657 /DB_XREF = gi: 8923608 /UG = Hs.164256 hypothetical protein FLJ20657 /FL = gb: NM_017921.1 |
| 203327_at | Consensus includes gb: N22903 /FEA = EST /DB_XREF = gi: 1137053 /DB_XREF = est: yx66e04.s1 /CLONE = IMAGE: 266718 /UG = Hs.1508 insulin-degrading |
| | enzyme /FL = gb: M21186.1 gb: NM_004969.1 |
| 208659_at | gb: AF034607.1 /DEF = Homo sapiens chloride channel ABP mRNA, complete cds. /FEA = mRNA /PROD = chloride channel ABP /DB_XREF = gi: 4426566 |
| | /UG = Hs.74276 chloride intracellular channel 1 /FL = gb: U93205.1 gb: AF034607.1 gb: AF109197.1 gb: NM_001288.2 |
| 222218_s_at | Consensus includes gb: AJ400843.1 /DEF = Homo sapiens partial mRNA for immunoglobulin-like cell surface receptor FDF03-M14, soluble |
| | alternative form. /FEA = mRNA /GEN = FDF03-M14 /PROD = cell surface receptor FDF03-M14 /DB_XREF = gi: 9715838 /UG = Hs.122591 paired |
| | immunoglobulin-like receptor alpha |
| 211862_x_at | gb: AF015451.1 /DEF = Homo sapiens Usurpin-beta mRNA, complete cds. /FEA = CDS /PROD = Usurpin-beta /DB_XREF = gi: 3133282 /UG = Hs.195175 CASP8 |
| | and FADD-like apoptosis regulator /FL = gb: AF015451.1 |
| 209080_x_at | gb: AF118652.1 /DEF = Homo sapiens PKCq-interacting protein PICOT (PICOT) mRNA, complete cds. /FEA = mRNA /GEN = PICOT /PROD = PKCq- |
| | interacting protein PICOT /DB_XREF = gi: 6840952 /UG = Hs.42644 thioredoxin-like /FL = gb: BC005289.1 gb: AF118649.1 gb: AF118652.1 |
| 200789_at | gb: NM_001398.1 /DEF = Homo sapiens enoyl Coenzyme A hydratase 1, peroxisomal (ECH1), mRNA. /FEA = mRNA /GEN = ECH1 /PROD = peroxisomal |
| | enoyl-coenzyme A hydratase-likeprotein /DB_XREF = gi: 4503446 /UG = Hs.196176 enoyl Coenzyme A hydratase 1, peroxisomal |
| | /FL = gb: NM_001398.1 gb: U16660.1 |
| 90610_at | Cluster Incl. AI654857: wb65b10.x1 Homo sapiens cDNA, 3 end /clone = IMAGE-2310523 /clone_end = 3 /gb = AI654857 /gi = 4738836 |
| | /ug = Hs.160115 /len = 621 |
| 201704_at | gb: NM_001247.1 /DEF = Homo sapiens ectonucleoside triphosphate diphosphohydrolase 6 (putative function) (ENTPD6), mRNA. |
| | /FEA = mRNA /GEN = ENTPD6 /PROD = ectonucleoside triphosphate diphosphohydrolase 6(putative function) /DB_XREF = gi: 4557422 |
| | /UG = Hs.12330 ectonucleoside triphosphate diphosphohydrolase 6 (putative function) /FL = gb: AF039916.1 gb: NM_001247.1 |
| 218026_at | gb: NM_014019.1 /DEF = Homo sapiens HSPC009 protein (HSPC009), mRNA. /FEA = mRNA /GEN = HSPC009 /PROD = HSPC009 protein |
| | /DB_XREF = gi: 7661731 /UG = Hs.16059 HSPC009 protein /FL = gb: BC002698.1 gb: AF070665.1 gb: NM_014019.1 |
| 214771_x_at | Consensus includes gb: AK025604.1 /DEF = Homo sapiens cDNA: FLJ21951 fis, clone HEP04968. /FEA = mRNA /DB_XREF = gi: 10438172 |
| | /UG = Hs.84883 KIAA0864 protein |
| 208760_at | Consensus includes gb: AL031714 /DEF = Human DNA sequence from clone LA16-358B7 on chromosome 16 Contains the UBE21 gene for |
| | ubiquitin-conjugating enzyme E2I (homologous to yeast UBC9), and an RPS20 (40S Ribosomal protein S20) pseudogene. Contains ESTs, |
| | STSs. GSSs and a putative CpG is . . . /FEA = mRNA /DB_XREF = gi: 4775608 /UG = Hs.84285 ubiquitin-conjugating enzyme E2I (homologous to |
| | yeast UBC9) /FL = gb: U45328.1 gb: U31933.1 gb: BC000427.1 gb: BC004429.1 gb: U31882.1 gb: U66818.1 gb: U66867.1 gb: U38785.1 gb: NM_003345.1 |
| | gb: U29092.1 |
| 212607_at | Consensus includes gb: N32526 /FEA = EST /DB_XREF = gi: 1152925 /DB_XREF = est: yy11f04.s1 /CLONE = IMAGE: 270943 /UG = Hs.300642 serologically |
| | defined colon cancer antigen 8 |
| 64418_at | Cluster Incl. AI472320: tj87c02.x1 Homo sapiens cDNA, 3 end /clone = IMAGE-2148482 /clone_end = 3 /gb = AI472320 /gi = 4334410 |
| | /ug = Hs.48504 /len = 548 |
| 205819_at | gb: NM_006770.1 /DEF = Homo sapiens macrophage receptor with collagenous structure (MARCO), mRNA. /FEA = mRNA /GEN = MARCO |
| | /PROD = macrophage receptor with collagenous structure /DB_XREF = gi: 5803079 /UG = Hs.67726 macrophage receptor with collagenous |
| | structure /FL = gb: AF035819.1 gb: NM_006770.1 |
| 218019_s_at | gb: NM_021941.1 /DEF = Homo sapiens hypothetical protein FLJ21324 (FLJ21324), mRNA. /FEA = mRNA /GEN = FLJ21324 /PROD = hypothetical |
| | protein FLJ21324 /DB_XREF = gi: 11345479 /UG = Hs.4746 hypothetical protein FLJ21324 /FL = gb: NM_021941.1 gb: BC003651.1 |
| 219220_x_at | gb: NM_020191.1 /DEF = Homo sapiens GK002 protein (GK002), mRNA. /FEA = mRNA /GEN = GK002 /PROD = GK002 protein /DB_XREF = gi: 9910243 |
| | /UG = Hs.107127 GK002 protein; gibt protein; chromosome 3 open reading frame 5 /FL = gb: AF321613.1 gb: AF226045.1 gb: NM_020191.1 |
| 212355_at | Consensus includes gb: AI075450 /FEA = EST /DB_XREF = gi: 3399805 /DB_XREF = est: oz82g10.x1 /CLONE = IMAGE: 1681890 |
| | /UG = Hs.7911 KIAA0323 protein |
| 214735_at | Consensus includes gb: AW166711 /FEA = EST /DB_XREF = gi: 6398236 /DB_XREF = est: xg27h02.x1 /CLONE = IMAGE: 2628819 |
| | /UG = Hs.185140 KIAA0403 protein |
| 219041_s_at | gb: NM_014374.1 /DEF = Homo sapiens zinc finger protein (AP4), mRNA. /FEA = mRNA /GEN = AP4 /PROD = zinc finger protein |
| | /DB_XREF = gi: 7656889 /UG = Hs.90693 zinc finger protein /FL = gb: BC000363.1 gb: NM_014374.1 |
| 209110_s_at | gb: AL050259.1 /DEF = Homo sapiens mRNA; cDNA DKFZp564D0782 (from clone DKFZp564D0782); complete cds. /FEA = mRNA /GEN = DKFZp564D0782 |
| | /PROD = hypothetical protein /DB_XREF = gi: 4886476 /UG = Hs.170160 RAB2, member RAS oncogene family-like /FL = gb: D85757.1 gb: NM_004761.1 |
| | gb: AL050259.1 |
| 215691_x_at | Consensus includes gb: AV702994 /FEA = EST /DB_XREF = gi: 10719324 /DB_XREF = est: AV702994 /CLONE = ADBAPB06 /UG = Hs.46967 HSPCO34 protein |
| 207614_s_at | gb: NM_003592.1 /DEF = Homo sapiens cullin 1 (CUL1), mRNA. /FEA = mRNA /GEN = CUL1 /PROD = cullin 1 /DB_XREF = gi: 4503160 |
| | /UG = Hs.14541 cullin 1 /FL = gb: U58087.1 gb: NM_003592.1 |
| 213405_at | Consensus includes gb: N95443 /FEA = EST /DB_XREF = gi: 1267753 /DB_XREF = est: zb81c12.s1 /CLONE = IMAGE: 310006 /UG = Hs.19180 Homo sapiens |
| | mRNA; cDNA DKFZp564E122 (from clone DKFZp564E122) |
| 208835_s_at | Consensus includes gb: AW089673 /FEA = EST /DB_XREF = gi: 6047017 /DB_XREF = est: xd21h11.x1 /CLONE = IMAGE: 2594469 /UG = Hs.3688 cisplatin |
| | resistance-associated overexpressed protein /FL = gb: AB034205.1 |
| 202225_at | Consensus includes gb: AW612311 /FEA = EST /DB_XREF = gi: 7317497 /DB_XREF = est: hg95e07.x1 /CLONE = IMAGE: 2953380 /UG = Hs.306088 v-crk avian |
| | sarcoma virus CT10 oncogene homolog /FL = gb: D10656.1 gb: NM_016823.1 |
| 218358_at | gb: NM_024324.1 /DEF = Homo sapiens hypothetical protein MGC11256 (MGC11256), mRNA. /FEA = mRNA /GEN = MGC11256 /PROD = hypothetical protein |
| | MGC11256 /DB_XREF = gi: 13236568 /UG = Hs.28029 hypothetical protein MGC11256 /FL = gb: BC002894.1 gb: NM_024324.1 |
| 213511_s_at | Consensus includes gb: AI167164 /FEA = EST /DB_XREF = gi: 3700334 /DB_XREF = est: oo08c10.x1 /CLONE = IMAGE: 1565586 /UG = Hs.23200 myotubularin |
| | related protein 1 |
| 218133_s_at | gb: NM_021824.1 /DEF = Homo sapiens NIF3 (Ngg1 interacting factor 3, S. pombe homolog)-like 1 (NIF3L1), mRNA. /FEA = mRNA /GEN = NIF3L1 |
| | /PROD = NIF3 (Ngg1 interacting factor 3, S. pombe homolog)-like 1 /DB_XREF = gi: 11141898 /UG = Hs.21943 NIF3 (Ngg1 interacting factor 3, |
| | S. pombe homolog)-like 1 /FL = gb: AF182416.1 gb: NM_021824.1 gb: AF060513.1 gb: AB038949.1 |
| 212007_at | Consensus includes gb: AI927512 /FEA = EST /DB_XREF = gi: 5663476 /DB_XREF = est: wo90g11.x1 /CLONE = IMAGE: 2462660 /UG = Hs.77495 UBX |
| | domain-containing 1 |
| 204690_at | gb: NM_004853.1 /DEF = Homo sapiens syntaxin 8 (STX8), mRNA. /FEA = mRNA /GEN = STX 8 /PROD = syntaxin 8 /DB_XREF = gi: 4759187 /UG = Hs.119525 |
| | syntaxin 8 /FL = gb: AF062077.1 gb: AF036715.1 gb: AF115323.1 gb: NM_004853.1 |
| 210042_s_at | gb: AF073890.1 /DEF = Homo sapiens cathepsin X precursor, mRNA, complete cds. /FEA = mRNA /PROD = cathepsin X precursor |
| | /DB_XREF = gi: 3650497 /UG = Hs.252549 cathepsin Z /FL = gb: AF032906.1 gb: AF073890.1 gb: NM_001336.1 gb: AF136273.1 |
| 32091_at | Cluster Incl. AB007915: Homo sapiens mRNA for KIAA0446 protein, complete cds /cds = (3531, 4286) /gb = AB007915 /gi = 3413853 |
| | /ug = Hs.158286 /len = 6944 |
| 201135_at | gb: NM_004092.2 /DEF = Homo sapiens enoyl Coenzyme A hydratase, short chain, 1, mitochondrial (ECHS1), nuclear gene encoding |
| | mitochondrial protein, mRNA. /FEA = mRNA /GEN = ECHS1 /PROD = mitochondrial short-chain enoyl-coenzyme Ahydratase 1 precursor |
| | /DB_XREF = gi: 12707569 /UG = Hs.76394 enoyl Coenzyme A hydratase, short chain, 1, mitochondrial /FL = gb: NM_004092.2 gb: D13900.1 |
| 220526_s_at | gb: NM_017971.1 /DEF = Homo sapiens hypothetical protein FLJ10024 (FLJ10024), mRNA. /FEA = mRNA /GEN = FLJ10024 /PROD = hypothetical protein |
| | FLJ10024 /DB_XREF = gi: 8922189 /UG = Hs.182698 hypothetical protein FLJ10024 /FL = gb: NM_017971.1 |
| 202531_at | gb: NM_002198.1 /DEF = Homo sapiens interferon regulatory factor 1 (IRF1), mRNA. /FEA = mRNA /GEN = IRF1 /PROD = interferon regulatory |
| | factor 1 /DB_XREF = gi: 4504720 /UG = Hs.80645 interferon regulatory factor 1 /FL = gb: NM_002198.1 |
| 213465_s_at | Consensus includes gb: BF718769 /FEA = EST /DB_XREF = gi: 12019514 /DB_XREF = est: KEST72 /CLONE = S90413.NIH-107-R.ab1 /UG = Hs.36587 |
| | protein phosphatase 1, regulatory subunit 7 |
| 202025_x_at | gb: NM_001607.2 /DEF = Homo sapiens acetyl-Coenzyme A acyltransferase 1 (peroxisomal 3-oxoacyl-Coenzyme A thiolase) (ACAA1), |
| | nuclear gene encoding mitochondrial protein, mRNA. /FEA = mRNA /GEN = ACAA1 /PROD = acetyl-Coenzyme A acyltransferase 1 |
| | /DB_XREF = gi: 6598316 /UG = Hs.166160 acetyl-Coenzyme A acyltransferase 1 (peroxisomal 3-oxoacyl-Coenzyme A thiolase) |
| | /FL = gb: BC000635.1 gb: NM_001607.2 |
| 203944_x_at | gb: NM_007049.1 /DEF = Homo sapiens butyrophilin, subfamily 2, member A1 (BTN2A1), mRNA. /FEA = mRNA /GEN = BTN2A1 /PROD = butyrophilin, |
| | subfamily 2, member A1 /DB_XREF = gi: 5921460 /UG = Hs.169963 butyrophilin, subfamily 2, member A1 /FL = gb: U90543.1 gb: NM_007049.1 |
| 214290_s_at | Consensus includes gb: AI313324 /FEA = EST /DB_XREF = gi: 4018929 /DB_XREF = est: ta77f02.x2 /CLONE = IMAGE: 2050107 /UG = Hs.795 H2A histone |
| | family, member O |
| 208415_x_at | gb: NM_005537.1 /DEF = Homo sapiens inhibitor of growth 1 family, member 1 (ING1), mRNA. /FEA = mRNA /GEN = ING1 /PROD = inhibitor of |
| | growth 1 family, member 1 /DB_XREF = gi: 5031792 /UG = Hs.46700 inhibitor of growth 1 family, member 1 /FL = gb: AF001954.1 gb: NM_005537.1 |
| 216396_s_at | Consensus includes gb: AF131850.1 /DEF = Homo sapiens clone 24988 mRNA sequence. /FEA = mRNA /DB_XREF = gi: 4406694 /UG = Hs.286027 |
| | etoposide-induced mRNA |
| 203033_x_at | gb: NM_000143.1 /DEF = Homo sapiens fumarate hydratase (FH), mRNA. /FEA = mRNA /GEN = FH /PROD = fumarate hydratase /DB_XREF = gi: 4503716 |
| | /UG = Hs.75653 fumarate hydratase /FL = gb: BC003108.1 gb: M15502.1 gb: U59309.1 gb: U48857.1 gb: NM_000143.1 |
| 203971_at | gb: NM_001859.1 /DEF = Homo sapiens solute carrier family 31 (copper transporters), member 1 (SLC31A1), mRNA. /FEA = mRNA |
| | /GEN = SLC31A1 /PROD = solute carrier family 31 (copper transporters), member 1 /DB_XREF = gi: 4507014 /UG = Hs.73614 solute carrier family |
| | 31 (copper transporters), member 1 /FL = gb: U83460.1 gb: NM_001859.1 |
| 203241_at | gb: NM_003369.1 /DEF = Homo sapiens UV radiation resistance associated gene (UVRAG), mRNA. /FEA = mRNA /GEN = UVRAG /PROD = UV radiation |
| | resistance associated gene /DB_XREF = gi: 4507860 /UG = Hs.13137 UV radiation resistance associated gene /FL = gb: NM_003369.1 gb: AB012958.1 |
| 201142_at | Consensus includes gb: AA577698 /FEA = EST /DB_XREF = gi: 2355882 /DB_XREF = est: nn22h05.s1 /CLONE = IMAGE: 1084665 /UG = Hs.151777 eukaryotic |
| | translation initiation factor 2, subunit 1 (alpha,: 35 kD) /FL = gb: BC002513.1 gb: J02645.1 gb: NM_004094.1 |
| 208818_s_at | gb: BC000419.1 /DEF = Homo sapiens, catechol-O-methyltransferase, clone MGC: 8663, mRNA, complete cds. /FEA = mRNA |
| | /PROD = catechol-O-methyltransferase /DB_XREF = gi: 12653300 /UG = Hs.240013 catechol-O-methyltransferase /FL = gb: BC000419.1 gb: M58525.1 |
| | gb: M65212.1 gb: NM_007310.1 gb: NM_000754.2 |
| 203436_at | gb: NM_006413.1 /DEF = Homo sapiens ribonuclease P (30 kD) (RPP30), mRNA. /FEA = mRNA /GEN = RPP30 /PROD = ribonuclease P (30 kD) |
| | /DB_XREF = gi: 5454023 /UG = Hs.139120 ribonuclease P (30 kD) /FL = gb: U77665.1 gb: NM_006413.1 |
| 201380_at | gb: NM_006371.1 /DEF = Homo sapiens cartilage associated protein (CRTAP), mRNA. /FEA = mRNA /GEN = CRTAP /PROD = cartilage associated |
| | protein /DB_XREF = gi: 5453600 /UG = Hs.155481 cartilage associated protein /FL = gb: NM_006371.1 |
| 203614_at | gb: NM_021645.1 /DEF = Homo sapiens KIAA0266 gene product (KIAA0266), mRNA. /FEA = mRNA /GEN = KIAA0266 /PROD = KIAA0266 gene product |
| | /DB_XREF = gi: 11063982 /UG = Hs.127376 KIAA0266 gene product /FL = gb: NM_021645.1 gb: D87455.1 |
| 208649_s_at | gb: AF100752.1 /DEF = Homo sapiens transitional endoplasmic reticulum ATPase mRNA, complete cds. /FEA = mRNA /PROD = transitional |
| | endoplasmic reticulum ATPase /DB_XREF = gi: 5410289 /UG = Hs.106357 valosin-containing protein /FL = gb: AF100752.1 gb: NM_007126.2 |
| 209012_at | Consensus includes gb: AV718192 /FEA = EST /DB_XREF = gi: 10815344 /DB_XREF = est: AV718192 /CLONE = FHTAABE08 /UG = Hs.171957 triple |
| | functional domain (PTPRF interacting) /FL = gb: AF091395.1 |
| 212100_s_at | Consensus includes gb: Z93241 /DEF = Human DNA sequence from clone 222E13 on chromosome 22. Contains three novel genes, an ATP |
| | Synthase G Chain, Mitochondrial (EC 3.6.1.34) pseudogene and the DIAl gene for diaphorase (NADH) (cytochrome b-5 reductase) |
| | (EC 1.6.2.2). Contains ESTs, STSs, . . . /FEA = mRNA 5 /DB_XREF = gi: 4826450 /UG = Hs.278314 hypothetical protein |
| 208756_at | gb: U36764.1 /DEF = Human TGF-beta receptor interacting protein 1 mRNA, complete cds. /FEA = mRNA /PROD = TGF-beta receptor interacting |
| | protein 1 /DB_XREF = gi: 1036804 /UG = Hs.192023 eukaryotic translation initiation factor 3, subunit 2 (beta, 36 kD) /FL = gb: U36764.1 |
| | gb: BC000413.1 gb: BC003140.1 gb: U39067.1 gb: NM_003757.1 gb: AF116697.1 |
| 213034_at | Consensus includes gb: AB023216.1 /DEF = Homo sapiens mRNA for KIAA0999 protein, partial cds. /FEA = mRNA /GEN = KIAA0999 |
| | /PROD = KIAA0999 protein /DB_XREF = gi: 4589641 /UG = Hs.4278 KIAA0999 protein |
| 200684_s_at | Consensus includes gb: AI819709 /FEA = EST /DB_XREF = gi: 5438788 /DB_XREF = est: wj77c07.x1 /CLONE = IMAGE: 2408844 /UG = Hs.108104 |
| | ubiquitin-conjugating enzyme E2L 3 /FL = gb: NM_003347.1 |
| 213019_at | Consensus includes gb: AI123233 /FEA = EST /DB_XREF = gi: 3538999 /DB_XREF = est: qa47e04.x1 /CLONE = IMAGE: 1689918 /UG = Hs.167496 |
| | RAN binding protein 6 |
| 218617_at | gb: NM_017646.1 /DEF = Homo sapiens tRNA isopentenylpyrophosphate transferase (IPT), mRNA. /FEA = mRNA /GEN = IPT |
| | /PROD = tRNA isopentenylpyrophosphate transferase /DB_XREF = gi: 8923064 /UG = Hs.288036 tRNA isopentenylpyrophosphate transferase |
| | /FL = gb: NM_017646.1 |
| 208308_s_at | gb: NM_000175.1 /DEF = Homo sapiens glucose phosphate isomerase (GPI), mRNA. /FEA = mRNA /GEN = GPI /PROD = glucose phosphate isomerase |
| | /DB_XREF = gi: 4504086 /UG = Hs.180532 glucose phosphate isomerase /FL = gb: BC004982.1 gb: K03515.1 gb: NM_000175.1 |
| 209429_x_at | gb: AF112207.1 /DEF = Homo sapiens translation initiation factor eIF-2b delta subunit mRNA, complete cds. /FEA = mRNA |
| | /PROD = translation initiation factor eIF-2b deltasubunit /DB_XREF = gi: 6563201 /UG = Hs.169474 DKFZP586J0119 protein |
| | /FL = gb: BC001870.1 gb: AF112207.1 |
| 221501_x_at | gb: AF229069.1 /DEF = Homo sapiens KIAA0220-like protein mRNA, complete cds. /FEA = mRNA /PROD = KIAA0220-like protein |
| | /DB_XREF = gi: 8980670 /UG = Hs.251928 nuclear pore complex interacting protein /FL = gb: AF229069.1 |
| 209161_at | Consensus includes gb: AI184802 /FEA = EST /DB_XREF = gi: 3735440 /DB_XREF = est: qd24g04.x1 /CLONE = IMAGE: 1724694 /UG = Hs.8551 PRP4STKWD |
| | splicing factor /FL = gb: AF016369.1 gb: U82756.1 gb: NM_004697.1 |
| 201414_s_at | gb: NM_005969.1 /DEF = Homo sapiens nucleosome assembly protein 1-like 4 (NAP1L4), mRNA. /FEA = mRNA /GEN = NAP1L4 |
| | /PROD = nucleosome assembly protein 1-like 4 /DB_XREF = gi: 5174612 /UG = Hs.78103 nucleosome assembly protein 1-like 4 |
| | /FL = gb: U77456.1 gb: NM_005969.1 |
| 212361_s_at | Consensus includes gb: AW190070 /FEA = EST /DB_XREF = gi: 6464550 /DB_XREF = est: x111f11.x1 /CLONE = IMAGE: 2675949 /UG = Hs.1526 ATPase, |
| | Ca++ transporting, cardiac muscle, slow twitch 2 |
| 204084_s_at | Consensus includes gb: AI911687 /FEA-EST /DB_XREF = gi: 5631542 /DB_XREF = est: wc71g01.x1 /CLONE = IMAGE: 2324112 /UG = Hs.30213 |
| | ceroid-lipofuscinosis, neuronal 5 /FL = gb: AF068227.1 gb: NM_006493.1 |
| 203200_s_at | gb: NM_024010.1 /DEF = Homo sapiens 5-methyltetrahydrofolate-homocysteine methyltransferase reductase (MTRR), |
| | transcript variant 2, mRNA. /FEA = mRNA /GEN = MTRR /PROD = methionine synthase reductase, isoform 2 /DB_XREF = gi: 13325067 |
| | /UG = Hs.153792 5-methyltetrahydrofolate-homocysteine methyltransferase reductase /FL = gb: NM_024010.1 gb: AF121214.1 |
| 201405_s_at | gb: NM_006833.1 /DEF = Homo sapiens COP9 subunit 6 (MOV34 homolog, 34 kD) (MOV34-34 KD), mRNA. /FEA = mRNA /GEN = MOV34-34 KD |
| | PROD = COP9 subunit 6 (MOV34 homolog, 34 kD) /DB_XREF = gi: 5803095 /UG = Hs.15591 COP9 subunit 6 (MOV34 homolog, 34 kD) |
| | /FL = gb: BC002520.1 gb: U70735.1 gb: NM_006833.1 |
| 218556_at | gb: NM_014182.1 /DEF = Homo sapiens HSPC160 protein (HSPC160), mRNA. /FEA = mRNA /GEN = HSPC160 /PROD = HSPC160 protein |
| | /DB_XREF = gi: 7661819 /UG = Hs.13144 HSPC160 protein /FL = gb: AF161509.1 gb: NM_014182.1 |
| 217756_x_at | gb: NM_005770.1 /DEF = Homo sapiens small EDRK-rich factor 2 (SERF2), mRNA. /FEA = mRNA /GEN = SERF2 /PROD = small EDRK-rich factor 2 |
| | /DB_XREF = gi: 5032084 /UG = Hs.323806 small EDRK-rich factor 2 /FL = gb: AF320073.1 gb: AF073298.1 gb: NM_005770.1 |
| 209206_at | Consensus includes gb: AV701283 /FEA = EST /DB_XREF = gi: 10717613 /DB_XREF = est: AV701283 /CLONE = ADAAGD06 /UG = Hs.50785 SEC22, vesicle |
| | trafficking protein (S. cerevisiae)-like 1 /FL = gb: BC001364.1 gb: AF047442.1 gb: NM_004892.1 |
| 212165_at | Consensus includes gb: AF070537.1 /DEF = Homo sapiens clone 24606 mRNA sequence. /FEA = mRNA /DB_XREF = gi: 3387894 /UG = Hs.17481 Homo |
| | sapiens clone 24606 mRNA sequence |
| 208983_s_at | gb: M37780.1 /DEF = Human leukocyte surface protein (CD31) mRNA, complete cds. /FEA = mRNA /GEN = CD31 /PROD = leukocyte surface protein |
| | /DB_XREF = gi: 187239 /UG = Hs.78146 plateletendothelial cell adhesion molecule (CD31 antigen) /FL = gb: M37780.1 gb: M28526.1 gb: NM_000442.1 |
| 202042_at | gb: NM_002109.2 /DEF = Homo sapiens histidyl-tRNA synthetase (HARS), mRNA. /FEA = mRNA /GEN = HARS /PROD = histidyl tRNA synthetase |
| | /DB_XREF = gi: 6996013 /UG = Hs.77798 histidyl-tRNA synthetase /FL = gb: NM_002109.2 |
| 202433_at | gb: NM_005827.1 /DEF = Homo sapiens UDP-galactose transporter related (UGTREL1), mRNA. /FEA = mRNA /GEN = UGTREL1 |
| | /PROD = UDP-galactose transporter related /DB_XREF = gi: 5032212 /UG = Hs.154073 UDP-galactose transporter related /FL = gb: D87989.1 |
| | gb: NM_005827.1 |
| 201145_at | gb: NM_006118.2 /DEF = Homo sapiens HS1 binding protein (HAX1), mRNA. /FEA = mRNA /GEN = HAX1 /PROD = HS1 binding protein |
| | /DB_XREF = gi: 13435355 /UG = Hs.15318 HS1 binding protein /FL = gb: NM_006118.2 gb: BC005240.1 gb: U68566.1 |
| 211975_at | Consensus includes gb: BE299671 /FEA = EST /DB_XREF = gi: 9183419 /DB_XREF = est: 600944342T1 /CLONE = IMAGE: 2960218 |
| | /UG = Hs.256310 protein kinase C and casein kinase substrate in neurons 3 |
| 200918_s_at | gb: NM_003139.1 /DEF = Homo sapiens signal recognition particle receptor (docking protein) (SRPR), mRNA. /FEA = mRNA /GEN = SRPR |
| | /PROD = signal recognition particle receptor (dockingprotein) /DB_XREF = gi: 4507222 /UG = Hs.75730 signal recognition particle receptor |
| | (docking protein) /FL = gb: BC001162.1 gb: NM_003139.1 |
| 203497_at | gb: NM_004774.1 /DEF = Homo sapiens PPAR binding protein (PPARBP), mRNA. /FEA = mRNA /GEN = PPARBP /PROD = thyroid hormone receptor |
| | interactor 2 /DB_XREF = gi: 4759265 /UG = Hs.15589 PPAR binding protein /FL = gb: AF055994.1 gb: NM_004774.1 gb: AF283812.1 |
| 209153_s_at | gb: M31523.1 /DEF = Human transcription factor (E2A) mREA, complete cds. /FEA = mRNA /GEN = TCF3 /DB_XREF = gi: 339477 /UG = Hs.101047 |
| | transcription factor 3 (E2A immunoglobulin enhancer binding factors E12E47) /FL = gb: M31523.1 |
| 220966_x_at | gb: NM_030978.1 /DEF = Homo sapiens hypothetical protein similar to actin related protein 23 complex, subunit 5 (MGC3038), mRNA. |
| | /FEA = mRNA /GEN = MGC3038 /PROD = hypothetical protein similar to actin relatedprotein 23 complex, subunit 5 /DB_XREF = gi: 13569955 |
| | /FL = gb: NM_030978.1 |
| 212785_s_at | Consensus includes gb: AA160181 /FEA = EST /DB_XREF = gi: 1734757 /DB_XREF = est: zo81d09.s1 /CLONE = IMAGE: 593297 |
| | /UG = Hs.278635 HDCMA18P protein |
| 219176_at | gb: NM_024520.1 /DEF = Homo sapiens hypothetical protein FLJ22555 (FLJ22555), mRNA. /FEA = mRNA /GEN = FLJ22555 |
| | /PROD = hypothetical protein FLJ22555 /DB_XREF = gi: 13375659 /UG = Hs.3592 hypothetical protein FLJ22555 /FL = gb: NM_024520.1 |
| 218679_s_at | gb: NM_016208.1 /DEF = Homo sapiens VPS28 protein (LOC51160), mRNA. /FEA = mRNA /GEN = LOC51160 /PROD = VPS28 protein |
| | /DB_XREF = gi: 7705884 /UG = Hs.293441 VPS28 protein /FL = gb: AF316887.1 gb: AF182844.1 gb: NM_016208.1 |
| 206991_s_at | gb: NM_000579.1 /DEF = Homo sapiens chemokine (C—C motif) receptor 5 (CCR5), mRNA. /FEA = mRNA /GEN = CCR5 /PROD = chemokine (C—C motif) |
| | receptor 5 /DB_XREF = gi: 4502638 /UG = Hs.54443 chemokine (C—C motif) receptor 5 /FL = gb: U54994.1 gb: U57840.1 gb: NM_000579.1 |
| 209770_at | gb: U90552.1 /DEF = Human butyrophilin (BTF5) mRNA, complete cds. /FEA = mRNA /GEN = BTF5 /PROD = butyrophilin /DB_XREF = gi: 2062705 |
| | /UG = Hs.284283 butyrophilin, subfamily 3, member A1 /FL = gb: U90552.1 |
| 214268_s_at | Consensus includes gb: AL042220 /FEA = EST /DB_XREF = gi: 5421564 /DB_XREF = est: DKFZp434H1920_s1 /CLONE = DKFZp434H1920 /UG = Hs.141727 |
| | myotubularin related protein 4 |
| 201885_s_at | gb: NM_000398.3 /DEF = Homo sapiens diaphorase (NADH) (cytochrome b-5 reductase) (DIA1), nuclear gene encoding mitochondrial |
| | protein, transcript variant M, mRNA. /FEA = mRNA /GEN = DIA1 /PROD = cytochrome b5 reductase, membrane-bound isoform |
| | /DB_XREF = gi: 6552326 /UG = Hs.274464 diaphorase (NADH) (cytochrome b-5 reductase) /FL = gb: BC004821.1 gb: NM_000398.3 |
| 209688_s_at | gb: BC005078.1 /DEF = Homo sapiens, clone MGC: 13033, mRNA, complete cds. /FEA = mRNA /PROD = Unknown (protein for MGC: 13033) |
| | /DB_XREF = gi: 13477224 /UG = Hs.26118 Homo sapiens clone 24766 mRNA sequence /FL = gb: BC005078.1 |
| 203487_s_at | gb: NM_015396.1 /DEF = Homo sapiens DKFZP434A043 protein (DKFZP434A043), mRNA. /FEA = mRNA /GEN = DKFZP434A043 /PROD = DKFZP434A043 protein /DB_XREF = gi: |
| | 7661561 /UG = Hs.102708 DKFZP434A043 protein /FL = gb: NM_015396.1 |
| 202155_s_at | gb: NM_005085.1 /DEF = Homo sapiens nucleoporin 214 kD (CAIN) (NUP214), mRNA. /FEA = mRNA /GEN = NUP214 /PROD = nucleoporin 214 kD (CAIN) |
| | /DB_XREF = gi: 4826873 /UG = Hs.170285 nucleoporin 214 kD (CAIN) /FL = gb: NM_005085.1 |
| 221786_at | Consensus includes gb: BF197222 /FEA = EST /DB_XREF = gi: 11085906 /DB_XREF = est: 7m88b07.x1 /CLONE = IMAGE: 3561949 /UG = Hs.12342 Homo sapiens |
| | clone 24538 mRNA sequence |
| 212846_at | Consensus includes gb: AA811192 /FEA = EST /DB_XREF = gi: 2880803 /DB_XREF = est: ob72b08.s1 /CLONE = IMAGE: 1336887 /UG = Hs.152629 KIAA0179 |
| | protein |
| 209444_at | gb: BC001851.1 /DEF = Homo sapiens, Similar to RAP1, GTP-GDP dissociation stimulator 1, clone MGC: 4525, mRNA, complete cds. |
| | /FEA = mRNA /PROD = Similar to RAP1, GTP-GDP dissociation stimulator1 /DB_XREF = gi: 12804812 /UG = Hs.7940 RAP1, GTP-GDP dissociation |
| | stimulator 1 /FL = gb: NM_021159.1 gb: BC001851.1 gb: BC001816.1 gb: AF215923.1 gb: AF237413.1 |
| 211937_at | Consensus includes gb: NM_001417.1 /DEF = Homo sapiens eukaryotic translation initiation factor 4B (EIF4B), mRNA. /FEA = CDS |
| | /GEN = EIF4B /PROD = eukaryotic translation initiation factor 4B /DB_XREF = gi: 4503532 /UG = Hs.93379 eukaryotic translation initiation |
| | factor 4B /FL = gb: NM_001417.1 |
| 201800_s_at | gb: AF185696.1 /DEF = Homo sapiens oxysterol-binding protein 1 (OSBP1) mRNA, complete cds. /FEA = mRNA /GEN = OSBP1 |
| | /PROD = oxysterol-binding protein 1 /DB_XREF = gi: 10441379 /UG = Hs.24734 oxysterol binding protein /FL = gb: AF185696.1 gb: M86917.1 |
| | gb: NM_002556.1 |
| 211730_s_at | gb: BC005903.1 /DEF = Homo sapiens, polymerase (RNA) II (DNA directed) polypeptide L (7.6 kD), clone MGC: 14494, mRNA, complete cds. |
| | /FEA = mRNA /PROD = polymerase (RNA) II (DNA directed) polypeptide L(7.6 kD) /DB_XREF = gi: 13543491 /FL = gb: BC005903.1 |
| 65635_at | Cluster Incl. AL044097: DKFZp434M1928_s1 Homo sapiens cDNA, 3 end /clone = DKFZp434M1928 /clone_end = 3 /gb = AL044097 /gi = 5432324 |
| | /ug = Hs.29288 /len = 605 |
| 207438_s_at | gb: NM_005701.1 /DEF = Homo sapiens RNA, U transporter 1 (RNUT1), mRNA. /FEA = mRNA /GEN = RNUT1 /PROD = RNA, U transporter 1 |
| | /DB_XREF = gi: 5031832 /UG = Hs.21577 RNA, U transporter 1 /FL = gb: AF039029.1 gb: NM_005701.1 |
| 221819_at | Consensus includes gb: BF791960 /FEA = EST /DB_XREF = gi: 12097014 /DB_XREF = est: 602252342F1 /CLONE = IMAGE: 4344539 /UG = Hs.94308 |
| | Homo sapiens cDNA FLJ10447 fis, clone NT2RP1000851 |
| 203912_s_at | gb: NM_006730.1 /DEF = Homo sapiens deoxyribonuclease I-like 1 (DNASE1L1), mRNA. /FEA = mRNA /GEN = DNASE1L1 /PROD = deoxyribonuclease |
| | I-like 1 /DB_XREF = gi: 5803006 /UG = Hs.77091 deoxyribonuclease I-like 1 /FL = gb: U06846.1 gb: NM_006730.1 |
| 216996_s_at | Consensus includes gb: AK021557.1 /DEF = Homo sapiens cDNA FLJ11495 fis, clone HEMBA1001950, highly similar to Homo sapiens mRNA |
| | for KIAA0971 protein. /FEA = mRNA /DB_XREF = gi: 10432760 /UG = Hs.84429 KIAA0971 protein |
| 202138_x_at | gb: NM_006303.2 /DEF = Homo sapiens JTV1 gene (JTV1), mRNA. /FEA = mRNA /GEN = JTV1 /PROD = JTV1 /DB_XREF = gi: 11125769 /UG = Hs.301613 JTV1 |
| | gene /FL = gb: NM_006303.2 gb: U24169.1 gb: BC002853.1 |
| 219007_at | gb: NM_024647.1 /DEF = Homo sapiens hypothetical protein FLJ13287 (FL13287), mRNA. /FEA = mRNA /GEN = FLJ13287 /PROD = hypothetical |
| | protein /FLJ13287 /DB_XREF = gi: 13375888 /UG = Hs.53263 hypothetical protein FLJ13287 /FL = gb: NM_024647.1 |
| 219714_s_at | gb: NM_018398.1 /DEF = Homo sapiens calcium channel alpha2-delta3 subunit (HSA272268), mRNA. /FEA = mRNA /GEN = HSA272268 |
| | /PROD = calcium channel alpha2-delta3 subunit /DB_XREF = gi: 8923764 /UG = Hs.22958 calcium channel, voltage-dependent, alpha 2delta 3 |
| | subunit /FL = gb: NM_018398.1 |
| 201263_at | gb: NM_003191.1 /DEF = Homo sapiens threonyl-tRNA synthetase (TARS), mRNA. /FEA = mRNA /GEN = TARS /PROD = threonyl-tRNA synthetase |
| | /DB_XREF = gi: 4507366 /UG = Hs.84131 threonyl-tRNA synthetase /FL = gb: BC000517.1 gb: M63180.1 gb: NM_003191.1 |
| 201157_s_at | Consensus includes gb: AF020500.1 /DEF = Homo sapiens myristoyl CoA: protein N-myristoyltransferase mRNA, complete cds. /FEA = CDS |
| | /PROD = myristoyl CoA: protein N-myristoyltransferase /DB_XREF = gi: 2760893 /UG = Hs.111039 N-myristoyltransferase 1 |
| | /FL = gb: NM_021079.1 gb: AF020500.1 gb: AF043324.1 |
| 221593_s_at | gb: BC001663.1 /DEF = Homo sapiens, Similar to ribosomal protein L31, clone MGC: 1641, mRNA, complete cds. /FEA = mRNA |
| | /PROD = Similar to ribosomal protein L31 /DB_XREF = gi: 12804504 /UG = Hs.164170 vascular Rab-GAPTBC-containing /FL = gb: BC001663.1 |
| 214202_at | Consensus includes gb: N21364 /FEA = EST /DB_XREF = gi: 1126534 /DB_XREF = est: yx50h10.s1 /CLONE = IMAGE: 265219 /UG = Hs.26968 Homo sapiens |
| | mRNA from chromosome 5q21-22, clone: 357Ex |
| 203142_s_at | gb: NM_003664.1 /DEF = Homo sapiens adaptor-related protein complex 3, beta 1 subunit (AP3B1), mRNA. /FEA = mRNA |
| | /GEN = AP3B1 /PROD = adaptor-related protein complex 3, beta 1subunit /DB_XREF = gi: 4501974 |
| | /UG = Hs.155172 adaptor-related protein complex 3, beta 1 subunit /FL = gb: U91931.1 gb: U81504.1 gb: NM_003664.1 |
| 222001_x_at | Consensus includes gb: AI160126 /FEA = EST /DB_XREF = gi: 3693506 /DB_XREF = est: qb51h08.x1 /CLONE = IMAGE: 1703679 /UG = Hs.295901 |
| | KIAA0493 protein |
| 213374_x_at | Consensus includes gb: AW000964 /FEA = EST /DB_XREF = gi: 5847880 /DB_XREF = est: wr90h10.x1 /CLONE = IMAGE: 2495011 /UG = Hs.236642 |
| | 3-hydroxyisobutyryl-Coenzyme A hydrolase |
| 201018_at | Consensus includes gb: AL079283.1 /DEF = Homo sapiens mRNA full length insert cDNA clone EUROIMAGE 39515. /FEA = mRNA |
| | /DB_XREF = gi: 5102744 /UG = Hs.4310 eukaryotic translation initiation factor 1A /FL = gb: BC000793.1 gb: L18960.1 gb: NM_001412.1 |
| 206206_at | gb: NM_005582.1 /DEF = Homo sapiens lymphocyte antigen 64 (mouse) homolog, radioprotective, 105 kD (LY64), mRNA. /FEA = mRNA |
| | /GEN = LY64 /PROD = lymphocyte antigen 64 (mouse) homolog, radioprotective, 105 kD /DB_XREF = gi: 5031894 |
| | /UG = Hs.87205 lymphocyte antigen 64 (mouse) homolog, radioprotective, 105 kD /FL = gb: D83597.1 gb: NM_005582.1 |
| 209566_at | Consensus includes gb: AL080184.1 /DEF = Homo sapiens mRNA; cDNA DKFZp434O071 (from clone DKFZp434O071). /FEA = mRNA |
| | /DB_XREF = gi: 5262661 /UG = Hs.7089 insulin induced protein 2 /FL = gb: AF125392.1 |
| 212194_s_at | Consensus includes gb: AI418892 /FEA = EST /DB_XREF = gi: 4264823 /DB_XREF = est: tf43c01.x1 /CLONE = IMAGE: 2098944 /UG = Hs.79305 KIAA0255 |
| | gene product |
| 204526_s_at | gb: NM_007063.1 /DEF = Homo sapiens vascular Rab-GAPTBC-containing (VRP), mRNA. /FEA = mRNA /GEN = VRP /PROD = vascular |
| | Rab-GAPTBC-containing /DB_XREF = gi: 5902153 /UG = Hs.164170 vascular Rab-GAPTBC-containing /FL = gb: AB024057.1 gb: NM_007063.1 |
| 201568_at | gb: NM_014402.1 /DEF = Homo sapiens low molecular mass ubiquinone-binding protein (9.5 kD) (QP-C), mRNA. /FEA = mRNA /GEN = QP-C |
| | /PROD = low molecular mass ubiquinone-binding protein /DB_XREF = gi: 7657485 /UG = Hs.3709 low molecular mass ubiquinone-binding |
| | protein (9.5 kD) /FL = gb: BC001390.1 gb: D50369.1 gb: NM_014402.1 |
| 221829_s_at | Consensus includes gb: AI307759 /FEA = EST /DB_XREF = gi: 4002363 /DB_XREF = est: tb24g08.x1 /CLONE = IMAGE: 2055326 /UG = Hs.168075 |
| | karyopherin (importin) beta 2 |
| 200613_at | gb: NM_004068.1 /DEF = Homo sapiens adaptor-related protein complex 2, mu 1 subunit (AP2M1), mRNA. /FEA = mRNA /GEN = AP2M1 |
| | /PROD = adaptor-related protein complex 2, mu 1 subunit /DB_XREF = gi: 4757993 /UG = Hs.152936 adaptor-related protein complex 2, |
| | mu 1 subunit /FL = gb: U36188.1 gb: BC004996.1 gb: D63475.1 gb: NM_004068.1 |
| 213160_at | Consensus includes gb: D86964.1 /DEF = Human mRNA for KIAA0209 gene, partial cds. /FEA = mRNA /GEN = KIAA0209 /DB_XREF = gi: 1504001 |
| | /UG = Hs.17211 dedicator of cyto-kinesis 2 |
| 211375_s_at | gb: AF141870.1 /DEF = Homo sapiens translational control protein 80 mRNA, complete cds. /FEA = mRNA /PROD = translational control protein |
| | 80 /DB_XREF = gi: 5006601 /UG = Hs.256583 interleukin enhancer binding factor 3, 90 kD /FL = gb: AF141870.1 |
| 202276_at | gb: NM_006304.1 /DEF = Homo sapiens Deleted in split-handsplit-foot 1 region (DSS1), mRNA. /FEA = mRNA /GEN = DSS1 /PROD = deleted in |
| | split-handsplit-foot 1 region /DB_XREF = gi: 5453639 /UG = Hs.85215 Deleted in split-handsplit-foot 1 region /FL = gb: U41515.1 |
| | gb: NM_006304.1 |
| 201892_s_at | gb: NM_000884.1 /DEF = Homo sapiens IMP (inosine monophosphate) dehydrogenase 2 (IMPDH2), mRNA. /FEA = mRNA /GEN = IMPDH2 /PROD = IMP |
| | (inosine monophosphate) dehydrogenase 2 /DB_XREF = gi: 4504688 /UG = Hs.75432 IMP (inosine monophoaphate) dehydrogenase 2 |
| | /FL = gb: J04208.1 gb: NM_000884.1 |
| 217905_at | gb: NM_024834.1 /DEF = Homo sapiens hypothetical protein FLJ13081 (FLJ13081), mRNA. /FEA = mRNA /GEN = FLJ13081 /PROD = hypothetical |
| | protein FLJ13081 /DB_XREF = gi: 13376242 /UG = Hs.180638 hypothetical protein FLJ13081 /FL = gb: BC004183.1 gb: NM_024834.1 |
| 209036_s_at | gb: BC001917.1 /DEF = Homo sapiens, malate dehydrogenase 2, NAD (mitochondrial), clone MGC: 3559, mRNA, complete cds. |
| | /FEA = mRNA /PROD = malate dehydrogenase 2, NAD (mitochondrial) /DB_XREF = gi: 12804928 /UG = Hs.111076 malate dehydrogenase 2, |
| | NAD (mitochondrial) /FL = gb: BC001917.1 gb: AF047470.1 gb: NM_005918.1 |
| 222010_at | Consensus includes gb: BF224073 /FEA = EST /DB_XREF = gi: 11131299 /DB_XREF = est: 7q83e05.x1 /CLONE = IMAGE: 3704936 /UG = Hs.278544 |
| | acetyl-Coenzyme A acetyltransferase 2 (acetoacetyl Coenzyme A thiolase) |
| 203825_at | gb: NM_007371.2 /DEF = Homo sapiens bromodomain-containing 3 (BRD3), mRNA. /FEA = mRNA /GEN = BRD3 /PROD = bromodomain-containing |
| | protein 3 /DB_XREF = gi: 12408642 /UG = Hs.86896 bromodomain-containing 3 /FL = gb: NM_007371.2 gb: D26362.1 |
| 202943_s_at | gb: M38083.1 /DEF = Human alpha-N-acetylgalactosaminidase mRNA, complete cds. /FEA = mRNA /PROD = alpha-N-acetylgalactosaminidase |
| | /DB_XREF = gi: 189054 /UG = Hs.75372 N-acetylgalactosaminidase, alpha- /FL = gb: BC000095.1 gb: M62783.1 gb: M38083.1 gb: NM_000262.1 |
| 210555_s_at | gb: U85430.1 /DEF = Human transcription factor NFATx4 mRNA, complete cds. /FEA = mRNA /PROD = transcription factor NFATx4 |
| | /DB_XREF = gi: 1835590 /UG = Hs.172674 nuclear factor of activated T-cells, cytoplasmic, calcineurin-dependent 3 /FL = gb: U85430.1 |
| 218370_s_at | gb: NM_022753.1 /DEF = Homo sapiens hypothetical protein FLJ12903 (FLJ12903), mRNA. /FEA = mRNA /GEN = FLJ12903 /PROD = hypothetical |
| | protein FLJ12903 /DB_XREF = gi: 12232418 /UG = Hs.14928 hypothetical protein FLJ12903 /FL = gb: NM_022753.1 |
| 208070_s_at | gb: NM_002912.1 /DEF = Homo sapiens REV3 (yeast homolog)-like, catalytic subunit of DNA polymerase zeta (REV3L), mRNA. /FEA = mRNA |
| | /GEN = REV3L /PROD = REV3 (yeast homolog)-like, catalytic subunit ofDNA polymerase zeta /DB_XREF = gi: 4506482 /UG = Hs.115521 REV3 |
| | (yeast homolog)-like, catalytic subunit of DNA polymerase zeta /FL = gb: AF078695.1 gb: NM_002912.1 gb: AF179428.1 |
| 201272_at | gb: NM_001628.1 /DEF = Homo sapiens aldo-keto reductase family 1, member B1 (aldose reductase) (AKR1B1), mRNA. /FEA = mRNA /GEN = AKR1B1 |
| | /PROD = aldo-keto reductase family 1, member B1 (aldosereductase) /DB_XREF = gi: 4502048 /UG = Hs.75313 aldo-keto reductase |
| | family 1, member B1 (aldose reductase) /FL = gb: BC000260.1 gb: BC005387.1 gb: J04795.1 gb: J05017.1 gb: J05474.1 gb: M34720.1 |
| | gb: NM_001628.1 |
| 209824_s_at | gb: AB000812.1 /DEF = Homo sapiens mRNA for BMAL1b, complete cds. /FEA = mRNA /PROD = BMAL1b /DB_XREF = gi: 2094734 /UG = Hs.74515 aryl |
| | hydrocarbon receptor nuclear translocator-like /FL = gb: AB000812.1 gb: AF044288.1 |
| 209064_x_at | gb: AL136920.1 /DEF = Homo sapiens mRNA; cDNA DKFZp586C051 (from clone DKFZp586C051); complete cds. /FEA = mRNA /GEN = DKFZp586C051 |
| | /PROD = hypothetical protein /DB_XREF = gi: 12053334 /UG = Hs.109643 polyadenylate binding protein-interacting protein 1 /FL = gb: AL136920.1 |
| 200759_x_at | gb: NM_003204.1 /DEF = Homo sapiens nuclear factor (erythroid-derived 2)-like 1 (NFE2L1), mRNA. /FEA = mRNA /GEN = NFE2L1 /PROD = transcription |
| | factor 11 (basic leucine zippertype) /DB_XREF = gi: 4505378 /UG = Hs.83469 nuclear factor (erythroid-derived 2)-like 1 |
| | /FL = gb: NM_003204.1 gb: U08853.1 |
| 221787_at | Consensus includes gb: BF431618 /FEA = EST /DB_XREF = gi: 11443732 /DB_XREF = est: 7o16e03.x1 /CLONE = IMAGE: 3574349 |
| | /UG = Hs.12342 Homo sapiens clone 24538 mRNA sequence |
| 211581_x_at | gb: AF000426.1 /DEF = Homo sapiens LST1 mRNA, cLST1E splice variant, complete cds. /FEA = mRNA /GEN = LST1 |
| | /DB_XREF = gi: 2145067 /UG = Hs.88411 lymphocyte antigen 117 /FL = gb: AF000426.1 |
| 37384_at | Cluster Incl. D13640: Human mRNA for KIAA0015 gene, complete cds /cds = (106, 1470) /gb = D13640 /gi = 286006 /ug = Hs.77961 /len = 5134 |
| 218423_x_at | gb: NM_016516.1 /DEF = Homo sapiens tumor antigen SLP-8p (HCC8), mRNA. /FEA = mRNA /GEN = HCC8 /PROD = tumor antigen SLP-8p |
| | /DB_XREF = gi: 7705396 /UG = Hs.48499 tumor antigen SLP-8p /FL = gb: AF102177.1 gb: NM_016516.1 |
| 210213_s_at | gb: AF022229.1 /DEF = Homo sapiens translation initiation factor 6 (eIF6) mRNA, complete cds. /FEA = mRNA /GEN = eIF6 |
| | /PROD = translation initiation factor 6 /DB_XREF = gi: 2809382 /UG = Hs.5215 integrin beta 4 binding protein /FL = gb: AF022229.1 |
| 201598_s_at | gb: NM_001567.2 /DEF = Homo sapiens inositol polyphosphate phosphatase-like 1 (INPPL1), mRNA. /FEA = mRNA /GEN = INPPL1 |
| | /PROD = inositol polyphosphate phosphatase-like 1 /DB_XREF = gi: 4755141 /UG = Hs.75339 inositol polyphosphate phosphatase-like 1 |
| | /FL = gb: NM_001567.2 gb: L24444.1 |
| 204301_at | gb: NM_014867.1 /DEF = Homo sapiens KIAA0711 gene product (KIAA0711), mRNA. /FEA = mRNA /GEN = KIAA0711 /PROD = KIAA0711 gene product |
| | /DB_XREF = gi: 7662259 /UG = Hs.5333 KIAA0711 gene product /FL = gb: AB018254.1 gb: NM_014867.1 |
| 202877_s_at | Consensus includes gb: W72082 /FEA = EST /DB_XREF = gi: 1382588 /DB_XREF = est: zd70c06.s1 /CLONE = IMAGE: 345994 /UG = Hs.97199 complement |
| | component C1g receptor /FL = gb: NM_012072.2 gb: U94333.1 |
| 208772_at | Consensus includes gb: AU160676 /FEA = EST /DB_XREF = gi: 11022197 /DB_XREF = est: AU160676 /CLONE = Y79AA1002208 /UG = Hs.301226 KIAA1085 |
| | protein /FL = gb: AL136943.1 |
| 201998_at | Consensus includes gb: AI743792 /FEA = EST /DB_XREF = gi: 5112080 /DB_XREF = est: wg53h11.x1 /CLONE = IMAGE: 2368869 /UG = Hs.2554 |
| | sialyltransferase 1 (beta-galactoside alpha-2, 6-sialytransferase) /FL = gb: NM_003032.1 |
| 208722_s_at | gb: BC001081.1 /DEF = Homo sapiens, anaphase-promoting complex subunit 5, clone MGC: 2750, mRNA, complete cds. /FEA = mRNA |
| | /PROD = anaphase-promoting complex subunit 5 /DB_XREF = gi: 12654502 /UG = Hs.7101 anaphase-promoting complex subunit 5 |
| | /FL = gb: BC001081.1 gb: BC001950.1 gb: AF191339.1 gb: NM_016237.1 |
| 55692_at | Cluster Incl. W22924: 75H3 Homo sapiens cDNA /clone = (not-directional) /gb = W22924 /gi = 1299757 /ug = Hs.96560 /len = 792 |
| 217954_s_at | gb: NM_015153.1 /DEF = Homo sapiens KIAA0244 protein (KIAA0244), mRNA. /FEA = mRNA /GEN = KIAA0244 /PROD = KIAA0244 protein |
| | /DB_XREF = gi: 7662017 /UG = Hs.78893 KIAA0244 protein /FL = gb: AF091622.1 gb: NM_015153.1 |
| 320307_at | gb: NM_016382.1 /DEF = Homo sapiens natural killer cell receptor 2B4 (CD244), mRNA. /FEA = mRNA /GEN = CD244 /PROD = natural killer cell |
| | receptor 2B4 /DB_XREF = gi: 7706528 /UG = Hs.157872 natural killer cell receptor 2B4 /FL = gb: AF242540.1 gb: AF105261.1 gb: AF145782.1 |
| | gb: AF107761.2 gb: AF117711.1 gb: NM_016382.1 |
| 211989_at | Consensus includes gb: NM_003079.1 /DEF = Homo sapiens SWISNF related, matrix associated, actin dependent regulator of chromatin, |
| | subfamily e, member 1 (SMARCE1), mRNA. /FEA = CDS /GEN = SMARCE1 /PROD = SWISNF related, matrix associated, actindependent regulator |
| | of chromatin, subfamily e, member 1 /DB_XREF = gi: 4507088 /UG = Hs.332848 SWISNF related, matrix associated, |
| | actin dependent regulator of chromatin, subfamily e, member 1 /FL = gb: NM_003079.1 |
| 209303_at | gb: BC005270.1 /DEF = Homo sapiens, NADH dehydrogenase (ubiquinone) Fe—S protein 4 (18 kD) (NADH-coenzyme Q reductase), |
| | clone MGC: 12313, mRNA, complete cds. /FEA = mRNA /PROD = NADH dehydrogenase (ubiquinone) Fe—S protein 4(18 kD) |
| | (NADH-coenzyme Q reductase) /DB_XREF = gi: 13528959 /UG = Hs.10758 NADH dehydrogenase (ubiquinone) Fe—S protein 4 (18 kD) |
| | (NADH-coenzyme Q reductase) /FL = gb: BC005270.1 gb: AF020351.1 gb: NM_002495.1 |
| 201729_s_at | gb: NM_014680.1 /DEF = Homo sapiens KIAA0100 gene product (KIAA0100), mRNA. /FEA = mRNA /GEN = KIAA0100 /PROD = KIAA0100 gene product |
| | /DB_XREF = gi: 7661903 /UG = Hs.151761 KIAA0100 gene product /FL = gb: D43947.1 gb: NM_014680.1 |
| 203530_s_at | gb: NM_004604.1 /DEF = Homo sapiens syntaxin 4A (placental) (STX4A), mRNA. /FEA = mRNA /GEN = STX4A /PROD = syntaxin 4A (placental) |
| | /DB_XREF = gi: 4759185 /UG = Hs.83734 syntaxin 4A (placental) /FL = gb: BC002436.1 gb: AF026007.1 gb: U07158.1 gb: NM_004604.1 |
| 201622_at | gb: NM_014390.1 /DEF = Homo sapiens EBNA-2 co-activator (100 kD) (p100), mRNA. /FEA = mRNA /GEN = p100 /PROD = EBNA-2 co-activator |
| | (100 kD) /DB_XREF = gi: 7657430 /UG = Hs.79093 EBNA-2 co-activator (100 kD) /FL = gb: NM_014390.1 gb: U22055.1 |

TABLE 2
Genes from Cluster Analysis 2

| Affymetrix-internal | |
|---|---|
| designation | Description of the sequence in the GeneBank data base |
| 200654_at | gb: J02783.1 /DEF = Human thyroid hormone binding protein (p55) mRNA, complete cds. /FEA = mRNA /GEN = P4HB |
| | /DB_XREF = gi: 339646 /UG = Hs.75655procollagen-proline, 2-oxoglutarate 4-dioxygenase (proline 4-hydroxylase), |
| | beta polypeptide (protein disulfide isomerase; thyroid hormone bindingprotein p55) /FL = gb: J02783.1 gb: NM_000918.1 |
| 203126_at | gb: NM_014214.1 /DEF = Homo sapiens inositol(myo)-1(or 4)-monophosphatase 2 (IMPA2), mRNA. /FEA = mRNA /GEN = IMPA2 |
| | /PROD = inositol(myo)-1(or 4)-monophosphatase 2 /DB_XREF = gi: 7657235 /UG = Hs.5753 inositol(myo)-1(or 4)-monophosphatase 2 |
| | /FL = gb: AF200432.1gb: NM_014214.1 gb: AF014398.2 |
| 203585_at | gb: NM_007150.1 /DEF = Homo sapiens zinc finger protein 185 (LIM domain) (ZNF185), mRNA. /FEA = mRNA /GEN = ZNF185 |
| | /PROD = zinc finger protein 185(LIM domain) /DB_XREF = gi: 6005971 /UG = Hs.16622 zinc finger protein 185 (LIM domain) /FL = gb: NM_007150.1 |
| 205220_at | gb: NM_006018.1 /DEF = Homo sapiens putative chemokine receptor; GTP-binding protein (HM74), mRNA. /FEA = mRNA |
| | /GEN = HM74 /PROD = putative chemokine receptor; GTP-bindingprotein /DB_XREF = gi: 5174460 /UG = Hs.137555 putative |
| | chemokine receptor; GTP-binding protein /FL = gb: NM_006018.1 |
| 207008_at | gb: NM_001557.1 /DEF = Homo sapiens interleukin 8 receptor, beta (IL8RB), mRNA. /FEA = mRNA /GEN = IL8RB /PROD = interleukin 8 receptor, |
| | beta /DB_XREF = gi: 4504682 /UG = Hs.846 interleukin 8 receptor, beta /FL = gb: M94582.1 gb: M73969.1 gb: NM_001557.1 gb: L19593.1 |
| 200897_s_at | gb: NM_016081.1 /DEF = Homo sapiens palladin (KIAA0992), mRNA. /FEA = mRNA /GEN = KIAA0992 /PROD = palladin /DB_XREF = gi: 7706354 |
| | /UG = Hs.194431 palladin /FL = gb: AF077041.1 gb: AF151909.1 gb: NM_016081.1 |
| 205568_at | gb: NM_020980.2 /DEF = Homo sapiens aquaporin 9 (AQP9), mRNA. /FEA = mRNA /GEN = AQP9 /PROD = aquaporin 9 /DB_XREF = gi: 11038652 |
| | /UG = Hs.104624 aquaporin 9 /FL = gb: NM_020980.2 gb: AB008775.1 gb: AF016495.1 |
| 214022_s_at | Consensus includes gb: AA749101 /FEA = EST /DB_XREF = gi: 2789059 /DB_XREF = est: ny11d02.s1 /CLONE = IMAGE: 1271427 |
| | /UG = Hs.146360 interferon induced transmembrane protein 1 (9-27) |
| 209762_x_at | gb: AF280094.1 /DEF = Homo sapiens transcriptional coactivator Sp110b mRNA, complete cds. /FEA = mRNA |
| | /PROD = transcriptional coactivator Sp110b /DB_XREF = gi: 9800493 /UG = Hs.38125 interferon-induced protein 75, 52 kD /FL = gb: AF280094.1 |
| 203234_at | gb: NM_003364.1 /DEF = Homo sapiens uridine phosphorylase (UP), MRNA. /FEA = mRNA /GEN = UP /PROD = uridine phosphorylase |
| | /DB_XREF = gi: 4507838 /UG = Hs.77573 uridine phosphorylase /FL = gb: BC001405.1 gb: NM_003364.1 |
| 201940_at | Consensus includes gb: AA897514 /FEA = EST /DB_XREF = gi: 3034134 /DB_XREF = est: aj62b12.s1 /CLONE = IMAGE: 1394879 |
| | /UG = Hs.5057 carboxypeptidase D /FL = gb: U65090.1 gb: D85390.1 gb: NM_001304.2 |
| 209310_s_at | gb: U25804.1 /DEF = Human Ich-2 cysteine protease mRNA, complete cds. /FEA = mRNA /PROD = Ich-2 /DB_XREF = gi: 886049 /UG = Hs.74122 caspase 4, apoptosis-related |
| | cysteine protease /FL = gb: U28976.1 gb: U28977.1 gb: U28978.1 gb: NM_001225.1 gb: U25804.1 gb: U28014.1 |
| 202128_at | gb: NM_014821.1 /DEF = Homo sapiens KIAA0317 gene product (KIAA0317), mRNA. /FEA = mRNA /GEN = KIAA0317 |
| | /PROD = KIAA0317 gene product /DB_XREF = gi: 7662051 /UG = HS.20126 KIAA0317 gene product /FL = gb: AB002315.1 gb: NM_014821.1 |
| 201192_s_at | gb: NM_006224.1 /DEF = Homo sapiens phosphotidylinositol transfer protein (PITPN), mRNA. /FEA = mRNA /GEN = PITPN /PROD = phosphotidylinositol transfer |
| | protein /DB_XREF = gi: 5453907 /UG = Hs.79709 phosphotidylinositol transfer protein /FL = gb: D30036.1 gb: M73704.1 gb: NM_006224.1 |
| 208012_x_at | gb: NM_004509.1 /DEF = Homo sapiens interferon-induced protein 41, 30 kD (IFI41), mRNA. /FEA = mRNA /GEN = IFI41 |
| | /PROD = interferon-induced protein 41, 30 kD /DB_XREF = gi: 4758585 /UG = Hs.241510 interferon-induced protein 41, 30 kD |
| | /FL = gb: L22342.1 gb: NM_004509.1 |
| 209137_s_at | gb: BC000263.1 /DEF = Homo sapiens, Similar to ubiquintin c-terminal hydrolase related polypeptide, clone MGC: 2621, mRNA, |
| | complete cds. /FEA = mRNA /PROD = Similar to ubiquintin c-terminal hydrolaserelated polypeptide /DB_XREF = gi: 12653004 |
| | /UG = Hs.78829 ubiquitin specific protease 10 /FL = gb: BC000263.1 |
| 218023_s_at | gb: NM_016605.1 /DEF = Homo sapiens putative nuclear protein (LOC51307), mRNA. /FEA = mRNA /GEN = LOC51307 |
| | /PROD = putative nuclear protein /DB_XREF = gi: 7706138 /UG = Hs.102469 putative nuclear protein /FL = gb: AF251040.1 gb: NM_016605.1 |
| 201190_s_at | Consensus includes gb: H15647 /FEA = EST /DB_XREF = gi: 880467 /DB_XREF = est: ym27b09.s1 /CLONE = IMAGE: 49287 |
| | /UG = Hs.79709 phosphotidylinositol transfer protein /FL = gb: D30036.1 gb: M73704.1 gb: NM_006224.1 |
| 210563_x_at | gb: U97075.1 /DEF = Homo sapiens FLICE-like inhibitory protein short form mRNA, complete cds. /FEA = mRNA |
| | /PROD = FLICE-like inhibitory protein short form /DB_XREF = gi: 2253680 /UG = Hs.195175 CASP8 and FADD-like apoptosis regulator /FL = gb: U97075.1 |
| 221827_at | Consensus includes gb: BE788439 /FEA = EST /DB_XREF = gi: 10209637 /DB_XREF = est: 601475616F1 /CLONE = IMAGE: 3878643 |
| | /UG = Hs.247280 HBV associated factor |
| 202041_s_at | gb: NM_004214.3 /DEF = Homo sapiens fibroblast growth factor (acidic) intracellular binding protein (FIBP), mRNA. |
| | /FEA = mRNA /GEN = FIBP /PROD = fibroblast growth factor (acidic) intracellularbinding protein /DB_XREF = gi: 7262377 |
| | /UG = Hs.7768 fibroblast growth factor (acidic) intracellular binding protein /FL = gb: AF171944.1 gb: AF171945.1 gb: AF171946.1 |
| | gb: AF010187.2 gb: NM_004214.3 |
| 201001_s_at | Consensus includes gb: BG164064 /FEA = EST /DB_XREF = gi: 12670767 /DB_XREF = est: 602341091F1 /CLONE = IMAGE: 4449022 |
| | /UG = Hs.75875 ubiquitin-conjugating enzyme E2 variant 1 /FL = gb: U39361.1 gb: NM_003349.2 gb: BC000468.1 |
| 212975_at | Consensus includes gb: AB020677.2 /DEF = Homo sapiens mRNA for KIAA0870 protein, partial cds. /FEA = mRNA /GEN = KIAA0870 |
| | /PROD = KIAA0870 protein /DB_XREF = gi: 6635136 /UG = Hs.18166 KIAA0870 protein |
| 201412_at | gb: NM_014045.1 /DEF = Homo sapiens DKFZP564C1940 protein (DKFZP564C1940), mRNA. /FEA = mRNA /GEN = DKFZP564C1940 |
| | /PROD = DKFZP564C1940 protein /DB_XREF = gi: 13027587 /UG = Hs.3804 DKFZP564C1940 protein /FL = gb: BC000424.1 gb: NM_014045.1 gb: AF131760.1 |
| 203708_at | gb: NM_002600.1 /DEF = Homo sapiens phosphodiesterase 4B, cAMP-specific (dunce (Drosophila)-homolog phosphodiesterase E4 (PDE4B) |
| | mRNA. /FEA = mRNA /GEN = PDE4B /PROD = phosphodiesterase 4B, cAMP-specific (dunce (Drosophila)-homolog phosphodiesterase E4 |
| | /DB_XREF = gi: 4505662 /UG = Hs.188 phosphodiesterase 4B, cAMP-specific (dunce (Drosophila)-homolog phosphodiesterase E4) |
| | /FL = gb: M97515.1 gb: L20971.1 gb: NM_002600.1 |
| 203907_s_at | gb: NM_014869.1 /DEF = Homo sapiens KIAA0763 gene product (KIAA0763), mRNA. /FEA = mRNA /GEN = KIAA0763 /PROD = KIAA0763 gene product /DB_XREF = gi: 7662289 |
| | /UG = Hs.4764 KIAA0763 gene product /FL = gb: AB018306.1 gb: NM_014869.1 |
| 209882_at | gb: AF084462.1 /DEF = Homo sapiens GTP-binding protein ROC1 (ROC1) mRNA, complete cds. /FEA = mRNA /GEN = ROC1 |
| | /PROD = GTP-binding protein ROC1 /DB_XREF = gi: 4234917 /UG = Hs.96038 Ric (Drosophila)-like, expressed in many tissues |
| | /FL = gb: U71203.1 gb: U78165.1 gb: AF084462.1 gb: NM_006912.1 |
| 204276_at | Consensus includes gb: BE895437 /FEA = EST /DB_XREF = gi: 10358829 /DB_XREF = est: 601437912F1 /CLONE = IMAGE: 3922971 |
| | /UG = Hs.274701 thymidine kinase 2, mitochondrial /FL = gb: NM_004614.1 gb: U77088.1 |
| 221984_s_at | Consensus includes gb: AL040896 /FEA = EST /DB_XREF = gi: 5409841 /DB_XREF = est: DKFZp434I2415_s1 /CLONE = DKFZp434I2415 |
| | /UG = Hs.22412 hypothetical protein MGC3035 |
| 218517_at | gb: NM_024900.1 /DEF = Homo sapiens hypothetical protein FLJ22479 (FLJ22479), mRNA. /FEA = mRNA /GEN = FLJ22479 |
| | /PROD = hypothetical protein FLJ22479 /DB_XREF = gi: 13376356 /UG = Hs.238246 hypothetical protein FLJ22479 /FL = gb: NM_024900.1 |
| 208740_at | Consensus includes gb: BF593650 /FEA = EST /DB_XREF = gi: 11685974 /DB_XREF = est: nac02d03.x1 /CLONE = IMAGE: 3275957 |
| | /UG = Hs.23964 sin3-associated polypeptide, 18 kD /FL = gb: NM_005870.2 gb: U96915.1 gb: AF153608.1 gb: U78303.1 |
| 221484_at | Consensus includes gb: BF691447 /FEA = EST /DB_XREF = gi: 11976855 /DB_XREF = est: 602247615F1 /CLONE = IMAGE: 4332866 |
| | /UG = Hs.107526 UDP-Gal: betaGlcNAc beta 1,4-galactosyltransferase, polypeptide 5 /FL = gb: AB004550.1 gb: AF038663.1 gb: NM_004776.1 |
| 209575_at | gb: BC001903.1 /DEF = Homo sapiens, Similar to interleukin 10 receptor, beta, clone MGC: 2210, mRNA, complete cds. |
| | /FEA = mRNA /PROD = Similar to interleukin 10 receptor, beta /DB_XREF = gi: 12804902 /UG = Hs.173936 interleukin 10 receptor, |
| | beta /FL = gb: BC001903.1 gb: NM_000628.1 |
| 200619_at | gb: NM_006842.1 /DEF = Homo sapiens splicing factor 3b, subunit 2, 145 kD (SF3B2), mRNA. /FEA = mRNA /GEN = SF3B2 |
| | /PROD = splicing factor 3b, subunit 2, 145 kD /DB_XREF = gi: 5803154 /UG = Hs.75916 splicing factor 3b, subunit 2, 145 kD |
| | /FL = gb: U41371.1 gb: NM_006842.1 |
| 217286_s_at | Consensus includes gb: BC001805.1 /DEF = Homo sapiens, clone IMAGE: 3543670, mRNA, partial cds. /FEA = mRNA |
| | /PROD = Unknown (protein for IMAGE: 3543670) /DB_XREF = gi: 12804742 /UG = Hs.240615 hypothetical protein FLJ13556 similar to N-myc downstream regulated 3 |
| 207842_s_at | gb: NM_007359.1 /DEF = Homo sapiens MLN51 protein (MLN51), mRNA. /FEA = mRNA /GEN = MLN51 /PROD = MLN51 protein |
| | /DB_XREF = gi: 6678887 /UG = Hs.83422 MLN51 protein /FL = gb: NM_007359.1 |
| 217788_s_at | gb: NM_004481.2 /DEF_Homo sapiens UDP-N-acetyl-alpha-D-galactosamine: polypeptide N-acetylgalactosaminyltransferase 2 |
| | (GalNAc-T2) (GALNT2), mRNA. /FEA = mRNA /GEN = GALNT2 /PROD = polypeptide N-acetylgalactosaminyltransferase 2 |
| | /DB_XREF = gi: 9945385 /UG = Hs.130181 UDP-N-acetyl-alpha-D-galactosamine: polypeptide N-acetylgalactosaminyltransferase 2 |
| | (GalNAc-T2) /FL = gb: NM_004481.2 |
| 48612_at | Cluster Incl. AA225490: nc25b01.r1 Homo sapiens cDNA /clone = IMAGE-1009129 /gb = AA225490 /gi = 1846817 /ug = Hs.80115 /len = 539 |
| 212689_s_at | Consensus includes gb: AA524505 /FEA = EST /DB_XREF = gi: 2265433 /DB_XREF = est: ng43g12.s1 /CLONE = IMAGE: 937606 |
| | /UG = Hs.321707 KIAA0742 protein |
| 222047_s_at | Consensus includes gb: AI523895 /FEA = EST /DB_XREF = gi: 4438030 /DB_XREF = est: tg97g03.x1 /CLONE = IMAGE: 2116756 |
| | /UG = Hs.111801 arsenate resistance protein ARS2 |
| 201666_at | gb: NM_003254.1 /DEF = Homo sapiens tissue inhibitor of metalloproteinase 1 (erythroid potentiating activity, collagenase |
| | inhibitor) (TIMP1), mRNA. /FEA = mRNA /GEN = TIMP1 /PROD = tissue inhibitor of metalloproteinase 1precursor |
| | /DB_XREF = gi: 4507508 /UG = Hs.5831 tissue inhibitor of metalloproteinase 1 (erythroid potentiating activity, collagenase |
| | inhibitor) /FL = gb: BC000866.1 gb: M12670.1 gb: M59906.1 gb: NM_003254.1 |
| 203218_at | Consensus includes gb: W37431 /FEA = EST /DB_XREF = gi: 1319025 /DB_XREF = est: zc11f11.s1 /CLONE = IMAGE: 322029 |
| | /UG = Hs.246857 mitogen-activated protein kinase 9 /FL = gb: U34821.1 gb: NM_002752.1 gb: L31951.1 gb: U09759.1 |
| 213118_at | Consensus includes gb: AL136821.1 /DEF = Homo sapiens mRNA; cDNA DKFZp434M1526 (from clone DKFZp434M1526). /FEA = mRNA |
| | /GEN = DKFZp434M1526 /PROD = hypothetical protein /DB_XREF = gi: 12053152 /UG = Hs.153293 KIAA0701 protein |
| 201057_s_at | gb: NM_004487.1 /DEF = Homo sapiens golgi autoantigen, golgin subfamily b, macrogolgin (with transmembrane signal), 1 (GOLGB1), |
| | mRNA. /FEA = mRNA /GEN = GOLGB1 /PROD = golgi autoantigen, golgin subfamily b, macrogolgin (with transmembrane signal), 1 |
| | /DB_XREF = gi: 4758453 /UG = Hs.7844 golgi autoantigen, golgin subfamily b, macrogolgin (with transmembrane signal), 1 /FL = gb: NM_004487.1 |
| 217738_at | Consensus includes gb: BF575514 /FEA = EST /DB_XREF = gi: 11649318 /DB_XREF = est: 602133090F1 /CLONE = IMAGE: 4288079 |
| | /UG = Hs.239138 pre-B-cell colony-enhancing factor /FL = gb: U02020.1 gb: NM_005746.1 |
| 208648_at | Consensus includes gb: W60953 /FEA = EST /DB_XREF = gi: 1367731 /DB_XREF = est: zc98b12.s1 /CLONE = IMAGE: 339167 |
| | /UG = Hs.106357 valosin-containing protein /FL = gb: AF100752.1 gb: NM_007126.2 |
| 212242_at | Consensus includes gb: AL565074 /FEA = EST /DB_XREF = gi: 12916087 /DB_XREF = est: AL565074 /CLONE = CS0DN003YF20 (3 prime) |
| | /UG = Hs.75318 tubulin, alpha 1 (testis specific) |
| 203897_at | Consensus includes gb: BE963444 /FEA = EST /DB_XREF = gi: 11766863 /DB_XREF = est: 601657224R1 /CLONE = IMAGE: 3866357 |
| | /UG = Hs.28607 hypothetical protein A-211C6.1 /FL = gb: NM_020424.1 |
| 209513_s_at | gb: BC004331.1 /DEF = Homo sapiens, Similar to RIKEN cDNA 2610207I16 gene, clone MGC: 10940, mRNA, complete cds. |
| | /FEA = mRNA /PROD = Similar to RIKEN cDNA 2610207I16 gene /DB_XREF = gi: 13279253 /UG = Hs.47986 Homo sapiens, |
| | Similar to RIKEN cDNA 2610207I16 gene, clone MGC: 10940, mRNA, complete cds /FL = gb: BC004331.1 |
| 211762_s_at | gb: BC005978.1 /DEF = Homo sapiens, karyopherin alpha 2 (RAG cohort 1, importin alpha 1), clone MGC: 14668, mRNA, complete cds. |
| | /FEA = mRNA /PROD = karyopherin alpha 2 (RAG cohort 1, importinalpha 1) /DB_XREF = gi: 13543656 /FL = gb: BC005978.1 |
| 219639_x_at | gb: NM_020213.1 /DEF = Homo sapiens hypothetical protein from EUROIMAGE 1977056 (LOC56965), mRNA. /FEA = mRNA |
| | /GEN = LOC56965 /PROD = hypothetical protein from EUROIMAGE 1977056 /DB_XREF = gi: 9910373 /UG = Hs.315687 hypothetical protein |
| | from EUROIMAGE 1977056 /FL = gb: NM_020213.1 |
| 217882_at | gb: NM_018447.1 /DEF = Homo sapiens 30 kDa protein (LOC55831), mRNA. /FEA = mRNA /GEN = LOC55831 /PROD = 30 kDa protein |
| | /DB_XREF = gi: 8923856 /UG = Hs.283714 30 kDa protein /FL = gb: AF157321.1 gb: NM_018447.1 |
| 204714_s_at | gb: NM_000130.2 /DEF = Homo sapiens coagulation factor V (proaccelerin, labile factor) (F5), mRNA. /FEA = mRNA /GEN = F5 |
| | /PROD = coagulation factor V precursor /DB_XREF = gi: 10518500 /UG = Hs.30054 coagulation factor V (proaccelerin, labile factor) |
| | /FL = gb: NM_000130.2 gb: M16967.1 gb: M14335.1 |
| 221547_at | gb: BC000794.1 /DEF = Homo sapiens, pre-mRNA splicing factor similar to S. cerevisiae Prp18, clone MGC: 5075, mRNA, |
| | complete cds. /FEA = mRNA /PROD = pre-mRNA splicing factor similar to S. cerevisiae Prp18 /DB_XREF = gi: 12653992 |
| | /UG = Hs.155244 pre-mRNA processing factor 18 /FL = gb: BC000794.1 gb: U51990.1 gb: NM_003675.1 |
| 210793_s_at | gb: U41815.1 /DEF = Human nucleoporin 98 (NUP98) mRNA, complete cds. /FEA = mRNA /GEN = NUP98 /PROD = nucleoporin 98 |
| | /DB_XREF = gi: 1184172 /UG = Hs.112255 nucleoporin 98 kD /FL = gb: U41815.1 |
| 206207_at | gb: NM_001828.3 /DEF = Homo sapiens Charot-Leyden crystal protein (CLC), mRNA. /FEA = mRNA /GEN = CLC |
| | /PROD = Charot-Leyden crystal protein /DB_XREF = gi: 6325464 /UG = Hs.889 Charot-Leyden crystal protein /FL = gb: L01664.1 gb: NM_001828.3 |
| 202595_s_at | gb: AF161461.1 /DEF = Homo sapiens HSPC112 mRNA, complete cds. /FEA = mRNA /PROD = HSPC112 /DB_XREF = gi: 6841445 |
| | /UG = Hs.11000 leptin receptor overlapping transcript-like 1 /FL = gb: BC000642.1 gb: AF063605.1 gb: AF161461.1 gb: NM_015344.1 |
| 219434_at | gb: NM_018643.1 /DEF = Homo sapiens triggering receptor expressed on myeloid cells 1 (TREM1), mRNA. /FEA = mRNA /GEN = TREM1 |
| | /PROD = triggering receptor expressed on myeloid cells1 /DB_XREF = gi: 8924261 /UG = Hs.283022 triggering receptor expressed on |
| | /myeloid cells 1 /FL = gb: AF196329.1 gb: NM_018643.1 gb: AF287008.1 |
| 205020_s_at | gb: NM_005738.1 /DEF = Homo sapiens ADP-ribosylation factor-like 4 (ARL4), mRNA. /FEA = mRNA /GEN = ARL4 |
| | /PROD = ADP-ribosylation factor-like 4 /DB_XREF = gi: 5031602 /UG = Hs.201672 ADP-ribosylation factor-like 4 /FL = gb: U73960.1 gb: NM_005738.1 |
| 212052_s_at | Consensus includes gb: AB014576.1 /DEF = Homo sapiens mRNA for KIAA0676 protein, partial cds. /FEA = mRNA |
| | /GEN = KIAA0676 /PROD = KIAA0676 protein /DB_XREF = gi: 3327165 /UG = Hs.155829 KIAA0676 protein |
| 202565_s_at | gb: NM_003174.2 /DEF = Homo sapiens supervillin (SVIL), transcript variant 1, mRNA. /FEA = mRNA /GEN = SVIL |
| | /PROD = supervillin, isoform 1 /DB_XREF = gi: 11496980 /UG = Hs.154567 supervillin /FL = gb: NM_003174.2 gb: AF051850.1 gb: AF051851.1 |
| 221524_s_at | gb: AF272036.1 /DEF = Homo sapiens Rag D mRNA, complete cds. /FEA = mRNA /PROD = Rag D /DB_XREF = gi: 11181619 |
| | /UG = Hs.238679 Rag D protein /FL = gb: NM_021244.1 gb: AF272036.1 gb: BC003088.1 |
| 218037_at | gb: NM_024293.1 /DEF = Homo sapiens hypothetical protein MGC3035 (MGC3035), mRNA. /FEA = mRNA /GEN = MGC3035 |
| | /PROD = hypothetical protein MGC3035 /DB_XREF = gi: 13236511 /UG = Hs.22412 hypothetical protein MGC3035 /FL = gb: AL136758.1 gb: BC002420.1 gb: NM_024293.1 |
| 214107_x_at | Consensus includes gb: AW340850 /FEA = EST /DB_XREF = gi: 6837476 /DB_XREF = est: xr31f01.x1 /CLONE = IMAGE: 2761753 /UG = Hs.326350 |
| | Homo sapiens cDNA FLJ11822 fis, clone HEMBA1006485, highly similar to PUROMYCIN-SENSITIVE AMINOPEPTIDASE (EC 3.4.11.—) |
| 210184_at | gb: M81695.1 /DEF = H. sapiens leukocyte adhesion glycoprotein p150, 95 mRNA, complete cds. /FEA = mRNA /GEN = ITGAX |
| | /DB_XREF = gi: 487829 /UG = Hs.51077 integrin, alpha X (antigen CD11C (p150), alpha polypeptide) /FL = gb: M81695.1 gb: NM_000887.2 |
| 209107_x_at | gb: U19179.1 /DEF = Human (Hin-2) mRNA, complete cds. /FEA = mRNA /GEN = Hin-2 /DB_XREF = gi: 726037 |
| | /UG = Hs.74002 nuclear receptor coactivator 1 /FL = gb: U19179.1 |
| 208749_x_at | gb: AF085357.1 /DEF = Homo sapiens flotillin mRNA, complete cds. /FEA = mRNA /PROD = flotillin /DB_XREF = gi: 5114048 |
| | /UG = Hs.179986 flotillin 1 /FL = gb: BC001146.1 gb: AF085357.1 gb: AF089750.2 gb: NM_005803.2 |
| 212561_at | Consensus includes gb: AA349595 /FEA = EST /DB_XREF = gi: 2001934 /DB_XREF = est: EST56429 /UG = Hs.26797 KIAA1091 protein |
| 212470_at | Consensus includes gb: AB011088.1 /DEF = Homo sapiens mRNA for KIAA0516 protein, partial cds. /FEA = mRNA /GEN = KIAA0516 |
| | /PROD = KIAA0516 protein /DB_XREF = gi: 3043555 /UG = Hs.129872 sperm associated antigen 9 |
| 214937_x_at | Consensus includes gb: AI924817 /FEA = EST /DB_XREF = gi: 5660781 /DB_XREF = est: wn23c11.x1 /CLONE = IMAGE: 2446292 |
| | /UG = Hs.75737 pericentriolar material 1 |
| 201783_s_at | gb: NM_021975.1 /DEF = Homo sapiens v-rel avian reticuloendotheliosis viral oncogene homolog A (nuclear factor of |
| | kappa light polypeptide gene enhancer in B-cells 3 (p65)) (RELA), mRNA. /FEA = mRNA /GEN = RELA |
| | /PROD = v-rel avian reticuloendotheliosis viral oncogenehomolog A (nuclear factor of kappa light polypeptide geneenhancer in |
| | B-cells 3 (p65)) /DB_XREF = gi: 11496238 /UG = Hs.75569 v-rel avian reticuloendotheliosis viral oncogene homolog A |
| | (nuclear factor of kappa light polypeptide gene enhancer in B-cells 3 (p65)) /FL = gb: NM_021975.1 gb: L19067.1 |
| 202010_s_at | gb: NM_021188.1 /DEF = Homo sapiens clones 23667 and 23775 zinc finger protein (LOC57862), mRNA. /FEA = mRNA /GEN = LOC57862 /PROD = clones 23667 and |
| | 23775 zinc finger protein /DB_XREF = gi: 10863994 /UG = Hs.7137 clones 23667 and 23775 zinc finger protein /FL = gb: NM_021188.1 gb: U90919.1 |
| 201601_x_at | gb: NM_003641.1 /DEF = Homo sapiens interferon induced transmembrane protein 1 (9-27) (IFITM1), mRNA. /FEA = mRNA /GEN = IFITM1 |
| | /PROD = interferon induced transmembrane protein 1(9-27) /DB_XREF = gi: 4504580 /UG = Hs.146360 interferon induced transmembrane |
| | protein 1 (9-27) /FL = gb: BC000897.1 gb: J04164.1 gb: NM_003641.1 |
| 203266_s_at | gb: NM_003010.1 /DEF_Homo sapiens mitogen-activated protein kinase kinase 4 (MAP2K4), mRNA. /FEA = mRNA /GEN = MAP2K4 /PROD = mitogen-activated protein |
| | kinase kinase 4 /DB_XREF = gi: 4506888 /UG = Hs.75217 mitogen-activated protein kinase kinase 4 /FL = gb: NM_003010.1 gb: L36870.1 gb: U17743.1 |
| 219863_at | gb: NM_016323.1 /DEF = Homo sapiens cyclin-E binding protein 1 (LOC51191), mRNA. /FEA = mRNA /GEN = LOC51191 |
| | /PROD = cyclin-E binding protein 1 /DB_XREF = gi: 7705930 /UG = Hs.26663 cyclin-E binding protein 1 /FL = gb: AB027289.1 gb: NM_016323.1 |
| 212322_at | Consensus includes gb: BE999972 /FEA = EST /DE_XREF = gi: 10700248 /DB_XREF = est: 7h15b02.x1 /CLONE = IMAGE: 3316011 |
| | /UG = Hs.186613 sphingosine-1-phosphate lyase 1 /FL = gb: AF144638.1 |
| 204206_at | gb: NM_020310.1 /DEF = Homo sapiens MAX binding protein (MNT), mRNA. /FEA = mRNA /GEN = MNT /PROD = MAX binding protein |
| | /DB_XREF = gi: 9945317 /UG = Hs.25497 MAX binding protein /FL = gb: NM_020310.1 |
| 203278_s_at | gb: NM_016621.1 /DEF = Homo sapiens hypothetical protein (LOC51317), mRNA. /FEA = mRNA /GEN = LOC51317 |
| | /PROD = hypothetical protein /DB_XREF = gi: 7706159 /UG = Hs.106826 KIAA1696 protein /FL = gb: AF208848.1 gb: NM_016621.1 |
| 206637_at | gb: NM_014879.1 /DEF = Homo sapiens KIAA0001 gene product; putative G-protein-coupled receptor; G protein coupled receptor |
| | for UDP-glucose (KIAA0001), mRNA. /FEA = mRNA /GEN = KIAA0001 /PROD = KIAA0001 gene product; putativeG-protein-coupled receptor; |
| | G protein coupled receptor forUDP-glucose /DB_XREF = gi: 7661847 /UG = Hs.2465 KIAA0001 gene product; putative G-protein-coupled |
| | receptor; G protein coupled receptor for UDP-glucose /FL = gb: D13626.1 gb: NM_014879.1 |
| 218924_s_at | gb: NM_004388.1 /DEF = Homo sapiens chitobiase, di-N-acetyl- (CTBS), mRNA. /FEA = mRNA /GEN = CTBS /PROD = chitobiase, |
| | di-N-acetyl- /DB_XREF = gi: 4758091 /UG = Hs.135578 chitobiase, di-N-acetyl- /FL = gb: M95767.1 gb: NM_004388.1 |

TABLE 3
Genes from Cluster Analysis 3

| Affymetrix | |
|---|---|
| internal | |
| designation | Description of the sequence in the GeneBank data base |
| 36711_at | Cluster Incl. AL021977: bK447C4.1 (novel MAFF (v-maf musculoaponeurotic fibrosarcoma (avian) oncogene family, protein F) |
| | protein) /cds = (0, 494) /gb = AL021977 /gi = 4914526 /ug = Hs.51305 /len = 2128 |
| 210845_s_at | gb: U08839.1 /DEF = Human urokinase-type plasminogen activator receptor mRNA, complete cds. /FEA = mRNA /PROD = urokinase-type |
| | plasminogen activator receptor /DB_XREF = gi: 517197 /UG = Hs.179657 plasminogen activator, urokinase receptor /FL = gb: U08839.1 |
| 202643_s_at | Consensus includes gb: AI738896 /FEA = EST /DB_XREF = gi: 5100877 /DB_XREF = est: wi22g02.x1 /CLONE = IMAGE: 2391026 /UG = Hs.211600 tumor |
| | necrosis factor, alpha-induced protein 3 /FL = gb: M59465.1 gb: NM_006290.1 |
| 205476_at | gb: NM_004591.1 /DEF = Homo sapiens small inducible cytokine subfamily A (Cys-Cys), member 20 (SCYA20), mRNA. /FEA = mRNA |
| | /GEN = SCYA20 /PROD = small inducible cytokine subfamily A (Cys-Cys), member 20 /DB_XREF = gi: 4759075 /UG = Hs.75498 small inducible |
| | cytokine subfamily A (Cys-Cys), member 20 /FL = gb: U64197.1 gb: U77035.1 gb: D86955.1 gb: NM_004591.1 |
| 205419_at | gb: NM_004951.1 /DEF = Homo sapiens Epstein-Barr virus induced gene 2 (lymphocyte-specific G protein-coupled receptor) (EBI2), |
| | mRNA. /FEA = mRNA /GEN = EBI2 /PROD = Epstein-Barr virus induced gene 2(lymphocyte-specific G protein-coupled receptor) |
| | /DB_XREF = gi: 4826705 /UG = Hs.784 Epstein-Barr virus induced gene 2 (lymphocyte-specific G protein-coupled receptor) |
| | /FL = gb: L08177.1 gb: NM_004951.1 |
| 209795_at | gb: L07555.1 /DEF = Homo sapiens early activation antigen CD69 mRNA, complete cds. /FEA = mRNA /PROD = early activation antigen CD69 |
| | /DB_XREF = gi: 291897 /UG = Hs.82401 CD69 antigen (p60, early T-cell activation antigen) /FL = gb: L07555.1 gb: NM_001781.1 |
| 205767_at | gb: NM_001432.1 /DEF = Homo sapiens epiregulin (EREG), mRNA. /FEA = mRNA /GEN = EREG /PROD = epiregulin precursor /DB_XREF = gi: 4557566 |
| | /UG = Hs.115263 epiregulin /FL = gb: D30783.1 gb: NM_001432.1 |
| 203821_at | gb: NM_001945.1 /DEF = Homo sapiens diphtheria toxin receptor (heparin-binding epidermal growth factor-like growth factor) (DTR), |
| | mRNA. /FEA = mRNA /GEN = DTR /PROD = diphtheria toxin receptor (heparin-bindingepidermal growth factor-like growth factor) |
| | /DB_XREF = gi: 4503412 /UG = Hs.799 diphtheria toxin receptor (heparin-binding epidermal growth factor-like growth factor) |
| | /FL = gb: M60278.1 gb: NM_001945.1 |
| 211924_s_at | gb: AY029180.1 /DEF = Homo sapiens soluble urokinase plasminogen activator receptor precursor (SUPAR) mRNA, complete cds. |
| | /FEA = CDS /GEN = SUPAR /PROD = soluble urokinase plasminogen activator receptorprecursor /DB_XREF = gi: 13641308 /FL = gb: AY029180.1 |
| 205403_at | gb: NM_004633.1 /DEF = Homo sapiens interleukin 1 receptor, type II (IL1R2), mRNA. /FEA = mRNA /GEN = IL1R2 |
| | /PROD = interleukin 1 receptor, type II /DB_XREF = gi: 4758597 /UG = Hs.25333 interleukin 1 receptor, type II |
| | /FL = gb: U74649.1 gb: NM_004633.1 |
| 204351_at | gb: NM_005980.1 /DEF = Homo sapiens S100 calcium-binding protein P (S100P), mRNA. /FEA = mRNA /GEN = S100P /PROD = S100 calcium-binding |
| | protein P /DB_XREF = gi: 5174662 /UG = Hs.2962 S100 calcium-binding protein P /FL = gb: NM_005980.1 |
| 206115_at | gb: NM_004430.1 /DEF = Homo sapiens early growth response 3 (EGR3), mRNA. /FEA = mRNA /GEN = EGR3 /PROD = early growth response 3 |
| | /DB_XREF = gi: 4758251 /UG = Hs.74088 early growth response 3 /FL = gb: NM_004430.1 |
| 204103_at | gb: NM_002984.1 /DEF = Homo sapiens small inducible cytokine A4 (homologous to mouse Mip-1b) (SCYA4), mRNA. /FEA = mRNA |
| | /GEN = SCYA4 /PROD = small inducible cytokine A4 (homologous to mouseMip-1b) /DB_XREF = gi: 4506844 /UG = Hs.75703 small inducible |
| | cytokine A4 (homologous to mouse Mip-1b) /FL = gb: J04130.1 gb: NM_002984.1 gb: M23502.1 gb: M25316.1 |
| 206522_at | gb: NM_004668.1 /DEF = Homo sapiens maltase-glucoamylase (alpha-glucosidase) (MGAM), mRNA. /FEA = mRNA /GEN = MGAM |
| | /PROD = alpha-glucosidase /DB_XREF = gi: 4758711 /UG = Hs.122785 maltase-glucoamylase (alpha-glucosidase) |
| | /FL = gb: AF016833.1 gb: NM_004668.1 |
| 202147_s_at | gb: NM_001550.1 /DEF = Homo sapiens interferon-related developmental regulator 1 (IFRD1), mRNA. /FEA = mRNA /GEN = IFRD1 |
| | /PROD = interferon-related developmental regulator 1 /DB_XREF = gi: 4504606 /UG = Hs.7879 interferon-related developmental |
| | regulator 1 /FL = gb: BC001272.1 gb: NM_001550.1 |
| 206515_at | gb: NM_000896.1 /DEF = Homo sapiens cytochrome P450, subfamily IVF, polypeptide 3 (leukotriene B4 omega hydroxylase) (CYP4F3), |
| | mRNA. /FEA = mRNA /GEN = CYP4F3 /PROD = cytochrome P450, subfamily IVF, polypeptide 3 /DB_XREF = gi: 4503240 /UG = Hs.106242 |
| | cytochrome P450, subfamily IVF, polypeptide 3 (leukotriene B4 omega hydroxylase) /FL = gb: AB002454.1 gb: D12620.1 gb: NM_000896.1 |
| 204614_at | gb: NM_002575.1 /DEF = Homo sapiens serine (or cysteine) proteinase inhibitor, clade B (ovalbumin), member 2 (SERPIMB2), mRNA. |
| | /FEA = mRNA /GEN = SERPINB2 /PROD = serine (or cysteine) proteinase inhibitor, cladeB (ovalbumin), member 2 /DB_XREF = gi: 4505594 |
| | /UG = Hs.75716 serine (or cysteine) proteinase inhibitor, clade B (ovalbumin), member 2 /FL = gb: J02685.1 gb: J03603.1 |
| | gb: M18082.1 gb: NM_002575.1 |
| 209959_at | gb: U12767.1 /DEF = Human mitogen induced nuclear orphan receptor (MINOR) mRNA, complete cds. /FEA = mRNA /GEN = MINOR |
| | /PROD = mitogen induced nuclear orphan receptor /DB_XREF = gi: 924281 /UG = Hs.80561 nuclear receptor subfamily 4, group A, |
| | member 3 /FL = gb: U12767.1 |
| 215078_at | Consensus includes gb: AL050388.1 /DEF = Homo sapiens mRNA; cDNA DKFZp564M2422 (from clone DKFZp564M2422); partial cds. |
| | /FEA = mRNA /GEN = DKFZp564M2422 /PROD = hypothetical protein /DB_XREF = gi: 4914612 /UG = Hs.306320 Homo sapiens mRNA; cDNA DKFZp564M2422 (from clone DKFZp564M2422); |
| | partial cds |
| 201890_at | Consensus includes gb: BE966236 /FEA = EST /DB_XREF = gi: 11771437 /DB_XREF = est: 601660172R1 /CLONE = IMAGE: 3905920 /UG = Hs.75319 |
| | ribonucleotide reductase M2 polypeptide /FL = gb: NM_001034.1 |
| 201489_at | gb: BC005020.1 /DEF = Homo sapiens, peptidylprolyl isomerase F (cyclophilin F), clone MGC: 11022, mRNA, complete cds. /FEA = mRNA |
| | /PROD = peptidylprolyl isomerase F (cyclophilin F) /DB_XREF = gi: 13477126 /UG = Hs.173125 peptidylprolyl isomerase F (cyclophilin F) |
| | /FL = gb: BC005020.1 gb: M80254.1 gb: NM_005729.1 |
| 203888_at | gb: NM_000361.1 /DEF = Homo sapiens thrombomodulin (THBD), mRNA. /FEA = mRNA /GEN = THBD /PROD = thrombomodulin /DB_XREF = gi: 4507482 |
| | /UG = Hs.2030 thrombomodulin /FL = gb: M16552.1 gb: NM_000361.1 |
| 218723_s_at | gb: NM_014059.1 /DEF = Homo sapiens RGC32 protein (RGC32), mRNA. /FEA = mRNA /GEN = RGC32 /PROD = RGC32 protein /DB_XREF = gi: 7662650 |
| | /UG = Hs.76640 RGC32 protein /FL = gb: AF036549.1 gb: NM_014059.1 |
| 206834_at | gb: NM_000519.2 /DEF = Homo sapiens hemoglobin, delta (HBD), mRNA. /FEA = mRNA /GEN = HBD /PROD = hemoglobin, delta /DB_XREF = gi: 6633803 |
| | /UG = Hs.36977 hemoglobin, delta /FL = gb: NM_000519.2 |
| 205479_s_at | gb: NM_002658.1 /DEF = Homo sapiens plasminogen activator, urokinase (PLAU), mRNA. /FEA = mRNA /GEN = PLAU /PROD = plasminogen activator, |
| | urokinase /DB_XREF = gi: 4505862 /UG = Hs.77274 plasminogen activator, urokinase /FL = gb: M15476.1 gb: NM_002658.1 |
| 202912_at | gb: NM_001124.1 /DEF = Homo sapiens adrenomedullin (ADM), mRNA. /FEA = mRNA /GEN = ADM /PROD = adrenomedullin /DB_XREF = gi: 4501944 |
| | /UG = Hs.394 adrenomedullin /FL = gb: NM_001124.1 gb: D14874.1 |
| 208869_s_at | gb: AF087847.1 /DEF = Homo sapiens GABA-A receptor-associated protein like 1 (GABARAPL1) mRNA, complete cds. /FEA = mRNA |
| | /GEN = GABARAPL1 /PROD = GABA-A receptor-associated protein like 1 /DB_XREF = gi: 13375570 /UG = Hs.282654 Homo sapiens mRNA; |
| | cDNA DKFZp564N1272 (from clone DKFZp564N1272); complete cds /FL = gb: AL136676.1 gb: AF087847.1 |
| 202193_at | gb: NM_005569.2 /DEF = Homo sapiens LIM domain kinase 2 (LIMK2), transcript variant 2a, mRNA. /FEA = mRNA /GEN = LIMK2 |
| | /PROD = LIM domain kinase 2 isoform 2a /DB_XREF = gi: 8051619 /UG = Hs.278027 LIM domain kinase 2 /FL = gb: D45906.1 gb: NM_005569.2 |
| 214696_at | Consensus includes gb: AF070569.1 /DEF = Homo sapiens clone 24659 mRNA sequence. /FEA = mRNA /DB_XREF = gi: 3387938 /UG = Hs.29206 |
| | Homo sapiens clone 24659 mRNA sequence |
| 38037_at | Cluster Incl. M60278: Human heparin-binding EGF-like growth factor mRNA, complete cds /cds = (261,887) /gb = M60278 /gi = 183866 |
| | /ug = Hs.799 /len = 2342 |
| 212723_at | Consensus includes gb: AK021780.1 /DEF = Homo sapiens cDNA FLJ11718 fis, clone HEMBA1005252, highly similar to Homo sapiens mRNA |
| | for KIAA0585 protein. /FEA = mRNA /DB_XREF = gi: 10433034 /UG = Hs.72660 phosphatidylserine receptor |
| 207802_at | gb: NM_006061.1 /DEF = Homo sapiens specific granule protein (28 kDa); cysteine-rich secretory protein-3 (SGP28), mRNA. /FEA = mRNA |
| | /GEN = SGP28 /PROD = specific granule protein (28 kDa); cysteine-richsecretory protein-3 /DB_XREF = gi: 5174674 /UG = Hs.54431 specific |
| | granule protein (28 kDa); cysteine-rich secretory protein-3 /FL = gb: NM_006061.1 |
| 209967_s_at | gb: D14826.1 /DEF = Human mRNA for hCREM (cyclic AMP-responsive element modulator) type 2 protein, complete cds. /FEA = mRNA |
| | /GEN = hCREM-2; hCREM-2; hCREM-2; hCREM-2 /PROD = hCREM 2beta-b protein; hCREM 2beta-a protein; hCREM 2alpha-b protein; |
| | hCREM 2alpha-a protein /DB_XREF = gi: 532036 /UG = Hs.155924 cAMP responsive element modulator /FL = gb: AF069065.1 gb: D14826.1 |
| 210119_at | gb: U73191.1 /DEF = Human inward rectifier potassium channel (Kir1.3), complete cds. /FEA = mRNA /GEN = Kir1.3 /PROD = inward rectifier |
| | potassium channel /DB_XREF = gi: 1765984 /UG = Hs.17287 potassium inwardly-rectifying channel, subfamily J, member 15 |
| | /FL = gb: U73191.1 gb: NM_002243.1 |
| 203435_s_at | gb: NM_007287.1 /DEF = Homo sapiens membrane metallo-endopeptidase (neutral endopeptidase, enkephalinase, CALLA, CD10) (MME), |
| | transcript variant lbis, mRNA. /FEA = mRNA /GEN = MME /PROD = membrane metallo-endopeptidase /DB_XREF = gi: 6042199 /UG = Hs.1298 |
| | membrane metallo-endopeptidase (neutral endopeptidase, enkephalinase, CALLA, CD10) /FL = gb: J03779.1 gb: NM_007287.1 gb: NM_007288.1 |
| 213515_x_at | Consensus includes gb: AI133353 /FEA = EST /DB_XREF = gi: 6360669 /DB_XREF = est: HA1957 /UG = Hs.298161 myosin, light polypeptide 4, |
| | alkali; atrial, embryonic |
| 209545_s_at | gb: AF064824.1 /DEF = Homo sapiens CARD-containing ICE associated kinase mRNA, complete cds. /FEA = mRNA /PROD = CARD-containing ICE |
| | associated kinase /DB_XREF = gi: 3290171 /UG = Hs.103755 receptor-interacting serine-threonine kinase 2 /FL = gb: BC004553.1 |
| | gb: AF027706.1 gb: AF064824.1 gb: AF078530.1 gb: NM_003821.1 |
| 212531_at | Consensus includes gb: NM_005564.1 /DEF = Homo sapiens lipocalin 2 (oncogene 24p3) (LCN2), mRNA. /FEA = CDS /GEN = LCN2 |
| | /PROD = lipocalin 2 (oncogene 24p3) /DB_XREF = gi: 5031852 /UG = Hs.204238 lipocalin 2 (oncogene 24p3) /FL = gb: NM_005564.1 |
| 208470_s_at | gb: NM_020995.1 /DEF = Homo sapiens haptoglobin-related protein (HPR), mRNA. /FEA = CDS /GEN = HPR /PROD = haptoglobin-related protein |
| | /DB_XREF = gi: 10337588 /UG = Hs.328822 haptoglobin-related protein /FL = gb: NM_020995.1 |
| 200773_s_at | gb: U48296.1 /DEF = Homo sapiens protein tyrosine phosphatase PTPCAAX1 (hPTPCAAX1) mRNA, complete cds. /FEA = mRNA /GEN = hPTPCAAX1 |
| | /PROD = protein tyrosine phosphatase PTPCAAX1 /DB_XREF = gi: 1777754 /UG = Hs.227777 protein tyrosine phosphatase type IVA, member 1 |
| | /FL = gb: U48296.1 gb: NM_003463.1 |
| 211372_s_at | gb: U64094.1 /DEF = Human soluble type II interleukin-1 receptor mRNA, complete cds. /FEA = mRNA /PROD = soluble type II interleukin-1 |
| | receptor /DB_XREF = gi: 1488065 /UG = Hs.25333 interleukin 1 receptor, type II /FL = gb: U64094.1 |
| 204794_at | gb: NM_004418.2 /DEF = Homo sapiens dual specificity phosphatase 2 (DUSP2), mRNA. /FEA = mRNA /GEN = DUSP2 /PROD = dual specificity |
| | phosphatase 2 /DB_XREF = gi: 12707563 /UG = Hs.1183 dual specificity phosphatase 2 /FL = gb: NM_004418.2 gb: L11329.1 |
| 206177_s_at | gb: NM_000045.2 /DEF = Homo sapiens arginase, liver (ARG1), mRNA. /FEA = mRNA /GEN = ARG1 /PROD = arginase, type I /DB_XREF = gi: 10947138 |
| | /UG = Hs.289057 arginase, liver /FL = gb: NM_000045.2 gb: M14502.1 |
| 205239_at | gb: NM_001657.1 /DEF = Homo sapiens amphiregulin (schwannoma-derived growth factor) (AREG), mRNA. /FEA = mRNA /GEN = AREG |
| | /PROD = amphiregulin (schwannoma-derived growth factor) /DB_XREF = gi: 4502198 /UG = Hs.270833 amphiregulin (schwannoma-derived growth |
| | factor) /FL = gb: M30704.1 gb: NM_001657.1 |
| 207094_at | gb: NM_000634.1 /DEF = Homo sapiens interleukin 8 receptor, alpha (IL8RA), mRNA. /FEA = mRNA /GEN = IL8RA /PROD = interleukin 8 receptor, |
| | alpha /DB_XREF = gi: 4504680 /UG = Hs.194778 interleukin 8 receptor, alpha /FL = gb: M68932.1 gb: NM_000634.1 gb: L19591.1 |
| 200776_s_at | Consensus includes gb: AL518328 /FEA = EST /DB_XREF = gi: 12781821 /DB_XREF = est: AL518328 /CLONE = CS0DA009YK18 (3 prime) /UG = Hs.155291 |
| | KIAA0005 gene product /FL = gb: D13630.1 gb: NM_014670.1 |
| 215009_s_at | Consensus includes gb: U92014.1 /DEF = Human clone 121711 defective mariner transposon Hsmar2 mRNA sequence. /FEA = mRNA |
| | /DB_XREF = gi: 2052456 /UG = Hs.153527 Homo sapiens pTM5 mariner-like transposon mRNA, partial sequence |
| 211302_s_at | gb: L20966.1 /DEF = Human phosphodiesterase mRNA, complete cds. /FEA = mRNA /PROD = phosphodiesterase /DB_XREF = gi: 347121 |
| | /UG = Hs.188 phosphodiesterase 4B, cAMP-specific (dunce (Drosophila)-homolog phosphodiesterase E4) /FL = gb: L20966.1 |
| 210512_s_at | gb: AF022375.1 /DEF = Homo sapiens vascular endothelial growth factor mRNA, complete cds. /FEA = mRNA /PROD = vascular endothelial |
| | growth factor /DB_XREF = gi: 3719220 /UG = Hs.73793 vascular endothelial growth factor /FL = gb: M32977.1 gb: AF022375.1 |
| | gb: NM_003376.1 gb: AB021221.1 gb: AF091352.1 |
| 212577_at | Consensus includes gb: AA868754 /FEA = EST /DB_XREF = gi: 2964199 /DB_XREF = est: ak52e09.s1 /CLONE = IMAGE: 1409608 |
| | /UG = Hs.8118 KIAA0650 protein |
| 214866_at | Consensus includes gb: X74039.1 /DEF = H. sapiens mRNA for urokinase plasminogen activator receptor. /FEA = mRNA /PROD = urokinase |
| | plasminogen activator receptor /DB_XREF = gi: 456192 /UG = Hs.179657 plasminogen activator, urokinase receptor |
| 219228_at | gb: NM_018555.2 /DEF = Homo sapiens C2H2-like zinc finger protein (ZNF361), mRNA. /FEA = mRNA /GEN = ZNF361 /PROD = C2H2-like zinc finger |
| | protein /DB_XREF = gi: 10092612 /UG = Hs.147644 zinc finger protein 331 /FL = gb: AF251515.2 gb: NM_018555.2 gb: AE272148.1 |
| 201574_at | gb: NM_004730.1 /DEF = Homo sapiens eukaryotic translation termination factor 1 (ETF1), mRNA. /FEA = mRNA /GEN = ETF1 |
| | /PROD = eukaryotic translation termination factor 1 /DB_XREF = gi: 4759033 /UG = Hs.77324 eukaryotic translation termination factor 1 |
| | /FL = gb: U90176.1 gb: M75715.1 gb: NM_004730.1 |
| 209498_at | Consensus includes gb: X16354.1 /DEF = Human mRNA for transmembrane carcinoembryonic antigen BGPa (formerly TM1-CEA). |
| | /FEA = mRNA /PROD = TM1-CEA preprotein /DB_XREF = gi: 37197 /UG = Hs.50964 carcinoembryonic antigen-related cell adhesion molecule 1 |
| | (biliary glycoprotein) /FL = gb: J03858.1 |
| 207630_s_at | gb: NM_001881.1 /DEF = Homo sapiens cAMP responsive element modulator (CREM), mRNA. /FEA = mRNA /GEN = CREM /PROD = cAMP responsive |
| | element modulator /DB_XREF = gi: 4503038 /UG = Hs.155924 cAMP responsive element modulator /FL = gb: NM_001881.1 gb: S68271.1 |
| 210873_x_at | gb: U03891.2 /DEF = Homo sapiens phorbolin I mRNA, complete cds. /FEA = mRNA /PROD = phorbolin I /DB_XREF = gi: 4895107 /UG = Hs.226307 |
| | phorbolin (similar to apolipoprotein B mRNA editing protein) /FL = gb: U03891.2 |
| 204419_x_at | gb: NM_000184.1 /DEF = Homo sapiens hemoglobin, gamma G (HBG2), mRNA. /FEA = mRNA /GEN = HBG2 /PROD = hemoglobin, gamma G |
| | /DB_XREF = gi: 6715606 /UG = Hs.283108 hemoglobin, gamma G /FL = gb: NM_000184.1 |
| 202988_s_at | gb: NM_002922.1 /DEF = Homo sapiens regulator of G-protein signalling 1 (RGS1), mRNA. /FEA = mRNA /GEN = RGS1 /PROD = regulator of |
| | G-protein signalling 1 /DB_XREF = gi: 4506514 /UG = Hs.75256 regulator of G-protein signalling 1 /FL = gb: NM_002922.1 |
| 209273_s_at | Consensus includes gb: BG387555 /FEA = EST /DB_XREF = gi: 13281001 /DB_HREF = est: 602412371F1 /CLONE = IMAGE: 4521017 /UG = Hs.177776 |
| | hypothetical protein MGC4276 similar to CG8198 /FL = gb: AF284752.1 gb: BC002675.1 |
| 202693_s_at | Consensus includes gb: AW194730 /FEA = EST /DB_XREF = gi: 6473630 /DB_XREF = est: xn43d11.x1 /CLONE = IMAGE: 2696469 /UG = Hs.9075 |
| | serinethreonine kinase 17a (apoptosis-inducing) /FL = gb: AB011420.1 gb: NM_004760.1 |
| 205270_s_at | gb: NM_005565.2 /DEF = Homo sapiens lymphocyte cytosolic protein 2 (SH2 domain-containing leukocyte protein of 76 kD) (LCP2), mRNA. |
| | /FEA = mRNA /GEN = LCP2 /PROD = lymphocyte cytosolic protein 2 /DB_XREF = gi: 7382491 /UG = Hs.2488 lymphocyte cytosolic protein 2 |
| | (SH2 domain-containing leukocyte protein of 76 kD) /FL = gb: NM_005565.2 gb: U20158.1 |
| 202284_s_at | gb: NM_000389.1 /DEF = Homo sapiens cyclin-dependent kinase inhibitor 1A (p21, Cip1) (CDKN1A), mRNA. /FEA = mRNA /GEN = CDKN1A |
| | /PROD = cyclin-dependent kinase inhibitor 1A (p21, Cip1) /DB_XREF = gi: 11386202 /UG = Hs.179665 cyclin-dependent kinase inhibitor 1A |
| | (p21, Cip1) /FL = gb: NM_000389.1 gb: BC000275.1 gb: BC001935.1 gb: U03106.1 gb: L26165.1 gb: L25610.1 gb: U09579.1 |
| 219471_at | gb: NM_025113.1 /DEF = Homo sapiens hypothetical protein FLJ21562 (FLJ21562), mRNA. /FEA = mRNA /GEN = FLJ21562 /PROD = hypothetical |
| | protein FLJ21562 /DB_XREF = gi: 13376686 /UG = Hs.288708 hypothetical protein FLJ21562 /FL = gb: NM_025113.1 |
| 208651_x_at | gb: M58664.1 /DEF = Homo sapiens CD24 signal transducer mRNA, complete cds. /FEA = mRNA /PROD = signal transducer CD24 |
| | /DB_XREF = gi: 180167 /UG = Hs.286124 CD24 antigen (small cell lung carcinoma cluster 4 antigen) /FL = gb: M58664.1 gb: L33930.1 |
| | gb: NM_013230.1 |
| 211434_s_at | gb: AF015524.1 /DEF = Homo sapiens putative chemokine receptor (CRAM-A) mRNA, complete cds. /FEA = mRNA /GEN = CRAM-A |
| | /PROD = putative chemokine receptor /DB_XREF = gi: 3550066 /UG = Hs.302043 chemokine (C—C motif) receptor-like 2 /FL = gb: |
| | AF015524.1 |
| 204285_s_at | Consensus includes gb: AI857639 /FEA = EST /DB_XREF = gi: 5511255 /DB_XREF = est: wk95g09.x1 /CLONE = IMAGE: 2423200 /UG = Hs.96 |
| | phorbol-12-myristate-13-acetate-induced protein 1 /FL = gb: NM_021127.1 |
| 221824_s_at | consensus includes gb: AA770170 /FEA = EST /DB_XREF = gi: 2821408 /DB_XREF = est: ah84d09.s1 /CLONE = 1322321 /UG = Hs.288156 Homo sapiens |
| | cDNA: FLJ21819 fis, clone HEP01185 |
| 219081_at | gb: NM_024668.1 /DEF = Homo sapiens hypothetical protein FLJ20288 (FLJ20288), mRNA. /FEA = mRNA /GEN = FLJ20288 /PROD = hypothetical |
| | protein FLJ11979 /DB_XREF = gi: 13386461 /UG = Hs.84045 hypothetical protein FLJ20288 /FL = gb: BC004457.1 gb: NM_024668.1 |
| 220528_at | gb: NM_018399.1 /DEF = Homo sapiens VNN3 protein (HSA238982), mRNA. /FEA = mRNA /GEN = HSA238982 /PROD = VNN3 protein |
| | /DB_XREF = gi: 9055235 /UG = Hs.183656 VNN3 protein /FL = gb: NM_018399.1 |
| 205896_at | gb: NM_003059.1 /DEF = Homo sapiens solute carrier family 22 (organic cation transporter), member 4 (SLC22A4), mRNA. |
| | /FEA = mRNA /GEN = SLC22A4 /PROD = solute carrier family 22 (organic cationtransporter), member 4 /DB_XREF = gi: 4507002 |
| | /UG = Hs.77239 solute carrier family 22 (organic cation transporter), member 4 /FL = gb: AB007448.1 gb: NM_003059.1 |
| 211445_x_at | gb: AF315951.1 /DEF = Homo sapiens FKSG17 (FKSG17) mRNA, complete cds. /FEA = mRNA /GEN = FKSG17 /PROD = FKSG17 /DB_XREF = gi: 12276119 |
| | /UG = Hs.307057 Homo sapiens FKSG17 (FKSG17) mRNA, complete cds /FL = gb: AF315951.1 |
| 204286_s_at | gb: NM_021127.1 /DEF = Homo sapiens phorbol-12-myristate-13-acetate-induced protein 1 (PMAIP1), mRNA. /FEA = mRNA /GEN = PMAIP1 |
| | /PROD = phorbol-12-myristate-13-acetate-induced protein1 /DB_XREF = gi: 10863922 /UG = Hs.96 phorbol-12-myristate-13-acetate-induced |
| | protein 1 /FL = gb: NM_021127.1 |
| 202503_s_at | gb: NM_014736.1 /DEF = Homo sapiens KIAA0101 gene product. (KIAA0101), mRNA. /FEA = mRNA /GEN = KIAA0101 /PROD = KIAA0101 gene product |
| | DB_XREF = gi: 7661905 /UG = Hs.81892 KIAA0101 gene product /FL = gb: D14657.1 gb: NM_014736.1 |
| 211560_s_at | gb: AF130113.1 /DEF = Homo sapiens clone FLB8929 PRO2399 mRNA, complete cds. /FEA = mRNA /PROD = PRO2399 /DB_XREF = gi: 11493529 |
| | /UG = Hs.79103 cytochrome b5 outer mitochondrial membrane precursor /FL = gb: AF130113.1 |
| 209945_s_at | gb: BC000251.1 /DEF = Homo sapiens, Similar, to glycogen synthase kinase 3 beta, clone MGC: 1736, mRNA, complete cds. |
| | /FEA = mRNA /PROD = Similar to glycogen synthase kinase 3 beta /DB_XREF = gi: 12652980 /UG = Hs.78802 glycogen synthase kinase 3 beta |
| | /FL = gb: BC000251.1 |
| 217997_at | Consensus includes gb: AI795908 /FEA = EST /DB_XREF = gi: 5361371 /DB_XREF = est: wh40a05.x1 /CLONE = IMAGE: 2383184 /UG = Hs.82101 pleckstrin |
| | homology-like domain, family A, member 1 /FL = gb: NM_007350.1 |
| 203757_s_at | gb: BC005008.1 /DEF = Homo sapiens, carcinoembryonic antigen-related cell, adhesion molecule 6 (non-specific cross reacting antigen), |
| | clone MGC: 10467, mRNA, complete cds. /FEA = mRNA /PROD = carcinoembryonic antigen-related cell adhesionmolecule 6 |
| | (non-specific cross reacting antigen) /DB_XREF = gi: 13477106 /UG = Hs.73848 carcinoembryonic antigen-related cell |
| | adhesion molecule 6 (non-specific cross reacting antigen) /FL = gb: BC005008.1 gb: M18216.1 gb: M29541.1 gb: NM_002483.1 |
| 205269_at | Consensus includes gb: AI123251 /FEA = EST /DB_XREF = gi: 3539017 /DB_XREF = est: qa47g03.x1 /CLONE = IMAGE: 1689940 |
| | /UG = Hs.2488 lymphocyte cytosolic protein 2 (SH2 domain-containing leukocyte protein of 76 kD) /FL = gb: NM_005565.2 gb: U20158.1 |
| 219049_at | gb: NM_018371.1 /DEF = Homo sapiens hypothetical protein FLJ11264 (FLJ11264), mRNA. /FEA = mRNA /GEN = FLJ11264 |
| | /PROD = hypothetical protein FLJ11264 /DB_XREF = gi: 8922959 /UG = Hs.11260 hypothetical protein FLJ11264 /FL = gb: NM_018371.1 |
| 209396_s_at | gb: M80927.1 /DEF = Human glycoprotein mRNA, complete cds. /FEA = mRNA /PROD = glycoprotein /DB_XREF = gi: 348911 |
| | /UG = Hs.75184 chitinase 3-like 1 (cartilage glycoprotein-39) /FL = gb: M80927.1 gb: NM_001276.1 |
| 202637_s_at | Consensus includes gb: AI608725 /FEA = EST /DB_XREF = gi: 4617892 /DB_XREF = est: tw90b01.x1 /CLONE = IMAGE: 2266921 |
| | /UG = Hs.168383 intercellular adhesion molecule 1 (CD54), human rhinovirus receptor /FL = gb: M24283.1 gb: J03132.1 gb: NM_000201.1 |
| 205557_at | gb: NM_001725.1 /DEF = Homo sapiens bactericidalpermeability-increasing protein (BPI), mRNA. |
| | /FEA = mRNA /GEN = BPI /PROD = bactericidalpermeability-increasing proteinprecursor /DB_XREF = gi: 4502446 |
| | /UG = Hs.89535 bactericidalpermeability-increasing protein /FL = gb: AF322588.1 gb: J04739.1 gb: NM_001725.1 |
| 207072_at | gb: NM_003853.1 /DEF = Homo sapiens interleukin 18 receptor accessory protein (IL18RAP), mRNA. |
| | /FEA = mRNA /GEN = IL18RAP /PROD = interleukin 18 receptor accessory protein /DB_XREF = gi: 4504656 |
| | /UG = Hs.158315 interleukin 18 receptor accessory protein /FL = gb: AF077346.1 gb: NM_003853.1 |
| 202498_s_at | Consensus includes gb: BE550486 /FEA = EST /DB_XREF = gi: 9792178 /DB_XREF = est: 7a27c01.x1 /CLONE = IMAGE: 3219936 |
| | /UG = Hs.7594 solute carrier family 2 (facilitated glucose transporter), member 3 /FL = gb: M20681.1 gb: NM_006931.1 |
| 218739_at | gb: NM_016006.1 /DEF = Homo sapiens CGI-58 protein (LOC51099), mRNA. /FEA = mRNA /GEN = LOC51099 /PROD = CGI-58 protein |
| | /DB_XREF = gi: 7705770 /UG = Hs.19385 CGI-58 protein /FL = gb: AF151816.1 gb: NM_016006.1 |
| 212722_s_at | Consensus includes gb: AK021780.1 /DEF = Homo sapiens cDNA FLJ11718 fis, clone HEMBA1005252, highly similar |
| | to Homo sapiens mRNA for KIAA0585 protein. /FEA = mRNA /DB_XREF = gi: 10433034 /UG = Hs.72660 phosphatidylserine receptor |
| 212930_at | Consensus includes gb: AW576457 /FEA = EST /DB_XREF = gi: 7247996 /DB_XREF = est: UI-HF-BR0p-aji-a-01-0-UI.s1 |
| | /CLONE = IMAGE: 3074568 /UG = Hs.20952 Homo sapiens clone 24411 mRNA sequence |
| 200985_s_at | gb: NM_000611.1 /DEF = Homo sapiens CD59 antigen p18-20 (antigen identified by monoclonal antibodies 16.3A5, EJ16, |
| | EJ30, EL32 and G344) (CD59), mRNA. /FEA = mRNA /GEN = CD59 /PROD = CD59 antigen p18-20 (antigen identified |
| | by monoclonal antibodies 16.3A5, EJ16, EJ30, EL32 and G344) /DB_XREF = gi: 10835164 /UG = Hs.119663 CD59 antigen p18-20 |
| | (antigen identified by monoclonal antibodies 16.3A5, EJ16, EJ30, EL32 and G344) /FL = gb: NM_000611.1 gb: M34671.1 |
| 206026_s_at | gb: NM_007115.1 /DEF = Homo sapiens tumor necrosis factor, alpha-induced protein 6 (TNFAIP6), mRNA. /FEA = mRNA |
| | /GEN = TNFAIP6 /PROD = tumor necrosis factor, alpha-induced protein 6 /DB_XREF = gi: 6005905 |
| | /UG = Hs.29352 tumor necrosis factor, alpha-induced protein 6 /FL = gb: NM_007115.1. |
| 210740_s_at | gb: AF279372.1 /DEF = Homo sapiens inositol 1,3,4-trisphosphate 56-kinase mRNA, complete cds. /FEA = mRNA /PROD = inositol |
| | 1,3,4-trisphosphate 56-kinase /DB_XREF = gi: 12006345 /UG = Hs.6453 inositol 1,3,4-triphosphate 56 kinase /FL = gb: AF279372.1 |
| 202333_s_at | Consensus includes gb: AA877765 /FEA = EST /DB_XREF = gi: 2986730 /DB_XREF = est: nr06f05.s1 /CLONE = IMAGE: 1161057 /UG = Hs.811 |
| | ubiquitin-conjugating enzyme E2B (RAD6 homolog) /FL = gb: M74525.1 gb: NM_003337.1 |
| 201566_x_at | gb: D13891.1 /DEF = Human mRNA for Id-2H, complete cds. /FEA = mRNA /GEN = Id-2H /PROD = Id-2H /DB_XREF = gi: 464183 /UG = Hs.180919 |
| | inhibitor of DNA binding 2, dominant negative helix-loop-helix protein /FL = gb: M97796.1 gb: NM_002166.1 gb: D13891.1 |
| 44790_s_at | Cluster Incl. AI129310: qc48a05.x1 Homo sapiens cDNA, 3 end /clone = IMAGE-1712816 /clone_end = 3 /gb = AI129310 /gi = 3597824 |
| | /ug = Hs.234923 /len = 811 |
| 213836_s_at | Consensus includes gb: AW052084 /FEA = EST /DB_XREF = gi: 5914443 /DB_XREF = est: wy86f07.x1 /CLONE = IMAGE: 2555461 /UG = Hs.279937 KIAA1001 protein |
| 205027_s_at | gb: NM_005204.1 /DEF = Homo sapiens mitogen-activated protein kinase kinase kinase 8 (MAP3K8), mRNA. /FEA = mRNA /GEN = MAP3K8 |
| | /PROD = mitogen-activated protein kinase kinase kinase8 /DB_XREF = gi: 4885146 /UG = Hs.248 mitogen-activated protein kinase kinase |
| | kinase 8 /FL = gb: D14497.1 gb: NM_005204.1 |
| 207329_at | gb: NM_002424.1 /DEF = Homo sapiens matrix metalloproteinase 8 (neutrophil collagenase) (MMP8), mRNA. |
| | /FEA = mRNA /GEN = MMP8 /PROD = matrix metalloproteinase 8 preproprotein /DB_XREF = gi: 4505220 |
| | /UG = Hs.73862 matrix metalloproteinase 8 (neutrophil collagenase) /FL = gb: J05556.1 gb: NM_002424.1 |
| 217996_at | Consensus includes gb: AA576961 /FEA = EST /DB_XREF = gi: 2354435 /DB_XREF = est: nm82d08.s1 /CLONE = IMAGE: 1074735 /UG = Hs.82101 |
| | pleckstrin homology-like domain, family A, member 1 /FL = gb: NM_007350.1 |
| 208632_at | Consensus includes gb: AL578551 /FEA = EST /DB_XREF = gi: 12942733 /DB_XREF = est: AL578551 /CLONE = CS0DK001YG01 (3 prime) |
| | /UG = Hs.5094 ring finger protein 10 /FL = gb: AB027196.1 |
| 206851_at | gb: NM_002935.1 /DEF = Homo sapiens ribonuclease, RNase A family, 3 (eosinophil cationic protein) (RNASE3), mRNA. |
| | /FEA = mRNA /GEN = RNASE3 /PROD = ribonuclease, RNase A family, 3 (eosinophilcationic protein) /DB_XREF = gi: 4506550 |
| | /UG = Hs.73839 ribonuclease, RNase A family, 3 (eosinophil cationic protein) /FL = gb: NM_002935.1 gb: M28128.1 |
| 203434_s_at | Consensus includes gb: AI433463 /FEA = EST /DB_XREF = gi: 4289355 /DB_XREF = est: ti65g11.x1 /CLONE = IMAGE: 2136932 /UG = Hs.1298 membrane |
| | metallo-endopeptidase (neutral endopeptidase, enkephalinase, CALLA, CD10) /FL = gb: J03779.1 gb: NM_007287.1 gb: NM_007288.1 |
| 216236_s_at | Consensus includes gb: AL110298.1 /DEF = Homo sapiens mRNA; cDNA DKFZp564K1672 (from clone DKFZp564K1672); partial cds. /FEA = mRNA |
| | /GEN = DKFZp564K1672 /PROD = hypothetical protein /DB_XREF = gi: 5817258 /UG = Hs.7594 solute carrier family 2 (facilitated glucose |
| | transporter), member 3 |
| 206342_x_at | gb: NM_006123.1 /DEF = Homo sapiens iduronate 2-sulfatase (Hunter syndrome) (IDS), transcript variant 2, mRNA. /FEA = mRNA /GEN = IDS |
| | /PROD = iduronate-2-sulfatase isoform b precursor /DB_XREF = gi: 5360207 /UG = Hs.172458 iduronate 2-sulfatase (Hunter syndrome) |
| | /FL = gb: L40586.1 gb: NM_006123.1 |
| 201329_s_at | gb: NM_005239.1 /DEF = Homo sapiens v-ets avian erythroblastosis virus E26 oncogene homolog 2 (ETS2), mRNA. /FEA = mRNA /GEN = ETS2 |
| | /PROD-v-ets avian erythroblastosis virus E26 oncogenehomolog 2 /DB_XREF = gi: 4885220 /UG = Hs.85146 v-ets avian erythroblastosis virus |
| | E26 oncogene homolog 2 /FL = gb: J04102.1 gb: NM_005239.1 |
| 200731_s_at | Consensus includes gb: AW165960 /FEA = EST /DB_XREF = gi: 6397485 /DB_XREF = est: xf43a12.x1 /CLONE = IMAGE: 2620798 /UG = Hs.227777 protein |
| | tyrosine phosphatase type IVA, member 1 /FL = gb: U48296.1 gb: NM_003463.1 |
| 212508_at | Consensus includes gb: AK024029.1 /DEF = Homo sapiens cDNA FLJ13967 fis, clone Y79AA1001402, weakly similar to Homo sapiens |
| | paraneoplastic cancer-testis-brain antigen (MA4) mRNA. /FEA = mRNA /DB_XREF = gi: 10436287 /UG = Hs.24719 modulator of apoptosis 1 |
| | /FL = gb: AF305550.1 gb: NM_022151.1 |
| 208868_s_at | Consensus includes gb: BF125756 /FEA = EST /DB_XREF = gi: 10964796 /DB_XREF = est: 601763146F1 /CLONE = IMAGE: 4026010 /UG = Hs.282654 Homo |
| | sapiens mRNA; cDNA DKFZp564N1272 (from clone DKFZp564N1272); complete cds /FL = gb: AL136676.1 gb: AF087847.1 |
| 204054_at | gb: NM_000314.1 /DEF = Homo sapiens phosphatase and tensin homolog (mutated in multiple advanced cancers 1) (PTEN), mRNA. |
| | /FEA = mRNA /GEN = PTEN /PROD = phosphatase and tensin homolog (mutated inmultiple advanced cancers 1) /DB_XREF = gi: 4506248 |
| | /UG = Hs.10712 phosphatase and tensin homolog (mutated in multiple advanced cancers 1) |
| | /FL = gb: U92436.1 gb: U93051.1 gb: U96180.1 gb: NM_000314.1 |
| 212262_at | Consensus includes gb: AA149639 /FEA = EST /DB_XREF = gi: 1720440 /DB_XREF = est: z139c06.s1 /CLONE = IMAGE: 504298 /UG = Hs.15020 homolog of |
| | mouse quaking QKI (KH domain RNA binding protein) /FL = gb: AF142419.1 gb: AF142422.1 |
| 208785_s_at | Consensus includes gb: BE893893 /FEA = EST /DB_XREF = gi: 10355716 /DB_XREF = est: 601436260F1 /CLONE = IMAGE: 3921446 /UG = Hs.121849 |
| | microtubule-associated proteins 1A1B light chain 3 /FL = gb: AF303888.1 gb: NM_022818.2 gb: AF183417.1 |
| 218660_at | gb: NM_003494.1 /DEF = Homo sapiens dysferlin, limb girdle muscular dystrophy 2B (autosomal recessive) (DYSF), mRNA. /FEA = mRNA |
| | /GEN = DYSF /PROD = dysferlin /DB_XREF = gi: 4503430 /UG = Hs.143897 dysferlin, limb girdle muscular dystrophy 2B (autosomal recessive) |
| | /FL = gb: AF075575.1 gb: NM_003494.1 |
| 221920_s_at | Consensus includes gb: BE677761 /FEA = EST /DB_XREF = gi: 10038376 /DB_XREF = est: 7f59h05.x1 /CLONE = IMAGE: 3299001 |
| | /UG = Hs.300496 mitochondrial solute carrier |
| 211982_x_at | Consensus includes gb: AL546600 /FEA = EST /DB_XREF = gi: 12879872 /DB_XREF = est: AL546600 /CLONE = CS0DI029YA23 (3 prime) |
| | /UG = Hs.70500 KIAA0370 protein |
| 218647_s_at | gb: NM_024640.1 /DEF = Homo sapiens hypothetical protein FLJ23476 (FLJ23476), mRNA. /FEA = mRNA /GEN = FLJ23476 |
| | PROD-hypothetical protein FLJ23476 /DB_XREF = gi: 13375875 /UG = Hs.46736 hypothetical protein FLJ23476 /FL = gb: NM_024640.1 |
| 206571_s_at | gb: NM_004834.1 /DEF = Homo sapiens mitogen-activated protein kinase kinase kinase kinase 4 (MAP4K4), mRNA. |
| | /FEA = mRNA /GEN = MAP4K4 /PROD = mitogen-activated protein kinase kinase kinasekinase 4 /DB_XREF = gi: 4758523 |
| | /UG = Hs.3628 mitogen-activated protein kinase kinese kinase kinase 4 /FL = gb: AF096300.1 gb: NM_004834.1 |
| 218332_at | gb: NM_018476.1 /DEF = Homo sapiens uncharacterized hypothalamus protein HBEX2 (HBEX2), mRNA. /FEA = mRNA /GEN = HBEX2 |
| | /PROD = uncharacterized hypothalamus protein HBEX2 /DB_XREF = gi: 8923715 /UG = Hs.283719 uncharacterized hypothalamus protein HBEX2 |
| | /FL = gb: AF220189.1 gb: NM_018476.1 gb: AF183416.1 gb: AF237783.1 |
| 207113_s_at | gb: NM_000594.1 /DEF = Homo sapiens tumor necrosis factor (TNF superfamily, member 2) (TNF), mRNA. /FEA = mRNA /GEN = TNF |
| | /PROD = tumor necrosis factor (cachectin) /DB_XREF = gi: 10835154 /UG = Hs.241570 tumor necrosis factor (TNF superfamily, member 2) |
| | /FL = gb: NM_000594.1 |
| 209388_at | gb: BC000927.1 /DEF = Homo sapiens, Similar to poly (A) polymerase, clone MGC: 5378, mRNA, complete cds. |
| | /FEA = mRNA /PROD = Similar to poly (A) polymerase /DB_XREF = gi: 12654216 /UG = Hs.49007 poly(A) polymerase alpha /FL = gb: BC000927.1 |
| 207610_s_at | gb: NM_013447.1 /DEF = Homo sapiens egf-like module containing, mucin-like, hormone receptor-like sequence 2 (EMR2), mRNA. |
| | /FEA = mRNA /GEN = EMR2 /PROD = egf-like module containing, mucin-like, hormonereceptor-like sequence 2 /DB_XREF = gi: 7305024 |
| | /UG = Hs.137354 egf-like module containing, mucin-like, hormone receptor-like sequence 2 /FL = gb: AF114491.1 gb: NM_013447.1 |
| 204258_at | gb: NM_001270.1 /DEF = Homo sapiens chromodomain helicase DNA binding protein 1 (CHD1), mRNA. /FEA = mRNA /GEN = CHD1 |
| | /PROD = chromodomain helicase DNA binding protein 1 /DB_XREF = gi: 4557446 /UG = Hs.22670 chromodomain helicase DNA |
| | binding protein 1 /FL = gb: AF006513.1 gb: NM_001270.1 |
| 200948_at | gb: NM_005439.1 /DEF = Homo sapiens myeloid leukemia factor 2 (MLF2), mRNA. /FEA = mRNA /GEN = MLF2 |
| | /PROD = myeloid leukemia factor 2 /DB_XREF = gi: 4885486 /UG = Hs.79026 myeloid leukemia factor 2 |
| | /FL = gb: BC000898.1 gb: BC002340.1 gb: U57342.1 gb: AF070539.1 gb: NM_005439.1 |
| 211506_s_at | gb: AF043337.1 /DEF = Homo sapiens interleukin 8 C-terminal variant (IL8) mRNA, complete cds. /FEA = mRNA |
| | /GEN = IL8 /PROD = interleukin 8 C-terminal variant /DB_XREF = gi: 12641914 /UG = Hs.624 interleukin 8 /FL = gb: AF043337.1 |
| 217966_s_at | gb: NM_022083.1 /DEF = Homo sapiens niban protein (NIBAN), mRNA. /FEA = mRNA /GEN = NIBAN /PROD = niban protein |
| | /DB_XREF = gi: 11545796 /UG = Hs.48778 niban protein /FL = gb: AB050477.1 gb: NM_022083.1 gb: AF288391.1 |
| 214151_s_at | Consensus includes gb: AU144243 /FEA = EST /DB_XREF = gi: 11005764 /DB_XREF = est: AU144243 /CLONE = HEMBA1001328 |
| | /UG = Hs.247118 phosphatidylinositol glycan, class B |
| 211810_s_at | gb: D25284.1 /DEF = Human mRNA for alternative spliced product of galactocerebrosidase, complete cds. |
| | /FEA = CDS /PROD = alternative spliced |
| | product of galactocerebrosidase /DB_XREF = gi: 457445 /UG = Hs.273 galactosylceramidase (Krabbe disease) /FL = gb: D25284.1 |
| 207890_s_at | gb: NM_022718.1 /DEF = Homo sapiens matrix metalloproteinase 25 (MMP25), transcript variant 2, mRNA. /FEA = mRNA |
| | /GEN = MMP25 /PROD = matrix metalloproteinase 25 preproprotein /DB_XREF = gi: 13027808 |
| | /UG = Hs.198265 matrix metalloproteinase 25 /FL = gb: NM_022718.1 |
| 37028_at | Cluster Incl. U83981: Homo sapiens apoptosis associated protein (GADD34) mRNA, complete cds /cds = (222, 2246) /gb = U83981 |
| | /gi = 3258617 /ug = Hs.76556 /len = 2331 |
| 40446_at | Cluster Incl. AL021366: cICK0721Q.4.1 (PHD finger protein 2) (isoform 2) /cds = (215, 1918) /gb = AL021366 /gi = 3169115 /ug = Hs.166204 |
| | /len = 2260 |
| 219062_s_at | gb: NM_017742.1 /DEF = Homo sapiens hypothetical protein FLJ20281 (FLJ20281), mRNA. /FEA = mRNA /GEN = FLJ20281 |
| | /PROD = hypothetical protein FLJ20281 /DB_XREF = gi: 8923259 /UG = Hs.18800 hypothetical protein FLJ20281 /FL = gb: NM_017742.1 |
| 202158_s_at | gb: NM_006561.1 /DEF = Homo sapiens CUG triplet repeat, RNA-binding protein 2 (CUGBP2), mRNA. /FEA = mRNA /GEN = CUGBP2 |
| | /PROD = CUG triplet repeat, RNA-binding protein 2 /DB_XREF = gi: 5729815 /UG = Hs.211610 CUG triplet repeat, |
| | RNA-binding protein 2 /FL = gb: U69546.1 gb: AF036956.1 gb: AF090694.1 gb: NM_006561.1 |
| 60084_at | Cluster Incl. AI453099: tj61e11.x1 Homo sapiens cDNA, 3 end /clone = IMAGE-2146028 /clone_end = 3 /gb = AI453099 |
| | /gi = 4307988 /ug = Hs.24668 /len = 554 |
| 210142_x_at | gb: AF117234.1 /DEF = Homo sapiens flotillin mRNA, complete cds. /FEA = mRNA /PROD = flotillin /DB_XREF = gi: 6563241 |
| | /UG = Hs.179986 flotillin 1 /FL = gb: AF117234.1 |
| 204490_s_at | gb: M24915.1 /DEF = Human CDw44 antigen, complete cds. /FEA = mRNA /DB_XREF = gi: 180196 /UG = Hs.169610 CD44 antigen |
| | (homing function and Indian blood group system) /FL = gb: NM_000610.1 gb: U40373.1 gb: M59040.1 gb: M24915.1 |
| 203949_at | gb: NM_000250.1 /DEF = Homo sapiens myeloperoxidase (MPO), nuclear gene encoding mitochondrial protein, mRNA. |
| | /FEA = mRNA /GEN = MPO /PROD = myeloperoxidase /DB_XREF = gi: 4557758 /UG = Hs.1817 myeloperoxidase /FL = gb: M19507.1 |
| | gb: J02694.1 gb: NM_000250.1 |
| 219259_at | gb: NM_022367.1 /DEF = Homo sapiens hypothetical protein FLJ12287 similar to semaphorins (FLJ12287), mRNA. |
| | /FEA = mRNA /GEN = FLJ12287 /PROD = hypothetical protein FLJ12287 similar tosemaphorins /DB_XREF = gi: 11641290 |
| | /UG = Hs.7634 hypothetical protein FLJ12287 similar to semaphorins /FL = gb: NM_022367.1 gb: AB029394.1 |
| 201751_at | gb: NM_014876.1 /DEF = Homo sapiens KIAA0063 gene product (KIAA0063), mRNA. /FEA = mRNA /GEN = KIAA0063 |
| | /PROD = KIAA0063 gene product /DB_XREF = gi: 7661887 /UG = Hs.3094 KIAA0063 gene product /FL = gb: D31884.1 gb: NM_014876.1 |
| 203153_at | gb: NM_001548.1 /DEF = Homo sapiens interferon-induced protein with tetratricopeptide repeats 1 (IFIT1), mRNA. |
| | /FEA = mRNA /GEN = IFIT1 /PROD = interferon-induced protein withtetratricopeptide repeats 1 /DB_XREF = gi: 4504584 |
| | /UG = Hs.20315 interferon-induced protein with tetratricopeptide repeats 1 /FL = gb: M24594.1 gb: NM_001548.1 |
| 210724_at | gb: AF239764.1 /DEF = Homo sapiens EGF-like module-containing mucin-like receptor EMR3 mRNA, complete cds. |
| | /FEA = mRNA /PROD = EGF-like module-containing mucin-like receptorEMR3 /DB_XREF = gi: 13183148 |
| | /UG = Hs.326777 Homo sapiens EGF-like module-containing mucin-like receptor EMR3 mRNA, complete cds /FL = gb: AF239764.1 |
| 202381_at | gb: NM_003816.1 /DEF = Homo sapiens a disintegrin and metalloproteinase domain 9 (meltrin gamma) (ADAM9), mRNA. |
| | /FEA = mRNA /GEN = ADAM9 /PROD = a disintegrin and metalloproteinase domain 9preproprotein /DB_XREF = gi: 4501914 |
| | /UG = Hs.2442 a disintegrin and metalloproteinase domain 9 (meltrin gamma) /FL = gb: U41766.1 gb: NM_003816.1 |
| 217824_at | Consensus includes gb: AW500009 /FEA = EST /DB_XREF = gi: 7112213 /DB_XREF = est: UI-HF-BN0-aki-e-09-0-UI.r1 |
| | /CLONE = IMAGE: 3077105 /UG = Hs.184325 CGI-76 protein /FL = gb: AF151834.1 gb: AF161502.1 gb: AF151039.1 gb: NM_016021.1 |
| 200984_s_at | Consensus includes gb: X16447.1 /DEF = Human mRNA for CD59, an LY-6-like protein regulating complement membrane attack. |
| | /FEA = mRNA /PROD = precursor polypeptide (AA −25 to 103) /DB_XREF = gi: 29805 /UG = Hs.119663 CD59 antigen p18-20 |
| | (antigen identified by monoclonal antibodies 16.3A5, EJ16, EJ30, EL32 and G344) /FL = gb: NM_000611.1 gb: M34671.1 |
| 219999_at | gb: NM_018621.1 /DEF = Homo sapiens hypothetical protein PRO2198 (PRO2198), mRNA. /FEA = mRNA /GEN = PRO2198 |
| | /PROD = hypothetical protein PRO2198 /DB_XREF = gi: 8924129 /UG = Hs.116459 hypothetical protein PRO2198 |
| | /FL = gb: AF116691.1 gb: NM_018621.1 |
| 212644_s_at | Consensus includes gb: AI671747 /FEA = EST /DB_XREF = gi: 4851478 /DB_XREF = est: wa05e06.x1 /CLONE = IMAGE: 2297218 |
| | /UG = Hs.81360 Homo sapiens cDNA: FLJ21927 fis, clone HEP04178, highly similar to HSU90909 Human clone 23722 mRNA sequence |
| 219622_at | gb: NM_017817.1 /DEF = Homo sapiens hypothetical protein: FLJ20429 (FLJ20429), mRNA. /FEA = mRNA /GEN = FLJ20429 |
| | /PROD = hypothetical protein FLJ20429 /DB_XREF = gi: 8923400 /UG = Hs.179791 hypothetical protein FLJ20429 /FL = gb: NM_017817.1 |
| 219190_s_at | gb: NM_017629.1 /DEF = Homo sapiens hypothetical protein FLJ20033 (FLJ20033), mRNA. /FEA = mRNA /GEN = FLJ20033 |
| | /PROD = hypothetical protein FLJ20033 /DB_XREF = gi: 8923033 /UG = Hs.134757 hypothetical protein FLJ20033 /FL = gb: NM_017629.1 |
| 208707_at | Consensus includes gb: BE552334 /FEA = EST /DB_XREF = gi: 9794026. /DB_XREF = est: hy06c06.x1 /CLONE = IMAGE: 3196522 |
| | /UG = Hs.286236 eukaryotic translation initiation factor 5 /FL = gb: AL080102.1 |
| 218506_x_at | gb: NM_018459.1 /DEF = Homo sapiens uncharacterized bone marrow protein BM045 (BM045), mRNA. /FEA = mRNA |
| | /GEN = BM045 /PROD = uncharacterized bone marrow protein BM045 /DB_XREF = gi: 8922103 |
| | /UG = Hs.8750 uncharacterized bone marrow protein BM045 /FL = gb: AF217521.1 gb: NM_018459.1 |
| 207545_s_at | gb: NM_003744.1 /DEF = Homo sapiens numb (Drosophila) homolog (NUMB), mRNA. /FEA = mRNA /GEN = NUMB /PROD = numb (Drosophila) homolog |
| | /DB_XREF = gi: 4505478 /UG = Hs.78890 numb (Drosophila) homolog /FL = gb: NM_003744.1 gb: L40393.1 |
| 212014_x_at | Consensus includes gb: AI493245 /FEA = EST /DB_XREF = gi: 4394248 /DB_XREF = est: ti30d08.x1 /CLONE = IMAGE: 2131983 |
| | /UG = Hs.169610 CD44 antigen (homing function and Indian blood group system) |
| 209039_x_at | gb: AF001434.1 /DEF = Human Hpast (HPAST) mRNA, complete cds. /FEA = mRNA /GEN = HPAST /PROD = Hpast /DB_XREF = gi: 2529706 |
| | /UG = Hs.155119 EH domain containing 1 /FL = gb: AF001434.1 |
| 208650_s_at | Consensus includes gb: BG327863 /FEA = EST /DB_XREF = gi: 13134301 /DB_XREF = est: 602426876F1 /CLONE = IMAGE: 4564675 |
| | /UG = Hs.286124 CD24 antigen (small cell lung carcinoma cluster 4 antigen) /FL = gb: M58664.1 gb: L33930.1 gb: NM_013230.1 |
| 209806_at | gb: BC000893.1 /DEF = Homo sapiens, H2B histone family, member A, clone MGC: 5132, mRNA, complete cds. /FEA = mRNA |
| | /PROD = H2B histone family, member A /DB_XREF = gi: 12654150 /UG = Hs.247817 H2B histone family, member A /FL = gb: BC000893.1 |
| 212573_at | Consensus includes gb: AF131747.1 /DEF = Homo sapiens clone 24951 mRNA sequence. /FEA = mRNA /DB_XREF = gi: 4406562 |
| | /UG = Hs.167115 KIAA0830 protein |
| 203845_at | Consensus includes gb: AV727449 /FEA = EST /DB_XREF = gi: 10836870 /DB_XREF = est: AV727449 /CLONE = HTCAYG01 |
| | /UG = Hs.199061 p300CBP-associated factor /FL = gb: U57317.2 gb: NM_003884.2 |
| 215806_x_at | Consensus includes gb: M13231.1 /DEF = Human T-cell receptor aberrantly rearranged gamma-chain mRNA from |
| | cell line HPB-MLT. /FEA = mRNA /DB_XREF = gi: 339168 /UG = Hs.274509 T cell receptor gamma constant 2 |
| 202195_s_at | gb: NM_016040.1 /DEF = Homo sapiens CGI-100 protein (LOC50999), mRNA. /FEA = mRNA /GEN = LOC50999 /PROD = CGI-100 protein |
| | /DB_XREF = gi: 7705583 /UG = Hs.296155 CGI-100 protein /FL = gb: AF151858.1 gb: NM_016040.1 |
| 201912_s_at | gb: NM_002094.1 /DEF = Homo sapiens G1 to S phase transition 1 (GSPT1), mRNA. /FEA = mRNA /GEN = GSPT1 |
| | /PROD = G1 to S phase transition 1 /DB_XREF = gi: 4504166 /UG = Hs.2707 G1 to S phase transition 1 /FL = gb: NM_002094.1 |
| 201712_s_at | gb: NM_006267.2 /DEF = Homo sapiens RAN binding protein 2 (RANBP2), mRNA. /FEA = mRNA /GEN = RANBP2 |
| | /PROD = RAN binding protein 2 /DB_XREF = gi: 6382078 /UG = Hs.199179 RAN binding protein 2 /FL = gb: NM_006267.2 gb: D42063.1 |
| 212864_at | Consensus includes gb: Y16521.1 /DEF = Homo sapiens mRNA for CDS2 protein. /FEA = mRNA /GEN = cds2 |
| | /PROD = CDS2 protein /DB_XREF = gi: 4186022 /UG = Hs.24812 CDP-diacylglycerol synthase (phosphatidate cytidylyltransferase) 2 |
| 203504_s_at | gb: NM_005502.1 /DEF = Homo sapiens ATP-binding cassette, sub-family A (ABC1), member 1 (ABCA1), mRNA. |
| | /FEA = mRNA /GEN = ABCA1 /PROD = ATP-binding cassette, sub-family A member 1 /DB_XREF = gi: 5915657 |
| | /UG = Hs.211562 ATP-binding cassette, sub-family A (ABC1), member 1 /FL = gb: AF165281.1 gb: NM_005502.1 gb: AF285167.1 |
| 212457_at | Consensus includes gb: AL161985.1 /DEF = Homo sapiens mRNA; cDNA DKFZp761J1810 (from clone DKEZp761J1810) |
| | /FEA = mRNA /DB_XREF = gi: 7328121 /UG = Hs.274184 transcription factor binding to IGHM enhancer 3 |
| 214683_s_at | Consensus includes gb: AI251890 /FEA = EST /DB_XREF = gi: 3848419 /DB_XREF = est: qu78d12.x1 /CLONE = IMAGE: 1978199 |
| | /UG = Hs.2083 CDC-like kinasel |
| 44783_s_at | Cluster Incl. R61374: yh15e02.s1 Homo sapiens cDNA, 3 end /clone = IMAGE-37665 /clone_end = 3 /gb = R61374 |
| | /gi = 832069 /ug = Hs.234434 /len = 434 |
| 202252_at | gb: NM_002870.1 /DEF = Homo sapiens RAB13, member RAS oncogene family (RAB13), mRNA. /FEA = mRNA /GEN = RAB13 /PROD = RAB13, |
| | member RAS oncogene family /DB_XREF = gi: 4506362 /UG = Hs.151536 RAB13, member RAS oncogene family /FL = gb: BC000799.1 |
| | gb: NM_002870.1 |
| 206025_s_at | Consensus includes gb: AW188198 /FEA = EST /DB_XREF = gi: 6462634 /DB_XREF = est: xj93f03.x1 /CLONE = IMAGE: 2664797 |
| | /UG = Hs.29352 tumor necrosis factor, alpha-induced protein 6 /FL = gb: NM_007115.1 |
| 203936_s_at | gb: NM_004994.1 /DEF = Homo sapiens matrix metalloproteinase 9 (gelatinase B, 92 kD gelatinase, 92 kD type IV collagenase) |
| | (MMP9), mRNA. /FEA = mRNA /GEN = MMP9 /PROD = matrix metalloproteinase 9 preproprotein /DB_XREF = gi: 4826835 /UG = Hs.151738 matrix |
| | metalloproteinase 9 (gelatinase B, 92 kD gelatinase, 92 kD type IV collagenase) /FL = gb: J05070.1 gb: NM_004994.1 |
| 221523_s_at | Consensus includes gb: AL138717 /DEF = Human DNA sequence from clone RP11-11DB on chromosome 6 Contains the 5 end |
| | of the gene for a yeast ubiquitin conjugating enzyme UBC6 homolog, the gene for a possible GTP binding protein, |
| | a NACA (nascent-polypeptide-associated complex alpha polype . . . /FEA = mRNA_2 /DB_XREF = gi: 8894207 |
| | /UG = Hs.238679 Rag D protein /FL = gb: NM_021244.1 gb: AF272036.1 gb: BC003088.1 |
| 215785_s_at | Consensus includes gb: AL161999.1 /DEF = Homo sapiens mRNA; cDNA DKFZp761H087 (from clone DKFZp761H087); |
| | partial cds. /FEA = mRNA /GEN = DKFZp761H087 /PROD = hypothetical protein /DB_XREF = gi: 7328000 /UG = Hs.258503 p53 inducible protein |
| 217822_at | gb: NM_016312.1 /DEF = Homo sapiens Npw38-binding protein NpwBP (LOC51729), mRNA. /FEA = mRNA /GEN = LOC51729 /PROD = Npw38-binding protein |
| | NpwBP /DB_XREF = gi: 7706500 /UG = Hs.16420 Npw38-binding protein NpwBP /FL = gb: BC001621.1 gb: AF118023.1 gb: AB029309.1 gb: NM_016312.1 |
| 202206_at | Consensus includes gb: AW450363 /FEA = EST /DB_XREF = gi: 6991139 /DB_XREF = est: UI-H-BI3-akn-d-02-0-UI.s1 /CLONE = IMAGE: 2734875 |
| | /UG = Hs.111554 ADP-ribosylation factor-like 7 /FL = gb: BC001051.1 gb: AB016811.1 gb: NM_005737.2 |
| 202439_s_at | gb: NM_000202.2 /DEF = Homo sapiens iduronate 2-sulfatase (Hunter syndrome) (IDS), transcript variant 1, mRNA. |
| | /FEA = mRNA /GEN = IDS /PROD = iduronate-2-sulfatase isoform a precursor /DB_XREF = gi: 5360215 /UG = Hs.172458 |
| | iduronate 2-sulfatase (Hunter syndrome) /FL = gb: M58342.1 gb: NM_000202.2 |
| 213056_at | Consensus includes gb: AU145019 /FEA = EST /DB_XREF = gi: 11006540 /DB_XREF = est: AU145019 /CLONE = HEMBA1003646 |
| | /UG = Hs.96427 KIAA1013 protein |
| 222142_at | Consensus includes gb: AK024212.1 /DEF = Homo sapiens cDNA FLJ14150 fis, clone MAMMA1003026, highly similar |
| | to Homo sapiens HSPC057 mRNA. /FEA = mRNA /DB_XREF = gi: 10436534 /UG = Hs.18827 KIAA0849 protein |
| 214784_x_at | Consensus includes gb: BE966299 /FEA = EST /DB_XREF = gi: 11771566 /DB_XREF = est: 601660539R1 /CLONE = IMAGE: 3906248 |
| | /UG = Hs.70500 KIAA0370 protein |
| 222035_s_at | Consensus includes gb: AI984479 /FEA = EST /DB_XREF = gi: 5811756 /DB_XREF = est: wr83e06.x1 /CLONE = IMAGE: 2494306 |
| | UG = Hs.49007 poly(A) polymerase alpha |
| 212219_at | Consensus includes gb: D38521.1 /DEF = Human mRNA for KIAA0077 gene, partial cds. /FEA = mRNA /GEN = KIAA0077 |
| | /DB_XREF = gi: 559329 /UG = Hs.112396 KIAA0077 protein |
| 214152_at | Consensus includes gb: AU144243 /FEA = EST /DB_XREF = gi: 11005764 /DB_XREF = est: AU144243 |
| | /CLONE = HEMBA1001328 /UG = Hs.247118 phosphatidylinositol glycan, class B |
| 202948_at | gb: NM_000877.1 /DEF = Homo sapiens interleukin 1 receptor, type I (IL1R1), mRNA. /FEA = mRNA /GEN = IL1R1 /PROD = interleukin 1 |
| | receptor, type I /DB_XREF = gi: 4504658 /UG = Hs.82112 interleukin 1 receptor, type I /FL = gb: M27492.1 gb: NM_000877.1 |
| 207978_s_at | gb: NM_006981.1 /DEF = Homo sapiens nuclear receptor subfamily 4, group A, member 3 (NR4A3), mRNA. /FEA = mRNA /GEN = NR4A3 |
| | /PROD = nuclear receptor subfamily 4, group A, member 3 /DB_XREF = gi: 11276070 /UG = Hs.80561 nuclear receptor subfamily 4, |
| | group A, member 3 /FL = gb: NM_006981.1 gb: D78579.1 |
| 209304_x_at | gb: AF087853.1 /DEF = Homo sapiens growth arrest and DNA damage inducible protein beta (GADD45B) mRNA, complete cds. |
| | /FEA = mRNA /GEN = GADD45B /PROD = growth arrest and DNA damage inducible proteinbeta /DB_XREF = gi: 12061050 |
| | /UG = Hs.110571 growth arrest and DNA-damage-inducible, beta /FL = gb: AF087853.1 gb: AF078077.1 |
| 214783_s_at | Consensus includes gb: BG177920 /FEA = EST /DB_XREF = gi: 12684623 /DB_XREF = est: 602327839F1 /CLONE = IMAGE: 4429256 |
| | /UG = Hs.75510 annexin A11 |
| 46270_at | Cluster Incl. AL039447: DKFZp434N1010_s1 Homo sapiens cDNA, 3 end /clone = DKFZp434N1010 /clone_end = 3 /gb = AL039447 |
| | /gi = 5408497 /ug = Hs.75425 /len = 841 |
| 221760_at | Consensus includes gb: BG287153 /FEA = EST /DB_XREF = gi: 13040709 /DB_XREF = est: 602381868F1 /CLONE = IMAGE: 4499393 |
| | /UG = Hs.25253 mannosidase, alpha, class 1A, member 1 |
| 210118_s_at | gb: M15329.1 /DEF = Human interleukin 1-alpha (IL1A) mRNA, complete cds. /FEA = mRNA /GEN = IL1A /PROD = interleukin 1-alpha |
| | /DB_XREF = gi: 186277 /UG = Hs.1722 interleukin 1, alpha /FL = gb: M15329.1 |
| 204781_s_at | gb: NM_000043.1 /DEF = Homo sapiens tumor necrosis factor receptor superfamily, member 6 (TNFRSF6), mRNA. /FEA = mRNA |
| | /GEN = TNFRSF6 /PROD = apoptosis (APO-1) antigen 1 /DB_XREF = gi: 4507582 /UG = Hs.82359 tumor necrosis factor receptor superfamily, |
| | membar 6 /FL = gb: M67454.1 gb: NM_000043.1 |
| 207677_s_at | gb: NM_013416.1 /DEF = Homo sapiens neutrophil cytosolic factor 4 (40 kD) (NCF4), transcript variant 2, mRNA. |
| | /FEA = mRNA /GEN = NCF4 /PROD = neutrophil cytosolic factor 4 (40 kD), isoform 2 /DB_XREF = gi: 7382492 |
| | /UG = Hs.196352 neutrophil cytosolic factor 4 (40 kD) /FL = gb: BC002798.1 gb: AB025219.1 gb: NM_013416.1 |
| 206302_s_at | gb: NM_019094.1 /DEF = Homo sapiens nudix (nucleoside diphosphate linked moiety X)-type motif 4 (NUDT4), mRNA. |
| | /FEA = mRNA /GEN = NUDT4 /PROD = nudix (nucleoside diphosphate linked moietyX)-type motif 4 /DB_XREF = gi: 10800135/UG = Hs.92381 |
| | nudix (nucleoside diphosphate linked moiety X)-type motif 4 /FL = gb: NM_019094.1 gb: AF191649.1 gb: AF191650.1 gb: AF191653.1 |
| 219460_s_at | gb: NM_017849.1 /DEF = Homo sapiens hypothetical protein FLJ20507 (FLJ20507), mRNA. /FEA = mRNA /GEN = FLJ20507 /PROD = hypothetical |
| | protein FLJ20507 /DB_XREF = gi: 8923465 /UG = Hs.202955 hypothetical protein FLJ20507 /FL = gb: NM_017849.1 |
| 212225_at | Consensus includes gb: AL516854 /FEA = EST /DB_XREF = gi: 12780347 /DB_XREF = est: AL516854 /CLONE = CS0DA007YB20 (5 prime) |
| | /UG = Hs.150580 putative translation initiation factor |
| 204479_at | gb: NM_012383.1 /DEF = Homo sapiens osteoclast stimulating factor 1 (OSTF1), mRNA. /FEA = mRNA /GEN = OSTF1 /PROD = osteoclast |
| | stimulating factor 1 /DB_XREF = gi: 6912563 /UG = Hs.95821 osteoclast stimulating factor 1 /FL = gb: U63717.1 gb: NM_012383.1 |
| 216457_s_at | Consensus includes gb: AK026080.1 /DEF = Homo sapiens cDNA: FLJ22427 fis, clone HRC09013. /FEA = mRNA /DB_XREF = gi: 10438814 |
| | /UG = Hs.288883 splicing factor 3a, subunit 1, 120 kD |
| 212676_at | Consensus includes gb: AW293356 /FEA = EST /DB_XREF = gi: 6699992 /DB_XREF = est: UI-H-BI2-ah1-c-11-0-UI.s1 /CLONE = IMAGE: 2727165 |
| | /UG = Hs.58220 Homo sapiens cDNA: FLJ23005 fis, clone LNG00396, highly similar to AF055023 Homo sapiens clone 24723 mRNA sequence |
| 203675_at | gb: NM_005013.1 /DEF = Homo sapiens nucleobindin 2 (NUCB2), mRNA. /FEA = mRNA /GEN = NUCB2 /PROD = nucleobindin 2 /DB_XREF = gi: 4826869 |
| | /UG = Hs.3164 nucleobindin 2 /FL = gb: AF052642.1 gb: AF052643.1 gb: AF052644.1 gb: NM_005013.1 |
| 221156_x_at | gb: NM_004748.1 /DEF = Homo sapiens cell cycle progression 8 protein (CPR8), mRNA. /FEA = mRNA /GEN = CPR8 /PROD = cell cycle |
| | progression 8 protein /DB_XREF = gi: 4758047 /UG = Hs.283753 cell cycle progression 8 protein /FL = gb: AF011794.1 gb: NM_004748.1 |
| 207791_s_at | gb: NM_004161.1 /DEF = Homo sapiens RAB1, member RAS oncogene family (RAB1), mRNA. /FEA = mRNA /GEN = RAB1 /PROD = RAB1, |
| | member RAS oncogene family /DB_XREF = gi: 4758987 /UG = Hs.3642 RAB1, member RAS oncogene family /FL = gb: NM_004161.1 gb: M28209.1 |
| 200692_s_at | gb: NM_004134.1 /DEF = Homo sapiens heat shock 70 kD protein 9B (mortalin-2) (HSPA9B), mRNA. /FEA = mRNA /GEN = HSPA9B |
| | /PROD = heat shock 70 kD protein 9B (mortalin-2) /DB_XREF = gi: 4758569 /UG = Hs.3069 heat shock 70 kD protein 9B (mortalin-2) |
| | /FL = gb: BC000478.1 gb: L15189.1 gb: NM_004134.1 |
| 218936_s_at | gb: NM_014167.1 /DEF = Homo sapiens HSPC128 protein (HSPC128), mRNA. /FEA = mRNA /GEN = HSPC128 /PROD = HSPC128 protein |
| | /DB_XREF = gi: 7661789 /UG = Hs.90527 HSPC128 protein /FL = gb: AF213377.1 gb: AF161477.1 gb: NM_014167.1 |
| 213931_at | Consensus includes gb: AI819238 /FEA = EST /DB_XREF = gi: 5438328 /DB_XREF = est: wj42g05.x1 /CLONE = IMAGE: 2405528 |
| | /UG = Hs.180919 inhibitor of DNA binding 2, dominant negative helix-loop-helix protein |
| 218247_s_at | gb: NM_016626.1 /DEF = Homo sapiens hypothetical protein (LOC51320), mRNA. /FEA = mRNA /GEN = LOC51320 /PROD = hypothetical protein |
| | /DB_XREF = gi: 7706165 /UG = Hs.12830 hypothetical protein /FL = gb: AF208855.1 gb: NM_016626.1 |
| 213935_at | Consensus includes gb: AF007132.1 /DEF = Homo sapiens clone 23551 mRNA sequence. /FEA = mRNA /DB_XREF = gi: 2852606 |
| | /UG = Hs.184019 Homo sapiens clone 23551 mRNA sequence |
| 208815_x_at | gb: AB023420.1 /DEF = Homo sapiens mRNA for heat shock protein apg-2, complete cds. /FEA = mRNA /GEN = apg-2 |
| | /PROD = apg-2 /DB_XREF = gi: 4579908 /UG = Hs.90093 heat shock 70 kD protein 4 /FL = gb: AB023420.1 |
| 218177_at | Consensus includes gb: AA293502 /FEA = EST /DB_XREF = gi: 1941036 /DB_XREF = est: zt53h06.r1 /CLONE = IMAGE: 726107 /UG = Hs.42733 |
| | CHMP1.5 protein /FL = gb: AF281064.1 gb: NM_020412.1 |
| 200648_s_at | gb: NM_002065.1 /DEF = Homo sapiens glutamate-ammonia ligase (glutamine synthase) (GLUL), mRNA. /FEA = mRNA /GEN = GLUL |
| | /PROD = glutamate-ammonia ligase (glutamine synthase) /DB_XREF = gi: 4504026 /UG = Hs.170171 |
| | glutamate-ammonia ligase (glutamine synthase) /FL = gb: NM_002065.1 |
| 202843_at | gb: NM_012328.1 /DEF = Homo sapiens microvascular endothelial differentiation gene 1 (MDG1), mRNA. /FEA = mRNA /GEN = MDG1 |
| | /PROD = microvascular endothelial differentiation gene1 /DB_XREF = gi: 9558754 /UG = Hs.6790 DnaJ (Hsp40) homolog, subfamily B, |
| | member 9 /FL = gb: AF083247.1 gb: AL080081.1 gb: AB026908.1 gb: NM_012328.1 |
| 214578_s_at | Consensus includes gb: AV683882 /FEA = EST /DB_XREF = gi: 10285745 /DB_XREF = est: AV683882 /CLONE = GKCAIC05 /UG = Hs.17820 |
| | Rho-associated, coiled-coil containing protein kinase 1 /FL = gb: U43195.1 gb: NM_005406.1 |
| 217473_x_at | Consensus includes gb: AF229163 /DEF = Homo sapiens natural resistance-associated macrophage protein 1 (SLC11A1) gene, |
| | complete cds, alternatively spliced; and nuclear LIM interactor-interacting factor (NLI-IF) gene, complete cds |
| | /FEA = mRNA_3 /DB_XREF = gi: 10257408 /UG = Hs.182611 solute carrier family 11 (proton-coupled divalent metal ion transporters), |
| | member 1 |
| 207624_s_at | gb: NM_000328.1 /DEF = Homo sapiens retinitis pigmentosa GTPase regulator (RPGR), mRNA. /FEA = mRNA /GEN = RPGR /PROD = retinitis |
| | pigmentosa GTPase regulator /DB_XREF = gi: 4506580 /UG = Hs.153614 retinitis pigmentosa GTPase regulator |
| | /FL = gb: U57629.1 gb: NM_000328.1 |
| 202778_s_at | gb: NM_003453.1 /DEF = Homo sapiens zinc finger protein 198 (ZNF198), mRNA. /FEA = mRNA /GEN = ZNF198 /PROD = zinc finger protein |
| | 198 /DB_XREF = gi: 4508010 /UG = Hs.109526 zinc finger protein 198 /FL = gb: AF035374.1 gb: AF060181.1 gb: NM_003453.1 |
| 200653_s_at | gb: M27319.1 /DEF = Human calmodulin mRNA, complete cds. /FEA = mRNA /PROD = calmodulin /DB_XREF = gi: 179809 /UG = Hs.177656 |
| | calmodulin 1 (phosphorylase kinase, delta) /FL = gb: M27319.1 gb: NM_006888.1 |
| 218627_at | gb: NM_018370.1 /DEF = Homo sapiens hypothetical protein FLJ11259 (FLJ11259), mRNA. /FEA = mRNA /GEN = FLJ11259 /PROD = hypothetical |
| | protein FLJ11259 /DB_XREF = gi: 8922957 /UG = Hs.184465 hypothetical protein FLJ11259 /FL = gb: NM_018370.1 |
| 202059_s_at | gb: NM_002264.1 /DEF = Homo sapiens karyopherin alpha 1 (importin alpha 5) (KPHA1), mRNA. /FEA = mRNA /GEN = KPNA1 /PROD = karyopherin |
| | alpha 1 /DB_XREF = gi: 4504894 /UG = Hs.169149 karyopherin alpha 1 (importin alpha 5) /FL = gb: BC002374.1 |
| | gb: BC003009.1 gb: NM_002264.1 |
| 218132_s_at | gb: NM_024075.1 /DEF = Homo sapiens LENG5 protein (LENG5), mRNA. /FEA = mRNA /GEN = LENG5 /PROD = LENG5 protein |
| | /DB_XREF = gi: 13129061 /UG = Hs.15580 LENG5 protein /FL = gb: BC000944.2 gb: NM_024075.1 |
| 203265_s_at | Consensus includes gb: AA810268 /FEA = EST /DB_XREF = gi: 2879627 /DB_XREF = est: od14f07.s1 /CLONE = IMAGE: 1367941 |
| | /UG = Hs.75217 mitogen-activated protein kinase kinase 4 /FL = gb: NM_003010.1 gb: L36870.1 gb: U17743.1 |
| 209694_at | gb: M97655.1 /DEF = Human 6-pyruvoyltetrahydropterin synthase (PTS) mRNA, complete cds. /FEA = mRNA /GEN = PTS |
| | /PROD = 6-pyruvoyltetrahydropterin synthase /DB_XREF = gi: 306438 /UG = Hs.366 6-pyruvoyltetrahydropterin synthase |
| | /FL = gb: M97655.1 gb: NM_000317.1 gb: D17400.1 |
| 217591_at | Consensus includes gb: BF725121 /FEA = EST /DB_XREF = gi: 12041032 /DB_XREF = est: bx12e01.x1 /CLONE = bx12e01 |
| | /UG = Hs.272108 ESTs |
| 222309_at | Consensus includes gb: AW972292 /FEA = EST /DB_XREF = gi: 8162138 /DB_XREF = est: EST384381 /UG = Hs.292998 ESTs |
| 201627_s_at | gb: NM_005542.1 /DEF = Homo sapiens insulin induced gene 1 (INSIG1), mRNA. /FEA = mRNA /GEN = INSIG1 |
| | /PROD = insulin induced gene 1 /DB_XREF = gi: 5031800 /UG = Hs.56205 insulin induced gene 1 /FL = gb: NM_005542.1 |
| 217249_x_at | Consensus includes gb: AC004544 /DEF = Homo sapiens BAC clone CTB-162B4 from 4 /FEA = CDS /DB_XREF = gi: 3041843 |
| | /UG = Hs.248095 Homo sapiens BAC clone CTB-162B4 from 4 |
| 220496_at | gb: NM_016509.1 /DEF = Homo sapiens C-type lectin-like receptor 2 (LOC51266), mRNA. /FEA = mRNA /GEN = LOC51266 |
| | /PROD = C-type lectin-like receptor-2 /DB_XREF = gi: 7706060 /UG = Hs.114231 C-type lectin-like receptor-2 /FL = gb: AF124841.1 |
| | gb: NM_016509.1 |
| 201096_s_at | Consensus includes gb: AL537042 /FEA = EST /DB_XREF = gi: 12800535 /DB_XREF = est: AL537042 /CLONE = CS0DF017YF17 (5 prime) |
| | /UG = Hs.75290 ADP-ribosylation factor 4 /FL = gb: BC003364.1 gb: M36341.1 gb: NM_001660.2 |
| 201580_s_at | Consensus includes gb: AL544094 /FEA = EST /DB_XREF = gi: 12876573 /DB_XREF = est: AL544094 /CLONE = CS0DI004YG20 (3 prime) |
| | /UG = Hs.169358 hypothetical protein /FL = gb: NM_021156.1 |
| 201109_s_at | Consensus includes gb: AV726673 /FEA = EST /DB_XREF = gi: 10836094 /DB_XREF = est: AV726673 /CLONE = HTCBGC12 /UG = Hs.87409 |
| | thrombospondin 1 /FL = gb: NM_003246.1 |
| 203080_s_at | gb: NM_013450.1 /DEF = Homo sapiens bromodomain adjacent to zinc finger domain, 2B (BAZ2B), mRNA. /FEA = mRNA /GEN = BAZ2B |
| | /PROD = bromodomain adjacent to zinc finger domain, 2B /DB_XREF = gi: 7304922 /UG = Hs.8383 bromodomain adjacent to zinc finger |
| | domain, 2B /FL = gb: AB032255.1 gb: NM_013450.1 |
| 202558_s_at | gb: NM_006948.1 /DEF = Homo sapiens stress 70 protein chaperone, microsome-associated, 60 kD (STCH), mRNA. /FEA = mRNA |
| | /GEN = STCH /PROD = stress 70 protein chaperone, microsome-associated, 60 kD /DB_XREF = gi: 5902125 /UG = Hs.288799 stress 70 |
| | protein chaperone, microsome-associated, 60 kD /FL = gb: U04735.1 gb: NM_006948.1 |
| 201349_at | gb: NM_004252.1 /DEF = Homo sapiens solute carrier family 9 (sodiumhydrogen exchanger), isoform 3 regulatory factor 1 |
| | (SLC9A3R1), mRNA. /FEA = mRNA /GEN = SLC9A3R1 /PROD = solute carrier family 9 (sodiumhydrogenexchanger), isoform 3 regulatory |
| | factor 1 /DB_XREF = gi: 4759139 /UG = Hs.184276 solute carrier family 9 (sodiumhydrogen exchanger), isoform 3 regulatory |
| | factor 1 /FL = gb: BC001443.1 gb: BC003361.1 gb: AF036241.1 gb: AF015926.1 gb: NM_004252.1 |
| 220603_s_at | gb: NM_018349.1 /DEF = Homo sapiens hypothetical protein FLJ11175 (FLJ11175), mRNA. /FEA = mRNA /GEN = FLJ11175 |
| | /PROD = hypothetical protein FLJ11175 /DB_XREF = gi: 8922916 /UG = Hs.33368 hypothetical protein FLJ11175 /FL = gb: NM_018349.1 |
| 35820_at | Cluster Incl. X62078: H. sapiens mRNA for GM2 activator protein /cds = UNKNOWN /gb = X62078 /gi = 313158 /ug = Hs.69743 /len = 2436 |
| 201731_s_at | gb: NM_003292.1 /DEF = Homo sapiens translocated promoter region (to activated MET oncogene) (TPR), mRNA. /FEA = mRNA |
| | /GEN = TPR /PROD = translocated promoter region (to activated METoncogene) /DB_XREF = gi: 4507658 /UG = Hs.169750 translocated |
| | promoter region (to activated MET oncogene) /FL = gb: NM_003292.1 |
| 202414_at | gb: NM_000123.1 /DEF = Homo sapiens excision repair cross-complementing rodent repair deficiency, complementation group 5 |
| | (xeroderma pigmentosum, complementation group G (Cockayne syndrome)) (ERCC5), mRNA. /FEA = mRNA /GEN = ERCC5 |
| | /PROD = XPG-complementing protein /DB_XREF = gi: 4503600 /UG = Hs.48576 excision repair cross-complementing rodent repair |
| | deficiency, complementation group 5 (xeroderma pigmentosum, complementation group G (Cockayne syndrome)) |
| | /FL = gb: D16305.1 gb: L20046.1 gb: NM_000123.1 |
| 210422_x_at | gb: D50402.1 /DEF = Human mRNA for NRAMP1, complete cds. /FEA = mRNA /PROD = Nramp /DB_XREF = gi: 1000996 /UG = Hs.182611 solute |
| | carrier family 11 (proton-coupled divalent metal ion transporters), member 1 /FL = gb: D50402.1 gb: D50403.1 |
| | gb: NM_000578.1 gb: L32185.1 |
| 58900_at | Cluster Incl. AW025284: wu95h10.x1 Homo sapiens cDNA, 3 end /clone = IMAGE-990806 /clone_end = 3 /gb = AW025284 |
| | /gi = 5878814 /ug = Hs.237946 /len = 454 |
| 219890_at | gb: NM_013252.1 /DEF = Homo sapiens C-type (calcium dependent, carbohydrate-recognition domain) lectin, superfamily member 5 |
| | (CLECSF5), mRNA. /FEA = mRNA /GEN = CLECSF5 /PROD = C-type (calcium dependent, carbohydrate-recognition domain) lectin, |
| | superfamilymember 5 /DB_XREF = gi: 10281668 /UG = Hs.126355 C-type (calcium dependent, carbohydrate-recognition domain) |
| | lectin, superfamily member 5 /FL = gb: NM_013252.1 gb: AF139768.1 |
| 208112_x_at | gb: NM_006795.1 /DEF = Homo sapiens EH domain containing 1 (EHD1), mRNA. /FEA = mRNA /GEN = EHD1 /PROD = EH domain containing 1 |
| | /DB_XREF = gi: 5803008 /UG = Hs.155119 EH domain containing 1 /FL = gb: AF099011.1 gb: NM_006795.1 |
| 218655_s_at | gb: NM_017748.1 /DEF = Homo sapiens hypothetical protein FLJ20291 (FLJ20291), mRNA. /FEA = mRNA /GEN = FLJ20291 |
| | /PROD = hypothetical protein FLJ20291 /DB_XREF = gi: 8923270 /UG = Hs.8928 hypothetical protein FLJ20291 /FL = gb: NM_017748.1 |
| 214230_at | Consensus includes gb: R37664 /FEA = EST /DB_XREF = gi: 795120 /DB_XREF = est: yf61e05.s1 /CLONE = IMAGE: 26577 |
| | /UG = Hs.146409 cell division cycle 42 (GTP-binding protein, 25 kD) |
| 201898_s_at | Consensus includes gb: AI126625 /FEA = EST /DB_XREF = gi: 3595139 /DB_XREF = est: qd83a12.x1 /CLONE = IMAGE: 1736062 |
| | /UG = Hs.80612 ubiquitin-conjugating enzyme E2A (RAD6 homolog) /FL = gb: M74524.1 gb: NM_003336.1 |
| 210773_s_at | gb: U81501.1 /DEF = Human lipoxin A4 receptor mRNA, complete cds. /FEA = mRNA /PROD = lipoxin A4 receptor /DB_XREF = gi: 1916075 |
| | /UG = Hs.99855 formyl peptide receptor-like 1 /FL = gb: M76672.1 gb: M84562.1 gb: M88107.1 gb: U81501.1 gb: AF054013.1 |
| | gb: NM_001462.1 |
| 32069_at | Cluster Incl. AB014515: Homo sapiens mRNA for KIAA0615 protein, complete cds /cds = (237, 2927) /gb = AB014515 /gi = 3327043 |
| | /ug = Hs.155972 /len = 3319 |
| 121_at | X69699 /FEATURE = /DEFINITION = HSPAX8A H. sapiens Pax8 mRNA |
| 202053_s_at | gb: L47162.1 /DEF = Human fatty aldehyde dehydrogenase (FALDH) mRNA, complete cds. /FEA = mRNA /GEN = FALDH /PROD = fatty |
| | aldehyde dehydrogenase /DB_XREF = gi: 1082035 /UG = Hs.159608 aldehyde dehydrogenase 3 family, member A2 /FL = gb: L47162.1 |
| | gb: U46689.1 gb: NM_000382.1 |
| 212335_at | Consensus includes gb: AW167793 /FEA = EST /DB_XREF = gi: 6399401 /DB_XREF = est: xg56d07.x1 /CLONE = IMAGE: 2632333 /UG = Hs.164036 |
| | Homo sapiens AKAP350C mRNA sequence, alternatively spliced |
| 209813_x_at | gb: M16768.1 /DEF = Human T-cell receptor gamma chain VJCI-CII-CIII region mRNA, complete cds. /FEA = mRNA /GEN = TCRG |
| | /DB_XREF = gi: 339399 /UG = Hs.112259 T cell receptor gamma locus /FL = gb: M16768.1 gb: AF151103.1 |
| 204747_at | gb: NM_001549.1 /DEF = Homo sapiens interferon-induced protein with tetratricopeptide repeats 4 (IFIT4), mRNA. /FEA = mRNA |
| | /GEN = IFIT4 /PROD = interferon-induced protein withtetratricopeptide repeats 4 /DB_XREF = gi: 4504586 /UG = Hs.181874 |
| | interferon-induced protein with tetratricopeptide repeats 4 /FL = gb: BC001383.1 gb: BC004977.1 gb: U52513.1 gb: AF026939.1 |
| | gb: AF083470.1 gb: NM_001549.1 |
| 212223_at | Consensus includes gb: AI926544 /FEA = EST /DB_XREF = gi: 5662508 /DB_XREF = est: wo46c12.x1 /CLONE = IMAGE: 2458390 /UG = Hs.303154 |
| | popeye protein 3 |
| 218880_at | Consensus includes gb: N36408 /FEA = EST /DB_XREF = gi: 1157550 /DB_XREF = est: yy33f03.s1 /CLONE = IMAGE: 273053 /UG = Hs.325364 |
| | hypothetical protein FLJ23306 /FL = gb: NM_024530.1 |
| 202460_s_at | gb: NM_014646.1 /DEF = Homo sapiens lipin 2 (LPIN2), mRNA. /FEA = mRNA /GEN = LPIN2 /PROD = lipin 2 /DB_XREF = gi: 7662021 |
| | /UG = Hs.166318 lipin 2 /FL = gb: D87436.1 gb: NM_014646.1 |
| 58780_s_at | Cluster Incl. R42449: yg02a07.s1 Homo sapiens cDNA, 3 end /clone = IMAGE-30831 /clone_end = 3 /gb = R42449 /gi = 817213 |
| | /ug = Hs.235831 /len = 431 |
| 205281_s_at | gb: NM_002641.1 /DEF = Homo sapiens phosphatidylinositol glycan, class A (paroxysmal nocturnal hemoglobinuria) (PIGA), |
| | transcript variant 1, mRNA. /FEA = mRNA /GEN = PIGA /PROD = phosphatidylinositol glycan, class A isoform 1 /DB_XREF = gi: 11863129 |
| | /UG = Hs.51 phosphatidylinositol glycan, class A (paroxysmal nocturnal hemoglobinuria) /FL = gb: NM_002641.1 gb: D11466.1 |
| 218251_at | gb: NM_021242.1 /DEF = Homo sapiens hypothetical protein STRAIT11499 (STRAIT11499), mRNA. /FEA = mRNA /GEN = STRAIT11499 |
| | /PROD = hypothetical protein STRAIT11499 /DB_XREF = gi: 10864050 /UG = Hs.236556 hypothetical protein STRAIT11499 /FL = gb: NM_021242.1 |
| 212481_s_at | Consensus includes gb: AI214061 /FEA = EST /DB_XREF = gi: 3777662 /DB_XREF = est: ap29d10.x1 /CLONE = IMAGE: 1956787 |
| | /UG = Hs.250641 tropomyosin 4 |
| 213292_s_at | Consensus includes gb: AA908770 /FEA = EST /DB_XREF = gi: 3048175 /DB_XREF = est: o106a11.s1 /CLONE = IMAGE: 1522652 |
| | /UG = Hs.283881 KIAA0713 protein |
| 221497_x_at | gb: BC005369.1 /DEF = Homo sapiens, chromosome 1 open reading frame 12, clone MGC: 12484, mRNA, complete cds. |
| | /FEA = mRNA /PROD = chromosome 1 open reading frame 12 /DB_XREF = gi: 13529208 /UG = Hs.6523 chromosome 1 open reading frame 12 |
| | /FL = gb: AF229245.1 gb: AF277176.1 gb: NM_022051.1 gb: BC005369.1 |
| 212185_x_at | Consensus includes gb: NM_005953.1 /DEF = Homo sapiens metallothionein 2A (MT2A), mRNA. /FEA = CDS /GEN = MT2A |
| | /PROD = metallothionain 2A /DB_XREF = gi: 5174763 /UG = Hs.118786 metallothionein 2A /FL = gb: NM_005953.1 |
| 203961_at | Consensus includes gb: AL157398 /DEF = Human DNA sequence from clone RP11-56H7 on chromosome 10. Contains ESTs, STS5 and GSSs. |
| | Contains the gene for the nebulette protein (NEBL, actin-binding Z-disc protein) /FEA = mRNA_1 /DB_XREF = gi: 10045326 |
| | /UG = Hs.5025 nebulette /FL = gb: NM_006393.1 |
| 210986_s_at | gb: Z24727.1 /DEF = H. sapiens tropomyosin isoform mRNA, complete CDS. /FEA = mRNA /PROD = tropomyosin isoform /DB_XREF = gi: 854188 |
| | /UG = Hs.77899 tropomyosin 1 (alpha) /FL = gb: Z24727.1 |
| 41386_i_at | Cluster Incl. AB002344: Human mRNA for KIAA0346 gene, partial cds /cds = (0, 4852) /gb = AB002344 /gi = 2280479 /ug = Hs.103915 |
| | /len = 6121 |
| 201874_at | Consensus includes gb: BF978611 /FEA = EST /DB_XREF = gi: 12345826 /DB_XREF = est: 602149061F1 /CLONE = IMAGE: 4307822 |
| | /UG = Hs.14891 hypothetical protein FLJ21047 /FL = gb: NM_024569.1 |
| 211781_x_at | gb: BC006164.1 /DEF = Homo sapiens, clone MGC: 13219, mRNA, complete cds. /FEA = mRNA /PROD = Unknown (protein for MGC: 13219) |
| | /DB_XREF = gi: 13544062 /FL = gb: BC006164.1 |
| 202459_s_at | Consensus includes gb: U55968 /FEA = EST /DB_XREF = gi: 1354524 /DB_XREF = est: HSU55968 /CLONE = 26508 /UG = Hs.166318 lipin 2 |
| | /FL = gb: D87436.1 gb: NM_014646.1 |
| 217908_s_at | gb: NM_018442.1 /DEF = Homo sapiens PC326 protein (PC326), mRNA. /FEA = mRNA /GEN = PC326 /PROD = PC326 protein /DB_XREF = gi: 8923955 |
| | /UG = Hs.279882 PC326 protein /FL = gb: AL136738.1 gb: AF150734.1 gb: NM_018442.1 |
| 202427_s_at | gb: NM_015415.1 /DEF = Homo sapiens DKFZP564B167 protein (DKFZP564B167), mRNA. /FEA = mRNA /GEN = DKFZP564B167 /PROD = DKFZP564B167 |
| | protein /DB_XREF = gi: 7661601 /UG = Hs.76285 DKFZP564B167 protein /FL = gb: AL110297.1 gb: NM_015415.1 |
| 213716_s_at | Consensus includes gb: BF939675 /FEA = EST /DB_XREF = gi: 12357086 /DB_XREF = est: nac79g07.x1 /CLONE = IMAGE: 3440820 /UG = Hs.326612 |
| | Homo sapiens secreted and transmembrane 1 (SECTM1), mRNA |
| 212550_at | Consensus includes gb: AI149535 /FEA = EST /DB_XREF = gi: 3678004 /DB_XREF = est: qc70f11.x1 /CLONE = IMAGE: 1714989 /UG = Hs.24064 Homo |
| | sapiens mRNA; cDNA DKFZp586N1323 (from clone DKFZp586N1323) |
| 33323_r_at | Cluster Incl. X57348: H. sapiens mRNA (clone 9112) /cds = (165, 911) /gb = X57348 /gi = 23939 /ug = Hs.184510 /len = 1407 |
| 220947_s_at | gb: NM_015527.1 /DEF = Homo sapiens DKFZP434P1750 protein (DKFZP434P1750), mRNA. /FEA = mRNA /GEN = DKFZP434P1750 |
| | /PROD = DKFZP434P1750 protein /DB_XREF = gi: 7661587 /UG = Hs.7274 DKFZP434P1750 protein /FL = gb: NM_015527.1 |
| 202665_s_at | gb: NM_003387.2 /DEF = Homo sapiens Wiskott-Aldrich syndrome protein interacting protein (WASPIP), mRNA. /FEA = mRNA |
| | /GEN = WASPIP /PROD = WASP-interacting protein /DB_XREF = gi: 8400739 /UG = Hs.24143 Wiskott-Aldrich syndrome protein |
| | interacting protein /FL = gb: NM_003387.2 |
| 220746_s_at | gb: NM_016290.1 /DEF = Homo sapiens retinoid x receptor interacting protein (LOC51720), mRNA. /FEA = mRNA /GEN = LOC51720 |
| | /PROD = retinoid x receptor interacting protein /DB_XREF = gi: 7706482 /UG = Hs.7889 retinoid x receptor interacting protein |
| | /FL = gb: AF113538.1 gb: NM_016290.1 |
| 218078_s_at | gb: NM_016598.1 /DEF = Homo sapiens DHHC1 protein (LOC51304), mRNA. /FEA = mRNA /GEN = LOC51304 /PROD = DHHC1 protein |
| | /DB_XREF = gi: 7706132 /UG = Hs.14896 DHHC1 protein /FL = gb: AF247703.1 gb: NM_016598.1 |
| 203429_s_at | gb: NM_016227.1 /DEF = Homo sapiens membrane protein CH1 (CH1), mRNA. /FEA = mRNA /GEN = CH1 /PROD = membrane protein CH1 |
| | /DB_XREF = gi: 7705321 /UG = Hs.108636 membrane protein CH1 /FL = gb: AF097535.1 gb: NM_016227.1 |
| 203120_at | gb: NM_005426.1 /DEF = Homo sapiens tumor protein p53-binding protein, 2 (TP53BP2), mRNA. /FEA = mRNA /GEN = TP53BP2 |
| | /PROD = tumor protein p53-binding protein, 2 /DB_XREF = gi: 4885642 /UG = Hs.44585 tumor protein p53-binding protein, 2 |
| | /FL = gb: U58334.1 gb: NM_005426.1 |
| 203143_s_at | Consensus includes gb: T79953 /FEA = EST /DB_XREF = gi: 698462 /DB_XREF = est: yd85c11.s1 /CLONE = IMAGE: 115028 /UG = Hs.158282 |
| | KIAA0040 gene product /FL = gb: D25539.1 gb: NM_014656.1 |
| 41387_r_at | Cluster Incl. AB002344: Human mRNA for KIAA0346 gene, partial cds /cds = (0.4852) /gb = AB002344 /gi = 2280479 /ug = Hs.103915 |
| | /len = 6121 |
| 202286_s_at | Consensus includes gb: J04152 /DEF = Human gastrointestinal tumor-associated antigen GA733-1 protein gene, complete cds, |
| | clone 05516 /FEA = mRNA /DB_XREF = gi: 182893 /UG = Hs.23582 tumor-associated calcium signal transducer 2 /FL = gb: NM_002353.1 |
| 200670_at | gb: NM_005080.1 /DEF = Homo sapiens X-box binding protein 1 (XBP1), mRNA. /FEA = mRNA /GEN = XBP1 /PROD = X-box binding protein 1 |
| | /DB_XREF = gi: 4827057 /UG = Hs.149923 X-box binding protein 1 /FL = gb: BC000938.1 gb: M31627.1 gb: NM_005080.1 |
| 211612_s_at | gb: U62858.1 /DEF = Human interleukin-13 receptor mRNA, complete cds. /FEA = mRNA /PROD = interleukin-13 receptor |
| | /DB_XREF = gi: 1695875 /FL = gb: U62858.1 |
| 203233_at | gb: NM_000418.1 /DEF = Homo sapiens interleukin 4 receptor (IL4R), mRNA. /FEA = mRNA /GEN = IL4R /PROD = interleukin 4 receptor |
| | precursor /DB_XREF = gi: 4557668 /UG = Hs.75545 interleukin 4 receptor /FL = gb: NM_000418.1 |
| 204507_s_at | gb: NM_000945.1 /DEF = Homo sapiens protein phosphatase 3 (formerly 2B), regulatory subunit B (19 kD), alpha isoform |
| | (calcineurin B, type I) (PPP3R1), mRNA. /FEA = mRNA /GEN = PPP3R1 /PROD = protein phosphatase 3 (formerly 2B), |
| | regulatorysubunit B (19 kD), alpha isoform (calcineurin B, type I) /DB_XREF = gi: 4506024 /UG = Hs.278540 protein phosphatase 3 |
| | (formerly 2B), regulatory subunit B (19 kD), alpha isoform (calcineurin B, type I) /FL = gb: M30773.1 gb: NM_000945.1 |
| 218136_s_at | gb: NM_018579.1 /DEF = Homo sapiens mitochondrial solute carrier (LOC51312), mRNA. /FEA = mRNA /GEN = LOC51312 /PROD = hypothetical |
| | protein PRO1278 /DB_XREF = gi: 8924027 /UG = Hs.300496 mitochondrial solute carrier /FL = gb: AF155660.1 gb: AF116630.1 |
| | gb: NM_018579.1 |
| 201582_at | Consensus, includes gb: AL121900 /DEF = Human DNA sequence from clone RP11-379J5 on chromosome 20 Contains the last exon of |
| | the SEC23B gene for Sec23 (S. cerevisiae) homolog B, a putative novel gene, the 5 end of the gene for a novel protein |
| | similar to bacterial histidyl-tRNA synthetas . . . /FEA = mRNA /DB_XREF = gi: 11121203 /UG = Hs.173497 Sec23 (S. cerevisiae) homolog |
| | B /FL = gb: BC005404.1 gb: NM_006363.1 |
| 209339_at | gb: U76248.1 /DEF = Human hSIAH2 mRNA, complete cds. /FEA = mRNA /PROD = hSIAH2 /DB_XREF = gi: 2673967 /UG = Hs.20191 seven in absentia |
| | (Drosophila) homolog 2 /FL = gb: U76248.1 gb: NM_005067.1 |
| 208030_s_at | gb: NM_001119.2 /DEF = Homo sapiens adducin 1 (alpha) (ADD1), transcript variant 1, mRNA. /FEA = mRNA /GEN = ADD1 /PROD = adducin 1 |
| | (alpha) isoform a /DB_XREF = gi: 7710113 /FL = gb: NM_001119.2 |
| 201195_s_at | gb: AB018009.1 /DEF = Homo sapiens mRNA for L-type amino acid transporter 1, complete cds. /FEA = mRNA /GEN = hLAT1 /PROD = L-type |
| | amino acid transporter 1 /DB_XREF = gi: 5926731 /UG = Hs.184601 solute carrier family 7 (cationic amino acid transporter, |
| | y+ system), member 5 /FL = gb: AF077866.1 gb: AB018542.1 gb: AF104032.1 gb: NM_003486.1 gb: AB017908.1 gb: AB018009.1 |
| 217918_at | gb: NM_014183.1 /DEF = Homo sapiens HSPC162 protein (HSPC162), mRNA. /FEA = mRNA /GEN = HSPC162 /PROD = HSPC162 protein |
| | /DB_XREF = gi: 7661821 /UG = Hs.100002 HSPC162 protein /FL = gb: BC002481.1 gb: AY026513.1 gb: AF161511.1 gb: NM_014183.1 |
| | gb: AF165516.1 |

TABLE 4
Genes from Cluster Analysis 4

| Affymetrix | |
|---|---|
| internal | |
| designation | Description of the sequence in the GeneBank data base |
| 221798_x_at | Consensus includes gb: AI183766 /FEA = EST /DB_XREF = gi: 3734404 /DB_XREF = est: qe17g06.x1 /CLONE = IMAGE: 1739290 /UG = Hs.182426 |
| | ribosomal |
| | protein S2 |
| 218231_at | gb: NM_017567.1 /DEF = Homo sapiens N-Acetylglucosamine kinase (HSA242910), mRNA. /FEA = mRNA /GEN = HSA242910 /PROD = N- |
| | Acetylglucosamine |
| | kinase /DB_XREF = gi: 8923736 /UG = Hs.7036 N-Acetylglucosamine kinase /FL = gb: BC001029.1 gb: BC005371.1 gb: NM_017567.1 |
| 201009_s_at | Consensus includes gb: AI439556 /FEA = EST /DB_XREF = gi: 4305149 /DB_XREF = est: tc90c12.x1 /CLONE = IMAGE: 2073430 /UG = Hs.179526 |
| | upregulated |
| | by 1,25-dihydroxyvitamin D-3 /FL = gb: NM_006472.1 gb: S73591.1 |
| 212998_x_at | Consensus includes gb: AI583173 /FEA = EST /DB_XREF = gi: 4569070 /DB_XREF = est: tq64e04.x1 /CLONE = IMAGE: 2213598 /UG = Hs.73931 |
| | major |
| | histocompatibility complex, class II, DQ beta 1 |
| 201464_x_at | Consensus includes gb: BG491844 /FEA = EST /DB_XREF = gi: 13453356 /DB_XREF = est: 602535931T1 /CLONE = IMAGE: 4684998 /UG = Hs.78465 |
| | v-jun avian |
| | sarcoma virus 17 oncogene homolog /FL = gb: BC002646.1 gb: NM_002228.2 |
| 214687_x_at | Consensus includes gb: AK026577.1 /DEF = Homo sapiens cDNA: FLJ22924 fis, clone KAT06977, highly similar to HSALDAR Human |
| | fibroblast |
| | mRNA for aldolase A. /FEA = mRNA /DB_XREF = gi: 10439461 /UG = Hs.273415 aldolase A, fructose-bisphosphate |
| 220532_s_at | gb: NM_014020.1 /DEF = Homo sapiens LR8 protein (LR8), mRNA. /FEA = mRNA /GEN = LR8 /PROD = LR8 protein /DB_XREF = gi: 7662497 |
| | /UG = Hs.190161 |
| | LR8 protein /FL = gb: AF115384.1 gb: NM_014020.1 |
| 205987_at | gb: NM_001765.1 /DEF = Homo sapiens CD1C antigen, c polypeptide (CD1C), mRNA. /FEA = mRNA /GEN = CD1C /PROD = CD1C antigen, c polypeptide |
| | /DB_XREF = gi: 4502646 /UG = Hs.1311 CD1C antigen, c polypeptide /FL = gb: M28827.1 gb: NM_001765.1 |
| 202658_at | .1 /DEF = Homo sapiens peroxisomal biogenesis factor 11B (PEX11B), mRNA. /FEA = mRNA /GEN = PEX11B /PROD = peroxisomal biogenesis |
| | factor 11B /DB_XREF = gi: 4505718 /UG = Hs.83023 peroxisomal biogenesis factor 11B /FL = gb: AF093670.1 gb: AB018080.1 |
| | gb: NM_003846.1 |
| 200991_s_at | gb: NM_014748.1 /DEF = Homo sapiens KIAA0064 gene product (KIAA0064), mRNA. /FEA = mRNA /GEN = KIAA0064 /PROD = KIAA0064 gene |
| | product |
| | /DB_XREF = gi: 7661889 /UG = Hs.278569 sorting nexin 17 /FL = gb: BC002524.1 gb: BC002610.1 gb: D31764.1 gb: NM_014748.1 |
| 209007_s_at | gb: AF267856.1 /DEF = Homo sapiens HT033 mRNA, complete cds. /FEA = mRNA /PROD = HT033 /DB_XREF = gi: 12006038 /UG = Hs.8084 hypothetical |
| | protein |
| | dJ465N24.2.1 /FL = gb: AF247168.1 gb: AF267856.1 |
| 213969_x_at | Consensus includes gb: BF683426 /FEA = EST /DB_XREF = gi: 11968834 /DB_XREF = est: 602139603F1 /CLONE = IMAGE: 4300777 /UG = Hs.183698 |
| | ribosomal protein L29 |
| 200094_s_at | Consensus includes gb: AI004246 /FEA = EST /DB_XREF = gi: 3213756 /DB_XREF = est: ou03g06.x1 /CLONE = IMAGE: 1625242 /UG = Hs.75309 |
| | eukaryotic translation elongation factor 2 |
| 208929_x_at | gb: BC004954.1 /DEF = Homo sapiens, clone MGC: 10897, mRNA, complete cds. /FEA = mRNA /PROD = Unknown (protein for MGC: 10897) |
| | /DB_XREF = gi: 13436331 /UG = Hs.180842 ribosomal protein L13 /FL = gb: BC000851.1 gb: BC004954.1 gb: NM_000977.1 |
| 206170_at | gb: NM_000024.2 /DEF = Homo sapiens adrenergic, beta-2-, receptor, surface (ADRB2), mRNA. /FEA = mRNA /GEN = ADRB2 |
| | /PROD = adrenergic, beta-2-, |
| | receptor, surface /DB_XREF = gi: 13162366 /UG = Hs.2551 adrenergic, beta-2-, receptor, surface /FL = gb: NM_000024.2 gb: M15169.1 |
| 212582_at | Consensus includes gb: AL049923.1 /DEF = Homo sapiens mRNA; cDNA DKFZp564E2282 (from clone DKFZp564E2282). /FEA = mRNA |
| | /DB_XREF = gi: 4884169 /UG = Hs.109694 KIAA1451 protein |
| 208630_at | Consensus includes gb: AI972144 /FEA = EST /DB_XREF = gi: 5768970 /DB_XREF = est: wr63b03.x1 /CLONE = IMAGE: 2492333 |
| | /UG = Hs.75860 hydroxyacyl-Coenzyme A dehydrogenase3-ketoacyl-Coenzyme A thiolaseenoyl-Coenzyme A hydratase |
| | (trifunctional protein), alpha subunit /FL = gb: NM_000182.1 gb: D16480.1 gb: U04627.1 |
| 214058_at | Consensus includes gb: M19720 /DEF = Human L-myc protein gene, complete cds /FEA = mRNA_2 /DB_XREF = gi: 188906 |
| | /UG = Hs.92137 v-myc avian myelocytomatosis viral oncogene homolog 1, lung carcinoma derived |
| 201991_s_at | Consensus includes gb: BF223224 /FEA = EST /DB_XREF = gi: 11130401 /DB_XREF = est: 7q30g05.x1 /CLONE = IMAGE: 3700017 |
| | /UG = Hs.149436 kinesin family member 5B /FL = gb: NM_004521.1 |
| 212179_at | Consensus includes gb: AW157501 /FEA = EST /DB_XREF = gi: 6228902 /DB_XREF = est: au83a02.x1 /CLONE = IMAGE: 2782826 |
| | /UG = Hs.18368 DKFZP564B0769 protein |
| 210949_s_at | gb: BC000533.1 /DEF = Homo sapiens, Similar to eukaryotic translation initiation factor 3, subunit 8 (110 kD), clone |
| | MGC: 8693, |
| | mRNA, complete cds. /FEA = mRNA /PROD = Similar to eukaryotic translation initiationfactor 3, subunit 8 (110 kD) |
| | /DB_XREF = gi: 12653522 /UG = Hs.4835 eukaryotic translation initiation factor 3, subunit 8 (110 kD) /FL = gb: BC000533.1 |
| 213142_x_at | Consensus includes gb: AV700415 /FEA = EST /DB_XREF = gi: 10302386 /DB_XREF = est: AV700415 /CLONE = GKCDGA04 /UG = Hs.12969 hypothetical |
| | protein |
| 217817_at | Consensus includes gb: BE891920 /FEA = EST /DB_XREF = gi: 10351728 /DB_XREF = est: 601435490F1 /CLONE = IMAGE: 3920590 |
| | /UG = Hs.323342 actin related protein 23 complex, subunit 4 (20 kD) /FL = gb: AF006087.1 gb: NM_005718.1 |
| 216041_x_at | Consensus includes gb: AK023348.1 /DEF = Homo sapiens cDNA FLJ13286 fis, clone OVARC1001154, highly similar to Homo sapiens |
| | clone 24720 epithelin 1 and 2 mRNA. /FEA = mRNA /DB_XREF = gi: 10435243 /UG = Hs.180577 granulin |
| 202687_s_at | gb: U57059.1 /DEF = Homo sapiens Apo-2 ligand mRNA, complete cds. /FEA = mRNA /PROD = Apo-2 ligand /DB_XREF = gi: 1336207 |
| | /UG = Hs.83429 tumor necrosis factor (ligand) superfamily, member 10 /FL = gb: U37518.1 gb: U57059.1 gb: NM_003810.1 |
| 215230_x_at | Consensus includes gb: AA679705 /FEA = EST /DB_XREF = gi: 2660227 /DB_XREF = est: ah15e04.s1 /CLONE = IMAGE: 1156734 |
| | /UG = Hs.4835 eukaryotic translation initiation factor 3, subunit 8 (110 kD) |
| 200647_x_at | gb: NM_003752.2 /DEF = Homo sapiens eukaryotic translation initiation factor 3, subunit 8 (110 kD) (EIF3S8), mRNA. |
| | /FEA = mRNA /GEN = EIF3S8 /PROD = eukaryotic translation initiation factor 3, subunit 8 (110 kD) /DB_XREF = gi: 5579457 |
| | /UG = Hs.4835 eukaryotic translation initiation factor 3, subunit 8 (110 kD) /FL = gb: NM_003752.2 |
| 200078_s_at | gb: BC005876.1 /DEF = Homo sapiens, ATPase, H+ transporting, lysosomal (vacuolar proton pump) 21 kD, clone MGC: 4498, mRNA, |
| | complete cds. /FEA = mRNA /PROD = ATPase, H+ transporting, lysosomal (vacuolarproton pump) 21 kD /DB_XREF = gi: 13543437 |
| | /FL = gb: BC005876.1 |
| 202169_s_at | gb: AF302110.1 /DEF = Homo sapiens alpha-aminoadipic semialdehyde dehydrogenase-phosphopantetheinyl transferase mRNA, |
| | complete cds. /FEA = mRNA /PROD = alpha-aminoadipic semialdehydedehydrogenase-phosphopantetheinyl transferase |
| | /DB_XREF = gi: 11120434 /UG = Hs.64595 aminoadipate-semialdehyde dehydrogenase-phosphopantetheinyl transferase |
| | /FL = gb: AF302110.1 gb: AF136978.1 gb: AF151838.1 gb: AF151057.1 gb: NM_015423.1 gb: AF201943.1 |
| 202659_at | gb: NM_002801.1 /DEF = Homo sapiens proteasome (prosome, macropain) subunit, beta type, 10 (PSMB10), mRNA. /FEA = mRNA |
| | /GEN = PSMB10 /PROD = proteasome (prosome, macropain) subunit, betatype, 10 /DB_XREF = gi: 4506190 /UG = Hs.9661 proteasome |
| | (prosome, macropain) subunit, beta type, 10 /FL = gb: NM_002801.1 |
| 221488_s_at | gb: AF230924.1 /DEF = Homo sapiens brain acetylcholinesterase putative membrane anchor mRNA, complete cds. /FEA = mRNA |
| | /PROD = brain acetylcholinesterase putative membraneanchor /DB_XREF = gi: 7341254 /UG = Hs.107187 divalent cation tolerant |
| | protein CUTA /FL = gb: AF230924.1 |
| 221622_s_at | gb: AF246240.1 /DEF = Homo sapiens HT026 mRNA, complete cds. /FEA = mRNA /PROD = HT026 /DB_XREF = gi: 12005514 |
| | /UG = Hs.24371 uncharacterized hypothalamus protein HT007 /FL = gb: AF246240.1 |
| 206050_s_at | gb: NM_002939.1 /DEF = Homo sapiens ribonucleaseangiogenin inhibitor (RNH), mRNA. /FEA = mRNA /GEN = RNH |
| | /PROD = ribonucleaseangiogenin |
| | inhibitor /DB_XREF = gi: 4506564 /UG = Hs.75108 ribonucleaseangiogenin inhibitor /FL = gb: M36717.1 gb: NM_002939.1 |
| 212199_at | Consensus includes gb: AL566962 /FEA = EST /DB_XREF = gi: 12919867 /DB_XREF = est: AL566962 /CLONE = CS0DF026YH08 (3 prime) |
| | /UG = Hs.284281 Human putative ribosomal protein S1 mRNA |
| 201861_s_at | Consensus includes gb: BF965566 /FEA = EST /DB_XREF = gi: 12332781 /DB_XREF = est: 602277032F1 /CLONE = IMAGE: 4364790 |
| | /UG = Hs.326159 leucine rich repeat (in FLII) interacting protein 1 /FL = gb: NM_004735.1 |
| 212795_at | Consensus includes gb: AL137753.1 /DEF = Homo sapiens mRNA; cDNA DKFZp434K1412 (from clone DKFZp434K1412). |
| | /FEA = mRNA /DB_XREF = gi: 6808455 /UG = Hs.12144 KIAA1033 protein |
| 213574_s_at | Consensus includes gb: AA861608 /FEA = EST /DB_XREF = gi: 2953748 /DB_XREF = est: ak34e01.s1 /CLONE = IMAGE: 1407864 |
| | /UG = Hs.180446 karyopherin (importin) beta 1 |
| 215171_s_at | Consensus includes gb: AK023063.1 /DEF = Homo sapiens cDNA FLJ13001 fis, clone NT2RP3000341, highly similar to Homo |
| | sapiens mitochondrial inner membrane preprotein translocase Tim17a mRNA, nuclear gene encoding mitochondrial protein. |
| | /FEA = mRNA /DB_XREF = gi: 10434808 /UG = Hs.20716 translocase of inner mitochondrial membrane 17 (yeast) homolog A |
| 210137_s_at | gb: BC001286.1 /DEF = Homo sapiens, Similar to dCMP deaminase, clone MGC: 5160, mRNA, complete cds. /FEA = mRNA |
| | /PROD = Similar to dCMP deaminase /DB_XREF = gi: 12654884 /UG = Hs.76894 dCMP deaminase /FL = gb: BC001286.1 |
| 212943_at | Consensus includes gb: AB011100.2 /DEF = Homo sapiens mRNA for KIAA0528 protein, partial cds. /FEA = mRNA /GEN = KIAA0528 |
| | /PROD = KIAA0528 protein /DB_XREF = gi: 6683714 /UG = Hs.30656 KIAA0528 gene product |
| 202164_s_at | gb: AF180476.1 /DEF = Homo sapiens CALIFp (CALIF) mRNA, complete cds. /FEA = mRNA /GEN = CALIF /PROD = CALIFp /DB_XREF = gi: 6856208 |
| | /UG = Hs.26703 CCR4-NOT transcription complex, subunit 8 /FL = gb: AF053318.1 gb: NM_004779.1 gb: AL122045.1 gb: AF180476.1 |
| 209005_at | gb: AF157323.1 /DEF = Homo sapiens p45SKP2-like protein mRNA, complete cds. /FEA = mRNA /PROD = p45SKP2-like protein |
| | /DB_XREF = gi: 7688696 /UG = Hs.5548 f-box and leucine-rich repeat protein 5 /FL = gb: AF199420.1 gb: AF142481.1 gb: AF157323.1 |
| 202899_s_at | gb: NM_003017.1 /DEF = Homo sapiens, splicing factor, arginineserine-rich 3 (SFRS3), mRNA. /FEA = mRNA /GEN = SFRS3 |
| | /PROD = splicing factor, arginineserine-rich 3 /DB_XREF = gi: 4506900 /UG = Hs.167460 splicing factor, arginineserine-rich 3 |
| | /FL = gb: L10838.1 gb: NM_003017.1 |
| 208777_s_at | gb: AF001212.1 /DEF = Homo sapiens 26S proteasome subunit 9 mRNA, complete cds. /FEA = mRNA /PROD = 26S proteasome subunit 9 |
| | /DB_XREF = gi: 2150045 /UG = Hs.90744 proteasome (prosome, macropain) 26S subunit, non-ATPase, 11 |
| | /FL = gb: BC000437.1 gb: BC004430.1 gb: AB003102.1 gb: AF001212.1 gb: NM_002815.1 |
| 219889_at | gb: NM_005479.1 /DEF = Homo sapiens frequently rearranged in advanced T-cell lymphomas (FRAT1), mRNA. /FEA = mRNA |
| | /GEN = FRAT1 /PROD = frequently rearranged in advanced T-celllymphomas /DB_XREF = gi: 4885246 |
| | /UG = Hs.126057 frequently rearranged in advanced T-cell lymphomas /FL = gb: U58975.2 gb: NM_005479.1 |
| 204961_s_at | gb: NM_000265.1 /DEF = Homo sapiens neutrophil cytosolic factor 1 (47 kD, chronic granulomatous disease, autosomal 1) (NCF1), |
| | mRNA. |
| | /FEA = mRNA /GEN = NCF1 /PROD = neutrophil cytosolic factor 1 /DB_XREF = gi: 4557784 /UG = Hs.1583 neutrophil cytosolic factor 1 |
| | (47 kD, chronic granulomatous disease, autosomal 1) /FL = gb: BC002816.1 gb: AF330627.1 gb: M55067.1 gb: M25665.1 gb: NM_000265.1 |
| 217106_x_at | Consensus includes gb: AF091078.1 /DEF = Homo sapiens clone 559 unknown mRNA, complete sequence. /FEA = mRNA |
| | /PROD = unknown /DB_XREF = gi: 3859993 /UG = Hs.125819 putative dimethyladenosine transferase |
| 212224_at | Consensus includes gb: NM_000689.1 /DEF = Homo sapiens aldehyde dehydrogenase 1, soluble (ALDH1), mRNA. /FEA = CDS /GEN = ALDH1 |
| | /PROD = aldehyde dehydrogenase 1, soluble /DB_XREF = gi: 4502030 /UG = Hs.76392 aldehyde dehydrogenase 1 family, |
| | member A1 /FL = gb: AF003341.1 gb: NM_000689.1 |
| 204125_at | gb: NM_016013.1 /DEF = Homo sapiens CGI-65 protein (LOC51103), mRNA. /FEA = mRNA /GEN = LOC51103 /PROD = CGI-65 protein |
| | /DB_XREF = gi: 7705778 /UG = Hs.106529 CGI-65 protein /FL = gb: BC000780.1 gb: AF151823.1 gb: NM_016013.1 |
| 217752_s_at | gb: NM_018235.1 /DEF = Homo sapiens hypothetical protein FLJ10830 (FLJ10830), mRNA. /FEA = mRNA /GEN = FLJ10830 |
| | /PROD = hypothetical protein |
| | FLJ10830 /DB_XREF = gi: 8922698 /UG = Hs.273230 hypothetical protein FLJ10830 /FL = gb: BC001375.1 gb: BC003176.1 gb: NM_018235.1 |
| 221087_s_at | gb: NM_014349.1 /DEF = Homo sapiens apolipoprotein L, 3 (APOL3), mRNA. /FEA = mRNA /GEN = APOL3 /PROD = apolipoprotein L, 3 |
| | /DB_XREF = gi: 7656972 /UG = Hs.241535 apolipoprotein L, 3 /FL = gb: AY014905.1 gb: AF070675.1 gb: NM_014349.1 |
| 218139_s_at | gb: NM_018229.1 /DEF = Homo sapiens hypothetical protein FLJ10813 (FLJ10813), mRNA. /FEA = mRNA /GEN = FLJ10813 |
| | /PROD = hypothetical protein FLJ10813 /DB_XREF = gi: 8922687 /UG = Hs.106210 hypothetical protein FLJ10813 |
| | /FL = gb: AL136685.1 gb: NM_018229.1 |
| 213878_at | Consensus includes gb: AI685944 /FEA = EST /DB_XREF = gi: 4897238 /DB_XREF = est: tu38g02.x1 /CLONE = IMAGE: 2253362 |
| | /UG = Hs.235069 RecQ protein-like (DNA helicase Q1-like) |
| 201346_at | gb: NM_024551.1 /DEF = Homo sapiens hypothetical protein FLJ21432 (FLJ21432), mRNA. /FEA = mRNA /GEN = FLJ21432 |
| | /PROD = hypothetical protein FLJ21432 /DB_XREF = gi: 13375714 /UG = Hs.11641 hypothetical protein FLJ21432 |
| | /FL = gb: NM_024551.1 gb: BC004906.1 |
| 206874_s_at | Consensus includes gb: AL138761 /DEF = Human DNA sequence from clone RP11-16H23 on chromosome 10. |
| | Contains the gene KIAA0204 (HSLK) for a protein kinase, the COL17A1 gene for collagen type XVII alpha 1 (BP180), |
| | ESTs and GSSs /FEA = mRNA_2 /DB_XREF = gi: 8573811 /UG = Hs.105751 Ste20-related serinethreonine kinase |
| | /FL = gb: D86959.1 gb: NM_014720.1 |
| 221808_at | Consensus includes gb: NM_004251.1 /DEF = Homo sapiens RAB9, member RAS oncogene family (RAB9), mRNA. /FEA = CDS /GEN = RAB9 |
| | /PROD = RAB9, member RAS oncogene family /DB_XREF = gi: 4759011 /UG = Hs.28726 RAB9, member RAS oncogene family |
| | /FL = gb: U44103.1 gb: NM_004251.1 |
| 211138_s_at | gb: BC005297.1 /DEF = Homo sapiens, Similar to kynurenine 3-monooxygenase (kynurenine 3-hydroxylase), clone MGC: 12362, mRNA, |
| | complete cds. /FEA = mRNA /PROD = Similar to kynurenine 3-monooxygenase(kynurenine 3-hydroxylase) /DB_XREF = gi: 13529016 |
| | /UG = Hs.107318 kynurenine 3-monooxygenase (kynurenine 3-hydroxylase) /FL = gb: BC005297.1 |
| 201386_s_at | gb: AF279891.1 /DEF = Homo sapiens dead box protein 15 mRNA, complete cds. /FEA = mRNA /PROD = dead box protein 15 |
| | /DB_XREF = gi: 9624452 /UG = Hs.5683 DEADH (Asp-Glu-Ala-AspHis) box polypeptide 15 /FL = gb: AB001636.1 gb: NM_001358.1 |
| | gb: AF279891.1 |
| 203037_s_at | gb: NM_014751.1 /DEF = Homo sapiens KIAA0429 gene product (KIAA0429), mRNA. /FEA = mRNA /GEN = KIAA0429 /PROD = KIAA0429 gene |
| | product |
| | /DB_XREF = gi: 7662113 /UG = Hs.77694 KIAA0429 gene product /FL = gb: AB007889.1 gb: NM_014751.1 |
| 218356_at | gb: NM_013393.1 /DEF = Homo sapiens cell division protein FtsJ (FJH1), mRNA. /FEA = mRNA /GEN = FJH1 /PROD = cell division protein |
| | FtsJ /DB_XREF = gi: 7019376 /UG = Hs.279877 cell division protein FtsJ /FL = gb: AF093415.1 gb: NM_013393.1 |
| 204405_x_at | gb: NM_014473.1 /DEF = Homo sapiens putative dimethyladenosine transferase (HSA9761), mRNA. /FEA = mRNA /GEN = HSA9761 |
| | /PROD = putative dimethyladenosine transferase /DB_XREF = gi: 7657197 /UG = Hs.125819 putative dimethyladenosine transferase |
| | /FL = gb: AF102147.1 gb: NM_014473.1 |
| 201608_s_at | gb: NM_007062.1 /DEF = Homo sapiens nuclear phosphoprotein similar to S. cerevisiae PWP1 (PWP1), mRNA. /FEA = mRNA /GEN = PWP1 |
| | /PROD = nuclear phosphoprotein similar to S. cerevisiae PWP1 /DB_XREF = gi: 5902033 |
| | /UG = Hs.172589 nuclear phosphoprotein similar to S. cerevisiae PWP1 /FL = gb: BC001652.1 gb: L07758.1 gb: NM_007062.1 |
| 201569_s_at | gb: NM_015380.1 /DEF = Homo sapiens CGI-51 protein (CGI-51), mRNA. /FEA = mRNA /GEN = CGI-51 /PROD = CGI-51 protein |
| | /DB_XREF = gi: 7661541 |
| | /UG = Hs.4877 CGI-51 protein /FL = gb: AF151809.1 gb: NM_015380.1 |
| 222244_s_at | Consensus includes gb: AK000749.1 /DEF = Homo sapiens cDNA FLJ20742 fis, clone HEP06891. /FEA = mRNA /DB_XREF = gi: 7021031 |
| | /UG = Hs.52184 hypothetical protein FLJ20618 |
| 201241_at | gb: NM_004939.1 /DEF = Homo sapiens DEADH (Asp-Glu-Ala-AspHis) box polypeptide 1 (DDX1), mRNA. /FEA = mRNA /GEN = DDX1 |
| | /PROD = DEADH (Asp-Glu-Ala-AspHis) box polypeptide 1 /DB_XREF = gi: 4826685 /UG = Hs.78580 DEADH (Asp-Glu-Ala-AspHis) box |
| | polypeptide 1 |
| | /FL = gb: X70649.1 gb: NM_004939.1 |
| 220731_s_at | gb: NM_018090.1 /DEF = Homo sapiens hypothetical protein FLJ10420 (FLJ10420), mRNA. /FEA = mRNA /GEN = FLJ10420 |
| | /PROD = hypothetical protein FLJ10420 /DB_XREF = gi: 8922415 /UG = Hs.289087 hypothetical protein FLJ10420 /FL = gb: NM_018090.1 |
| 208799_at | gb: BC004146.1 /DEF = Homo sapiens, proteasome (prosome, macropain) subunit, beta type, 5, clone MGC: 2175, mRNA, complete |
| | cds. |
| | /FEA = mRNA /PROD = proteasome (prosome, macropain) subunit, betatype, 5 /DB_XREF = gi: 13278740 /UG = Hs.78596 proteasome |
| | (prosome, macropain) subunit, beta type, 5 /FL = gb: BC004146.1 gb: NM_002797.1 gb: D29011.1 |
| 221718_s_at | gb: M90360.1 /DEF = Human type II cAMP-dependent protein kinase (Ht31) mRNA, complete cds. /FEA = CDS /GEN = Ht31 |
| | /PROD = protein kinase /DB_XREF = gi: 184434 /FL = gb: M90360.1 |
| 208662_s_at | Consensus includes gb: AI885338 /FEA = EST /DB_XREF = gi: 5590502 /DB_XREF = est: w192e09.x1 /CLONE = IMAGE: 2432392 |
| | /UG = Hs.118174 tetratricopeptide repeat domain 3 /FL = gb: D84294.1 |
| 202118_s_at | Consensus includes gb: AA541758 /FEA = EST /DB_XREF = gi: 2288192 /DB_XREF = est: ni87d05.s1 /CLONE = IMAGE: 983817 |
| | /UG = Hs.14158 copine III /FL = gb: AB014536.1 gb: NM_003909.1 gb: AF077226.2 |
| 201740_at | gb: NM_004551.1 /DEF = Homo sapiens NADH dehydrogenase (ubiquinone) Fe—S protein 3 (30 kD) (NADH-coenzyme Q reductase) |
| | (NDUFS3), mRNA. /FEA = mRNA /GEN = NDUFS3 /PROD = NADH dehydrogenase (ubiquinone) Fe—S protein 3(30 kD) (NADH-coenzyme Q reductase) |
| | /DB_XREF = gi: 4758787 /UG = Hs.5273 NADH dehydrogenase (ubiquinone) Fe—S protein 3 (30 kD) (NADH-coenzyme Q reductase) |
| | /FL = gb: BC000617.1 gb: AF067139.1 gb: NM_004551.1 gb: AF100743.1 |
| 215952_s_at | Consensus includes gb: AF090094.1 /DEF = Homo sapiens clone IMAGE 172979. /FEA = mRNA /DB_XREF = gi: 4063629 |
| | /UG = Hs.125078 ornithine decarboxylase antizyme 1 |
| 212796_s_at | Consensus includes gb: BF195608 /FEA = EST /DB_XREF = gi: 11082676 /DB_XREF = est: 7n85f03.x1 /CLONE = IMAGE: 3571349 |
| | /UG = Hs.126084 KIAA1055 protein |
| 210561_s_at | gb: AL110243.1 /DEF = Homo sapiens mRNA; cDNA DKFZp564B0482 (from clone DKFZp564B0482); complete cds. /FEA = mRNA |
| | /GEN = DKFZp564B0482 /PROD = hypothetical protein /DB_XREF = gi: 5817189 /UG = Hs.187991 DKFZP564A122 protein |
| | /FL = gb: AF072880.1 gb: AF106683.1 gb: AL110243.1 gb: AF112205.1 gb: AF069313.2 |
| 202220_at | gb: NM_014949.1 /DEF = Homo sapiens KIAA0907 protein (KIAA0907), mRNA. /FEA = mRNA /GEN = KIAA0907 /PROD = KIAA0907 protein |
| | /DB_XREF = gi: 7662371 /UG = Hs.24656 KIAA0907 protein /FL = gb: AB020714.1 gb: NM_014949.1 |
| 203739_at | gb: NM_006526.1 /DEF = Homo sapiens zinc finger protein 217 (ZNF217), mRNA. /FEA = mRNA /GEN = ZNF217 /PROD = zinc finger protein |
| | 217 |
| | /DB_XREF = gi: 5730123 /UG = Hs.155040 zinc finger protein 217 /FL = gb: AF041259.1 gb: NM_006526.1 |
| 211036_x_at | gb: BC006301.1 /DEF = Homo sapiens, anaphase-promoting complex subunit 5, clone MGC: 13295, mRNA, complete cds. /FEA = mRNA |
| | /PROD = anaphase-promoting complex subunit 5 /DB_XREF = gi: 13623410 /FL = gb: BC006301.1 |
| 204060_s_at | gb: NM_005044.1 /DEF = Homo sapiens protein kinase, X-linked (PRKX), mRNA. /FEA = mRNA /GEN = PRKX /PROD = protein kinase, X- |
| | linked |
| | /DB_XREF = gi: 4826947 /UG = Hs.147996 protein kinase, X-linked /FL = gb: NM_005044.1 |
| 64064_at | Cluster Incl. AI435089: th95b11.x1 Homo sapiens cDNA, 3 end /clone = IMAGE-2126397 /clone_end = 3 /gb = AI435089 /gi = 4300737 |
| | /ug = Hs.26194 /len = 891 |
| 201515_s_at | gb: NM_004622.1 /DEF = Homo sapiens translin (TSN), mRNA. /FEA = mRNA /GEN = TSN /PROD = translin /DB_XREF = gi: 4759269 |
| | /UG = Hs.75066 translin /FL = gb: NM_004622.1 |
| 219093_at | gb: NM_017933.1 /DEF = Homo sapiens hypothetical protein FLJ20701 (FLJ20701), mRNA. /FEA = mRNA /GEN = FLJ20701 |
| | /PROD = hypothetical protein FLJ20701 /DB_XREF = gi: 8923631 /UG = Hs.169764 hypothetical protein FLJ20701 /FL = gb: NM_017933.1 |
| 221739_at | Consensus includes gb: AL524093 /FEA = EST /DB_XREF = gi: 12787586 /DB_XREF = est: AL524093 /CLONE = CS0DC002YI04 (5 prime) |
| | /UG = Hs.10927 hypothetical protein EUROIMAGE1875335 |
| 208822_s_at | gb: U18321.1 /DEF = Human ionizing radiation resistance conferring protein mRNA, complete cds. /FEA = mRNA /PROD = ionizing |
| | radiation resistance conferringprotein /DB_XREF = gi: 603763 /UG = Hs.159627 death associated protein 3 /FL = gb: U18321.1 |
| 218160_at | gb: NM_014222.1 /DEF = Homo sapiens NADH dehydrogenase (ubiquinone) 1 alpha subcomplex, 8 (19 kD, PGIV) (NDUFA8), mRNA. |
| | /FEA = mRNA /GEN = NDUFA8 /PROD = NADH dehydrogenase (ubiquinone) 1 alphasubcomplex, 8 (19 kD, PGIV) /DB_XREF = gi: 7657368 |
| | /UG = Hs.31547 NADH dehydrogenase (ubiquinone) 1 alpha subcomplex, 8 (19 kD, PGIV) /FL = gb: BC001016.1 gb: AF044953.1 |
| | gb: NM_014222.1 |
| 221728_x_at | Consensus includes gb: AA628440 /FEA = EST /DB_XREF = gi: 2540827 /DB_XREF = est: af26f02.s1 /CLONE = IMAGE: 1032795 |
| | /UG = Hs.83623 nuclear receptor subfamily 1, group I, member 3 |
| 210943_s_at | gb: U84744.1 /DEF = Human Chediak-Higashi syndrome protein short isoform (LYST) mRNA, complete cds. /FEA = mRNA /GEN = LYST |
| | /PROD = Chediak-Higashi syndrome protein short isoform /DB_XREF = gi: 2654473 /UG = Hs.36508 Chediak-Higashi syndrome 1 |
| | /FL = gb: U84744.1 |
| 209104_s_at | gb: BC000009.1 /DEF = Homo sapiens, likely homolog of yeast Nhp2, component of the HACA snoRNP; hypothetical protein |
| | FLJ20479, |
| | clone MGC: 1038, mRNA, complete cds. /FEA = mRNA /PROD = likely homolog of yeast Nhp2, component of theHACA snoRNP; |
| | hypothetical protein FLJ20479 /DB_XREF = gi: 12652540 /UG = Hs.23990 nucleolar protein family A, member 2 (HACA small nucleolar |
| | RNPs) |
| | /FL = gb: BC000009.1 gb: NM_017838.1 |
| 217987_at | gb: NM_019048.1 /DEF = Homo sapiens hypothetical protein (FLJ20752), mRNA. /FEA = mRNA /GEN = FLJ20752 /PROD = hypothetical protein |
| | /DB_XREF = gi: 9506696 /UG = Hs.101364 hypothetical protein /FL = gb: BC001243.1 gb: NM_019048.1 |
| 201699_at | gb: NM_002806.1 /DEF = Homo sapiens proteasome (prosome, macropain) 26S subunit, ATPase, 6 (PSMC6), mRNA. /FEA = mRNA |
| | /GEN = PSMC6 |
| | /PROD = proteasome (prosome, macropain) 26S subunit, ATPase, 6 /DB_XREF = gi: 4506214 /UG = Hs.79357 proteasome |
| | (prosome, macropain) 26S subunit, ATPase, 6 /FL = gb: BC005390.1 gb: D78275.1 gb: AF006305.1 gb: NM_002806.1 |
| 201359_at | gb: NM_016451.1 /DEF = Homo sapiens coatomer protein complex, subunit beta (COPB), mRNA. /FEA = mRNA /GEN = COPB |
| | /PROD = coatomer protein complex, subunit beta /DB_XREF = gi: 7705368 /UG = Hs.3059 coatomer protein complex, subunit beta |
| | /FL = gb: AF084457.1 gb: AL136593.1 gb: NM_016451.1 |
| 210027_s_at | gb: M80261.1 /DEF = Human apurinic endonuclease (APE) mRNA, complete cds. /FEA = mRNA /GEN = APE /PROD = apurinic endonuclease |
| | /DB_XREF = gi: 178742 /UG = Hs.73722 APEX nuclease (multifunctional DNA repair enzyme) /FL = gb: BC004979.1 gb: M80261.1 |
| 202096_s_at | gb: NM_000714.2 /DEF = Homo sapiens benzodiazapine receptor (peripheral) (BZRP), nuclear gene encoding mitochondrial protein, |
| | transcript variant PBR, mRNA. /FEA = mRNA /GEN = BZRP /PROD = peripheral benzodiazapine receptor /DB_XREF = gi: 6382068 |
| | /UG = Hs.202 benzodiazapine receptor (peripheral) /FL = gb: BC001110.1 gb: M36035.1 gb: NM_000714.2 |
| 214948_s_at | Consensus includes gb: AL050136.1 /DEF = Homo sapiens mRNA; cDNA DKFZp586L141 (from clone DKFZp586L141). |
| | /FEA = mRNA /DB_XREF = gi: 4884346 /UG = Hs.140945 Homo sapiens mRNA; cDNA DKFZp586L141 (from clone DKFZp586L141) |
| 200042_at | gb: NM_014306.1 /DEF = Homo sapiens hypothetical protein (HSPC117), mRNA. /FEA = mRNA /GEN = HSPC117 /PROD = similar to |
| | C. elegens hypothetical 55.2 kD protein F16A11.2 /DB_XREF = gi: 7657014 /UG = Hs.10729 hypothetical protein |
| | /FL = gb: BC000151.1 gb: BC002970.1 gb: AF161466.1 gb: NM_014306.1 gb: AF155658.1 |
| 219035_s_at | gb: NM_025126.1 /DEF = Homo sapiens hypothetical protein FLJ21786 (FLJ21786), mRNA. /FEA = mRNA /GEN = FLJ21786 |
| | /PROD = hypothetical protein FLJ21786 /DB_XREF = gi: 13376704 /UG = Hs.316809 hypothetical protein FLJ21786 /FL = gb: NM_025126.1 |
| 200041_s_at | gb: NM_004640.1 /DEF = Homo sapiens HLA-B associated transcript-1 (D6S81E), mRNA. /FEA = mRNA /GEN = D6S81E /PROD = HLA-B associated |
| | transcript-1 /DB_XREF = gi: 4758111 /UG = Hs.55296 HLA-B associated transcript-1 /FL = gb: BC004350.1 gb: NM_004640.1 |
| 216321_s_at | Consensus includes gb: X03348.1 /DEF = Human mRNA for beta-glucocorticoid receptor (clone OB10). /FEA = mRNA |
| | /PROD = beta-glucocorticoid receptor /DB_XREF = gi: 31681 /UG = Hs.75772 nuclear receptor subfamily 3, group C, member 1 |
| 214574_x_at | Consensus includes gb: NM_007161.1 /DEF = Homo sapiens DNA segment on chromosome 6 (unique) 49 expressed sequence, |
| | NK cell triggering receptor, p30 (D6S49E), mRNA. /FEA = CDS /GEN = D6S49E /PROD = leukocyte-specific transcript 1 |
| | /DB_XREF = gi: 6005740 /UG = Hs.88411 lymphocyte antigen 117 /FL = gb: NM_007161.1 |
| 205370_x_at | gb: NM_001918.1 /DEF = Homo sapiens dihydrolipoamide branched chain transacylase (E2 component of branched chain keto acid |
| | dehydrogenase complex; maple syrup urine disease) (DET), mRNA. /FEA = mRNA /GEN = DBT /PROD = dihydrolipoamide branched chain |
| | transacylase (E2component of branched chain keto acid dehydrogenasecomplex; maple syrup urine disease) |
| | /DB_XREF = gi: 4503264 /UG = Hs.139410 dihydrolipoamide branched chain transacylase (E2 component of branched chain |
| | keto acid dehydrogenase complex; maple syrup urine disease) /FL = gb: J03208.1 gb: NM_001918.1 gb: M27093.1 |
| 213872_at | Consensus includes gb: BE465032 /FEA = EST /DB_XREF = gi: 9510807 /DB_XREF = est: hv76g09.x1 /CLONE = IMAGE: 3179392 |
| | /UG = Hs.173685 hypothetical protein FLJ12619 |
| 201576_s_at | gb: NM_000404.1 /DEF = Homo sapiens galactosidase, beta 1 (GLB1), mRNA. /FEA = mRNA /GEN = GLB1 /PROD = galactosidase, |
| | beta 1 /DB_XREF = gi: 10834965 /UG = Hs.79222 galactosidase, beta 1 /FL = gb: NM_000404.1 gb: M27507.1 gb: M22590.1 gb: M34423.1 |
| 202548_s_at | gb: NM_003899.1 /DEF = Homo sapiens PAK-interacting exchange factor beta (P85SPR), mRNA. /FEA = mRNA /GEN = P85SPR |
| | /PROD = PAK-interacting exchange factor beta /DB_XREF = gi: 4505572 /UG = Hs.172813 PAK-interacting exchange factor beta |
| | /FL = gb: D63476.1 gb: NM_003899.1 |
| 204028_s_at | gb: NM_012197.2 /DEF = Homo sapiens rab6 GTPase activating protein (GAP and centrosome-associated) (GAPCENA), mRNA. |
| | /FEA = mRNA /GEN = GAPCENA /PROD = rab6 GTPase activating protein (GAP andcentrosome-associated) /DB_XREF = gi: 12232372 |
| | /UG = Hs.55099 rab6 GTPase activating protein (GAP and centrosome-associated) /FL = gb: NM_012197.2 |
| 202435_s_at | Consensus includes gb: AU154504 /FEA = EST /DB_XREF = gi: 11016025 /DB_XREF = est: AU154504 /CLONE = NT2RP4001328 |
| | /UG = Hs.154654 cytochrome P450, subfamily I (dioxin-inducible), polypeptide 1 (glaucoma 3, primary infantile) |
| | /FL = gb: NM_000104.2 gb: U03688.1 |
| 217980_s_at | gb: NM_017840.1 /DEF = Homo sapiens hypothetical protein FLJ20484 (FLJ20484), mRNA. /FEA = mRNA /GEN = FLJ20484 |
| | /PROD = hypothetical protein FLJ20484 /DB_XREF = gi: 8923447 /UG = Hs.5080 hypothetical protein FLJ20484 |
| | /FL = gb: AF275806.1 gb: BC001040.1 gb: NM_017840.1 gb: AF183428.1 |
| 201112_s_at | gb: NM_001316.1 /DEF = Homo sapiens chromosome segregation 1 (yeast homolog)-like (CSE1L), mRNA. /FEA = mRNA |
| | /GEN = CSE1L /PROD = chromosome segregation 1 (yeast homolog)-like /DB_XREF = gi: 4503072 /UG = Hs.90073 chromosome segregation 1 |
| | (yeast homolog)-like /FL = gb: U33286.1 gb: AF053641.1 gb: NM_001316.1 |
| 202322_s_at | gb: NM_004837.1 /DEF = Homo sapiens geranylgeranyl diphosphate synthase 1 (GGPS1), mRNA. /FEA = mRNA /GEN = GGPS1 |
| | /PROD = geranylgeranyl diphosphate synthase 1 /DB_XREF = gi: 4758429 /UG = Hs.55498 geranylgeranyl diphosphate synthase 1 |
| | /FL = gb: AF057698.1 gb: BC005252.1 gb: AB017971.1 gb: AB016043.1 gb: AB019036.1 gb: NM_004837.1 gb: AF125394.1 |
| 203545_at | gb: NM_024079.1 /DEF = Homo sapiens hypothetical protein MGC2840 similar to a putative glucosyltransferase (MGC2840), mRNA. |
| | /FEA = mRNA /GEN = MGC2840 /PROD = hypothetical protein MGC2840 similar to aputative glucosyltransferase /DB_XREF = gi: 13129069 |
| | /UG = Hs.155356 hypothetical protein MGC2840 similar to a putative glucosyltransferase /FL = gb: BC001133.1 gb: NM_024079.1 |
| 212646_at | Consensus includes gb: D42043.1 /DEF = Human mRNA for KIAA0084 gene, partial cds. /FEA = mRNA /GEN = KIAA0084 /DB_XREF = gi: 577298 |
| | /UG = Hs.79123 KIAA0084 protein |
| 212591_at | Consensus includes gb: AA887480 /FEA = EST /DB_XREF = gi: 3003168 /DB_XREF = est: oj54a12.s1 /CLONE = IMAGE: 1502110 |
| | /UG = Hs.17428 RBP1-like protein |
| 218005_at | Consensus includes gb: AA744771 /FEA = EST /DB_XREF = gi: 2783535 /DB_XREF = est: ny71d07.s1 /CLONE = IMAGE: 1283725 |
| | /UG = Hs.108642 zinc finger protein 22 (KOX 15) /FL = gb: NM_006963.1 |
| 201165_s_at | Consensus includes gb: BE670915 /FEA = EST /DB_XREF = gi: 10031456 /DB_XREF = est: 7e43a09.x1 /CLONE = IMAGE: 3285208 |
| | /UG = Hs.153834 pumilio (Drosophila) homolog 1 /FL = gb: AF315592.1 gb: NM_014676.1 |
| 212239_at | Consensus includes gb: AI680192 /FEA = EST /DB_XREF = gi: 4890374 /DB_XREF = est: tw66a10.x1 /CLONE = IMAGE: 2264634 |
| | /UG = Hs.6241 phosphoinositide-3-kinase, regulatory subunit, polypeptide 1 (p85 alpha) |
| 204386_s_at | Consensus includes gb: BF303597 /FEA = EST /DB_XREF = gi: 11250218 /DB_XREF = est: 601886230F2 /CLONE = IMAGE: 4120446 |
| | /UG = Hs.182695 hypothetical protein MGC3243 /FL = gb: BC000002.1 gb: NM_024026.1 |
| 214853_s_at | Consensus includes gb: AI091079 /FEA = EST /DB_XREF = gi: 3430138 /DB_XREF = est: qa53e05.s1 /CLONE = IMAGE: 1690496 |
| | /UG = Hs.81972 SHC (Src homology 2 domain-containing) transforming protein 1 |
| 210580_x_at | gb: L25275.1 /DEF = Human estrogen sulfotransferase mRNA, complete cds. /FEA = mRNA /PROD = estrogen sulfotransferase |
| | /DB_XREF = gi: 463124 /UG = Hs.274614 sulfotransferase family, cytosolic, 1A, phenol-preferring, member 3 /FL = gb: L25275.1 |
| 222150_s_at | Consensus includes gb: AK026747.1 /DEF = Homo sapiens cDNA: FLJ23094 fis, clone LNG07379, highly similar to HST000007 Homo |
| | sapiens |
| | mRNA full length insert cDNA clone EUROIMAGE 293605. /FEA = mRNA /DB_XREF = gi: 10439670 /UG = Hs.12969 hypothetical protein |
| 209265_s_at | gb: BC001650.1 /DEF = Homo sapiens, Similar to putative methyltransferase, clone MGC: 2708, mRNA, complete cds. /FEA = mRNA |
| | /PROD = Similar to putative methyltransferase /DB_XREF = gi: 12804482 /UG = Hs.268149 putative methyltransferase |
| | /FL = gb: BC003031.1 gb: BC001650.1 gb: NM_019852.1 |
| 218807_at | gb: NM_006113.2 /DEF = Homo sapiens vav 3 oncogene (VAV3), mRNA. /FEA = mRNA /GEN = VAV3 /PROD = vav 3 oncogene |
| | /DB_XREF = gi: 7262390 |
| | /UG = Hs.267659 vav 3 oncogene /FL = gb: AF067817.1 gb: AF118887.1 gb: NM_006113.2 |
| 209043_at | gb: AF033026.1 /DEF = Homo sapiens bifunctional ATP sulfurylaseadenosine 5-phosphosulfate kinase mRNA, complete cds. |
| | /FEA = mRNA /PROD = bifunctional ATP sulfurylaseadenosine5-phosphosulfate kinase /DB_XREF = gi: 3378100 |
| | /UG = Hs.3833 3-phosphoadenosine 5-phosphosulfate synthase 1 /FL = gb: AF033026.1 gb: AF016496.1 gb: NM_005443.1 gb: AF105227.1 |
| 219777_at | gb: NM_024711.1 /DEF = Homo sapiens hypothetical protein FLJ22690 (FLJ22690), mRNA. /FEA = mRNA /GEN = FLJ22690 |
| | /PROD = hypothetical protein FLJ22690 /DB_XREF = gi: 13376008 /UG = Hs.105468 hypothetical protein FLJ22690 /FL = gb: NM_024711.1 |
| 218048_at | gb: NM_012071.1 /DEF = Homo sapiens BUP protein (BUP), mRNA. /FEA = mRNA /GEN = BUP /PROD = BUP protein /DB_XREF = gi: 6912277 |
| | /UG = Hs.35660 BUP protein /FL = gb: AF078848.1 gb: AF201948.1 gb: NM_012071.1 |
| 201986_at | Consensus includes gb: AB011165.1 /DEF = Homo sapiens mRNA for KIAA0593 protein, partial cds. /FEA = mRNA /GEN = KIAA0593 |
| | /PROD = KIAA0593 protein /DB_XREF = gi: 3043709 /UG = Hs.11861 thyroid hormone receptor-associated protein, 240 kDa subunit |
| | /FL = gb: AF117754.1 gb: NM_005121.1 |
| 219799_s_at | gb: NM_005771.1 /DEF = Homo sapiens retinol dehydrogenase homolog (RDHL), mRNA. /FEA = mRNA /GEN = RDHL |
| | /PROD = retinol dehydrogenase homolog /DB_XREF = gi: 5032034 /UG = Hs.179608 retinol dehydrogenase homolog /FL = gb: AF067174.1 |
| | gb: NM_005771.1 |
| 208688_x_at | gb: U78525.1 /DEF = Homo sapiens eukaryotic translation initiation factor (eIF3) mRNA, complete cds. /FEA = mRNA /GEN = eIF3 |
| | /PROD = eukaryotic translation initiation factor /DB_XREF = gi: 2558667 /UG = Hs.57783 eukaryotic translation initiation factor |
| | 3, |
| | subunit 9 (eta, 116 kD) /FL = gb: U78525.1 |
| 200001_at | gb: NM_001749.1 /DEF = Homo sapiens calpain 4, small subunit (30K) (CAPN4), mRNA. /FEA = mRNA /GEN = CAPN4 /PROD = calpain 4 small |
| | subunit |
| | /DB_XREF = gi: 4502564 /UG = Hs.74451 calpain 4, small subunit (30K) /FL = gb: BC000592.1 gb: NM_001749.1 |
| 218118_s_at | gb: NM_006327.1 /DEF = Homo sapiens translocase of inner mitochondrial membrane 23 (yeast) homolog (TIM23), mRNA. /FEA = mRNA |
| | /GEN = TIM23 /PROD = translocase of inner mitochondrial membrane 23(yeast) homolog /DB_XREF = gi: 5454121 |
| | /UG = Hs.283684 translocase of inner mitochondrial membrane 23 (yeast) homolog /FL = gb: AF030162.1 gb: NM_006327.1 |
| 201840_at | gb: NM_006156.1 /DEF = Homo sapiens neural precursor cell expressed, developmentally down-regulated 8 (NEDD8), mRNA. |
| | /FEA = mRNA /GEN = NEDD8 /PROD = neural precursor cell expressed, developmentallydown-regulated 8 /DB_XREF = gi: 5453759 |
| | /UG = Hs.75512 neural precursor cell expressed, developmentally down-regulated 8 /FL = gb: D23662.1 gb: NM_006156.1 |
| 38710_at | Cluster Incl. AL096714: Homo sapiens mRNA; cDNA DKFZp564E242 (from clone DKFZp564E242) /cds = UNKNOWN /gb = AL096714 |
| | /gi = 5419847 /ug = Hs.108504 /len = 1904 |
| 212287_at | Consensus includes gb: BF382924 /FEA = EST /DB_XREF = gi: 11364313 /DB_XREF = est: 601816985F1 /CLONE = IMAGE: 4050909 |
| | /UG = Hs.197803 KIAA0160 protein |
| 203098_at | Consensus includes gb: AL050164.1 /DEF = Homo sapiens mRNA; cDNA DKFZp586C1622 (from clone DKFZp586C1622); partial cds. |
| | /FEA = mRNA /GEN = DKFZp586C1622 /PROD = hypothetical protein /DB_XREF = gi: 4884378 /UG = Hs.16081 chromodomain protein, |
| | Y chromosome-like /FL = gb: AF081258.1 gb: AF081259.1 gb: NM_004824.1 |
| 218905_at | gb: NM_017864.1 /DEF = Homo sapiens hypothetical protein FLJ20530 (FLJ20530), mRNA. /FEA = mRNA /GEN = FLJ20530 |
| | /PROD = hypothetical protein FLJ20530 /DB_XREF = gi: 8923495 /UG = Hs.279521 hypothetical protein FLJ20530 /FL = gb: NM_017864.1 |
| 218303_x_at | gb: NM_016618.1 /DEF = Homo sapiens hypothetical protein (LOC51315), mRNA. /FEA = mRNA /GEN = LOC51315 /PROD = hypothetical protein |
| | /DB_XREF = gi: 7706155 /UG = Hs.5721 hypothetical protein /FL = gb: AF208845.1 gb: AF217520.1 gb: NM_016618.1 |
| 208705_s_at | Consensus includes gb: BG481972 /FEA = EST /DB_XREF = gi: 13414251 /DB_XREF = est: 602526894F1 /CLONE = IMAGE: 4650406 |
| | /UG = Hs.286236 eukaryotic translation initiation factor 5 /FL = gb: AL080102.1 |
| 218715_at | gb: NM_018428.1 /DEF = Homo sapiens hepatocellular carcinoma-associated antigen 66 (HCA66), mRNA. /FEA = mRNA /GEN = HCA66 |
| | /PROD = hepatocellular carcinoma-associated antigen 66 /DB_XREF = gi: 8923721 /UG = Hs.30670 hepatocellular |
| | carcinoma-associated antigen 66 /FL = gb: AF244135.1 gb: AF116631.1 gb: NM_018428.1 |
| 202184_s_at | gb: NM_018230.1 /DEF = Homo sapiens hypothetical protein FLJ10814 (FLJ10814), mRNA. /FEA = mRNA /GEN = FLJ10814 |
| | /PROD = hypothetical protein FLJ10814 /DB_XREF = gi: 8922689 /UG = Hs.12457 hypothetical protein FLJ10814 /FL = gb: NM_018230.1 |
| 202004_x_at | gb: NM_003001.2 /DEF = Homo sapiens succinate dehydrogenase complex, subunit C, integral membrane protein, 15 kD (SDHC), |
| | nuclear gene encoding mitochondrial protein, mRNA. /FEA = mRNA /GEN = SDHC /PROD = succinate dehydrogenase complex, |
| | subunit Cprecursor /DB_XREF = gi: 9257243 /UG = Hs.3577 succinate dehydrogenase complex, subunit C, integral membrane protein, |
| | 15 kD /FL = gb: U57877.1 gb: NM_003001.2 |
| 209435_s_at | gb: BC000265.1 /DEF = Homo sapiens, clone MGC: 3182, mRNA, complete cds. /FEA = mRNA /PROD = Unknown (protein for MGC: 3182) |
| | /DB_XREF = gi: 12653008 /UG = Hs.315417 Homo sapiens, clone MGC: 3182, mRNA, complete cds: /FL = gb: BC000265.1 |
| 221044_s_at | gb: NM_021616.1 /DEF = Homo sapiens ring finger protein 21, interferon-responsive (RNF21), mRNA. /FEA = mRNA /GEN = RNF21 |
| | /PROD = ring finger protein 21, interferon-responsive /DB_XREF = gi: 11055977 /UG = Hs.125300 ring finger protein 21, |
| | interferon-responsive /FL = gb: AB039903.1 gb: NM_021616.1 |
| 200992_at | Consensus includes gb: AL137335.1 /DEF = Homo sapiens mRNA; cDNA DKFZp434A179 (from clone DKFZp434A179); partial cds. |
| | /FEA = mRNA /GEN = DKFZp434A179 /PROD = hypothetical protein /DB_XREF = gi: 6807827 /UG = Hs.5151 RAN binding protein 7 |
| | /FL = gb: AF098799.1 gb: NM_006391.1 |
| 201900_s_at | gb: NM_006066.1 /DEF = Homo sapiens aldo-keto reductase family 1, member A1 (aldehyde reductase) (AKR1A1), mRNA. /FEA = mRNA |
| | /GEN = AKR1A1 /PROD = aldo-keto reductase family 1, member A1(aldehyde reductase) /DB_XREF = gi: 5174390 |
| | /UG = Hs.89529 aldo-keto reductase family 1, member A1 (aldehyde reductase) /FL = gb: BC000670.1 gb: J04794.1 gb: NM_006066.1 |
| 202560_s_at | gb: NM_015607.1 /DEF = Homo sapiens DKFZP547E1010 protein (DKFZP547E1010), mRNA. /FEA = mRNA /GEN = DKFZP547E1010 |
| | /PROD = DKFZP547E1010 protein /DB_XREF = gi: 7661589 /UG = Hs.323817 DKFZP547E1010 protein /FL = gb: NM_015607.1 |
| 201105_at | gb: NM_002305.2 /DEF = Homo sapiens lectin, galactoside-binding, soluble, 1 (galectin 1) (LGALS1), mRNA. /FEA = mRNA |
| | /GEN = LGALS1 /PROD = beta-galactosidase binding lectin precursor /DB_XREF = gi: 6006015 /UG = Hs.227751 lectin, galactoside- |
| | binding, soluble, 1 (galectin 1) /FL = gb: BC001693.1 gb: J04456.1 gb: NM_002305.2 |
| 204546_at | gb: NM_014732.1 /DEF = Homo sapiens KIAA0513 gene product (KIAA0513), mRNA. /FEA = mRNA /GEN = KIAA0513 /PROD = KIAA0513 gene |
| | product /DB_XREF = gi: 7662163 /UG = Hs.301658 KIAA0513 gene product /FL = gb: AB011085.1 gb: NM_014732.1 |
| 214181_x_at | Consensus includes gb: AI735692 /FEA = EST /DB_XREF = gi: 5057216 /DB_XREF = est: at20b12.x1 /CLONE = IMAGE: 2355647 |
| | /UG = Hs.88411 lymphocyte antigen 117 |
| 219032_x_at | gb: NM_014322.1 /DEF = Homo sapiens opsin 3 (encephalopsin) (OPN3), mRNA. /FEA = mRNA /GEN = OPN3 /PROD = opsin 3 |
| | (encephalopsin) /DB_XREF = gi: 7657070 /UG = Hs.279926 opsin 3 (encephalopsin) /FL = gb: AF140242.1 gb: NM_014322.1 |
| 205306_x_at | Consensus includes gb: AI074145 /FEA = EST /DB_XREF = gi: 3400789 /DB_XREF = est: ov13a06.x1 /CLONE = IMAGE: 1637170 |
| | /UG = Hs.107318 kynurenine 3-monooxygenase (kynurenine 3-hydroxylase) /FL = gb: AF056032.1 gb: NM_003679.1 |
| 202837_at | gb: NM_006700.1 /DEF = Homo sapiens FLN29 gene product (FLN29), mRNA. /FEA = mRNA /GEN = FLN29 /PROD = FLN29 gene product |
| | /DB_XREF = gi: 5729827 /UG = Hs.5148 FLN29 gene product /FL = gb: BC003553.1 gb: AB007447.1 gb: NM_006700.1 |
| 202153_s_at | gb: NM_016553.1 /DEF = Homo sapiens hypothetical protein (DKFZp547L134), mRNA. /FEA = mRNA /GEN = DKFZp547L134 |
| | /PROD = hypothetical protein /DB_XREF = gi: 7705354 /UG = Hs.9877 hypothetical protein /FL = gb: AL162061.1 gb: NM_016553.1 |
| 209341_s_at | Consensus includes gb: AU153366 /FEA = EST /DB_XREF = gi: 11014887 /DB_XREF = est: AU153366 /CLONE = NT2RP3002988 /UG = Hs.226573 |
| | inhibitor of kappa light polypeptide gene enhancer in B-cells, kinase beta /FL = gb: AF031416.1 gb: AF080158.1 |
| 212493_s_at | Consensus includes gb: AI761110 /FEA = EST /DB_XREF = gi: 5176777 /DB_XREF = est: wi69h02.x1 /CLONE = IMAGE: 2398611 /UG = Hs.6947 |
| | HSPC069 protein |
| 201600_at | gb: NM_007273.1 /DEF = Homo sapiens B-cell associated protein (REA), mRNA. /FEA = mRNA /GEN = REA /PROD = B-cell associated protein |
| | /DB_XREF = gi: 6005853 /UG = Hs.7771 B-cell associated protein /FL = gb: AF150962.1 gb: NM_007273.1 gb: AF126021.1 |
| | gb: AF178980.1 |
| 201144_s_at | gb: NM_004094.1 /DEF = Homo sapiens eukaryotic translation initiation factor 2, subunit 1 (alpha, 35 kD) (EIF2S1), mRNA. |
| | /FEA = mRNA /GEN = EIF2S1 /PROD = eukaryotic translation initiation factor 2, subunit 1 (alpha, 35 kD) /DB_XREF = gi: 4758255 |
| | /UG = Hs.151777 eukaryotic translation initiation factor 2, subunit 1 (alpha, 35 kD) /FL = gb: BC002513.1 gb: J02645.1 |
| | gb: NM_004094.1 |
| 211985_s_at | Consensus includes gb: AI653730 /FEA = EST /DB_XREF = gi: 4737709 /DB_XREF = est: wb36f12.x1 /CLONE = IMAGE: 2307791 |
| | /UG = Hs.279009 matrix Gla protein |
| 201226_at | gb: NM_005004.1 /DEF = Homo sapiens NADH dehydrogenase (ubiquinone) 1 beta subcomplex, 8 (19 kD, ASHI) (NDUFB8), mRNA. |
| | /FEA = mRNA /GEN = NDUFB8 /PROD = NADH dehydrogenase (ubiquinone) 1 betasubcomplex, 8 (19 kD, ASHI) /DB_XREF = gi: 4826853 |
| | /UG = Hs.198273 NADH dehydrogenase (ubiquinone) 1 beta subcomplex, 8 (19 kD, ASHI) |
| | /FL = gb: BC000466.1 gb: AF044958.1 gb: AF077028.1 gb: NM_005004.1 gb: AL080056.1 |
| 208828_at | gb: BC004170.1 /DEF = Homo sapiens, histone fold protein CHRAC17; DNA polymerase epsilon p17 subunit, clone MGC: 2725, mRNA, |
| | complete cds. /FEA = mRNA /PROD = histone fold protein CHRAC17; DNA polymeraseepsilon p17 subunit /DB_XREF = gi: 13278800 |
| | /UG = Hs.108112 histone fold protein CHRAC17; DNA polymerase epsilon p17 subunit /FL = gb: BC003166.1 gb: BC004170.1 |
| | gb: AF226077.1 gb: NM_017443.1 |
| 204020_at | Consensus includes gb: BF739943 /FEA = EST /DB_XREF = gi: 12066607 /DB_XREF = est: 7o41b04.x1 /CLONE = IMAGE: 3576534 |
| | /UG = Hs.29117 purine-rich element binding protein A /FL = gb: M96684.1 gb: NM_005859.1 |
| 217942_at | gb: NM_021821.1 /DEF = Homo sapiens MDS023 protein (MDS023), mRNA. /FEA = mRNA /GEN = MDS023 /PROD = MDS023 protein |
| | /DB_XREF = gi: 11141894 /UG = Hs.10724 MDS023 protein /FL = gb: AF182422.1 gb: NM_021821.1 |
| 203514_at | Consensus includes gb: BF971923 /FEA = EST /DB_XREF = gi: 12339138 /DB_XREF = est: 602240326F1 /CLONE = IMAGE: 4328791 |
| | /UG = Hs.29282 mitogen-activated protein kinase kinase kinase 3 /FL = gb: U78876.1 gb: NM_002401.1 |
| 204334_at | Consensus includes gb: AA488672 /FEA = EST /DB_XREF = gi: 2216103 /DB_XREF = est: ab40d10.s1 /CLONE = IMAGE: 843283 |
| | /UG = Hs.21599 Kruppel-like factor 7 (ubiquitous) /FL = gb: AB015132.1 gb: NM_003709.1 |
| 203531_at | Consensus includes gb: BF435809 /FEA = EST /DB_XREF = gi: 11448124 /DB_XREF = est: nab42a09.x1 /CLONE = IMAGE: 3268504 |
| | /UG = Hs.101299 cullin 5 /FL = gb: AF327710.1 gb: AF017061.1 gb: NM_003478.1 |
| 205022_s_at | gb: NM_005197.1 /DEF = Homo sapiens checkpoint suppressor 1 (CHES1), mRNA. /FEA = mRNA /GEN = CHES1 /PROD = checkpoint suppressor |
| | 1 /DB_XREF = gi: 4885136 /UG = Hs.211773 checkpoint suppressor 1 /FL = gb: U68723.1 gb: NM_005197.1 |
| 201532_at | gb: NM_002788.1 /DEF = Homo sapiens proteasome (prosome, macropain) subunit, alpha type, 3 (PSMA3), mRNA. /FEA = mRNA |
| | /GEN = PSMA3 /PROD = proteasome (prosome, macropain) subunit, alphatype, 3 /DB_XREF = gi: 4506182 /UG = Hs.167106 proteasome |
| | (prosome, macropain) subunit, alpha type, 3 /FL = gb: BC005265.1 gb: NM_002788.1 |
| 204972_at | gb: NM_016817.1 /DEF = Homo sapiens 2-5oligoadenylate synthetase 2 (OAS2), transcript variant 1, mRNA. /FEA = mRNA /GEN = OAS2 |
| | /PROD = 2-5oligoadenylate synthetase 2, isoform p71 /DB_XREF = gi: 8051624 /UG = Hs.264981 2-5oligoadenylate synthetase 2 |
| | /FL = gb: M87434.1 gb: NM_016817.1 |
| 209200_at | Consensus includes gb: AL536517 /FEA = EST /DB_XREF = gi: 12800010 /DB_XREF = est: AL536517 /CLONE = CS0DF038YH13 (5 prime) |
| | /UG = Hs.78995 MADS box transcription |
| | enhancer factor 2, polypeptide C (myocyte enhancer factor 2C) /FL = gb: L08895.1 |
| 217945_at | gb: NM_025238.1 /DEF = Homo sapiens BTB (POZ) domain containing 1 (BTBD1), mRNA. /FEA = mRNA /GEN = BTBD1 /PROD = BTB (POZ) |
| | domain containing 1 /DB_XREF = gi: 13376847 /UG = Hs.21332 BTB (POZ) domain containing 1 /FL = gb: AL136853.1 |
| | gb: AF257241.1 gb: NM_025238.1 gb: AF355402.1 |
| 201491_at | gb: NM_012111.1 /DEF = Homo sapiens chromosome 14 open reading frame 3 (C14ORF3), mRNA. /FEA = mRNA /GEN = C14ORF3 |
| | /PROD = chromosome 14 open reading frame 3 /DB_XREF = gi: 6912279 /UG = Hs.204041 chromosome 14 open reading frame 3 |
| | /FL = gb: BC000321.1 gb: NM_012111.1 gb: AF164791.1 |
| 204868_at | gb: NM_001545.1 /DEF = Homo sapiens immature colon carcinoma transcript 1 (ICT1), mRNA. /FEA = mRNA /GEN = ICT1 |
| | /PROD = immature colon carcinoma transcript 1 /DB_XREF = gi: 4557656 /UG = Hs.9078 immature colon carcinoma transcript 1 |
| | /FL = gb: NM_001545.1 |
| 217826_s_at | gb: NM_016021.1 /DEF = Homo sapiens CGI-76 protein (LOC51632), mRNA. /FEA = mRNA /GEN = LOC51632 /PROD = CGI-76 protein |
| | /DB_XREF = gi: 7706311 /UG = Hs.184325 CGI-76 protein /FL = gb: AF151834.1 gb: AF161502.1 gb: AF151039.1 gb: NM_016021.1 |
| 212534_at | Consensus includes gb: AU144066 /FEA = EST /DB_XREF = gi: 11005587 /DB_XREF = est: AU144066 /CLONE = HEMBA1000798 |
| | /UG = Hs.285519 Homo sapiens cDNA FLJ11904 fis, clone HEMBB1000048 |
| 211284_s_at | gb: BC000324.1 /DEF = Homo sapiens, Similar to granulin, clone MGC: 8480, mRNA, complete cds. /FEA = mRNA /PROD = Similar to |
| | granulin /DB_XREF = gi: 12653114 /UG = Hs.180577 granulin /FL = gb: BC000324.1 |
| 212188_at | Consensus includes gb: AA551075 /FEA = EST /DE_XREF = gi: 2321327 /DB_XREF = est: nk74h06.s1 /CLONE = IMAGE: 1019291 |
| | /UG = Hs.109438 Homo sapiens clone 24775 mRNA sequence |
| 201458_s_at | gb: NM_004725.1 /DEF = Homo sapiens BUB3 (budding uninhibited by benzimidazoles 3, yeast) homolog (BUB3), mRNA. /FEA = mRNA |
| | /GEN = BUB3 /PROD = BUB3 (budding uninhibited by benzimidazoles 3, yeast) homolog /DB_XREF = gi: 4757879 /UG = Hs.40323 BUB3 |
| | (budding uninhibited by benzimidazoles 3, yeast) homolog /FL = gb: BC005138.1 gb: AF047472.1 gb: AF053304.1 gb: AF081496.1 |
| | gb: NM_004725.1 |
| 201773_at | gb: NM_015339.1 /DEF = Homo sapiens activity-dependent neuroprotective protein (ADNP), mRNA. /FEA = mRNA /GEN = ADNP |
| | /PROD = activity-dependent neuroprotective protein /DB_XREF = gi: 12229216 /UG = Hs.3657 activity-dependent neuroprotective protein |
| | /FL = gb: AF250860.1 gb: NM_015339.1 |
| 218751_s_at | gb: NM_018315.1 /DEF = Homo sapiens hypothetical protein FLJ11071 (FLJ11071), mRNA. /FEA = mRNA /GEN = FLJ11071 |
| | /PROD = hypothetical protein FLJ11071 /DB_XREF = gi: 8922851 /UG = Hs.31945 hypothetical protein FLJ11071 /FL = gb: NM_018315.1 |
| 201350_at | gb: NM_004475.1 /DEF = Homo sapiens flotillin 2 (FLOT2), mRNA. /FEA = mRNA /GEN = FLOT2 /PROD = flotillin 2 /DB_XREF = gi: 4758393 |
| | /UG = Hs.184488 flotillin 2 FL = gb: NM_004475.1 gb: M60922.1 |
| 201098_at | gb: NM_004766.1 /DEF = Homo sapiens coatomer protein complex, subunit beta 2 (beta prime) (COPB2), mRNA. /FEA = mRNA |
| | /GEN = COPB2 |
| | /PROD = coatomer protein complex, subunit beta 2 (betaprime) /DB_XREF = gi: 4758031 /UG = Hs.75724 coatomer protein complex, |
| | subunit beta 2 (beta prime) /FL = gb: BC000326.1 gb: NM_004766.1 |
| 203732_at | gb: NM_016213.1 /DEF = Homo sapiens thyroid hormone receptor interactor 4 (TRIP4), mRNA. /FEA = mRNA /GEN = TRIP4 |
| | /PROD = activating signal cointegrator 1 /DB_XREF = gi: 7706430 /UG = Hs.116784 thyroid hormone receptor interactor 4 |
| | /FL = gb: AF168418.1 gb: NM_016213.1 |
| 202846_s_at | gb: NM_002642.1 /DEF = Homo sapiens phosphatidylinositol glycan, class C (PIGC), mRNA. /FEA = mRNA /GEN = PIGC |
| | /PROD = phosphatidylinositol glycan, class C /DB_XREF = gi: 4505794 /UG = Hs.75790 phosphatidylinositol glycan, |
| | class C /FL = gb: D85418.1 gb: NM_002642.1 |
| 205936_s_at | gb: NM_002115.1 /DEF = Homo sapiens hexokinase 3 (white cell) (HK3), mRNA. /FEA = mRNA /GEN = HK3 /PROD = hexokinase 3 (white |
| | cell) /DB_XREF = gi: 4504394 /UG = Hs.159237 hexokinase 3 (white cell) /FL = gb: U51333.1 gb: NM_002115.1 |
| 201209_at | gb: NM_004964.2 /DEF = Homo sapiens histone deacetylase 1 (HDAC1), mRNA. /FEA = mRNA /GEN = HDAC1 /PROD = histone deacetylase 1 |
| | /DB_XREF = gi: 13128859 /UG = Hs.88556 histone deacetylase 1 /FL = gb: BC000301.1 gb: U50079.1 gb: NM_004964.2 gb: D50405.1 |
| 217798_at | Consensus includes gb: AI123426 /FEA = EST /DB_XREF = gi: 3539192 /DB_XREF = est: qa49c09.x1 /CLONE = IMAGE: 1690096 |
| | /UG = Hs.239720 CCR4-NOT transcription complex, subunit 2 /FL = gb: AF113226.1 gb: AF180473.1 gb: NM_014515.1 |
| 209455_at | Consensus includes gb: BE963245 /FEA = EST /DB_XREF = gi: 11766663 /DB_XREF = est: 601656874R1 /CLONE = IMAGE: 3865699 |
| | /UG = Hs.21229 f-box and WD-40 domain protein 1B /FL = gb: AF176022.1 gb: AB033281.1 |
| 212397_at | Consensus includes gb: AL137751.1 /DEF = Homo sapiens mRNA; cDNA DKFZp434I0812 (from clone DKFZp434I0812); partial cds. |
| | /FEA = mRNA /GEN = DKFZp434I0812 /PROD = hypothetical protein /DB_XREF = gi: 6808387 /UG = Hs.263671 Homo sapiens mRNA; |
| | cDNA DKFZp434I0812 (from clone DKFZp434I0812); partial cds |
| 218025_s_at | gb: NM_006117.1 /DEF = Homo sapiens peroxisomal D3,D2-enoyl-CoA isomerase (PECI), mRNA. /FEA = mRNA /GEN = PECI |
| | /PROD = peroxisomal D3,D2-enoyl-CoA isomerase /DB_XREF = gi: 5174624 /UG = Hs.15250 peroxisomal D3,D2-enoyl-CoA isomerase |
| | /FL = gb: AL136642.1 gb: BC002668.1 gb: AF069301.1 gb: AF153612.1 gb: NM_006117.1 gb: AF244138.1 |
| 203217_s_at | gb: NM_003896.1 /DEF = Homo sapiens sialyltransferase 9 (CMP-NeuAc: lactosylceramide alpha-2,3-sialyltransferase; GM3 synthase) |
| | (SIAT9), mRNA. /FEA = mRNA /GEN = SIAT9 /PROD = sialyltransferase 9 (CMP-NeuAc: lactosylceramidealpha-2,3- |
| | sialyltransferase; GM3 synthase) /DB_XREF = gi: 4506954 /UG = Hs.225939 sialyltransferase 9 (CMP-NeuAc: lactosylceramide alpha- |
| | 2,3-sialyltransferase; GM3 synthase) /FL = gb: AB018356.1 gb: NM_003896.1 gb: AF119415.1 |
| 212812_at | Consensus includes gb: AI700633 /FEA = EST /DB_XREF = gi: 4988533 /DB_XREF = est: we38g03.x1 /CLONE = IMAGE: 2343412 /UG = Hs.288232 |
| | Homo sapiens cDNA: FLJ22642 fis, clone HSI06970 |
| 207830_s_at | gb: NM_002713.1 /DEF = Homo sapiens protein phosphatase 1, regulatory (inhibitor) subunit 8 (PPP1RB), mRNA. /FEA = mRNA |
| | /GEN = PPP1R8 /PROD = protein phosphatase 1, regulatory (inhibitor) subunit 8 /DB_XREF = gi: 4506014 /UG = Hs.78961 protein phosphatase |
| | 1, regulatory (inhibitor) subunit 8 /FL = gb: NM_002713.1 gb: U14575.1 |
| 204061_at | gb: NM_005044.1 /DEF = Homo sapiens protein kinase, X-linked (PRKX), mRNA. /FEA = mRNA /GEN = PRKX /PROD = protein kinase, X- |
| | linked /DB_XREF = gi: 4826947 /UG = Hs.147996 protein kinase, X-linked /FL = gb: NM_005044.1 |
| 215633_x_at | Consensus includes gb: AV713720 /FEA = EST /DB_XREF = gi: 10795237 /DB_XREF = est: AV713720 /CLONE = DCBBJA12 /UG = Hs.306434 Homo |
| | sapiens mRNA for LST-1N protein |
| 208660_at | gb: BC000105.1 /DEF = Homo sapiens, Similar to CG14740 gene product, clone MGC: 2503, mRNA, complete cds. /FEA = mRNA |
| | /PROD = Similar to CG14740 gene product /DB_XREF = gi: 12652712 /UG = Hs.239760 citrate synthase /FL = gb: BC000105.1 gb: AF047042.1 |
| | gb: NM_004077.1 |
| 218805_at | gb: NM_018384.1 /DEF = Homo sapiens hypothetical protein FLJ11296 (FLJ11296), mRNA. /FEA = mRNA /GEN = FLJ11296 |
| | /PROD = hypothetical protein FLJ11296 /DB_XREF = gi: 8922984 /UG = Hs.26194 hypothetical protein FLJ11296 /FL = gb: NM_018384.1 |
| 217725_x_at | gb: NM_015640.1 /DEF = Homo sapiens PAI-1 mRNA-binding protein (PAI-RBP1), mRNA. /FEA = mRNA /GEN = PAI-RBP1 /PROD = PAI-1 mRNA- |
| | binding protein /DB_XREF = gi: 7661625 /UG = Hs.165998 PAI-1 mRNA-binding protein /FL = gb: AL080119.1 gb: NM_015640.1 |
| 200802_at | gb: NM_006513.1 /DEF = Homo sapiens seryl-tRNA synthetase (SARS), mRNA. /FEA = mRNA /GEN = SARS /PROD = seryl-tRNA synthetase |
| | /DB_XREF = gi: 5730028 /UG = Hs.4888 seryl-tRNA synthetase /FL = gb: BC000716.1 gb: NM_006513.1 gb: D49914.1 |
| 213203_at | Consensus includes gb: AI633709 /FEA = EST /DB_XREF = gi: 4685039 /DB_XREF = est: th71f03.x1 /CLONE = IMAGE: 2124125 /UG = Hs.30174 |
| | small nuclear RNA activating complex, polypeptide 5, 19 kD |
| 204640_s_at | gb: NM_003563.1 /DEF = Homo sapiens speckle-type POZ protein (SPOP), mRNA. /FEA = mRNA /GEN = SPOP /PROD = speckle-type POZ protein |
| | /DB_XREF = gi: 4507182 /UG = Hs.129951 speckle-type POZ protein /FL = gb: BC003385.1 gb: NM_003563.1 |
| 202324_s_at | gb: NM_022735.1 /DEF = Homo sapiens golgi resident protein GCP60 (GCP60), mRNA. /FEA = mRNA /GEN = GCP60 /PROD = golgi resident |
| | protein GCP60 /DB_XREF = gi: 12232390 /UG = Hs.6831 golgi resident protein GCP60 /FL = gb: AB043587.1 gb: NM_022735.1 |
| 211661_x_at | gb: M80436.1 /DEF = Human platelet activating factor receptor mRNA, complete cds. /FEA = mRNA /GEN = PTAFR /PROD = platelet activating |
| | factor receptor /DB_XREF = gi: 189537 /FL = gb: M80436.1 |
| 208969_at | gb: AF050641.1 /DEF = Homo sapiens NADH-ubiquinone oxidoreductase 39 kDa subunit mRNA, nuclear gene encoding mitochondrial |
| | protein, complete cds. /FEA = mRNA /PROD = NADH-ubiquinone oxidoreductase 39 kDa subunit /DB_XREF = gi: 5326822 /UG = Hs.75227 |
| | NADH dehydrogenase (ubiquinone) 1 alpha, subcomplex, 9 |
| | (39 kD) /FL = gb: AF050641.1 gb: NM_005002.1 |
| 202227_s_at | gb: NM_006696.1 /DEF = Homo sapiens thyroid hormone receptor coactivating protein (SMAP), mRNA. /FEA = mRNA /GEN = SMAP |
| | /PROD = thyroid hormone receptor coactivating protein /DB_XREF = gi: 5730052 /UG = Hs.5464 thyroid hormone receptor coactivating |
| | protein /FL = gb: AF016270.1 gb: NM_006696.1 |
| 221989_at | Consensus includes gb: AW057781 /FEA = EST /DB_XREF = gi: 5933420 /DB_XREF = est: wx03f09.x1 /CLONE = IMAGE: 2542601 /UG = Hs.29797 |
| | ribosomal protein L10 |
| 202545_at | gb: NM_006254.1 /DEF = Homo sapiens protein kinase C, delta (PRKCD), mRNA. /FEA = mRNA /GEN = PRKCD /PROD = protein kinase C, |
| | delta /DB_XREF = gi: 5453969 /UG = Hs.155342 protein kinase C, delta /FL = gb: L07860.1 gb: L07861.1 gb: D10495.1 gb: NM_006254.1 |
| 202214_s_at | gb: NM_003588.1 /DEF = Homo sapiens cullin 4B (CUL4B), mRNA. /FEA = mRNA /GEN = CUL4B /PROD = cullin 4B /DB_XREF = gi: 13270466 |
| | /UG = Hs.155976 cullin 4B /FL = gb: NM_003588.1 gb: AB014595.1 |
| 218366_x_at | gb: NM_022734.1 /DEF = Homo sapiens hypothetical protein FLJ20859 (FLJ20859), mRNA. /FEA = mRNA /GEN = FLJ20859 |
| | /PROD = hypothetical protein FLJ20859 /DB_XREF = gi: 12232388 /UG = Hs.6311 hypothetical protein FLJ20859 /FL = gb: NM_022734.1 |
| 218592_s_at | gb: NM_017829.1 /DEF = Homo sapiens hypothetical protein FLJ20454 (FLJ20454), mRNA. /FEA = mRNA /GEN = FLJ20454 |
| | /PROD = hypothetical protein FLJ20454 /DB_XREF = gi: 8923424 /UG = Hs.26890 hypothetical protein FLJ20454 /FL = gb: AF273271.1 |
| | gb: NM_017829.1 |
| 209658_at | gb: AF164598.1 /DEF = Homo sapiens cell division control protein 16 (CDC16) mRNA, complete cds. /FEA = mRNA /GEN = CDC16 |
| | /PROD = cell division control protein 16 /DB_XREF = gi: 5533374 /UG = Hs.1592 CDC16 (cell division cycle 16, S. cerevisiae , |
| | homolog) /FL = gb: AF164598.1 |
| 202217_at | gb: NM_004649.1 /DEF = Homo sapiens ES1 (zebrafish) protein, human homolog of (C21ORF33), mRNA. /FEA = mRNA /GEN = C21ORF33 |
| | /PROD = ES1 (zebrafish) protein, human homolog of /DB_XREF = gi: 5031690 /UG = Hs.182423 ES1 (zebrafish) protein, |
| | human homolog of /FL = gb: BC002370.1 gb: BC003587.1 gb: D86061.1 gb: U53003.1 gb: NM_004649.1 |
| 54970_at | Cluster Incl. AA868332: ak40h05.s1 Homo sapiens cDNA, 3 end /clone = IMAGE-1408473 /clone_end = 3 /gb = AA868332 |
| | /gi = 2963777 /ug = Hs.77978 /len = 481 |
| 202441_at | Consensus includes gb: AL568449 /FEA = EST /DB_XREF = gi: 12922799 /DB_XREF = est: AL568449 /CLONE = CS0DE001YC12 (3 prime) |
| | /UG = Hs.285818 similar to Caenorhabditis elegans protein C42C1.9 /FL = gb: AF064093.1 gb: NM_006459.1 |
| 211383_s_at | gb: AL136827.1 /DEF = Homo sapiens mRNA; cDNA DKFZp434F2427 (from clone DKFZp434F2427); complete cds. /FEA = mRNA |
| | /GEN = DKFZp434F2427 /PROD = hypothetical protein /DB_XREF = gi: 6807664 /UG = Hs.27207 KIAA0982 protein /FL = gb: AL136827.1 |
| 201903_at | gb: NM_003365.1 /DEF = Homo sapiens ubiquinol-cytochrome c reductase core protein I (UQCRC1), mRNA. /FEA = mRNA /GEN = UQCRC1 |
| | /PROD = ubiquinol-cytochrome c reductase core protein I /DB_XREF = gi: 4507840 /UG = Hs.119251 ubiquinol-cytochrome c |
| | reductase core protein I /FL = gb: L16842.1 gb: NM_003365.1 gb: D26485.1 |
| 201827_at | gb: AF113019.1 /DEF = Homo sapiens PRO2451 mRNA, complete cds. /FEA = mRNA /PROD = PRO2451 /DB_XREF = gi: 6642761 /UG = Hs.250581 |
| | SWISNF related, matrix associated, actin dependent regulator of chromatin, subfamily d, member 2 |
| | /FL = gb: U66618.1 gb: NM_003077.1 gb: AF113019.1 |
| 201583_s_at | gb: NM_006363.1 /DEF = Homo sapiens Sec23 (S. cerevisiae) homolog B (SEC23B), mRNA. /FEA = mRNA /GEN = SEC23B /PROD = Sec23 (S. cerevisiae) |
| | homolog B /DB_XREF = gi: 5454043 /UG = Hs.173497 Sec23 (S. cerevisiae) homolog B /FL = gb: BC005404.1 gb: NM_006363.1 |
| 210825_s_at | gb: AF130103.1 /DEF = Homo sapiens clone FLB2914 PRO0720 mRNA, complete cds. /FEA = mRNA /PROD = PRO0720 /DB_XREF = gi: 11493509 |
| | /UG = Hs.160483 erythrocyte membrane protein band 7.2 (stomatin) /FL = gb: AF130103.1 |
| 212696_s_at | Consensus includes gb: BF968633 /FEA = EST /DB_XREF = gi: 12335848 /DB_XREF = est: 602271068F1 /CLONE = IMAGE: 4359209 |
| | /UG = Hs.66394 ring finger protein 4 |
| 218229_s_at | gb: NM_017542.1 /DEF = Homo sapiens KIAA1513 protein (KIAA1513), mRNA. /FEA = mRNA /GEN = KIAA1513 /PROD = KIAA1513 protein |
| | /DB_XREF = gi: 8923835 /UG = Hs.284227 KIAA1513 protein /FL = gb: NM_017542.1 |
| 210111_s_at | gb: AF277175.1 /DEF = Homo sapiens PNAS-138 mRNA, complete cds. /FEA = mRNA /PROD = PNAS-138 /DB_XREF = gi: 12751080 |
| | /UG = Hs.326790 Homo sapiens PNAS-138 mRNA, complete cds /FL = gb: AF277175.1 |
| 201268_at | gb: NM_002512.1 /DEF = Homo sapiens non-metastatic cells 2, protein (NM23B) expressed in (NME2), |
| | nuclear gene encoding mitochondrial protein, mRNA. /FEA = mRNA /GEN = NME2 /PROD = non-metastatic cells 2, |
| | protein (NM23B) expressed in /DB_XREF = gi: 4505408 /UG = Hs.275163 non-metastatic cells 2, protein (NM23B) |
| | expressed in /FL = gb: BC002476.1 gb: M36981.1 gb: L16785.1 gb: NM_002512.1 |
| 201199_s_at | gb: NM_002807.1 /DEF = Homo sapiens proteasome (prosome, macropain) 26S subunit, non-ATPase, 1 (PSMD1), mRNA. /FEA = mRNA |
| | /GEN = PSMD1 /PROD = proteasome (prosome, macropain) 26S subunit, non-ATPase, 1 /DB_XREF = gi: 4506224 /UG = Hs.3887 proteasome |
| | (prosome, macropain) 26S subunit, non-ATPase, 1 /FL = gb: D44466.1 gb: NM_002807.1 |
| 218242_s_at | gb: NM_017635.1 /DEF = Homo sapiens hypothetical protein FLJ20039 (FLJ20039), mRNA. /FEA = mRNA /GEN = FLJ20039 |
| | /PROD = hypothetical protein FLJ20039 /DB_XREF = gi: 8923045 /UG = Hs.267448 hypothetical protein FLJ20039 /FL = gb: NM_017635.1 |
| 201232_s_at | gb: NM_002817.1 /DEF = Homo sapiens proteasome (prosome, macropain) 26S subunit, non-ATPase, 13 (PSMD13), mRNA. /FEA = mRNA |
| | /GEN = PSMD13 /PROD = proteasome (prosome, macropain) 26S subunit, non-ATPase, 13 /DB_XREF = gi: 4506222 /UG = Hs.279554 proteasome |
| | (prosome, macropain) 26S subunit, non-ATPase, 13 /FL = gb: BC001100.1 gb: BC001747.1 gb: AB009398.1 |
| | gb: NM_002817.1 gb: AF083245.1 gb: AF107837.1 |
| 220044_x_at | gb: NM_016424.1 /DEF = Homo sapiens cisplatin resistance-associated overexpressed protein (LUC7A), mRNA. /FEA = mRNA |
| | /GEN = LUC7A /PROD = cisplatin resistance-associated overexpressedprotein /DB_XREF = gi: 7706534 /UG = Hs.3688 |
| | cisplatin resistance-associated overexpressed protein /FL = gb: NM_016424.1 |
| 210835_s_at | gb: AF222711.1 /DEF = Homo sapiens ribeye mRNA, complete cds. /FEA = mRNA /PROD = ribeye /DB_XREF = gi: 12034652 /UG = Hs.171391 |
| | C-terminal binding protein 2 /FL = gb: AF222711.1 gb: NM_022802.1 |
| 211271_x_at | gb: BC004383.1 /DEF = Homo sapiens, Similar to polypyrimidine tract binding protein (heterogeneous nuclear ribonucleoprotein |
| | I), clone MGC: 10830, mRNA, complete cds. /FEA = mRNA /PROD = Similar to polypyrimidine tract binding protein (heterogeneous |
| | nuclear ribonucleoprotein I) /DB_XREF = gi: 13325139 /UG = Hs.172550 polypyrimidine tract binding protein |
| | (heterogeneous nuclear ribonucleoprotein I) /FL = gb: BC004383.1 |
| 218998_at | gb: NM_017832.1 /DEF = Homo sapiens hypothetical protein FLJ20457 (FLJ20457), mRNA. /FEA = mRNA /GEN = FLJ20457 |
| | /PROD = hypothetical protein FLJ20457 /DB_XREF = gi: 8923430 /UG = Hs.29276 hypothetical protein FLJ20457 |
| | /FL = gb: AF153417.1 gb: NM_017832.1 |
| 201765_s_at | Consensus includes gb: AL523158 /FEA = EST /DB_XREF = gi: 12786651 /DB_XREF = est: AL523158 /CLONE = CS0DC001YM06 (3 prime) |
| | /UG = Hs.119403 hexosaminidase A (alpha polypeptide) /FL = gb: NM_000520.2 |
| 204837_at | Consensus includes gb: AL080178.1 /DEF = Homo sapiens mRNA; cDNA DKFZp434K171 (from clone DKFZp434K171); partial cds. |
| | /FEA = mRNA /GEN = DKFZp434K171 /PROD = hypothetical protein /DB_XREF = gi: 5262652 /UG = Hs.27194 DKFZP434K171 protein |
| | /FL = gb: NM_015458.1 |
| 201221_s_at | gb: NM_003089.1 /DEF = Homo sapiens small nuclear ribonucleoprotein 70 kD polypeptide (RNP antigen) (SNRP70), mRNA. /FEA = mRNA /GEN = SNRP70 |
| | /PROD = small nuclear ribonucleoprotein 70 kD polypeptide(RNP antigen) /DB_XREF = gi: 4507118 /UG = Hs.174051 small nuclear ribonucleoprotein 70 kD |
| | polypeptide (RNP antigen) /FL = gb: BC000342.1 gb: M22636.1 gb: NM_003089.1 |
| 209092_s_at | gb: AF061730.1 /DEF = Homo sapiens clone 016b03 My027 protein mRNA, complete cds. /FEA = mRNA /PROD = My027 protein /DB_XREF = gi: 12001995 |
| | /UG = Hs.279061 CGI-150 protein /FL = gb: AF061730.1 gb: AF151908.1 gb: NM_016080.1 |
| 202265_at | gb: NM_005180.1 /DEF = Homo sapiens murine leukemia viral (bmi-1) oncogene homolog (BMI1), mRNA. /FEA = mRNA /GEN = BMI1 /PROD = murine leukemia |
| | viral (bmi-1) oncogene homolog /DB_XREF = gi: 4885094 /UG = Hs.431 murine leukemia viral (bmi-1) oncogene homolog /FL = gb: L13689.1 gb: NM_005180.1 |
| 203721_s_at | gb: NM_016001.1 /DEF = Homo sapiens CGI-48 protein (LOC51096), mRNA. /FEA = mRNA /GEN = LOC51096 /PROD = CGI-48 protein /DB_XREF = gi: 7705764 |
| | /UG = Hs.6153 CGI-48 protein /FL = gb: AF151806.1 gb: NM_016001.1 |
| 202475_at | gb: NM_006326.1 /DEF = Homo sapiens seven transmembrane domain protein (NIFIE14), mRNA. /FEA = mRNA /GEN = NIFIE14 /PROD = seven transmembrane |
| | domain protein /DB_XREF = gi: 5453781 /UG = Hs.9234 seven transmembrane domain protein /FL = gb: BC001118.1 gb: NM_006326.1 |
| 213165_at | Consensus includes gb: AI041204 /FEA = EST /DB_XREF = gi: 3280398 /DB_XREF = est: ov77g06.x1 /CLONE = IMAGE: 1643386 /UG = Hs.323748 Homo sapiens clone |
| | CDABP0086 mRNA sequence |
| 213198_at | Consensus includes gb: AL117643.1 /DEF = Homo sapiens mRNA; cDNA DKFZp434M245 (from clone DKFZp434M245). /FEA = mRNA /DB_XREF = gi: 5912233 |
| | /UG = Hs.5288 Homo sapiens mRNA; cDNA DKFZp434M245 (from clone DKFZp434M245) |
| 212837_at | Consensus includes gb: D63877.1 /DEF = Human mRNA for KIAA0157 gene, partial cds. /FEA = mRNA /GEN = KIAA0157 /DB_XREF = gi: 961445 /UG = Hs.82324 |
| | KIAA0157 protein |
| 206592_s_at | gb: NM_003938.1 /DEF = Homo sapiens adaptor-related protein complex 3, delta 1 subunit (AP3D1), mRNA. /FEA = mRNA /GEN = AP3D1 /PROD = adaptor- |
| | related protein complex 3, delta 1subunit /DB_XREF = gi: 4501976 /UG = Hs.75056 adaptor-related protein complex 3, delta 1 subunit |
| | /FL = gb: U91930.1 gb: NM_003938.1 |
| 212694_s_at | Consensus includes gb: NM_000532.1 /DEF = Homo sapiens propionyl Coenzyme A carboxylase, beta polypeptide (PCCB), nuclear gene encoding mitochondrial |
| | protein, mRNA. /FEA = CDS /GEN = PCCB /PROD = propionyl Coenzyme A carboxylase, betapolypeptide /DB_XREF = gi: 4557043 /UG = Hs.63788 |
| | propionyl Coenzyme A carboxylase, beta polypeptide /FL = gb: NM_000532.1 |
| 211759_x_at | gb: BC005969.1 /DEF = Homo sapiens, clone MGC: 14625, mRNA, complete cds. /FEA = mRNA /PROD = Unknown (protein for MGC: 14625) /DB_XREF = gi: 13543641 |
| | /FL = gb: BC005969.1 |
| 212714_at | Consensus includes gb: AL050205.1 /DEF = Homo sapiens mRNA; cDNA DKFZp586F1323 (from clone DKFZp586F1323). /FEA = mRNA /DB_XREF = gi: 4884444 |
| | /UG = Hs.26613 Homo sapiens mRNA; cDNA DKFZp586F1323 (from clone DKFZp586F1323) |
| 201528_at | Consensus includes gb: BG398414 /FEA = EST /DB_XREF = gi: 13291862 /DB_XREF = est: 602439888F1 /CLONE = IMAGE: 4566380 /UG = Hs.84318 replication protein |
| | A1 (70 kD) /FL = gb: M63488.1 gb: NM_002945.1 |
| 211795_s_at | gb: AF198052.1 /DEF = Homo sapiens EVH1 domain binding protein mRNA, complete cds. /FEA = CDS /PROD = EVH1 domain binding protein |
| | /DB_XREF = gi: 7416992 /UG = Hs.58435 FYN-binding protein (FYB-120130) /FL = gb: AF198052.1 |
| 218633_x_at | gb: NM_018394.1 /DEF = Homo sapiens hypothetical protein FLJ11342 (FLJ11342), mRNA. /FEA = mRNA /GEN = FLJ11342 /PROD = hypothetical protein |
| | FLJ11342 /DB_XREF = gi: 8923000 /UG = Hs.266514 hypothetical protein FLJ11342 /FL = gb: NM_018394.1 |
| 200800_s_at | gb: NM_005345.3 /DEF = Homo sapiens heat shock 70 kD protein 1A (HSPA1A), mRNA. /FEA = mRNA /GEN = HSPA1A /PROD = heat shock 70 kD protein 1A |
| | /DB_XREF = gi: 5579469 /UG = Hs.8997 heat shock 70 kD protein 1A /FL = gb: BC002453.1 gb: NM_005345.3 |
| 214719_at | Consensus includes gb: AK026720.1 /DEF = Homo sapiens cDNA: FLJ23067 fis, clone LNG04993. /FEA = mRNA /DB_XREF = gi: 10439638 /UG = Hs.117167 Homo |
| | sapiens cDNA: FLJ23067 fis, clone LNG04993 |
| 213655_at | Consensus includes gb: AA502643 /FEA = EST /DB_XREF = gi: 2237610 /DB_XREF = est: ne42g05.s1 /CLONE = IMAGE: 900056 /UG = Hs.79474 tyrosine 3- |
| | monooxygenasetryptophan |
| | 5-monooxygenase activation protein, epsilon polypeptide |
| 212371_at | Consensus includes gb: AL049397.1 /DEF = Homo sapiens mRNA; cDNA DKFZp586C1019 (from clone DKFZp586C1019). /FEA = mRNA /DB_XREF = gi: 4500188 |
| | /UG = Hs.12314 |
| | Homo sapiens mRNA; cDNA DKFZp586C1019 (from clone DKFZp586C1019) |
| 210166_at | gb: AF051151.1 /DEF = Homo sapiens Tollinterleukin-1 receptor-like protein 3 (TIL3) mRNA, complete cds. /FEA = mRNA /GEN = TIL3 |
| | /PROD = Tollinterleukin-1 |
| | receptor-like protein 3 /DB_XREF = gi: 3132525 /UG = Hs.114408 toll-like receptor 5 /FL = gb: AF051151.1 |
| 217958_at | gb: NM_016146.1 /DEF = Homo sapiens PTD009 protein (PTD009), mRNA. /FEA = mRNA /GEN = PTD009 /PROD = PTD009 protein /DB_XREF = gi: 7706666 |
| | /UG = Hs.279901 PTD009 |
| | /UG = Hs.279901 PTD009 |
| | protein /FL = gb: AF151862.1 gb: AF078862.1 gb: AF161520.1 gb: NM_016146.1 |
| 219269_at | gb: NM_024567.1 /DEF = Homo sapiens hypothetical protein FLJ21616 (FLJ21616), mRNA. /FEA = mRNA /GEN = FLJ21616 /PROD = hypothetical protein |
| | FLJ21616 /DB_XREF = gi: 13375737 /UG = Hs.14562 hypothetical protein FLJ21616 /FL = gb: NM_024567.1 |
| 219574_at | gb: NM_017923.1 /DEF = Homo sapiens hypothetical protein FLJ20668 (FLJ20668), mRNA. /FEA = mRNA /GEN = FLJ20668 /PROD = hypothetical protein |
| | FLJ20668 /DB_XREF = gi: 8923612 /UG = Hs.12920 hypothetical protein FLJ20668 /FL = gb: NM_017923.1 |
| 210183_x_at | gb: AF112222.1 /DEF = Homo sapiens nuclear protein SDK3 mRNA, complete cds. /FEA = mRNA /PROD = nuclear protein SDK3 /DB_XREF = gi: 6563229 |
| | /UG = Hs.44499 pinin, desmosome associated protein /FL = gb: AF112222.1 |
| 211582_x_at | gb: AF000424.1 /DEF = Homo sapiens LST1 mRNA, cLST1C splice variant, complete cds. /FEA = mRNA /GEN = LST1 /DB_XREF = gi: 2145063 /UG = Hs.88411 |
| | lymphocyte antigen 117 /FL = gb: AF000424.1 |
| 212557_at | Consensus includes gb: AB011148.1 /DEF = Homo sapiens mRNA for KIAA0576 protein, partial cds. /FEA = mRNA /GEN = KIAA0576 /PROD = KIAA0576 protein |
| | /DB_XREF = gi: 3043675 /UG = Hs.172329 KIAA0576 protein |
| 213387_at | Consensus includes gb: AB033066.1 /DEF = Homo sapiens mRNA for KIAA1240 protein, partial cds. /FEA = mRNA /GEN = KIAA1240 /PROD = KIAA1240 protein |
| | /DB_XREF = gi: 6330790 /UG = Hs.62576 KIAA1240 protein |
| 218143_s_at | gb: NM_005697.2 /DEF = Homo sapiens secretory carrier membrane protein 2 (SCAMP2), mRNA. /FEA = mRNA /GEN = SCAMP2 /PROD = secretory carrier membrane |
| | protein 2 /DB_XREF = gi: 5730030 /UG = Hs.238030 secretory carrier membrane protein 2 /FL = gb: BC001376.1 gb: BC004385.1 gb: AF005038.2 |
| | gb: NM_005697.2 |
| 211615_s_at | gb: M92439.1 /DEF = Human leucine-rich protein mRNA, complete cds. /FEA = mRNA /PROD = leucine-rich protein; leucine-rich protein |
| | /DB_XREF = gi: 177109 /FL = gb: M92439.1 |
| 213359_at | Consensus includes gb: W74620 /FEA = EST /DB_XREF = gi: 1384833 /DB_XREF = est: zd77e04.s1 /CLONE = IMAGE: 346686 /UG = Hs.303627 heterogeneous nuclear |
| | ribonucleoprotein D (AU-rich element RNA-binding protein 1, 37 kD) |
| 217842_at | gb: NM_016019.1 /DEF = Homo sapiens CGI-74 protein (LOC51631), mRNA. /FEA = mRNA /GEN = LOC51631 /PROD = CGI-74 protein /DB_XREF = gi: 7706309 |
| | /UG = Hs.7194 CGI-74 protein /FL = gb: AF151832.1 gb: NM_016019.1 |
| 207170_s_at | gb: NM_015416.1 /DEF = Homo sapiens DKFZP586A011 protein (DKFZP586A011), mRNA. /FEA = mRNA /GEN = DKFZP586A011 /PROD = DKFZP586A011 protein |
| | /DB_XREF = gi: 7661659 /UG = Hs.75884 DKFZP586A011 protein /FL = gb: NM_015416.1 |
| 214352_s_at | Consensus includes gb: BF673699 /FEA = EST /DB_XREF = gi: 11947594 /DB_XREF = est: 602136427F1 /CLONE = IMAGE: 4272774 /UG = Hs.184050 v-Ki-ras2 Kirsten |
| | rat sarcoma 2 viral oncogene homolog |
| 218287_s_at | gb: NM_012199.1 /DEF = Homo sapiens eukaryotic translation initiation factor 2C, 1 (EIF2C1), mRNA. /FEA = mRNA /GEN = EIF2C1 /PROD = eukaryotic |
| | translation initiation factor 2C, 1 /DB_XREF = gi: 6912351 /UG = Hs.14520 eukaryotic translation initiation factor 2C, 1 /FL = gb: AF093097.1 |
| | gb: NM_012199.1 |
| 204661_at | gb: NM_001803.1 /DEF = Homo sapiens CDW52 antigen (CAMPATH-1 antigen) (CDW52), mRNA. /FEA = mRNA /GEN = CDW52 /PROD = CDW52 antigen (CAMPATH-1 antigen) |
| | /DB_XREF = gi: 4502760 /UG = Hs.276770 CDW52 antigen (CAMPATH-1 antigen) /FL = gb: BC000644.1 gb: NM_001803.1 |
| 205545_x_at | gb: NM_014280.1 /DEF = Homo sapiens splicing factor similar to dnaJ (SPF31), mRNA. /FEA = mRNA /GEN = SPF31 /PROD = splicing factor similar to dnaJ |
| | /DB_XREF = gi: 7657610 /UG = Hs.74711 DnaJ (Hsp40) homolog, subfamily C, member 8 /FL = gb: AF083190.1 gb: NM_014280.1 |
| 213750_at | Consensus includes gb: AA928506 /FEA = EST /DB_XREF = gi: 3076797 /DB_XREF = est: om17g03.s1 /CLONE = IMAGE: 1541332 /UG = Hs.10762 ESTs |
| 221970_s_at | Consensus includes gb: AU158148 /FEA = EST /DB_XREF = gi: 11019669 /DB_XREF = est: AU158148 /CLONE = PLACE1011221 /UG = Hs.321105 Homo sapiens cDNA: |
| | FLJ21737 fis, clone COLF3396 |
| 218684_at | gb: NM_018103.1 /DEF = Homo sapiens hypothetical protein FLJ10470 (FLJ10470), mRNA. /FEA = mRNA /GEN = FLJ10470 /PROD = hypothetical protein |
| | FLJ10470 /DB_XREF = gi: 8922441 /UG = Hs.44672 hypothetical-protein FLJ10470 /FL = gb: NM_018103.1 |
| 200628_s_at | gb: M61715.1 /DEF = Human tryptophanyl-tRNA synthetase (WRS) mRNA, complete cds. /FEA = mRNA /GEN = WARS /PROD = transfer RNA-Trp synthetase |
| | /DB_XREF = gi: 340367 /UG = Hs.82030 tryptophanyl-tRNA synthetase /FL = gb: M77804.1 gb: M61715.1 gb: NM_004184.2 |
| 203905_at | gb: NM_002582.1 /DEF = Homo sapiens poly(A)-specific ribonuclease (deadenylation nuclease) (PARN), mRNA. /FEA = mRNA /GEN = PARN /PROD = poly(A)- |
| | specific ribonuclease (deadenylationnuclease) /DB_XREF = gi: 4505610 /UG = Hs.43445 poly(A)-specific ribonuclease (deadenylation nuclease) |
| | /FL = gb: NM_002582.1 |
| 204039_at | gb: NM_004364.1 /DEF = Homo sapiens CCAATenhancer binding protein (CEBP), alpha (CEBPA), mRNA. /FEA = mRNA /GEN = CEBPA /PROD = CCAATenhancer binding |
| | protein (CEBP), alpha /DB_XREF = gi: 4757971 /UG = Hs.76171 CCAATenhancer binding protein (CEBP), alpha /FL = gb: NM_004364.1 |
| 63009_at | Cluster Incl. AI188402: qd08e03.x1 Homo sapiens cDNA, 3 end /clone = IMAGE-1723132 /clone_end = 3 /gb = AI188402 /gi = 3739611 /ug = Hs.93391 |
| | /len = 506 |
| 211922_s_at | gb: AY028632.1 /DEF = Homo sapiens catalase (CAT) mRNA, complete cds. /FEA = CDS /GEN = CAT /PROD = catalase/DB_XREF = gi: 13562131 /FL = gb: AY028632.1 |
| 212861_at | Consensus includes gb: BF690150 /FEA = EST /DB_XREF = gi: 11975558 /DB_XREF = est: 602186478T1 /CLONE = IMAGE: 4298635 /UG = Hs.19210 ESTs |
| 206715_at | gb: NM_012252.1 /DEF = Homo sapiens transcription factor EC (TFEC), mRNA. /FEA = mRNA /GEN = TFEC /PROD = transcription factor EC |
| | /DB_XREF = gi: 6912701 /UG = Hs.113274 transcription factor EC /FL = gb: D43945.1 gb: NM_012252.1 |
| 213123_at | Consensus includes gb: BE222709 /FEA = EST /DB_XREF = gi: 8910027 /DB_XREF = est: hu51g06.x1 /CLONE = IMAGE: 3173626 /UG = Hs.28785 microfibrillar- |
| | associated protein 3 |
| 204049_s_at | gb: NM_014721.1 /DEF = Homo sapiens KIAA0680 gene product (KIAA0680), mRNA. /FEA = mRNA /GEN = KIAA0680 /PROD = KIAA0680 gene product |
| | /DB_XREF = gi: 7662247 /UG = Hs.102471 KIAA0680 gene product /FL = gb: AB014580.1 gb: NM_014721.1 |
| 201985_at | gb: NM_014846.1 /DEF = Homo sapiens KIAA0196 gene product (KIAA0196), mRMA. /FEA = mRNA /GEN = KIAA0196 /PROD = KIAA0196 gene product |
| | /DB_XREF = gi: 7661987 /UG = Hs.8294 KIAA0196 gene product /FL = gb: D83780.1 gb: NM_014846.1 |
| 208773_s_at | gb: AL136943.1 /DEF = Homo sapiens mRNA; cDNA DKFZp586G1024 (from clone DKFZp586G1024); complete cds. /FEA = mRNA /GEN = DKFZp586G1024 |
| | /PROD = hypothetical protein /DB_XREF = gi: 12053380 /UG = Hs.301226 KIAA1085 protein /FL = gb: AL136943.1 |
| 219563_at | gb: NM_024633.1 /DEF = Homo sapiens hypothetical protein FLJ21276 (FLJ21276), mRNA. /FEA = mRNA /GEN = FLJ21276 /PRO = hypothetical protein |
| | FLJ21276 /DB_XREF = gi: 13375863 /UG = Hs.41502 by hypothetical protein FLJ21276 /FL = gb: NM_024633.1 |
| 218501_at | gb: NM_019555.1 /DEF = Homo sapiens Rho guanine nucleotide exchange factor (GEF) 3 (ARHGEF3), mRNA. /FEA = mRNA /GEN = ARHGEF3 /PROD = Rho guanine |
| | nucleotide exchange factor (GEF) 3 /DB_XREF = gi: 9506400 /UG = Hs.25951 Rho guanine nucleotide exchange factor (GEF) 3 /FL = gb: AF249744.1 |
| | gb: NM_019555.1 |
| 212833_at | Consensus includes gb: M74089.1 /DEF = Human TB1 gene mRNA, 3 end. /FEA = mRNA /GEN = TB1 /DB_XREF = gi: 182400 /UG = Hs.75639 Human TB1 gene mRNA, 3 |
| | end |
| 209623_at | Consensus includes gb: AW439494 /FEA = EST /DB_XREF = gi: 6974800 /DB_XREF = est: xt19c01.x1 /CLONE = IMAGE: 2779584 /UG = Hs.167531 methylcrotonoyl- |
| | Coenzyme A carboxylase 2 (beta) /FL = gb: AB050049.1 gb: AF310971.1 gb: AF301000.1 gb: NM_022132.2 |
| 209969_s_at | gb: BC002704.1 /DEF = Homo sapiens, Similar to signal transducer and activator of transcription 1, 91 kD, clone MGC: 3493, mRNA, complete cds. |
| | /FEA = mRNA /PROD = Similar to signal transducer and activator oftranscription 1, 91 kD /DB_XREF = gi: 12803734 /UG = Hs.21486 signal transducer and |
| | activator of transcription 1, 91 kD /FL = gb: BC002704.1 |
| 219966_x_at | gb: NM_017869.1 /DEF = Homo sapiens BANP homolog, SMAR1 homolog (FLJ20538), mRNA. /FEA = mRNA /GEN = FLJ20538 /PROD = BANP homolog, SMAR1 homolog |
| | /DB_XREF = gi: 8923506 /UG = Hs.194637 BANP homolog, SMAR1 homolog /FL = gb: NM_017869.1 |
| 213275_x_at | Consensus includes gb: W47179 /FEA = EST /DB_XREF = gi: 1332046 /DB_XREF = est: zc34d07.s1 /CLONE = IMAGE: 324205 /UG = Hs.297939 cathepsin B |
| 210231_x_at | gb: D45198.1 /DEF = Human mRNA for template acyivating factor-I alpha complete cds. /FEA = mRNA /GEN = set /PROD = template acyivating factor-I |
| | alpha /DB_XREF = gi: 971271 /UG = Hs.145279 SET translocation (myeloid leukemia-aasociated) /FL = gb: D45198.1 |
| 212474_at | Consensus includes gb: D87682.1 /DEF = Human mRNA for KIAA0241 gene, partial cds. /FEA = mRNA /GEN = KIAA0241 /DB_XREF = gi: 1663699 /UG = Hs.150275 |
| | KIAA0241 protein |
| 208717_at | gb: BC001669.1 /DEF = Homo sapiens, Similar to oxidase (cytochrome c) assembly 1-like, clone MGC: 2171, mRNA, complete cds. /FEA = mRNA |
| | /PROD = Similar to oxidase (cytochrome c) assembly-like /DB_XREF = gi: 12804516 /UG = Hs.151134 oxidase (cytochrome c) assembly 1-like |
| | /FL = gb: BC001669.1 gb: NM_005015.1 |
| 217527_s_at | Consensus includes gb: AI478300 /FEA = EST /DB_XREF = gi: 4371526 /DB_XREF = est: tm39e01.x1 /CLONE = IMAGE: 2160504 /UG = Hs.192789 ESTs, Weakly similar |
| | to ALU6 HUMAN ALU SUBFAMILY SP SEQUENCE CONTAMINATION WARNING ENTRY H. sapiens |
| 220495_s_at | gb: NM_024715.1 /DEF = Homo sapiens hypothetical protein FLJ22625 (FLJ22625), mRNA. /FEA = mRNA /GEN = FLJ22625 /PROD = hypothetical protein |
| | FLJ22625 /DB_XREF = gi: 13376016 /UG = Hs.106534 hypothetical protein FLJ22625 /FL = gb: NM_024715.1 |
| 200892_s_at | gb: BC000451.1 /DEF = Homo sapiens, splicing factor, arginineserine-rich (transformer 2 Drosophila homolog) 10, clone MGC: 8454, mRNA, complete |
| | cds. /FEA = mRNA /PROD = splicing factor, arginineserine-rich(transformer 2 Drosophila homolog) 10 /DB_XREF = gi: 12653362 /UG = Hs.30035 splicing |
| | factor, arginineserine-rich (transformer 2 Drosophila homolog) 10 /FL = gb: BC000160.1 gb: BC000451.1 gb: U61267.1 gb: U68063.1 gb: NM_004593.1 |
| 201798_s_at | gb: NM_013451.1 /DEF = Homo sapiens fer-1 (C. elegans)-like 3 (myoferlin) (FER1L3), mRNA. /FEA = mRNA /GEN = FER1L3 /PROD = fer-1 (C. elegans)-like 3 |
| | (myoferlin) /DB_XREF = gi: 7305052 /UG = Hs.234680 fer-1 (C. elegens) -like 3 (myoferlin) /FL = gb: AF182316.1 gb: NM_013451.1 |
| 202529_at | gb: NM_002766.1 /DEF = Homo sapiens phosphoribosyl pyrophosphate synthetase-associated protein 1 (PRPSAP1), mRNA. /FEA = mRNA /GEN = PRPSAP1 |
| | /PROD = phosphoribosyl pyrophosphatesynthetese-associated protein 1 /DB_XREF = gi: 4506130 /UG = Hs.77498 phosphoribosyl pyrophosphate synthetase- |
| | associated protein 1 /FL = gb: D61391.1 gb: NM_002766.1 |
| 208897_s_at | gb: BC003360.1 /DEF = Homo sapiens, DEADH (Asp-Glu-Ala-AspHis) box polypeptide 18 (Myc-regulated), clone MGC: 5316, mRNA, complete cds. |
| | /FEA = mRNA /PROD = DEADH (Asp-Glu-Ala-AspHis) box polypeptide 18(Myc-regulated) /DB_XREF = gi: 13097182 /UG = Hs.100555 DEADH (Asp-Glu-Ala-AspHis) |
| | box polypeptide 18 (Myc-regulated) /FL = gb: BC001238.1 gb: BC003360.1 |
| 210296_s_at | gb: BC005375.1 /DEF = Homo sapiens, peroxisomal membrane protein 3 (35 kD, Zellweger syndrome), clone MGC: 12491, mRNA, complete cds. /FEA = mRNA |
| | /PROD = peroxisomal membrane protein 3 (35 kD, Zellwegersyndrome) /DB_XREF = gi: 13529226 /UG = Hs.180612 peroxisomal membrane protein 3 (35 kD, |
| | Zellweger syndrome) /FL = gb: BC005375.1 |
| 212460_at | Consensus includes gb: BE738425 /FEA = EST /DB_XREF = gi: 10152417 /DB_XREF = est: 601572441T1 /CLONE = IMAGE: 3839147 /UG = Hs.241507 ribosomal protein |
| | S6 |
| 211800_s_at | gb: AF017306.1 /DEF = Homo sapiens deubiquitinating enzyme UnpES (UNP) mRNA, complete cds. /FEA = CDS /GEN = UNP /PROD = UnpES /DB_XREF = gi: 2656142 |
| | /UG = Hs.77500 ubiquitin specific protease 4 (proto-oncogene) /FL = gb: AF017306.1 |
| 202850_at | gb: NM_002858.2 /DEF = Homo sapiens ATP-binding cassette, sub-family D (ALD), member 3 (ABCD3), mRNA. /FEA = mRNA /GEN = ABCD3 /PROD = ATP-binding |
| | cassette, sub-family D, member 3 /DB_XREF = gi: 10947125 /UG = Hs.76781 ATP-binding cassette, sub-family D (ALD), member 3 /FL = gb: NM_002858.2 |
| | gb: M81182.1 |
| 212202_s_at | Consensus includes gb: BG493972 /FEA = EST /DB_XREF = gi: 13455486 /DB_XREF = est: 602542252F1 /CLONE = IMAGE: 4673316 /UG = Hs.16492 DKFZP564G2022 protein |
| 209337_at | gb: AF063020.1 /DEF = Homo sapiens lens epithelium-derived growth factor mRNA, complete cds. /FEA = mRNA /PROD = lens epithelium-derived growth |
| | factor /DB_XREF = gi: 3283351 /UG = Hs.82110 PC4 and SFRS1 interacting protein 1 /FL = gb: NM_021144.1 gb: AF063020.1 |
| 207616_s_at | gb: NM_004180.1 /DEF = Homo sapiens TRAF family member-associated NFKB activator (TANK), mRNA. /FEA = mRNA /GEN = TANK /PROD = TRAF family member- |
| | associated NFKB activator /DB_XREF = gi: 4759249 /UG = Hs.146847 TRAF family member-associated NFKB activator /FL = gb: U63830.1 gb: NM_004180.1 |
| 204630_s_at | gb: NM_004871.1 /DEF = Homo sapiens golgi SNAP receptor complex member 1 (GOSR1), mRNA. /FEA = mRNA /GEN = GOSR1 /PROD = golgi SNAP receptor complex |
| | member 1 /DB_XREF = gi: 4758455 /UG = Hs.8868 golgi SNAP receptor complex member 1 /FL = gb: AF073926.1 gb: NM_004871.1 |
| 209422_at | Consensus includes gb: AL109965 /DEF = Human DNA sequence from clone RP5-1121G12 on chromosome 20 Contains the 3 end of a gene encoding two |
| | isoforms (the hepatocellular carcinoma-associated antigen 58 (HCA58) and a putative novel protein containing a PHD finger domain), the |
| | SCAND1 gen. . . /FEA = mRNA 2 /DB_XREF = gi: 9663113 /UG = Hs.112594 hypothetical protein DKFZp434F0272 /FL = gb: AY027523.1 gb: AF348207.1 |
| 203658_at | gb: BC001689.1 /DEF = Homo sapiens, carnitineacylcarnitine translocase, clone MGC: 1207, mRNA, complete cds. /FEA = mRNA |
| | /PROD = carnitineacylcarnitine translocase /DB_XREF = gi: 12804552 /UG = Hs.13845 solute carrier family 25 (carnitineacylcarnitine translocase), |
| | member 20 /FL = gb: BC001689.1 gb: NM_000387.2 |
| 212015_x_at | Consensus includes gb: BF690062 /FEA = EST /DB_XREF = gi: 11975470 /DB_XREF = est: 602186366T1 /CLONE = IMAGE: 4298440 /UG = Hs.172550 polypyrimidine |
| | tract binding protein (heterogeneous nuclear ribonucleoprotein I) |
| 222122_s_at | Consensus includes gb: BG403671 /FEA = EST /DB_XREF = gi: 13297119 /DB_XREF = est: 602419393F1 /CLONE = IMAGE: 4526326 /UG = Hs.16411 hypothetical protein |
| | LOC57187 |
| 202171_at | Consensus includes gb: AU146275 /FEA = EST /DB_XREF = gi: 11007796 /DB_XREF = est: AU146275 /CLONE = HEMBB1000004 /UG = Hs.6557 zinc finger protein 161 |
| | /FL = gb: D28118.1 gb: NM_007146.1 |
| 210460_s_at | gb: AB033605.1 /DEF = Homo sapiens mRNA for pUb-R5, complete cds. /FEA = mRNA /GEN = hpUb-R5 /PROD = pUb-R5 /DB_XREF = gi: 8918352 /UG = Hs.148495 proteasome |
| | (prosome, macropain) 26S subunit, non-ATPase, 4 /FL = gb: AB033605.1 |
| 207551_s_at | gb: NM_006800.1 /DEF = Homo sapiens male-specific lethal-3 (Drosophila)-like 1 (MSL3L1), mRNA. /FEA = mRNA /GEN = MSL3L1 /PROD = male-specific |
| | lethal-3 (Drosophila)-like 1 /DB_XREF = gi: 5803103 /UG = HS.88764 male-specific lethal-3 (Drosophila)-like 1 /FL = gb: AF117065.1 gb: NM_006800.1 |
| 201104_x_at | gb: NM_015383.1 /DEF = Homo sapiens hypothetical protein (DJ328E19.C1.1), mRNA. /FEA = mRNA /GEN = DJ328E19.C1.1 /PROD = hypothetical protein |
| | /DB_XREF = gi: 7657016 /UG = Hs.218329 hypothetical protein /FL = gb: NM_015383.1 |
| 200812_at | gb: NM_006429.1 /DEF = Homo sapiens chaperonin containing TCP1, subunit 7 (eta) (CCT7), mRNA. /FEA = mRNA /GEN = CCT7 /PROD = chaperonin containing |
| | TCP1, subunit 7 (eta) /DB_XREF = gi: 5453606 /UG = Hs.108809 chaperonin containing TCP1, subunit 7 (eta) /FL = gb: AF026292.1 gb: NM_006429.1 |
| 203494_s_at | gb: NM_014679.1 /DEF = Homo sapiens KIAA0092 gene product (KIAA0092), mRNA. /FEA = mRNA /GEN = KIAA0092 /PROD = KIAA0092 gene product |
| | /DB_XREF = gi: 7661899 /UG = Hs.151791 KIAA0092 gene product /FL = gb: D42054.1 gb: NM_014679.1 |
| 202127_at | Consensus includes gb: AB011108.1 /DEF = Homo sapiens mRNA for KIAA0536 protein, partial cds. /FEA = mRNA /GEN = KIAA0536 /PROD = KIAA0536 protein |
| | /DB_XREF = gi: 3043595 /UG = Hs.198891 serinethreonine-protein kinase PRP4 homolog /FL = gb: U48736.1 gb: NM_003913.1 |
| 212532_s_at | Consensus includes gb: AW873564 /FEA = EST /DB_XREF = gi: 8007617 /DB_XREF = est: ho62c10.x1 /CLONE = IMAGE: 3041970 /UG = Hs.155485 huntingtin interacting |
| | protein 2 |
| 218357_s_at | gb: NM_012459.1 /DEF = Homo sapiens translocase of inner mitochondrial membrane 8 (yeast) homolog B (TIMM8B), mRNA. /FEA = mRNA /GEN = TIMM8B |
| | /PROD = translocase of inner mitochondrial membrane 8(yeast) homolog B /DB_XREF = gi: 6912711 /UG = Hs.279915 translocase of inner mitochondrial |
| | membrane 8 |
| | (yeast) homolog B /FL = gb: AF150087.1 gb: AF152350.1 gb: NM_012459.1 |
| 208847_s_at | gb: M29872.1 /DEF = Human alcohol dehydrogenase class III (ADH5) mRNA, complete cds. /FEA = mRNA /GEN = ADH5 /DB_XREF = gi: 178131 /UG = Hs.78989 alcohol |
| | dehydrogenase 5 (class III), chi polypeptide /FL = gb: NM_000671.2 gb: M29872.1 gb: M30471.1 |
| 207730_x_at | gb: NM_017932.1 /DEF = Homo sapiens hypothetical protein FLJ20700 (FLJ20700), mRNA. /FEA = mRNA /GEN = FLJ20700 /PROD = hypothetical protein |
| | FLJ20700 /DB_XREF = gi: 8923629 /UG = Hs.272222 hypothetical protein FLJ20700 /FL = gb: NM_017932.1 |
| 212896_at | Consensus includes gb: D29641.2 /DEF = Homo sapien mRNA for KIAA0052 protein, partial cds. /FEA = mRNA /GEN = KIAA0052 /PROD = KIAA0052 protein |
| | /DB_XREF = gi: 6633994 /UG = Hs.278608 KIAA0052 protein |
| 203156_at | gb: NM_016248.1 /DEF = Homo sapiens A-kinase anchoring protein 220 (LOC51707), mRNA. /FEA = mRNA /GEN = LOC51707 /PROD = A-kinase anchoring protein |
| | 220 /DB_XREF = gi: 7706456 /UG = Hs.232076 A kinase (PRKA) anchor protein 11 /FL = gb: AF176555.1 gb: NM_016248.1 |
| 220066_at | gb: NM_022162.1 /DEF = Homo sapiens NOD2 protein (NOD2), mRNA. /FEA = mRNA /GEN = NOD2 /PROD = NOD2 protein /DB_XREF = gi: 11545911 /UG = Hs.135201 NOD2 |
| | protein /FL = gb: AF178930.1 gb: NM_022162.1 |
| | protein /FL = gb: AF178930.1 gb: NM_022162.1 |
| 218583_s_at | gb: NM_020640.1 /DEF = Homo sapiens RP42 homolog (RP42), mRNA. /FEA = mRNA /GEN = RP42 /PROD = RP42 homolog /DB_XREF = gi: 10190677 /UG = Hs.104613 RP42 |
| | homolog /FL = gb: NM_020640.1 gb: AF292100.2 |
| 221689_s_at | gb: AB035745.1 /DEF = Homo sapiens mRNA for DSCR5b, complete cds. /FEA = mRNA /GEN = DSCR5b /PROD = DSCR5b /DB_XREF = gi: 7798596 /UG = Hs.66493 Down |
| | syndrome critical region gene 5 /FL = gb: AB035745.1 gb: AB037163.1 gb: AF237812.1 |
| 205412_at | gb: NM_000019.1 /DEF = Homo sapiens acetyl-Coenzyme A acetyltransferase 1 (acetoacetyl Coenzyme A thiolase) (ACAT1), nuclear gene encoding |
| | mitochondrial protein, mRNA. /FEA = mRNA /GEN = ACAT1 /PROD = acetyl-Coenzyme A acetyltransferase 1 precursor /DB_XREF = gi: 4557236 /UG = Hs.37 |
| | acetyl-Coenzyme A acetyltransferase 1 (acetoacetyl Coenzyme A thiolase) /FL = gb: NM_000019.1 |
| 202542_s_at | gb: NM_004757.1 /DEF = Homo sapiens small inducible cytokine subfamily E, member 1 (endothelial monocyte-activating) (SCYE1), mRNA. /FEA = mRNA |
| | /GEN = SCYE1 /PROD = small inducible cytokine subfamily E, member 1 /DB_XREF = gi: 4758265 /UG = Hs.146401 small inducible cytokine subfamily E, |
| | member 1 (endothelialmonocyte-activating) /FL = gb: NM_004757.1 gb: U10117.1 |
| 202521_at | gb: NM_006565.1 /DEF = Homo sapiens CCCTC-binding factor (zinc finger protein) (CTCF), mRNA. /FEA = mRNA /GEN = CTCF /PROD = CCCTC-binding factor |
| | (zinc finger protein) /DB_XREF = gi: 5729789 /UG = Hs.57419 CCCTC-binding factor (zinc finger protein) /FL = gb: NM_006565.1 gb: U25435.1 |
| 204391_x_at | gb: NM_015905.1 /DEF = Homo sapiens transcriptional intermediary factor 1 (TIF1), mRNA. /FEA = mRNA /GEN = TIF1 /PROD = transcriptional intermediary |
| | factor 1 alpha /DB_XREF = gi: 7706233 /UG = Hs.183858 transcriptional intermediary factor 1 /FL = gb: AF009353.1 gb: AF119042.1 gb: NM_003852.1 |
| | gb: NM_015905.1 |
| 214733_s_at | Consensus includes gb: AL031427 /DEF = Human DNA sequence from clone 167A19 on chromosome 1p32.1-33. Contains three genes for novel proteins, |
| | the DIO1 gene for type I iodothyronine deiodinase (EC 3.8.1.4, TXDI1, ITDI1) and an HNRNP A3 (Heterogenous Nuclear Ribonucleoprotein A3, |
| | FBRNP). . . /FEA = mRNA 6 /DB_XREF = gi: 4835258 /UG = Hs.11923 hypothetical protein |
| 217864_s_at | gb: NM_016166.1 /DEF = Homo sapiens DEADH (Asp-Glu-Ala-AspHis) box binding protein 1 (DDXBP1), mRNA. /FEA = mRNA /GEN = DDXBP1 /PROD = DEADH (Asp- |
| | Glu-Ala-AspHis) box binding protein1 /DB_XREF = gi: 7706636 /UG = Hs.75251 DEADH (Asp-Glu-Ala-AspHis) box binding protein 1 /FL = gb: AF077951.1 |
| | gb: AF167160.1 gb: NM_016166.1 |
| 212904_at | Consensus includes gb: AB033011.1 /DEF = Homo sapiens mRNA for KIAA1185 protein, partial cds. /FEA = mRNA /GEN = KIAA1185 /PROD = KIAA1185 protein |
| | /DB_XREF = gi: 6330301 /UG = Hs.268488 KIAA1185 protein |
| 202126_at | Consensus includes gb: AA156948 /FEA = EST /DB_XREF = gi: 1728563 /DB_XREF = est: z119f02.s1 /CLONE = IMAGE: 502395 /UG = Hs.198891 serinethreonine- |
| | protein kinase PRP4 homolog /FL = gb: U48736.1 gb: NM_003913.1 |
| 203484_at | gb: NM_014302.1 /DEF = Homo sapiens Sec61 gamma (SEC61G), mRNA. /FEA = mRNA /GEN = SEC61G /PROD = Sec61 gamma /DB_XREF = gi: 7657545 /UG = Hs.9950 Sec61 |
| | gamma /FL = gb: AF054184.1 gb: NM_014302.1 |
| 203345_s_at | Consensus includes gb: AI566096 /FEA = EST /DB_XREF = gi: 4524548 /DB_XREF = est: tn53d02.x1 /CLONE = IMAGE: 2172099 /UG = Hs.31016 putative DNA binding |
| | protein /FL = gb: AF072814.1 gb: NM_007358.1 |
| 213238_at | Consensus includes gb: AI478147 /FEA = EST /DB_XREF = gi: 4371373 /DB_XREF = est: tm34f06.x1 /CLONE = IMAGE: 2160035 /UG = Hs.173540 ATPase, Class V, |
| | type 10D |
| 202680_at | gb: NM_002095.1 /DEF = Homo sapiens general transcription factor IIE, polypeptide 2 (beta subunit, 34 kD) (GTF2E2), mRNA. /FEA = mRNA |
| | /GEN = GTF2E2 /PROD = general transcription factor IIE, polypeptide 2(beta subunit, 34 kD) /DB_XREF = gi: 4504194 /UG = Hs.77100 general transcription |
| | factor IIE, |
| | polypeptide 2 (beta subunit, 34 kD) /FL = gb: NM_002095.1 |
| 218117_at | gb: NM_014248.1 /DEF = Homo sapiens ring-box 1 (RBX1), mRNA. /FEA = mRNA /GEN = RBX1 /PROD = ring-box 1 /DB_XREF = gi: 7657507 /UG = Hs.279919 ring-box 1 /FL = gb: |
| | BC001466.1 gb: AF140598.1 gb: AF142059.1 gb: NM_014248.1 |
| 218768_at | gb: NM_020401.1 /DEF = Homo sapiens nuclear pore complex protein (NUP107), mRNA. /FEA = mRNA /GEN = NUP107 /PROD = nuclear pore complex protein |
| | /DB_XREF = gi: 9966880 /UG = Hs.236204 nuclear pore complex protein /FL = gb: NM_020401.1 |
| 202271_at | Consensus includes gb: AB007952.1 /DEF = Homo sapiens mRNA for KIAA0483 protein, partial cds. /FEA = mRNA /GEN = KIAA0483 /PROD = KIAA0483 protein |
| | /DB_XREF = gi: 3413925 /UG = Hs.64691 KIAA0483 protein /FL = gb: NM_015176.1 |
| 218543_s_at | gb: NM_022750.1 /DEF = Homo sapiens hypothetical protein FLJ22693 (FLJ22693), mRNA. /FEA = mRNA /GEN = FLJ22693 /PROD = hypothetical protein |
| | FLJ22693 /DB_XREF = gi: 12232412 /UG = Hs.12646 hypothetical protein FLJ22693 /FL = gb: AL136766.1 gb: NM_022750.1 |
| 203146_s_at | gb: NM_001470.1 /DEF = Homo sapiens gamma-aminobutyric acid (GABA) B receptor, 1 (GABBR1), transcript variant 1, mRNA. /FEA = mRNA /GEN = GABBR1 |
| | /PROD = gamma-aminobutyric acid (GABA) B receptor 1, isoform a precursor /DB_XREF = gi: 10835014 /UG = Hs.167017 gamma-aminobutyric acid (GABA) B |
| | receptor, 1 /FL = gb: NM_001470.1 gb: AF301005.1 gb: AF099148.1 |
| 218140_x_at | gb: NM_021203.1 /DEF = Homo sapiens APMCF1 protein (APMCF1), mRNA. /FEA = mRNA /GEN = APMCF1 /PROD = APMCF1 protein /DB_XREF = gi: 10864014 |
| | /UG = Hs.12152 APMCF1 protein /FL = gb: NM_021203.1 gb: AF141882.1 |
| 40420_at | Cluster Incl. AB015718: Homo sapiens lok mRNA for protein kinase, complete cds /cds = (50,2956) /gb = AB015718 /gi = 4001687 /ug = Hs.16134 |
| | /len = 4221 |
| 209971_x_at | Consensus includes gb: AI928526 /FEA = EST /DB_XREF = gi: 5664490 /DB_XREF = est: wp59a06.x1 /CLONE = IMAGE: 2466034 /UG = Hs.258730 heme-regulated initiation |
| | factor 2-alpha kinase /FL = gb: AF116615.1 |
| 48580_at | Cluster Incl. U55777: HSU55777 Homo sapiens cDNA /clone = 37698 /gb = U55777 /gi = 1354513 /ug = Hs.180933 /len = 1593 |
| 202416_at | gb: NM_003315.1 /DEF = Homo sapiens tetratricopeptide repeat domain 2 (TTC2), mRNA. /FEA = mRNA /GEN = TTC2 /PROD = tetratricopeptide repeat domain |
| | 2 /DB_XREF = gi: 4507712 /UG = Hs.5542 DnaJ (Hsp40) homolog, subfamily C, member 7 /FL = gb: U46571.1 gb: NM_003315.1 |
| 211727_s_at | gb: BC005895.1 /DEF = Homo sapiens, COX11 (yeast) homolog, cytochrome C oxidase assembly protein, clone MGC: 14469, mRNA, complete cds. |
| | /FEA = mRNA /PROD = COX11 (yeast) homolog, cytochrome c oxidaseassembly protein /DB_XREF = gi: 13543474 /FL = gb: BC005895.1 |
| 202125_s_at | gb: NM_015049.1 /DEF = Homo sapiens amyotrophic lateral sclerosis 2 (juvenile) chromosome region, candidate 3 (ALS2CR3), mRNA. /FEA = mRNA |
| | /GEN = ALS2CR3 /PROD = amyotrophic lateral sclerosis 2 (juvenile)chromosome region, candidate 3 /DB_XREF = gi: 13027379 /UG = Hs.154248 amyotrophic |
| | lateral sclerosis 2 (juvenile) chromosome region, candidate 3 /FL = gb: AB038951.1 gb: NM_015049.1 |
| 209451_at | gb: U59863.1 /DEF = Human TRAF-interacting protein I-TRAF mRNA, complete cds. /FEA = mRNA /PROD = I-TRAF /DB_XREF = gi: 1518017 /UG = Hs.146847 TRAF |
| | family member-associated NFKB activator /FL = gb: U59863.1 |
| 209440_at | gb: BC001605.1 /DEF = Homo sapiens, phosphoribosyl pyrophosphate synthetase 1, clone MGC: 2256, mRNA, complete cds. /FEA = mRNA |
| | /PROD = phosphoribosyl pyrophosphate synthetase 1 /DB_XREF = gi: 12804406 /UG = Hs.56 phosphoribosyl pyrophosphate synthetase 1 /FL = gb: BC001605.1 |
| | gb: D00860.1 |
| 218189_s_at | gb: NM_018946.2 /DEF = Homo sapiens N-acetylneuraminic acid phosphate synthase; sialic acid synthase (SAS), mRNA. /FEA = mRNA /GEN = SAS |
| | /PROD = N-acetylneuraminic acid phosphate synthase /DB_XREF = gi: 12056472 /UG = Hs.274424 N-acetylneuraminic acid phosphate synthase; sialic acid |
| | synthase /FL = gb: NM_018946.2 gb: BC000008.1 gb: AF257466.1 |
| 200947_s_at | gb: NM_005271.1 /DEF = Homo sapiens glutamate dehydrogenase 1 (GLUD1), mRNA. /FEA = mRNA /GEN = GLUD1 /PROD = glutamate dehydrogenase 1 |
| | /DB_XREF = gi: 4885280 /UG = Hs.77508 glutamate dehydrogenase 1 /FL = gb: J03248.1 gb: M37154.1 gb: M20867.1 gb: NM_005271.1 |
| 202798_at | gb: NM_006323.1 /DEF = Homo sapiens SEC24 (S. cerevisiae) related gene family, member B (SEC24B), mRNA. /FEA = mRNA /GEN = SEC24B /PROD = SEC24 (S. cerevisiae) |
| | related gene family, member B /DB_XREF = gi: 5454045 /UG = Hs.7239 SEC24 (S. cerevisiae) related gene family, member B |
| | /FL = gb: NM_006323.1 |
| 205323_s_at | gb: NM_005955.1 /DEF = Homo sapiens metal-regulatory transcription factor 1 (MTF1), mRNA. /FEA = mRNA /GEN = MTF1 /PROD = metal-regulatory transcription |
| | factor 1 /DB_XREF = gi: 5174588 /UG = Hs.211581 metal-regulatory transcription factor 1 /FL = gb: NM_005955.1 |
| 212622_at | Consensus includes gb: N64760 /FEA = EST /DB_XREF = gi: 1212589 /DB_XREF = est: yz30c06.s1 /CLONE = IMAGE: 284554 /UG = Hs.174905 KIAA0033 protein |
| 212033_at | Consensus includes gb: BF055107 /FEA = EST /DB_XREF = gi: 10809003 /DB_XREF = est: 7j75a05.x1 /CLONE = IMAGE: 3392240 /UG = Hs.180789 S164 protein |
| 218919_at | gb: NM_024699.1 /DEF = Homo sapiens hypothetical protein FLJ14007 (FLJ14007), mRNA. /FEA = mRNA /GEN = FLJ14007 /PROD = hypothetical protein |
| | FLJ14007 /DB_XREF = gi: 13375984 /UG = Hs.99519 hypothetical protein FLJ14007 /FL = gb: NM_024699.1 |
| 209271_at | gb: AB032251.1 /DEF = Homo sapiens BPTF mRNA for bromodomain PHD finger transcription factor, complete cds. /FEA = mRNA /GEN = BPTF |
| | /PROD = bromodomain PHD finger transcription factor /DB_XREF = gi: 6683491 /UG = Hs.99872 fetal Alzheimer antigen /FL = gb: AB032251.1 |
| 213322_at | Consensus includes gb: AL031778 /DEF = Human DNA sequence from clone 34B21 on chromosome 6p12.1-21.1. Contains part of a gene for a novel |
| | protein with ZU5 |
| | domain similar to part of Tight Junction Protein ZO1 (TJP1) and UNC5 Homologs, the gene for a novel BZRP (peripheral benzodiazapine. . . |
| | /FEA = mRNA_2 /DB_XREF = gi: 4153958 /UG = Hs.183056 Human DNA sequence from clone 34B21 on chromosome 6p12.1-21.1. Contains part of a gene for a |
| | novel protein with ZU5 domain similar to part of Tight Junction Protein ZO1 (TJP1) and UNC5 Homologs, the gene for a novel BZRP (peripheral |
| | benzodiazapine receptor |
| 221895_at | Consensus includes gb: AW469184 /FEA = EST /DB_XREF = gi: 7039290 /DB_XREF = est: hc78g04.x1 /CLONE = IMAGE: 2898870 /UG = Hs.65406 ESTs |
| 217760_at | Consensus includes gb: AA176780 /FEA = EST /DB_XREF = gi: 1757929 /DB_XREF = est: zp32a10.s1 /CLONE = IMAGE: 611130 /UG = Hs.14512 DIPB protein |
| | /FL = gb: NM_017583.1 |
| 201448_at | Consensus includes gb: AL046419 /FEA = EST /DB_XREF = gi: 5434493 /DB_XREF = est: DKFZp434N247_s1 /CLONE = DKFZp434N247 /UG = Hs.239489 TIA1 cytotoxic |
| | granule-associated RNA-binding protein /FL = gb: NM_022037.1 gb: M77142.1 |
| 218465_at | gb: NM_018126.1 /DEF = Homo sapiens hypothetical protein FLJ10525 (FLJ10525), mRNA. /FEA = mRNA /GEN = FLJ10525 /PROD = hypothetical protein |
| | FLJ10525 /DB_XREF = gi: 8922490 /UG = Hs.31082 hypothetical protein FLJ10525 /FL = gb: BC000948.1 gb: NM_018126.1 |
| 212880_at | Consensus includes gb: AB011113.1 /DEF = Homo sapiens mRNA for KIAA0541 protein, partial cds. /FEA = mRNA /GEN = KIAA0541 /PROD = KIAA0541 protein |
| | /DB_XREF = gi: 3043605 /UG = Hs.10881 WD repeat domain 7 |
| 218142_s_at | gb: NM_016302.1 /DEF = Homo sapiens protein x 0001 (LOC51185), mRNA. /FEA = mRNA /GEN = LOC51185 /PROD = protein x 0001 /DB_XREF = gi: 10047097 |
| | /UG = Hs.18925 protein x 0001 /FL = gb: NM_016302.1 gb: AF117230.1 |
| 202078_at | gb: NM_003653.1 /DEF = Homo sapiens COP9 (constitutive photomorphogenic, Arabidopsis, homolog) subunit 3 (COPS3), mRNA. /FEA = mRNA /GEN = COPS3 |
| | /PROD = COP9 (constitutive photomorphogenic, Arabidopsis, homolog) subunit 3 /DB_XREF = gi: 4502974 /UG = Hs.6076 COP9 (constitutive photomorphogenic, |
| | genic, Arabidopsis, homolog) subunit 3 /FL = gb: BC001891.1 gb: AF031647.1 gb: NM_003653.1 gb: AF098109.1 |
| 219069_at | gb: NM_017704.1 /DEF = Homo sapiens hypothetical protein FLJ20189 (FLJ20189), mRNA. /FEA = mRNA /GEN = FLJ20189 /PROD = hypothetical protein |
| | FLJ20189 /DB_XREF = gi: 8923180 /UG = Hs.29052 hypothetical protein FLJ20189 /FL = gb: NM_017704.1 |
| 218333_at | gb: NM_016041.1 /DEF = Homo sapiens CGI-101 protein (LOC51009), mRNA. /FEA = mRNA /GEN = LOC51009 /PROD = CGI-101 protein /DB_XREF = gi: 7705603 |
| | /UG = Hs.286131 CGI-101 protein /FL = gb: AF132289.1 gb: AF151859.1 gb: NM_016041.1 gb: AF242523.1 |
| 213082_s_at | Consensus includes gb: AJ005866.1 /DEF = Homo sapiens mRNA for putative Sqv-7-like protein, partial. /FEA = mRNA /PROD = Sqv-7-like protein |
| | /DB_XREF = gi: 4008516 /UG = Hs.90078 nucleotide-sugar transporter similar to C. elegens sqv-7 |
| 221452_s_at | gb: NM_030969.1 /DEF = Homo sapiens hypothetical protein MGC1223 (MGC1223), mRNA. /FEA = CDS /GEN = MGC1223 /PROD = hypothetical protein MGC1223 |
| | /DB_XREF = gi: 13624338 /FL = gb: NM_030969.1 |
| 213733_at | Consensus includes gb: BF740152 /FEA = EST /DB_XREF = gi: 12066828 /DB_XREF = est: 7n13g08.x1 /CLONE = IMAGE: 3564495 /UG = Hs.121555 myosin IE |
| 201225_s_at | gb: NM_005839.1 /DEF = Homo sapiens SerArg-related nuclear matrix protein (plenty of prolines 101-like) (SRM160), mRNA. /FEA = mRNA /GEN = SRM160 |
| | /PROD = SerArg-related nuclear matrix protein (plentyof prolines 101-like) /DB_XREF = gi: 5032118 /UG = Hs.18192 SerArg-related nuclear matrix |
| | protein (plenty of prolines 101-like) /FL = gb: AF048977.1 gb: NM_005839.1 |
| 219392_x_at | gb: NM_018304.1 /DEF = Homo sapiens hypothetical protein FLJ11029 (FLJ11029), mRNA. /FEA = mRNA /GEN = FLJ11029 /PROD = hypothetical protein |
| | FLJ11029 /DB_XREF = gi: 8922831 /UG = Hs.274448 hypothetical protein FLJ11029 /FL = gb: NM_018304.1 |
| 219212_at | gb: NM_016299.1 /DEF = Homo sapiens heat shock protein hsp70-related protein (LOC51182), mRNA. /FEA = mRNA /GEN = LOC51182 /PROD = heat shock protein |
| | hsp70-related protein /DB_XREF = gi: 10047093 /UG = Hs.44581 heat shock protein hsp70-related protein /FL = gb: NM_016299.1 gb: AF112210.1 |
| | gb: AF143723.1 |
| 217907_at | gb: NM_014161.1 /DEF = Homo sapiens HSPC071 protein (HSPC071), mRNA. /FEA = mRNA /GEN = HSPC071 /PROD = HSPC071 protein /DB_XREF = gi: 7661777 |
| | /UG = Hs.23038 HSPC071 protein /FL = gb: AL136633.1 gb: BC001623.1 gb: AF161556.1 gb: NM_014161.1 |
| 200727_s_at | Consensus includes gb: AA699583 /FEA = EST /DB_XREF = gi: 2703730 /DB_XREF = est: zi42g07.s1 /CLONE = IMAGE: 433500 /UG = Hs.42915 ARP2 |
| | (actin-related protein 2, yeast) homolog /FL = gb: AF006082.1 gb: NM_005722.1 |
| 209068_at | gb: D89678.1 /DEF = Homo sapiens mRNA for A + U-rich element RNA binding factor, complete cds. /FEA = mRNA /PROD = A + U-rich element RNA binding |
| | factor /DB_XREF = gi: 3218539 /UG = Hs.170311 heterogeneous nuclear ribonucleoprotein D-like /FL = gb: D89092.1 gb: D89678.1 |
| 46256_at | Cluster Incl. AA522670: ni39a05.s1 Homo sapiens cDNA, 3 end /clone = IMAGE-979184 /clone_end = 3 /gb = AA522670 /gi = 2263382 /ug = Hs.7247 /len = 684 |
| 218068_s_at | gb: NM_024836.1 /DEF = Homo sapiens hypothetical protein FLJ22301 (FLJ22301), mRNA. /FEA = mRNA /GEN = FLJ22301 /PROD = hypothetical protein |
| | FLJ22301 /DB_XREF = gi: 13376246 /UG = Hs.181406 hypothetical protein FLJ22301 /FL = gb: NM_024836.1 |
| 208843_s_at | gb: BC001408.1 /DEF = Homo sapiens, clone MGC: 1233, mRNA, complete cds. /FEA = mRNA /PROD = Unknown (protein for MGC: 1233) /DB_XREF = gi: 12655112 |
| | /UG = Hs.6880 DKFZP434D156 protein /FL = gb: BC001408.1 |
| 202869_at | gb: NM_016816.1 /DEF = Homo sapiens 2,5-oligoadenylate synthetase 1 (40-46 kD) (OAS1), transcript variant E18, mRNA. /FEA = mRNA /GEN = OAS1 |
| | /PROD = 2,5-oligoadenylate synthetase 1, isoform E18 /DB_XREF = gi: 8051620 /UG = Hs.82396 2,5-oligoadenylate synthetase 1 (40-46 kD) |
| | /FL = gb: NM_016816.1 |
| 213546_at | Consensus includes gb: AL050378.1 /DEF = Homo sapiens mRNA; cDNA DKFZp586I1420 (from clone DKFZp586I1420); partial cds. /FEA = mRNA |
| | /GEN = DKFZp586I1420 /PROD = hypothetical protein /DB_XREF = gi: 4914581 /UG = Hs.112423 Homo sapiens mRNA; cDNA DKFZp586I1420 (from clone |
| | DKFZp586I1420); partial cds |
| 214789_x_at | Consensus includes gb: AA524274 /FEA = EST /DB_XREF = gi: 2265202 /DB_XREF = est: ng34d08.s1 /CLONE = IMAGE: 936687 /UG = Hs.155160 Splicing factor, |
| | arginineserine-rich, 46 kD |
| 213649_at | Consensus includes gb: AA524053 /FEA = EST /DB_XREF = gi: 2264981 /DB_XREF = est: ng33b07.s1 /CLONE = IMAGE: 936565 /UG = Hs.184167 splicing factor, |
| | arginineserine-rich 7 (35 kD) |
| 212251_at | Consensus includes gb: AI972475 /FEA = EST /DB_XREF = gi: 5769391 /DB_XREF = est: wr40b09.x1 /CLONE = IMAGE: 2490137 /UG = Hs.243901 Homo sapiens cDNA |
| | FLJ20738 fis, clone HEP08257 |
| 209166_s_at | gb: U68567.1 /DEF = Human lysosomal acid alpha-mannosidase mRNA, complete cds. /FEA = mRNA /PROD = lysosomal acid alpha-mannosidase |
| | /DB_XREF = gi: 1658373 /UG = Hs.279854 mannosidase, alpha, class 2B, member 1 /FL = gb: NM_000528.1 gb: BC000736.1 gb: U05572.1 gb: U68567.1 |
| | gb: U60266.1 |
| 200683_s_at | Consensus includes gb: BE964689 /FEA = EST /DB_XREF = gi: 11768267 /DB_XREF = est: 601658226R1 /CLONE = IMAGE: 3885630 /UG = Hs.108104 ubiquitin- |
| | conjugating enzyme E2L 3 /FL = gb: NM_003347.1 |
| 219913_s_at | gb: NM_016652.2 /DEF = Homo sapiens CGI-201 protein (LOC51340), mRNA. /FEA = mRNA /GEN = LOC51340 /PROD = CGI-201 protein /DB_XREF = gi: 11072090 |
| | /UG = Hs.268281 crooked neck protein (crn) /FL = gb: AF255443.2 gb: NM_016652.2 gb: AF318302.1 |
| 218104_at | gb: NM_017746.1 /DEF = Homo sapiens hypothetical protein FLJ20287 (FLJ20287), mRNA. /FEA = mRNA /GEN = FLJ20287 /PROD = hypothetical protein |
| | FLJ20287 /DB_XREF = gi: 8923268 /UG = Hs.26369 hypothetical protein FLJ20287 /FL = gb: NM_017746.1 |
| 212345_s_at | Consensus includes gb: BE675139 /FEA = EST /DB_XREF = gi: 10035680 /DB_XREF = est: 7f03b12.x1 /CLONE = IMAGE: 3293567 /UG = Hs.13659 hypothetical protein |
| | DKFZp586F2423 |
| 213982_s_at | Consensus includes gb: BG107203 /FEA = EST /DB_XREF = gi: 12601049 /DB_XREF = est: 602290933F1 /CLONE = IMAGE: 4385577 /UG = Hs.242271 KIAA0471 gene |
| | product |
| 202519_at | gb: NM_014938.1 /DEF = Homo sapiens KIAA0867 protein (MONDOA), mRNA. /FEA = mRNA /GEN = MONDOA /PROD = MondoA protein /DB_XREF = gi: 7662347 |
| | /UG = Hs.52081 KIAA0867 protein /FL = gb: AB020674.1 gb: NM_014938.1 |
| 202892_at | gb: NM_004661.1 /DEF = Homo sapiens CDC23 (cell division cycle 23, yeast, homolog) (CDC23), mRNA. /FEA = mRNA /GEN = CDC23 /PROD = cell division |
| | cycle 23, yeast homolog; CDC23 /DB_XREF = gi: 4757947 /UG = Hs.153546 CDC23 (cell division cycle 23, yeast, homolog) /FL = gb: AF053977.1 |
| | gb: AB011472.1 gb: NM_004661.1 gb: AF191341.1 |
| 213876_x_at | Consensus includes gb: AW089584 /FEA = EST /DB_XREF = gi: 6046928 /DB_XREF = est: xd20f04.x1 /CLONE = IMAGE: 2594335 /UG = Hs.171909 U2 small nuclear |
| | ribonucleoprotein auxiliary factor, small subunit 2 |
| 202054_s_at | gb: NM_000382.1 /DEF = Homo sapiens aldehyde dehydrogenase 3 family, member A2 (ALDH3A2), mRNA. /FEA = mRNA /GEN = ALDH3A2 /PROD = aldehyde dehydrogenase |
| | 3A2 /DB_XREF = gi: 4557302 /UG = Hs.159608 aldehyde dehydrogenase 3 family, member A2 /FL = gb: L47162.1 gb: U46689.1 gb: NM_000382.1 |
| 217776_at | gb: AF167438.1 /DEF = Homo sapiens androgen-regulated short-chain dehydrogenasereductase 1 (ARSDR1) mRNA, complete cds. /FEA = mRNA /GEN = ARSDR1 |
| | /PROD = androgen-regulated short-chaindehydrogenasereductase 1 /DB_XREF = gi: 9622123 /UG = Hs.179817 CGI-82 protein /FL = gb: BC000112.1 |
| | gb: AF151840.1 gb: NM_016026.1 gb: AF167438.1 |
| 208758_at | gb: D89976.1 /DEF = Homo sapiens mRNA for 5-aminoimidazole-4-carboxamide ribonucleotide transformylase, complete cds. /FEA = mRNA |
| | /PROD = 5-aminoimidazole-4-carboxamide ribonucleotidetransformylase /DB_XREF = gi: 2317691 /UG = Hs.90280 5-aminoimidazole-4-carboxamide |
| | ribonucleotide formyltransferaseIMP cyclohydrolase /FL = gb: U37436.1 gb: D82348.1 gb: D89976.1 gb: NM_004044.1 |
| 212749_s_at | Consensus includes gb: AI096477 /FEA = EST /DB_XREF = gi: 3445974 /DB_XREF = est: qa03c06.x1 /CLONE = IMAGE: 1685674 /UG = Hs.48297 DKFZP586C1620 protein |
| 200890_s_at | Consensus includes gb: AW006345 /FEA = EST /DB_XREF = gi: 5855123 /DB_XREF = est: wt04d05.x1 /CLONE = IMAGE: 2506473 /UG = Hs.250773 signal sequence |
| | receptor, |
| | alpha (translocon-associated protein alpha) /FL = gb: AF156965.1 gb: NM_003144.2 |
| 211675_s_at | gb: AF054589.1 /DEF = Homo sapiens HIC protein isoform p40 and HIC protein isoform p32 mRNAs, complete cds. /FEA = mRNA /PROD = HIC protein isoform |
| | p32; HIC protein isoform p40 /DB_XREF = gi: 3426297 /FL = gb: AF054589.1 |
| 206792_x_at | gb: NM_000923.1 /DEF = Homo sapiens phosphodiesterase 4C, cAMP-specific (dunce (Drosophila)-homolog phosphodiesterase E1) (PDE4C), mRNA. |
| | /FEA = mRNA /GEN = PDE4C /PROD = phosphodiesterase 4C, cAMP-specific (dunce(Drosophila)-homolog phosphodiesterase E1) /DB_XREF = gi: 4505664 |
| | /UG = Hs.189 phosphodiesterase 4C, |
| | cAMP-specific (dunce (Drosophila)-homolog phosphodiesterase E1) /FL = gb: NM_000923.1 |
| 218718_at | gb: NM_016205.1 /DEF = Homo sapiens platelet derived growth factor C (PDGFC), mRNA. /FEA = mRNA /GEN = PDGFC /PROD = secretory growth factor-like |
| | protein fallotein /DB_XREF = gi: 9994186 /UG = Hs.43080 platelet derived growth factor C /FL = gb: AF091434.1 gb: AF244813.1 gb: AB033831.1 |
| | gb: NM_016205.1 |
| 201742_x_at | gb: NM_006924.1 /DEF = Homo sapiens splicing factor, arginineserine-rich 1 (splicing factor 2, alternate splicing factor) (SFRS1), mRNA. |
| | /FEA = mRNA /GEN = SFRS1 /PROD = splicing factor, arginineserine-rich 1(splicing factor 2, alternate splicing factor) /DB_XREF = gi: 5902075 |
| | /UG = Hs.73737 splicing factor, arginineserine-rich 1 (splicing, factor 2, alternate splicing factor) /FL = gb: M69040.1 gb: NM_006924.1 |
| 218622_at | gb: NM_024057.1 /DEF = Homo sapiens hypothetical protein MGC5585 (MGC5585), mRNA. /FEA = mRNA /GEN = MGC5585 /PROD = hypothetical protein MGC5585 |
| | /DB_XREF = gi: 13129027 /UG = Hs.5152 hypothetical protein MGC5585 /FL = gb: BC000861.1 gb: NM_024057.1 |
| 212176_at | Consensus includes gb: AA902326 /FEA = EST /DB_XREF = gi: 3037233 /DB_XREF = est: ol:92b01.s1 /CLONE = IMAGE: 1521385 /UG = Hs.18368 DKFZP564B0769 protein |
| 218970_s_at | gb: NM_015960.1 /DEF = Homo sapiens CGI-32 protein (LOC51076), mRNA. /FEA = mRNA /GEN = LOC51076 /PROD = CGI-32 protein /DB_XREF = gi: 7705727 |
| | /UG = Hs.16606 CGI-32 protein /FL = gb: AF132966.1 gb: NM_015960.1 |
| 201696_at | gb: NM_005626.1 /DEF = Homo sapiens splicing factor, arginineserine-rich 4 (SFRS4), mRNA. /FEA = mRNA /GEN = SFRS4 /PROD = splicing factor, arginineserine- |
| | rich 4 /DB_XREF = gi: 5032088 /UG = Hs.76122 splicing factor, arginineserine-rich 4 /FL = gb: BC002781.1 gb: L14076.1 gb: NM_005626.1 |
| 222103_at | Consensus includes gb: AI434345 /FEA = EST /DB_XREF = gi: 4295529 /DB_XREF = est: ti48b06.x1 /CLONE = IMAGE: 213368 /UG = Hs.36908 activating transcription |
| | factor 1 /FL = gb: NM_005171.1 |
| 204036_at | Consensus includes gb: AW269335 /FEA = EST /DB_XREF = gi: 6656365 /DB_XREF = est: xs47d05.x1 /CLONE = IMAGE: 2772777 /UG = Hs.75794 endothelial differentiation, |
| | lysophosphatidic acid G-protein-coupled receptor, 2 /FL = gb: U78192.1 gb: U80811.1 gb: NM_001401.1 |
| 218283_at | gb: NM_016305.1 /DEF = Homo sapiens kiaa-iso protein (LOC51188), mRNA. /FEA = mRNA /GEN = LOC51188 /PROD = kiaa-iso protein /DB_XREF = gi: 10047103 |
| | /UG = Hs.9774 kiaa-iso protein /FL = gb: NM_016305.1 gb: AF201950.1 |
| 209654_at | gb: BC004902.1 /DEF = Homo sapiens, clone MGC: 4271, mRNA, complete cds. /FEA = mRNA /PROD = Unknown (protein for MGC: 4271) /DB_XREF = gi: 13436178 |
| | /UG = Hs.5070 KIAA0947 protein /FL = gb: BC004902.1 |
| 209083_at | gb: U34690.1 /DEF = Human coronin-like protein (HCORO1) mRNA, complete cds. /FEA = mRNA /GEN = HCORO1 /PROD = coronin-like protein |
| | /DB_XREF = gi: 1002922 /UG = Hs.109606 coronin, actin-binding protein, 1A /FL = gb: U34690.1 gb: NM_007074.1 gb: D44497.1 |
| 221873_at | Consensus includes gb: AW162015 /FEA = EST /DB_XREF = gi: 6301048 /DB_XREF = est: au72d09.x1 /CLONE = IMAGE: 2781809 /UG = Hs.154095 zinc finger protein |
| | 143 (clone pHZ-1) |
| 213573_at | Consensus includes gb: AA861608 /FEA = EST /DB_XREF = gi: 2953748 /DB_XREF = est: ak34e01.s1 /CLONE = IMAGE: 1407864 /UG = Hs.180446 karyopherin (importin) |
| | beta 1 |
| 209580_s_at | gb: AF114784.1 /DEF = Homo sapiens methyl-CpG binding endonuclease (MED1) mRNA, complete cds. /FEA = mRNA /GEN = MED1 /PROD = methyl-CpG binding |
| | endonuclease /DB_XREF = gi: 4539758 /UG = Hs.35947 methyl-CpG binding domain protein 4 /FL = gb: AF072250.1 gb: NM_003925.1 gb: AF114784.1 |
| 219097_x_at | gb: NM_024104.1 /DEF = Homo sapiens hypothetical protein MGC2747 (MGC2747), mRNA. /FEA = mRNA /GEN = MGC2747 /PROD = hypothetical protein MGC2747 |
| | /DB_XREF = gi: 13129111 /UG = Hs.227203 hypothetical protein MGC2747 /FL = gb: BC001680.1 gb: BC001948.1 gb: NM_024104.1 |
| 206513_at | gb: NM_004833.1 /DEF = Homo sapiens absent in melanoma 2 (AIM2), mRNA. /FEA = mRNA /GEN = AIM2 /PROD = absent in melanoma 2 /DB_XREF = gi: 4757733 |
| | /UG = Hs.105115 absent in melanoma 2 /FL = gb: AF024714.1 gb: NM_004833.1 |
| 209268_at | gb: AF165513.1 /DEF = Homo sapiens vacuolar protein sorting 45 isoform (VPS45) mRNA, complete cds. /FEA = mRNA /GEN = VPS45 /PROD = vacuolar protein |
| | sorting 45 isoform /DB_XREF = gi: 9294732 /UG = Hs.6650 vacuolar protein sorting 45B (yeast homolog) /FL = gb: NM_007259.1 gb: AF165513.1 |
| 219329_s_at | gb: NM_016985.1 /DEF = Homo sapiens apoptosis related protein APR-3 (APR-3), mRNA. /FEA = mRNA /GEN = APR-3 /PROD = apoptosis related protein APR-3 |
| | /DB_XREF = gi: 7706360 /UG = Hs.9527 apoptosis related protein APR-3 /FL = gb: AF144055.2 gb: NM_016085.1 |
| 202051_s_at | gb: NM_005095.1 /DEF = Homo sapiens zinc finger protein 262 (ZNF262), mRNA. /FEA = mRNA /GEN = ZNF262 /PROD = zinc finger protein 262 |
| | /DB_XREF = gi: 4827068 |
| | /UG = Hs.150390 zinc finger protein 262 /FL = gb: AB007885.1 gb: NM_005095.1 |
| 200749_at | Consensus includes gb: BF112006 /FEA = EST /DB-XREF = gi: 10941619 /DB_XREF = est: 7137e05.x1 /CLONE = IMAGE: 3523665 /UG = Hs.10842 RAN, member RAS |
| | oncogene family |
| | /FL = gb: BC000852.1 gb: BC004272.1 gb: M31469.1 gb: AF052578.1 gb: AF054183.1 gb: NM_006325.2 |
| 213373_s_at | Consensus includes gb: BF439983 /FEA = EST /DB_XREF = gi: 11452500 /DB_XREF = est: nac51f11.x1 /CLONE = IMAGE: 3406220 /UG = Hs.19949 caspase 8, apoptosis- |
| | related |
| | cysteine protease /FL = gb: U58143.1 |
| 212673_at | Consensus includes gb: D42084.1 /DEF = Human mRNA for KIAA0094 gene, partial cds. /FEA = mRNA /GEN = KIAA0094 /DB_XREF = gi: 577324 /UG = Hs.82007 |
| | KIAA0094 protein |
| 201771_at | gb: NM_005698.1 /DEF = Homo sapiens secretory carrier membrane protein 3 (SCAMP3), mRNA. /FEA = mRNA /GEN = SCAMP3 /PROD = secretory carrier membrane |
| | protein 3 /DB_XREF = gi: 5032076 /UG = Hs.200600 secretory carrier membrane protein 3 /FL = gb: BC000161.2 gb: BC005135.1 gb: AF005039.1 |
| | gb: NM_005698.1 |
| 209607_x_at | gb: U08032.1 /DEF = Human thermolabile (monoamine, M form) phenol sulfotransferase (STM) mRNA, complete cds. /FEA = mRNA /GEN = STM |
| | /PROD = thermolabile (monoamine, M form) phenolsulfotransferase /DB_XREF = gi: 468256 /UG = Hs.274614 sulfotransferase family, cytosolic, 1A, |
| | phenol-preferring, member 3 /FL = gb: NM_003166.1 gb: L19956.1 gb: U34199.1 gb: U08032.1 |
| 217317_s_at | Consensus includes gb: AB002391.2 /DEF = Homo sapiens mRNA for KIAA0393 protein, partial cds. /FEA = mRNA /GEN = KIAA0393 /PROD = KIAA0393 protein |
| | /DB_XREF = gi: 6683696 /UG = Hs.266933 hect domain and RLD 2 |
| 202272_s_at | gb: NM_015176.1 /DEF = Homo sapiens KIAA0483 protein (KIAA0483), mRNA. /FEA = mRNA /GEN = KIAA0483 /PROD = KIAA0483 protein /DB_XREF = gi: 7662157 |
| | /UG = Hs.64691 KIAA0483 protein /FL = gb: NM_015176.1 |
| 210629_x_at | gb: AF000425.1 /DEF = Homo sapiens LST1 mRNA, cLST1A splice variant, complete cds. /FEA = mRNA /GEN = LST1 /DB_XREF = gi: 2145065 /UG = Hs.88411 |
| | lymphocyte antigen 117 /FL = gb: AF000425.1 |
| 205329_s_at | gb: NM_003794.1 /DEF = Homo sapiens sorting nexin 4 (SNX4), mRNA. /FEA = mRNA /GEN = SNX4 /PROD = sorting nexin 4 /DB_XREF = gi: 4507144 /UG = Hs.267812 |
| | sorting nexin 4 /FL = gb: AF130078.1 gb: AF065485.1 gb: NM_003794.1 |
| 218194_at | gb: NM_015523.1 /DEF = Homo sapiens small fragment nuclease (DKFZP566E144), mRNA. /FEA = mRNA /GEN = DKFZP566E144 /PROD = small fragment nuclease |
| | /DB_XREF = gi: 7661645 /UG = Hs.7527 small fragment nuclease /FL = gb: AF151872.1 gb: AL110239.1 gb: NM_015523.1 |
| 220560_at | gb: NM_014144.1 /DEF = Homo sapiens SMS3 protein (SMS3), mRNA. /FEA = mRNA /GEN = SMS3 /PROD = SMS3 protein /DB_XREF = gi: 7662662 /UG = Hs.272100 SMS3 |
| | protein /FL = gb: AB029488.1 gb: NM_014144.1 |
| 208270_s_at | gb: NM_020216.2 /DEF = Homo sapiens arginyl aminopeptidase (aminopeptidase B) (RNPEP), mRNA. /FEA = mRNA /GEN = RNPEP /PROD = arginyl aminopeptidase |
| | (aminopeptidase B) /DB_XREF = gi: 13443030 /UG = Hs.283667 arginyl aminopeptidase (aminopeptidase B) /FL = gb: NM_020216.2 |
| 220175_s_at | gb: NM_020667.1 /DEF = Homo sapiens hypothetical protein from clone 1659351 (LOC57397), mRNA. /FEA = mRNA /GEN = LOC57397 /PROD = hypothetical |
| | protein from clone 1659351 /DB_XREF = gi: 10190707 /UG = Hs.288838 hypothetical protein from clone 1659351 /FL = gb: NM_020667.1 |
| 212429_s_at | Consensus includes gb: AW194657 /FEA = EST /DB_XREF = gi: 6473537 /DB_XREF = est: xb28b12.x1 /CLONE = IMAGE: 2577599 /UG = Hs.75782 general transcription |
| | factor IIIC, polypeptide 2 (beta subunit, 110 kD) |
| 209058_at | gb: AB002282.1 /DEF = Homo sapiens mRNA for hMBFlalpha, complete cds. /FEA = mRNA /PROD = hMBFlalpha /DB_XREF = gi: 6526354 /UG = Hs.174050 endothelial |
| | differentiation-related factor 1 /FL = gb: NM_003792.1 gb: AB002282.1 |
| 212535_at | Consensus includes gb: AA142929 /FEA = EST /DB_XREF = gi: 1712307 /DB_XREF = est: z140g07.s1 /CLONE = IMAGE: 504444 /UG = Hs.288993 ESTs |
| 201524_x_at | gb: NM_003348.1 /DEF = Homo sapiens ubiquitin-conjugating enzyme E2N (homologous to yeast UBC13) (UBE2N), mRNA. /FEA = mRN /GEN = UBE2N |
| | /PROD = ubiquitin-conjugating enzyme E2N (homologous toyeast UBC13) /DB_XREF = gi: 4507792 /UG = Hs.75355 ubiquitin-conjugating enzyme E2N (homologous |
| | to yeast UBC13) /FL = gb: D83004.1 gb: BC000396.1 gb: BC003365.1 gb: NM_003348.1 |
| 201716_at | gb: NM_003099.1 /DEF = Homo sapiens sorting nexin 1 (SNX1), mRNA. /FEA = mRNA /GEN = SNX1 /PROD = sorting nexin 1 /DB_XREF = gi: 4507138 /UG = Hs.75283 |
| | sorting nexin 1 /FL = gb: BC000357.1 gb: U53225.1 gb: AF065483.1 gb: NM_003099.1 |
| 213097_s_at | Consensus includes gb: AI338837 /FEA = EST /DB_XREF = gi: 4075764 /DB_XREF = est: qq28f09.x1 /CLONE = IMAGE: 1933865 /UG = Hs.82254 zuotin related factor 1 |
| 213532_at | Consensus includes gb: AI797833 /FEA = EST /DB_XREF = gi: 5363390 /DB_XREF = est: wh79e10.x1 /CLONE = IMAGE: 2386986 /UG = Hs.52438 ESTs, Weakly similar |
| | to ORF YOR126c S. cerevisiae |
| 210766_s_at | gb: AF053640.1 /DEF = Homo sapiens trachea cellular apoptosis susceptibility protein (CSE1) mRNA, complete cds. /FEA = mRNA /GEN = CSE1 |
| | /PROD = cellular apoptosis susceptibility protein /DB_XREF = gi: 3560554 /UG = Hs.90073 chromosome segregation 1 (yeast homolog)-like |
| | /FL = gb: AF053640.1 |
| 209523_at | Consensus includes gb: AK001618.1 /DEF = Homo sapiens cDNA FLJ10756 fis, clone NT2RP3004572, highly similar to Homo sapiens cofactor of initiator |
| | function mRNA. /FEA = mRNA /DB_XREF = gi: 7022983 /UG = Hs.122752 TATA box binding protein (TBP)-associated factor, RNA polymerase II, B, |
| | 150 kD /FL = gb: AF026445.1 gb: AF040701.1 gb: AF057694.1 gb: NM_003184.1 |
| 209276_s_at | gb: AF162769.1 /DEF = Homo sapiens thioltransferase mRNA, complete cds. /FEA = mRNA /PROD = thioltransferase /DB_XREF = gi: 5442445 /UG = Hs.28988 |
| | glutaredoxin (thioltransferase) /FL = gb: BC005304.1 gb: AF162769.1 gb: D21238.1 |
| 200848_at | Consensus includes gb: AA479488 /FEA = EST /DB_XREF = gi: 2208044 /DB_XREF = est: zv21c09.s1 /CLONE = IMAGE: 754288 /UG = Hs.4113 S-adenosylhomocysteine |
| | hydrolase-like 1 /FL = gb: U82761.1 gb: NM_006621.1 |
| 212406_s_at | Consensus includes gb: AB028973.1 /DEF = Homo sapiens mRNA for KIAA1050 protein, partial cds. /FEA = mRNA /GEN = KIAA1050 /PROD = KIAA1050 protein |
| | /DB_XREF = gi: 5689436 /UG = Hs.184628 hypothetical protein FLJ10883 |
| 212072_s_at | Consensus includes gb: AL049761 /DEF = Human DNA sequence from clone RP5-863C7 on chromosome 20p12.3-13. Contains the CSNK2A1 gene for casein |
| | kinase 2 alpha 1 polypeptide (EC 2.7.1.37), ESTs, STSs and GSSs /FEA = mRNA_2 /DB_XREF = gi: 5738437 /UG = Hs.155140 casein kinase 2, alpha 1 |
| | polypeptide |
| 203159_at | gb: NM_014905.1 /DEF = Homo sapiens glutaminase (GLS), mRNA. /FEA = mRNA /GEN = GLS /PROD = glutaminase C /DB_XREF = gi: 7662327 /UG = Hs.239189 glutaminase |
| | /FL = gb: AF327434.1 gb: AB020645.1 gb: AF097493.1 gb: AF223943.1 gb: NM_014905.1 |
| 203521_s_at | gb: NM_014345.1 /DEF = Homo sapiens endocrine regulator (HRIHFB2436), mRNA. /FEA = mRNA /GEN = HRIHFB2436 /PROD = endocrine regulator |
| | /DB_XREF = gi: 7657183 /UG = Hs.48433 endocrine regulator /FL = gb: AF121141.1 gb: NM_014345.1 |
| 204439_at | gb: NM_006820.1 /DEF = Homo sapiens hypothetical protein, expressed in osteoblast (GS3686), mRNA. /FEA = mRNA /GEN = GS3686 /PROD = hypothetical |
| | protein, expressed in osteoblast /DB_XREF = gi: 5803026 /UG = Hs.75470 hypothetical protein, expressed in osteoblast /FL = gb: AB000115.1 |
| | gb: NM_006820.1 |
| 218646_at | gb: NM_017867.1 /DEF = Homo sapiens hypothetical protein FLJ20534 (FLJ20534), mRNA. /FEA = mRNA /GEN = FLJ20534 /PROD = hypothetical protein |
| | FLJ20534 /DB_XREF = gi: 8923502 /UG = Hs.44344 hypothetical protein FLJ20534 /FL = gb: AL136673.1 gb: NM_017867.1 |
| 213168_at | Consensus includes gb: AU145005 /FEA = EST /DB_XREF = gi: 11006526 /DB_XREF = est: AU145005 /CLONE = HEMBA1003603 /UG = Hs.44450 Sp3 transcription |
| | factor |
| 210371_s_at | gb: BC003092.1 /DEF = Homo sapiens, Similar to retinoblastoma binding protein 4, clone MGC: 1393, mRNA, complete cds. /FEA = mRNA /PROD = Similar |
| | to retinoblastoma binding protein 4 /DB_XREF = gi: 13111850 /UG = Hs.16003 retinoblastoma-binding protein 4 /FL = gb: BC003092.1 gb: NM_005610.1 |
| 200052_s_at | gb: NM_004515.1 /DEF = Homo sapiens interleukin enhancer binding factor 2, 45 kD (ILF2), mRNA. /FEA = mRNA /GEN = ILF2 /PROD = interleukin enhancer |
| | binding factor 2, 45 kD /DB_XREF = gi: 4758601 /UG = Hs.75117 interleukin enhancer binding factor 2, 45 kD /FL = gb: BC000382.1 gb: NM_004515.1 |
| | gb: U10323.1 |
| 201019_s_at | gb: NM_001412.1 /DEF = Homo sapiens eukaryotic translation initiation factor 1A (EIF1A), mRNA. /FEA = mRNA /GEN = EIF1A /PROD = eukaryotic translation |
| | initiation factor 1A /DB_XREF = gi: 4503498 /UG = Hs.4310 eukaryotic translation initiation factor 1A /FL = gb: BC000793.1 gb: L18960.1 |
| | gb: NM_001412.1 |
| 219892_at | gb: NM_023003.1 /DEF = Homo sapiens transmembrane 6 superfamily member 1 (TM6SF1), mRNA. /FEA = mRNA /GEN = TM6SF1 /PROD = transmembrane 6 superfamily |
| | member 1 /DB_XREF = gi: 13194198 /UG = Hs.133865 transmembrane 6 superfamily member 1 /FL = gb: AF255922.1 gb: NM_023003.1 |
| 218095_s_at | gb: NM_018475.1 /DEF = Homo sapiens uncharacterized hypothalamus protein HTMP (LOC55858), mRNA. /FEA = mRNA /GEN = LOC55858 /PROD = uncharacterized |
| | hypothalamus protein HTMP /DB_XREF = gi: 8923860 /UG = Hs.236510 uncharacterized hypothalamus protein HTMP /FL = gb: BC003545.1 gb: AF220188.1 |
| | gb: NM_018475.1 gb: AF183409.1 |
| 218042_at | gb: NM_016129.1 /DEF = Homo sapiens COP9 complex subunit 4 (LOC51138), mRNA. /FEA = mRNA /GEN = LOC51138 /PROD = COP9 complex subunit 4 |
| | /DB_XREF = gi: 7705844 /UG = Hs.6671 COP9 complex subunit 4 /FL = gb: BC004302.1 gb: AF100757.1 gb: NM_016129.1 |
| 202172_at | Consensus includes gb: BG035116 /FEA = EST /DB_XREF = gi: 12428927 /DB_XREF = est: 602324811F1 /CLONE = IMAGE: 4412907 /UG = Hs.6557 zinc finger protein |
| | 161 /FL = gb: D28118.1 gb: NM_007146.1 |
| 218108_at | gb: NM_018108.1 /DEF = Homo sapiens hypothetical protein FLJ10483 (FLJ10483), mRNA. /FEA = mRNA /GEN = FLJ10483 /PROD = hypothetical protein |
| | FLJ10483 /DB_XREF = gi: 8922451 /UG = Hs.6877 hypothetical protein FLJ10483 /FL = gb: NM_018108.1 |
| 212539_at | Consensus includes gb: AI422099 /FEA = EST /DB_XREF = gi: 4268030 /DB_XREF = est: tf57h09.x1 /CLONE = IMAGE: 2103425 /UG = Hs.14570 hypothetical protein |
| | FLJ22530 |
| 212635_at | Consensus includes gb: AW161626 /FEA = EST /DB_XREF = gi: 6300659 /DB_XREF = est: au68b11.x1 /CLONE = IMAGE: 2781405 /UG = Hs.21739 Homo sapiens mRNA; |
| | cDNA DKFZp586I1518 (from clone DKFZp586I1518) |
| 203356_at | Consensus includes gb: BE349584 /FEA = EST /DB_XREF = gi: 9261437 /DB_XREF = est: ht55h12.x1 /CLONE = IMAGE: 3150695 /UG = Hs.7145 calpain 7 |
| | /FL = gb: AB028639.1 gb: NM_014296.1 |
| 208174_x_at | gb: NM_005089.1 /DEF = Homo sapiens U2 small nuclear ribonucleoprotein auxiliary factor, small subunit 2 (U2AF1RS2), mRNA. /FEA = mRNA |
| | /GEN = U2AF1RS2 |
| | /PROD = U2 small nuclear ribonucleoprotein auxiliaryfactor, small subunit-related protein 2 /DB_XREF = gi: 4827045 /UG = Hs.171909 U2 small nuclear |
| | ribonucleoprotein auxiliary factor, small subunit 2 /FL = gb: D49677.1 gb: NM_005089.1 |
| 53912_at | Cluster Incl. AI668643: zb13f10.x5 Homo sapiens cDNA, 3 end /clone = IMAGE-301963 /clone_end = 3 /gb = AI668643 /gi = 4827951 /ug = Hs.15827 |
| | /len = 601 |
| 221736_at | Consensus includes gb: AA156777 /FEA = EST /DB_XREF = gi: 1728392 /DB_XREF = est: z118c08.s1 /CLONE = IMAGE: 502286 /UG = Hs.25431 KIAA1219 protein |
| 209475_at | gb: AF106069.1 /DEF = Homo sapiens deubiquitinating enzyme (UNPH4) mRNA, complete cds. /FEA = mRNA /GEN = UNPH4 /PROD = deubiquitinating enzyme |
| | /DB_XREF = gi: 5814096 /UG = Hs.23168 ubiquitin specific protease 15 /FL = gb: AF013990.1 gb: AF106069.1 |
| 204366_s_at | gb: NM_001521.1 /DEF = Homo sapiens general transcription factor IIIC, polypeptide 2 (beta subunit, 110 kD) (GTF3C2), mRNA. /FEA = mRNA |
| | /GEN = GTF3C2 /PROD = general transcription factor IIIC, polypeptide 2(beta subunit, 110 kD) /DB_XREF = gi: 4504204 /UG = Hs.75782 general transcription |
| | factor IIIC, polypeptide 2 (beta subunit, 110 kD) /FL = gb: D13636.1 gb: NM_001521.1 |
| 212584_at | Consensus includes gb: BG260519 /FEA = EST /DB_XREF = gi: 12770335 /DB_XREF = est: 602372065F1 /CLONE = IMAGE: 4480024 /UG = Hs.129952 KIAA0560 gene |
| | product |
| 212140_at | Consensus includes gb: AB014548.1 /DEF = Homo sapiens mRNA for KIAA0648 protein, partial cds. /FEA = mRNA /GEN = KIAA0648 /PROD = KIAA0648 protein |
| | /DB_XREF = gi: 3327109 /UG = Hs.31921 KIAA0648 protein |
| 219083_at | gb: NM_018130.1 /DEF = Homo sapiens hypothetical protein FLJ10539 (FLJ10539), mRNA. /FEA = mRNA /GEN = FLJ10539 /PROD = hypothetical protein |
| | FLJ10539 /DB_XREF = gi: 8922499 /UG = Hs.93391 hypothetical protein FLJ10539 /FL = gb: NM_018130.1 |
| 215772_x_at | Consensus includes gb: AL050226.1 /DEF = Homo sapiens mRNA; cDNA DKFZp586M2023 (from clone DKFZp586M2023); partial cds. /FEA = mRNA |
| | /GEN = DKFZp586M2023 /PROD = hypothetical protein /DB_XREF = gi: 4884469 /UG = Hs.247309 succinate-CoA ligase, GDP-forming, beta subunit |
| 216194_s_at | Consensus includes gb: AD001527 /DEF = Homo sapiens DNA from chromosome 19-cosmid f24590 containing CAPNS and POL2RI, genomic sequence |
| | /FEA = CDS 3 /DB_XREF = gi: 1905899 /UG = Hs.31053 cytoskeleton-associated protein 1 |
| 204373_s_at | gb: NM_014810.1 /DEF = Homo sapiens KIAA0480 gene product (KIAA0480), mRNA. /FEA = mRNA /GEN = KIAA0480 /PROD = KIAA0480 gene product |
| | /DB_XREF = gi: 7662155 /UG = Hs.92200 KIAA0480 gene product /FL = gb: AB007949.1 gb: NM_014810.1 |
| 208927_at | Consensus includes gb: BF673888 /FEA = EST /DB_XREF = gi: 11947783 /DB_XREF = est: 602137554F1 /CLONE = IMAGE: 4274077 /UG = Hs.129951 speckle-type POZ |
| | protein /FL = gb: BC001269.1 |
| 220146_at | gb: NM_016562.1 /DEF = Homo sapiens toll-like receptor 7 (LOC51284), mRNA. /FEA = mRNA /GEN = LOC51284 /PROD = toll-like receptor 7 |
| | /DB_XREF = gi: 7706092 /UG = Hs.179152 toll-like receptor 7 /FL = gb: AF240467.1 gb: NM_016562.1 gb: AF245702.1 |
| 219694_at | gb: NM_019018.1 /DEF = Homo sapiens hypothetical protein (FLJ11127), mRNA. /FEA = mRNA /GEN = FLJ11127 /PROD = hypothetical protein |
| | /DB_XREF = gi: 9506640 /UG = Hs.91165 hypothetical protein /FL = gb: NM_019018.1 |
| 213229_at | Consensus includes gb: BF590131 /FEA = EST /DB_XREF = gi: 11682455 /DB_XREF = est: nab19e04.x1 /CLONE = IMAGE: 3266383 /UG = Hs.87889 helicase-moi |
| 214356_s_at | Consensus includes gb: AI272899 /FEA = EST /DB_XREF = gi: 3895167 /DB_XREF = est: q147c07.x1 /CLONE = IMAGE: 1875468 /UG = Hs.3852 KIAA0368 protein |
| 213153_at | Consensus includes gb: AB028999.1 /DEF = Homo sapiens mRNA for KIAA1076 protein, partial cds. /FEA = mRNA /GEN = KIAA1076 /PROD = KIAA1076 protein |
| | /DB_XREF = gi: 5689488 /UG = Hs.154525 KIAA1076 protein |
| 218294_s_at | gb: AF267865.1 /DEF = Homo sapiens DC41 mRNA, complete cds. /FEA = mRNA /PROD = DC41 /DB_XREF = gi: 12006056 /UG = Hs.271623 nucleoporin 50 kD |
| | /FL = gb: AF267865.1 gb: AF107840.1 gb: NM_007172.1 gb: AF116624.1 |
| 221522_at | gb: AL136784.1 /DEF = Homo sapiens mRNA; cDNA DKFZp434L0718 (from clone DKFZp434L0718; complete cds. /FEA = mRNA /GEN = DKFZp434L0718 |
| | /PROD = hypothetical |
| | protein /DB_XREF = gi: 12053080 /UG = Hs.59236 Homo sapiens mRNA; cDNA DKFZp434L0718 (from clone DKFZp434L0718); complete cds /FL = gb: AL136784.1 |
| 205930_at | gb: NM_005513.1 /DEF = Homo sapiens general transcription factor IIE, polypeptide 1 (alpha subunit, 56 kD) (GTF2E1), mRNA. /FEA = mRNA |
| | /GEN = GTF2E1 |
| | /PROD = general transcription factor IIE, polypeptide 1 (alpha subunit, 56 kD) /DB_XREF = gi: 5031726 /UG = Hs.145381 general transcription factor |
| | IIE, polypeptide |
| | 1 (alpha subunit, 56 kD) /FL = gb: NM_005513.1 |
| 210312_s_at | gb: BC002640.1 /DEF = Homo sapiens, Similar to uterine protein, clone MGC: 4279, mRNA, complete cds. /FEA = mRNA /PROD = Similar to uterine protein |
| | /DB_XREF = gi: 12803610 /UG = Hs.4187 hypothetical protein 24636 /FL = gb: BC002640.1 |
| 213838_at | Consensus includes gb: AA191426 /FEA = EST /DB_XREF = gi: 1780105 /DB_XREF = est: zp83g09.s1 /CLONE = IMAGE: 626848 /UG = Hs.279886 RAN binding protein 9 |
| 202520_s_at | gb: NM_000249.1 /DEF = Homo sapiens mutL (E. coli) homolog 1 (colon cancer, nonpolyposis type 2) (MLH1), mRNA. /FEA = mRNA /GEN = MLH1 /PROD = mutL |
| | homolog 1 /DB_XREF = gi: 4557756 /UG = Hs.57301 mutL (E. coli) homolog 1 (colon cancer, nonpolyposis type 2) /FL = gb: NM_000249.1 gb: U07343.1 |
| | gb: U07418.1 |
| 221834_at | Consensus includes gb: AV700132 /FEA = EST /DB_XREF = gi: 10302103 /DB_XREF = est: AV700132 /CLONE = GKCGSE03 /UG = Hs.295923 seven in absentia (Drosophila) |
| | homolog 1 |
| 210942_s_at | gb: AB022918.1 /DEF = Homo sapiens mRNA for alpha2,3-sialyltransferase ST3Gal VI, complete cds. /FEA = mRNA /GEN = ST3Gal VI /PROD = alpha2,3- |
| | sialyltransferase ST3Gal VI /DB_XREF = gi: 4827246 /UG = Hs.34578 alpha2,3-sialyltransferase /FL = gb: AB022918.1 |
| 202396_at | gb: NM_006706.1 /DEF = Homo sapiens transcription factor CA150 (CA150), mRNA. /FEA = mRNA /GEN = CA150 /PROD = transcription factor CA150 |
| | /DB_XREF = gi: 5729753 /UG = Hs.13063 transcription factor CA150 /FL = gb: AF017789.1 gb: NM_006706.1 |
| 219598_s_at | gb: NM_016104.1 /DEF = Homo sapiens PTD013 protein (PTD013), mRNA. /FEA = mRNA /GEN = PTD013 /PROD = PTD013 protein /DB_XREF = gi: 7706668 |
| | /UG = Hs.279857 PTD013protein /FL = gb: AF092134.1 gb: NM_016104.1 |
| 202502_at | gb: NM_000016.1 /DEF = Homo sapiens acyl-Coenzyme A dehydrogenase, C-4 to C-12 straight chain (ACADM), nuclear gene encoding mitochondrial |
| | protein, mRNA. /FEA = mRNA /GEN = ACADM /PROD = acyl-Coenzyme A dehydrogenase, C-4 to C-12straight chain proenzyme /DB_XREF = gi: 4557230 |
| | /UG = Hs.79158 acyl-Coenzyme A dehydrogenase, C-4 to C-12 straight chain /FL = gb: BC005377.1 gb: M16827.1 gb: NM_000016.1 gb: AF251043.1 |
| 211968_s_at | Consensus includes gb: AI962933 /FEA = EST /DB_XREF = gi: 5755646 /DB_XREF = est: wt25a07.x1 /CLONE = IMAGE: 2508468 /UG = Hs.289088 heat shock 90 kD |
| | protein 1, alpha /FL = gb: NM_005348.1 |
| 218027_at | gb: NM_014175.1 /DEF = Homo sapiens HSPC145 protein (HSPC145), mRNA. /FEA = mRNA /GEN = HSPC145 /PROD = HSPC145 protein /DB_XREF = gi: 7661805 |
| | /UG = Hs.18349 HSPC145 protein /FL = gb: AL136665.1 gb: BC000891.1 gb: AF161494.1 gb: NM_014175.1 |
| 217792_at | gb: NM_014426.1 /DEF = Homo sapiens sorting nexin 5 (SNX5), mRNA. /FEA = mRNA /GEN = SNX5 /PROD = sorting nexin 5 /DB_XREF = gi: 7657598 /UG = Hs.13794 |
| | sorting nexin 5 /FL = gb: BC000100.1 gb: AF121855.1 gb: NM_014426.1 |
| 221036_s_at | gb: NM_031301.1 /DEF = Homo sapiens hypothetical protein DKFZp564D0372 (DKFZP564D0372), mRNA. /FEA = mRNA /GEN = DKFZP564D0372 |
| | /PROD = hypothetical protein DKFZp564D0372 /DB_XREF = gi: 13775223 /FL = gb: NM_031301.1 |
| 201723_s_at | gb: U41514.1 /DEF = Human UDP-GalNAc: polypeptide N-acetylgalactosaminyltransferase mRNA, complete cds. /FEA = mRNA /PROD = UDP-GalNAc: |
| | polypeptideN-acetylgalactosaminyltransferase /DB_XREF = gi: 1136284 /UG = Hs.80120 UDP-N-acetyl-alpha-D-galactosamine: polypeptide |
| | N-acetylgalactosaminyltransferase 1 (GalNAc-T1) /FL = gb: U41514.1 gb: NM_020474.2 |
| 202351_at | Consensus includes gb: AI093579 /FEA = EST /DB_XREF = gi: 3432555 /DB_XREF = est: qb15g06.x1 /CLONE = IMAGE: 1696378 /UG = Hs.295726 integrin, |
| | alpha V (vitronectin receptor, alpha polypeptide, antigen CD51) /FL = gb: M14648.1 gb: NM_002210.1 |
| 208853_s_at | gb: L18887.1 /DEF = Human calnexin mRNA, complete cds. /FEA = mRNA /PROD = calnexin /DB_XREF = gi: 306480 /UG = Hs.155560 calnexin /FL = gb: NM_001746.1 |
| | gb: BC003552.1 gb: M94859.1 gb: M98452.1 gb: L10284.1 gb: L18887.1 |
| 213278_at | Consensus includes gb: AW014788 /FEA = EST /DB_XREF = gi: 5863545 /DB_XREF = est: UI-H-BI0-aae-h-10-0-UI.s1 /CLONE = IMAGE: 2709354 /UG = Hs.48802 Homo |
| | sapiens clone 23632 mRNA sequence |
| 203447_at | Consensus includes gb: AU157008 /FEA = EST /DB_XREF = gi: 11018529 /DB_XREF = est: AU157008 /CLONE = PLACE1005711 /UG = Hs.193725 proteasome (prosome, |
| | macropain) 26S subunit, non-ATPase, 5 /FL = gb: NM_005047.1 |
| 212459_x_at | Consensus includes gb: BF593940 /FEA = EST /DB_XREF = gi: 11686264 /DB_XREF = est: nab48f10.x1 /CLONE = IMAGE: 3269154 /UG = Hs.247309 succinate-CoA |
| | ligase, GDP-forming, beta subunit |
| 218514_at | gb: NM_018149.1 /DEF = Homo sapiens hypothetical protein FLJ10587 (FLJ10587), mRNA. /FEA = mRNA /GEN = FLJ10587 /PROD = hypothetical protein |
| | FLJ10587 /DB_XREF = gi: 8922539 /UG = Hs.7296 hypothetical protein FLJ10587 /FL = gb: NM_018149.1 |
| 212648_at | Consensus includes gb: AL079292.1 /DEF = Homo sapiens mRNA full length insert cDNA clone EUROIMAGE 48814. /FEA = mRNA /PROD = hypothetical protein, |
| | similar to (AC007017)putative RNA helicase A Arabidopsis thaliana /DB_XREF = gi: 5102732 /UG = Hs.95665 hypothetical protein |
| 200708_at | gb: NM_002080.1 /DEF = Homo sapiens glutamic-oxaloacetic transaminase 2, mitochondrial (aspartate aminotransferase 2) (GOT2), nuclear gene |
| | encoding mitochondrial protein, mRNA. /FEA = mRNA /GEN = GOT2 /PROD = aspartate aminotransferase 2 precursor /DB_XREF = gi: 4504068 /UG = Hs.170197, |
| | glutamic-oxaloacetic transaminase 2, mitochondrial (aspartate aminotransferase 2) /FL = gb: BC000525.1 gb: M22632.1 gb: NM_002080.1 |
| 201064_s_at | gb: NM_003819.2 /DEF = Homo sapiens poly(A)-binding protein, cytoplasmic 4 (inducible form) (PABPC4), mRNA. /FEA = mRNA /GEN = PABPC4 |
| | /PROD = poly(A)-binding protein, cytoplasmic 4(inducible form) /DB_XREF = gi: 6552335 /UG = Hs.169900 poly(A)-binding protein, cytoplasmic 4 (inducible |
| | form) /FL = gb: NM_003819.2 |
| 218604_at | gb: NM_014319.2 /DEF = Homo sapiens integral inner nuclear membrane protein (MAN1), mRNA. /FEA = mRNA /GEN = MAN1 /PROD = integral inner nuclear |
| | membrane protein /DB_XREF = gi: 7706606 /UG = Hs.7256 integral inner nuclear membrane protein /FL = gb: AF112299.2 gb: NM_014319.2 |
| 214864_s_at | Consensus includes gb: AK024386.1 /DEF = Homo sapiens cDNA FLJ14324 fis, clone PLACE4000100, highly similar to Homo sapiens hydroxypyruvate |
| | reductase(GRHPR) gene. /FEA = mRNA /DB_XREF = gi: 10436760 /UG = Hs.155742 glyoxylate reductasehydroxypyruvate reductase |
| 202717_s_at | gb: NM_003903.1 /DEF = Homo sapiens CDC16 (cell division cycle 16, S. cerevisiae , homolog) (CDC16), mRNA: /FEA = mRNA /GEN = CDC16 /PROD = CDC16 |
| | (cell division cycle 16, S. cerevisiae , homolog) /DB_XREF = gi: 4502700 /UG = Hs.1592 CDC16 (cell division cycle 16, S. cerevisiae , homolog) |
| | /FL = gb: NM_003903.1 gb: U18291.1 |
| 201643_x_at | gb: NM_016604.1 /DEF = Homo sapiens putative zinc finger protein (LOC51780), mRNA. /FEA = mRNA /GEN = LOC51780 /PROD = putative zinc finger protein |
| | /DB_XREF = gi: 7706598 /UG = Hs.24125 putative zinc finger protein /FL = gb: AF251039.1 gb: NM_016604.1 |
| 213803_at | Consensus includes gb: BG545463 /FEA = EST /DB_XREF = gi: 13544128 /DB_XREF = est: 602572695F1 /CLONE = IMAGE: 4701118 /UG = Hs.180446 karyopherin (importin) |
| | beta 1 |
| 204689_at | gb: NM_001529.1 /DEF = Homo sapiens hematopoietically expressed homeobox (HHEX), mRNA. /FEA = mRNA /GEN = HHEX /PROD = hematopoietically expressed |
| | homeobox /DB_XREF = gi: 10835016 /UG = Hs.118651 hematopoietically expressed homeobox /FL = gb: NM_001529.1 gb: L16499.1 gb: NM_002729.1 |
| 218228_s_at | gb: NM_025235.1 /DEF = Homo sapiens tankyrase 2 (TNKL), mRNA. /FEA = mRNA /GEN = TNKL /PROD = tankyrase 2 /DB_XREF = gi: 13376841 /UG = Hs.280776 tankyrase |
| | 2 /FL = gb: AF264912.1 gb: AF329696.1 gb: NM_025235.1 gb: AF342982.1 |
| 202918_s_at | gb: AF151853.1 /DEF = Homo sapiens CGI-95 protein mRNA, complete cds. /FEA = mRNA /PROD = CGI-95 protein /DB_XREF = gi: 4929658 /UG = Hs.107942 |
| | DKFZP564M112 protein /FL = gb: AB015441.1 gb: BC005237.1 gb: AF151853.1 gb: AL080070.1 gb: NM_015387.1 |
| 212263_at | Consensus includes gb: AI114716 /FEA = EST /DB_XREF = gi: 6360061 /DB_XREF = est: HA1315 /UG = Hs.15020 homolog of mouse quaking QKI (KH domain RNA |
| | binding protein) /FL = gb: AF142419.1 gb: AF142422.1 |
| 221825_at | Consensus includes gb: AU151793 /FEA = EST /DB_XREF = gi: 11013314 /DB_XREF = est: AU151793 /CLONE = NT2RP2006115 /UG = Hs.157078 Homo sapiens cDNA |
| | FLJ12793 fis, clone NT2RP2002033 |
| 211047_x_at | gb: BC006337.1 /DEF = Homo sapiens, clone MGC: 12798, mRNA, complete cds. /FEA = mRNA /PROD = Unknown (protein for MGC: 12798) /DB_XREF = gi: 13623468 |
| | /FL = gb: BC006337.1 |
| 209798_at | gb: D83243.1 /DEF = Human NPAT mRNA, complete cds. /FEA = mRNA /GEN = NPAT /DB_XREF = gi: 1304113 /UG = Hs.89385 nuclear protein, ataxia-telangiectasia |
| | locus /FL = gb: D83243.1 gb: NM_002519.1 |
| 204361_s_at | gb: AB014486.1 /DEF = Homo sapiens mRNA for RA70, complete cds. /FEA = mRNA /GEN = RA70 /PROD = RA70 /DB_XREF = gi: 4062959 /UG = Hs.52644 SKAP55 homologue |
| | /FL = gb: BC002893.1 gb: AF072166.1 gb: AB014486.1 gb: AF051323.1 gb: NM_003930.1 |
| 218127_at | Consensus includes gb: AI804118 /FEA = EST /DB_XREF = gi: 5369590 /DB_XREF = est: tc68e01.x1 /CLONE = IMAGE: 2069784 /UG = Hs.84928 nuclear transcription |
| | factor Y, beta /FL = gb: NM_006166.2 gb: BC005316.1 gb: BC005317.1 gb: L06145.1 |
| 220355_s_at | gb: NM_018165.1 /DEF = Homo sapiens hypothetical protein FLJ10645 (FLJ10645), mRNA. /FEA = mRNA /GEN = FLJ10645 /PROD = hypothetical protein |
| | FLJ10645 /DB_XREF = gi: 8922564 /UG = Hs.44143 polybromo 1 /FL = gb: AF177387.1 gb: NM_018165.1 |
| 202930_s_at | gb: NM_003850.1 /DEF = Homo sapiens succinate-CoA ligase, ADP-forming, beta subunit (SUCLA2), mRNA. /FEA = mRNA /GEN = SUCLA2 /PROD = succinate-CoA |
| | ligase, ADP-forming, beta subunit /DB_XREF = gi: 11321582 /UG = Hs.182217 succinate-CoA ligase, ADP-forming, beta subunit /FL = gb: NM_003850.1 |
| | gb: AB035863.1 |
| 203745_at | Consensus includes gb: AI801013 /FEA = EST /DB_XREF = gi: 5366485 /DB_XREF = est: wg15d09.x1 /CLONE = IMAGE: 2365169 /UG = Hs.211571 holocytochrome c |
| | synthase (cytochrome c heme-lyase) /FL = gb: U36787.1 gb: NM_005333.1 |
| 200597_at | Consensus includes gb: AI123320 /FEA = EST /DB_XREF = gi: 3539086 /DB_XREF = est: qa48a10.x1 /CLONE = IMAGE: 1689978 /UG = Hs.198899 eukaryotic translation |
| | initiation factor 3, subunit 10 (theta, 150170 kD) /FL = gb: D50929.1 gb: U58046.1 gb: U78311.1 gb: NM_003750.1 |
| 218519_at | gb: NM_017945.1 /DEF = Homo sapiens hypothetical protein FLJ20730 (FLJ20730), mRNA. /FEA = mRNA /GEN = FLJ20730 /PROD = hypothetical protein |
| | FLJ20730 /DB_XREF = gi: 8923656 /UG = Hs.237480 hypothetical protein FLJ20730 /FL = gb: BC005207.1 gb: NM_017945.1 |
| 203947_at | gb: NM_001326.1 /DEF = Homo sapiens cleavage stimulation factor, 3 pre-RNA, subunit 3, 77 kD (CSTF3), mRNA. /FEA = mRNA /GEN = CSTF3 |
| | /PROD = cleavage stimulation factor subunit 3 /DB_XREF = gi: 4557494 /UG = Hs.180034 cleavage stimulation factor, 3 pre-RNA, subunit 3, 77 kD |
| | /FL = gb: NM_001326.1 gb: U15782.1 |
| 219002_at | gb: NM_024622.1 /DEF = Homo sapiens hypothetical protein FLJ21901 (FLJ21901), mRNA. /FEA = mRNA /GEN = FLJ21901 /PROD = hypothetical protein |
| | FLJ21901 /DB_XREF = gi: 13375843 /UG = Hs.32646 hypothetical protein FLJ21901 /FL = gb: NM_024622.1 |
| 220122_at | gb: NM_024717.1 /DEF = Homo sapiens hypothetical protein FLJ22344 (FLJ22344), mRNA. /FEA = mRNA /GEN = FLJ22344 /PROD = hypothetical protein |
| | FLJ22344 /DB_XREF = gi: 13376020 /UG = Hs.107716 hypothetical protein FLJ22344 /FL = gb: NM_024717.1 |
| 203882_at | gb: NM_006084.1 /DEF = Homo sapiens interferon-stimulated transcription factor 3, gamma (48 kD) (ISGF3G), mRNA. /FEA = mRNA /GEN = ISGF3G |
| | /PROD = interferon-stimulated transcription factor 3, gamma (48 kD) /DB_XREF = gi: 5174474 /UG = Hs.1706 interferon-stimulated transcription factor |
| | 3, gamma (48 kD) /FL = gb: M87503.1 gb: NM_006084.1 |
| 212174_at | Consensus includes gb: W02312 /FEA = EST /DB_XREF = gi: 1274291 /DB_XREF = est: za08d08.r1 /CLONE = IMAGE: 291951 /UG = Hs.171811 adenylate kinase 2 |
| 202567_at | gb: NM_004175.1 /DEF = Homo sapiens small nuclear ribonucleoprotein D3 polypeptide (18 kD) (SNRPD3), mRNA. /FEA = mRNA /GEN = SNRPD3 /PROD = small |
| | nuclear ribonucleoprotein D3 polypeptide(18 kD) /DB_XREF = gi: 4759159 /UG = Hs.1575 small nuclear ribonucleoprotein D3 polypeptide (18 kD) |
| | /FL = gb: BC000457.1 gb: BC003150.1 gb: NM_004175.1 gb: U15009.1 |
| 218557_at | gb: NM_020202.1 /DEF = Homo sapiens Nit protein 2 (NIT2), mRNA. /FEA = mRNA /GEN = NIT2 /PROD = Nit protein 2 /DB_XREF = gi: 9910459 /UG = Hs.15627 Nit |
| | protein 2 /FL = gb: AF260334.1 gb: AF284574.1 gb: NM_020202.1 |
| 211976_at | Consensus includes gb: AK026168.1 /DEF = Homo sapiens cDNA: FLJ22515 fis, clone HRC12122, highly similar to AF052101 Homo sapiens clone 23872 |
| | mRNA sequence. /FEA = mRNA /DB_XREF = gi: 10438931 /UG = Hs.188882 Homo sapiens cDNA: FLJ21862 fis, clone HEP02321, highly similar to AF052101 |
| | Homo sapiens clone 23872 mRNA sequence |
| 201990_s_at | gb: NM_001310.1 /DEF = Homo sapiens cAMP responsive element binding protein-like 2 (CREBL2), mRNA. /FEA = mRNA /GEN = CREBL2 /PROD = cAMP responsive |
| | element binding protein-like 2 /DB_XREF = gi: 4503034 /UG = Hs.13313 cAMP responsive element binding protein-like 2 /FL = gb: AF039081.1 |
| | gb: NM_001310.1 |
| 208956_x_at | gb: U62891.1 /DEF = Human deoxyuridine triphosphatase (DUT) mRNA, complete cds. /FEA = mRNA /GEN = DUT /PROD = deoxyuridine triphosphatase |
| | /DB_XREF = gi: 1421817 /UG = Hs.82113 dUTP pyrophosphatase /FL = gb: AB049113.1 gb: U31930.1 gb: U62891.1 gb: M89913.1 gb: NM_001948.1 |
| 213939_s_at | Consensus includes gb: AI871641 /FEA = EST /DB_XREF = gi: 5545690 /DB_XREF = est: we28f04.x1 /CLONE = IMAGE: 2342431 /UG = Hs.306623 Homo sapiens cDNA |
| | FLJ12012 fis, clone HEMBB1001668 |
| 201198_s_at | Consensus includes gb: AI860431 /FEA = EST /DB_XREF = gi: 5514047 /DB_XREF = est: w113h07.x1 /CLONE = IMAGE: 2424829 /UG = Hs.3887 proteasome (prosome, |
| | macropain) 26S subunit, non-ATPase, 1 /FL = gb: D44466.1 gb: NM_002807.1 |
| 203010_at | gb: NM_003152.1 /DEF = Homo sapiens signal transducer and activator of transcription 5A (STAT5A), mRNA. /FEA = mRNA /GEN = STAT5A /PROD = signal |
| | transducer and activator of transcription5A /DB_XREF = gi: 4507256 /UG = Hs.167503 signal transducer and activator of transcription 5A |
| | /FL = gb: U43185.1 gb: NM_003152.1 gb: L41142.1 |
| 218067_s_at | gb: NM_018011.1 /DEF = Homo sapiens hypothetical protein FLJ10154 (FLJ10154), mRNA. /FEA = mRNA /GEN = FLJ10154 /PROD = hypothetical protein |
| | FLJ10154 /DB_XREF = gi: 8922258 /UG = Hs.179972 hypothetical protein FLJ10154 /FL = gb: NM_018011.1 |
| 219077_s_at | gb: NM_016373.1 /DEF = Homo sapiens WW domain-containing oxidoreductase (WWOX), mRNA. /FEA = mRNA /GEN = WWOX /PROD = FOR II /DB_XREF = gi: 7706522 |
| | /UG = Hs.519 WW domain-containing oxidoreductase /FL = gb: AF211943.1 gb: NM_016373.1 gb: AF227527.1 |
| 215424_s_at | Consensus includes gb: AV689564 /FEA = EST /DB_XREF = gi: 10291427 /DB_XREF = est: AV689564 /CLONE = GKCEAA06 /UG = Hs.79008 SKI-INTERACTING PROTEIN |
| 219043_s_at | gb: NM_024065.1 /DEF = Homo sapiens hypothetical protein MGC3062 (MGC3062), mRNA. /FEA = mRNA /GEN = MGC3062 /PROD = hypothetical protein MGC3062 |
| | /DB_XREF = gi: 13129043 /UG = Hs.94576 hypothetical protein MGC3062 /FL = gb: AF267853.1 gb: BC001021.1 gb: NM_024065.1 |
| 209313_at | gb: AB044661.1 /DEF = Homo sapiens XAB1 mRNA for XPA binding protein 1, complete cds. /FEA = mRNA /GEN = XAB1 /PROD = XPA binding protein 1 |
| | /DB_XREF = gi: 11094140 /UG = Hs.18259 XPA binding protein 1; putative ATP(GTP)-binding protein /FL = gb: AB044661.1 |
| 202944_at | gb: NM_000262.1 /DEF = Homo sapiens N-acetylgalactosaminidase, alpha-(NAGA), mRNA. /FEA = mRNA /GEN = NAGA /PROD = alpha-N-acetylgalactosaminidase |
| | precursor /DB_XREF = gi: 4557780 /UG = Hs.75372 N-acetylgalactosaminidase, alpha- /FL = gb: BC000095.1 gb: M62783.1 gb: M38083.1 gb: NM_000262.1 |
| 208985_s_at | gb: BC002719.1 /DEF = Homo sapiens, eukaryotic translation initiation factor 3, subunit 1 (alpha, 35 kD), clone MGC: 3801, mRNA, complete cds. |
| | /FEA = mRNA /PROD = eukaryotic translation initiation factor 3, subunit 1 (alpha, 35 kD) /DB_XREF = gi: 12803762 /UG = Hs.173987 eukaryotic translation |
| | initiation factor 3, subunit 1 (alpha, 35 kD) /FL = gb: BC002719.1 gb: AF090923.1 |
| 211656_x_at | gb: M32577.1 /DEF = Human MHC HLA-DQ beta mRNA, complete cds. /FEA = mRNA /GEN = HLA-DQB1 /DB_XREF = gi: 188194 /FL = gb: M32577.1 |
| 209358_at | gb: AF118094.1 /DEF = Homo sapiens PRO2134 mRNA, complete cds. /FEA = mRNA /PROD = PRO2134 /DB_XREF = gi: 6650833 /UG = Hs.83126 TATA box binding protein |
| | (TBP)-associated factor, RNA polymerase II, I, 28 kD /FL = gb: D63705.1 gb: NM_005643.1 gb: AF118094.1 |
| 207431_s_at | gb: NM_003676.1 /DEF = Homo sapiens degenerative spermatocyte (homolog Drosophila; lipid desaturase) (DEGS), mRNA. /FEA = mRNA /GEN = DEGS |
| | /PROD = degenerative spermatocyte /DB_XREF = gi: 4505192 /UG = Hs.185973 degenerative spermatocyte (homolog Drosophila; lipid desaturase) |
| | /FL = gb: AF002668.1 gb: NM_003676.1 |
| 213070_at | Consensus includes gb: AV692436 /FEA = EST /DB_XREF = gi: 10294299 /DB_XREF = est: AV682436 /CLONE = GKBABE08 /UG = Hs.5848 Homo sapiens mRNA; cDNA |
| | DKFZp564L222 (from clone DKFZp564L222) |
| 201111_at | gb: AF053641.1 /DEF = Homo sapiens brain cellular apoptosis susceptibility protein (CSE1) mRNA, complete cds. /FEA = mRNA /GEN = CSE1 |
| | /PROD = cellular apoptosis susceptibility protein /DB_XREF = gi: 3560556 /UG = Hs.90073 chromosome segregation 1 (yeast homolog)-like |
| | /FL = gb: U33286.1 gb: AF053641.1 gb: NM_001316.1 |
| 204370_at | gb: NM_006831.1 /DEF = Homo sapiens ATPGTP-binding protein (HEAB), mRNA. /FEA = mRNA /GEN = HEAB /PROD = ATPGTP-binding protein /DB_XREF = gi: 5803028 |
| | /UG = Hs.87465 ATPGTP-binding protein /FL = gb: BC000446.1 gb: U73524.1 gb: NM_006831.1 |
| 202911_at | gb: NM_000179.1 /DEF = Homo sapiens mutS (E. coli) homolog 6 (MSH6), mRNA. /FEA = mRNA /GEN = MSH6 /PROD = mutS (E. coli) homolog 6 |
| | /DB_XREF = gi: 4504190 /UG = Hs.3248 mutS (E. coli) homolog 6 /FL = gb: U28946.1 gb: BC004246.1 gb: NM_000179.1 gb: U54777.2 |
| 204197_s_at | gb: NM_004350.1 /DEF = Homo sapiens runt-related transcription factor 3 (RUNX3), mRNA. /FEA = mRNA /GEN = RUNX3 /PROD = runt-related transcription |
| | factor 3 /DB_XREF = gi: 4757917 /UG = Hs.170019 runt-related transcription factor 3 /FL = gb: NM_004350.1 |
| 217809_at | gb: NM_014038.1 /DEF = Homo sapiens HSPC028 protein (HSPC028), mRNA. /FEA = mRNA /GEN = HSPC028 /PROD = HSPC028 protein /DB_XREF = gi: 7661743 |
| | /UG = Hs.5216 HSPC028 protein /FL = gb: AF110323.1 gb: BC003056.1 gb: AF083246.1 gb: NM_014038.1 |
| 201054_at | Consensus includes gb: BE966599 /FEA = EST /DB_XREF = gi: 11772191 /DB_XREF = est: 601660283R1 /CLONE = IMAGE: 3906044 /UG = Hs.77492 heterogeneous nuclear |
| | ribonucleoprotein A0 /FL = gb: BC001008.1 gb: NM_006805.1 gb: U23803.1 |
| 200994_at | Consensus includes gb: BG291787 /FEA = EST /DB_XREF = gi: 13050002 /DB_XREF = est: 602386007F1 /CLONE = IMAGE: 4515240 /UG = Hs.5151 RAN binding protein |
| | 7 /FL = gb: AF098799.1 gb: NM_006391.1 |
| 201572_x_at | gb: NM_001921.1 /DEF = Homo sapiens dCMP deaminase (DCTD), mRNA. /FEA = mRNA /GEN = DCTD /PROD = dCMP deaminase /DB_XREF = gi: 4503276 /UG = Hs.76894 |
| | dCMP deaminase /FL = gb: L12136.1 gb: NM_001921.1 |
| 218195_at | gb: NM_024573.1 /DEF = Homo sapiens hypothetical protein FLJ12910 (FLJ12910), mRNA. /FEA = mRNA /GEN = FLJ12910 /PROD = hypothetical protein |
| | FLJ12910 |
| | /DB_XREF = gi: 13375745 /UG = Hs.15929 hypothetical protein FLJ12910 /FL = gb: NM_024573.1 |
| 219905_at | gb: NM_018538.1 /DEF = Homo sapiens hypothetical protein PRO2801 (PRO2801), mRNA. /FEA = mRNA /GEN = PRO2801 /PROD = hypothetical protein PRO2801 |
| | /DB_XREF = gi: 8924201 /UG = Hs.181223 hypothetical protein PRO2801 /FL = gb: AF119899.1 gb: NM_018538.1 |
| 209362_at | Consensus includes gb: AI688580 /FEA = EST /DB_XREF = gi: 4899874 /DB_XREF = est: wd39h05.x1 /CLONE = IMAGE: 2330553 /UG = Hs.286145 SRB7 (suppressor of |
| | RNA polymerase B, yeast) homolog /FL = gb: U46837.1 gb: U52960.1 gb: NM_004264.1 |
| 203983_at | gb: NM_005999.1 /DEF = Homo sapiens translin-associated factor X (TSNAX), mRNA. /FEA = mRNA /GEN = TSNAX /PROD = translin-associated factor X |
| | /DB_XREF = gi: 5174730 /UG = Hs.96247 translin-associated factor X /FL = gb: NM_005999.1 |
| 212074_at | Consensus includes gb: BE972774 /FEA = EST /DB_XREF = gi: 10586110 /DB_XREF = est: 601652004F1 /CLONE = IMAGE: 3935281 /UG = Hs.7531 KIAA0810 protein |
| 204859_s_at | gb: NM_013229.1 /DEF = Homo sapiens apoptotic protease activating factor (APAF1), transcript variant 1, mRNA. /FEA = mRNA /GEN = APAF1 |
| | /PROD = apoptotic protease activating factor isoform a /DB_XREF = gi: 7108332 /UG = Hs.77579 apoptotic protease activating factor |
| | /FL = gb: AB007873.1 gb: AF134397.1 gb: NM_013229.1 |
| 205707_at | gb: NM_014339.1 /DEF = Homo sapiens interleukin 17 receptor (IL17R), mRNA. /FEA = mRNA /GEN = IL17R /PROD = interleukin 17 receptor |
| | /DB_XREF = gi: 7657229 /UG = Hs.129751 interleukin 17 receptor /FL = gb: U58917.1 gb: NM_014339.1 |
| 218716_x_at | gb: NM_012123.1 /DEF = Homo sapiens CGI-02 protein (CGI-02), mRNA. /FEA = mRNA /GEN = CGI-02 /PROD = CGI-02 protein /DB_XREF = gi: 6912299 /UG = Hs.33979 |
| | CGI-02 protein /FL = gb: AF319422.1 gb: AF132937.1 gb: NM_012123.1 |
| 217940_s_at | gb: NM_018210.1 /DEF = Homo sapiens hypothetical protein FLJ10769 (FLJ10769), mRNA. /FEA = mRNA /GEN = FLJ10769 /PROD = hypothetical protein |
| | FLJ10769 /DB_XREF = gi: 8922653 /UG = Hs.8083 hypothetical protein FLJ10769 /FL = gb: AF151071.1 gb: NM_018210.1 |
| 219802_at | gb: NM_024854.1 /DEF = Homo sapiens hypothetical protein FLJ22028 (FLJ22028), mRNA. /FEA = mRNA /GEN = FLJ22028 /PROD = hypothetical protein |
| | FLJ22028 /DB_XREF = gi: 13376278 /UG = Hs.192570 hypothetical protein FLJ22028 /FL = gb: NM_024854.1 |
| 209259_s_at | gb: AF020043.1 /DEF = Homo sapiens chromosome-associated polypeptide (HCAP) mRNA, complete cds. /FEA = mRNA /GEN = HCAP /PROD = chromosome- |
| | associated polypeptide /DB_XREF = gi: 3089367 /UG = Hs.24485 chondroitin sulfate proteoglycan 6 (bamacan) /FL = gb: AF020043.1 gb: NM_005445.1 |
| | gb: AF067163.1 |
| 212037_at | Consensus includes gb: BF508848 /FEA = EST /DB_XREF = gi: 11592146 /DB_XREF = est: UI-H-BI4-aor-e-06-0-UI.s1 /CLONE = IMAGE: 3085907 /UG = Hs.44499 |
| | pinin, desmosome associated protein |
| 211063_s_at | gb: BC006403.1 /DEF = Homo sapiens, NCK adaptor protein 1, clone MGC: 12668, mRNA, complete cds. /FEA = mRNA /PROD = NCK adaptor protein 1 |
| | /DB_XREF = gi: 13623576 /FL = gb: BC006403.1 |
| 203791_at | gb: NM_005509.2 /DEF = Homo sapiens Dmx-like 1 (DMXL1), mRNA. /FEA = mRNA /GEN = DMXL1 /PROD = Dmx-like 1 /DB_XREF = gi: 9961348 /UG = Hs.181042 Dmx-like |
| | 1 /FL = gb: NM_005509.2 |
| 203405_at | gb: NM_003720.1 /DEF = Homo sapiens Down syndrome critical region gene 2 (DSCR2), mRNA. /FEA = mRNA /GEN = DSCR2 /PROD = Down syndrome critical |
| | region protein 2 /DB_XREF = gi: 4505022 /UG = Hs.5198 Down syndrome critical region gene 2 /FL = gb: BC003619.1 gb: NM_003720.1 |
| 201604_s_at | gb: NM_002480.1 /DEF = Homo sapiens myosin phosphatase, target subunit 1 (MYPT1), mRNA. /FEA = mRNA /GEN = MYPT1 /PROD = myosin phosphatase target |
| | subunit 1 /DB_XREF = gi: 4505316 /UG = Hs.16533 myosin phosphatase, target subunit 1 /FL = gb: NM_002480.1 |
| 218348_s_at | gb: NM_014153.1 /DEF = Homo sapiens HSPC055 protein (HSPC055), mRNA. /FEA = mRNA /GEN = HSPC055 /PROD = HSPC055 protein /DB_XREF = gi: 7661761 |
| | /UG = Hs.179898 HSPC055 protein /FL = gb: AF161540.1 gb: NM_014153.1 |
| 205659_at | gb: NM_014707.1 /DEF = Homo sapiens histone deacetylase 7B (HDAC7B-PENDING), mRNA. /FEA = mRNA /GEN = HDAC7B-PENDING /PROD = histone deacetylase 7B |
| | /DB_XREF = gi: 7662279 /UG = Hs.116753 histone deacetylase. 7B /FL = gb: AB018287.1 gb: NM_014707.1 |
| 202268_s_at | gb: NM_003905.1 /DEF = Homo sapiens amyloid beta precursor protein-binding protein 1, 59 kD (APPBP1), mRNA. /FEA = mRNA /GEN = APPBP1 |
| | /PROD = Amyloid beta precursor protein-binding protein1 /DB_XREF = gi: 4502168 /UG = Hs.61828 amyloid beta precursor protein-binding protein 1, |
| | 59 kD /FL = gb: AL136798.1 gb: BC000480.1 gb: U50939.1 gb: NM_003905.1 |
| 202399_s_at | gb: NM_005829.1 /DEF = Homo sapiens adaptor-related protein complex 3, sigma 2 subunit (AP3S2), mRNA. /FEA = mRNA /GEN = AP3S2 /PROD = adaptor- |
| | related protein complez 3, sigma 2subunit /DB_XREF = gi: 5031580 /UG = Hs.154782 adaptor-related protein complex 3, sigma 2 subunit |
| | /FL = gb: BC002785.1 gb: NM_005829.1 |
| 218515_at | gb: NM_016631.1 /DEF = Homo sapiens hypothetical protein (LOC51325), mRNA. /FEA = mRNA /GEN = LOC51325 /PROD = hypothetical protein |
| | /DB_XREF = gi: 7706175 /UG = Hs.26461 hypothetical protein /FL = gb: AF208862.1 gb: NM_016631.1 |
| 209180_at | gb: U49245.1 /DEF = Human geranylgeranyl transferase type II beta-subunit mRNA, complete cds. /FEA = mRNA /PROD = geranylgeranyl transferase type |
| | II beta-subunit /DB_XREF = gi: 1216503 /UG = Hs.78948 Rab geranylgeranyltransferase, beta subunit /FL = gb: U49245.1 gb: NM_004582.1 |
| 205105_at | gb: NM_002372.1 /DEF = Homo sapiens mannosidase, alpha, class 2A, member 1 (MAN2A1), mRNA. /FEA = mRNA /GEN = MAN2A1 /PROD = mannosidase, alpha, |
| | class 2A, member 1 /DB_XREF = gi: 4758697 /UG = Hs.32965 mannosidase, alpha, class 2A, member 1 /FL = gb: U31520.1 gb: NM_002372.1 gb: D63998.1 |
| 200666_s_at | gb: NM_006145.1 /DEF = Homo sapiens heat shock 40 kD protein 1 (HSPF1), mRNA. /FEA = mRNA /GEN = HSPF1 /PROD = heat shock 40 kD protein 1 |
| | /DB_XREF = gi: 5453689 /UG = Hs.82646 DnaJ (Hsp40) homolog, subfamily B, member 1 /FL = gb: BC002352.1 gb: NM_006145.1 gb: D49547.1 |
| 200972_at | gb: BC000704.1 /DEF = Homo sapiens, tetraspan 3, clone MGC: 965, mRNA, complete cds. /FEA = mRNA /PROD = tetraspan 3 /DB_XREF = gi: 12653830 |
| | /UG = Hs.100090 tetraspan 3 /FL = gb: BC000704.1 gb: BC004280.1 gb: AF054840.1 gb: NM_005724.1 gb: AF133423.1 |
| 218352_at | gb: NM_018191.1 /DEF = Homo sapiens hypothetical protein FLJ10716 (FLJ10716), mRNA. /FEA = mRNA /GEN = FLJ10716 /PROD = hypothetical protein |
| | FLJ10716 /DB_XREF = gi: 8922616 /UG = Hs.24129 hypothetical protein FLJ10716 /FL = gb: NM_018191.1 |
| 217728_at | gb: NM_014624.2 /DEF = Homo sapiens S100 calcium-binding protein A6 (calcyclin) (S100A6), mRNA. /FEA = mRNA /GEN = S100A6 /PROD = S100 calcium- |
| | binding protein A6 /DB_XREF = gi: 9845517 /UG = Hs.275243 S100 calcium-binding protein A6 (calcyclin) /FL = gb: BC001431.1 gb: NM_014624.2 |
| 211971_s_at | Consensus includes gb: AI653608 /FEA = EST /DB_XREF = gi: 4737587 /DB_XREF = est: tz21a06.x1 /CLONE = IMAGE: 2289202 /UG = Hs.182490 leucine-rich protein |
| | mRNA |
| 212500_at | Consensus includes gb: AL049319.1 /DEF = Homo sapiens mRNA; cDNA DKFZp564C046 (from clone DKFZp564C046) /FEA = mRNA /DB_XREF = gi: 4500092 |
| | /UG = Hs.99821 Homo sapiens mRNA; cDNA DKFZp564C046 (from clone DKFZp564C046) |
| 218473_s_at | gb: NM_024656.1 /DEF = Homo sapiens hypothetical protein FLJ22329 (FLJ22329), mRNA. /FEA = mRNA /GEN = FLJ22329 /PROD = hypothetical protein |
| | FLJ22329 /DB_XREF = gi: 13375904 /UG = Hs.61478 hypothetical protein FLJ22329 /FL = gb: NM_024656.1 |
| 203580_s_at | gb: NM_003983.1 /DEF = Homo sapiens solute carrier family 7 (cationic amino acid transporter, y+ system), member 6 (SLC7A6), mRNA. /FEA = mRNA |
| | /GEN = SLC7A6 /PROD = solute carrier family 7 (cationic amino acidtransporter, y+ system), member 6 /DB_XREF = gi: 4507052 /UG = Hs.10315 solute |
| | carrier family 7 (cationic amino acid transporter, y+ system), member 6 /FL = gb: D87432.1 gb: NM_003983.1 |
| 200900_s_at | Consensus includes gb: AI583537 /FEA = EST /DB_XREF = gi: 4569434 /DB_XREF = est: ts12d03.x1 /CLONE = IMAGE: 2228357 /UG = Hs.75709 mannose-6-phosphate |
| | receptor (cation dependent) /FL = gb: NM_002355.2 gb: M16985.1 |
| 221652_s_at | gb: AF274950.1 /DEF = Homo sapiens PNAS-25 mRNA, complete cds. /FEA = mRNA /PROD = PNAS-25 /DB_XREF = gi: 12751064 /UG = Hs.22595 hypothetical protein |
| | FLJ10637 /FL = gb: AF274950.1 |
| 217750_s_at | gb: NM_023079.1 /DEF = Homo sapiens hypothetical protein FLJ13855 (FLJ13855), mRNA. /FEA = mRNA /GEN = FLJ13855 /PROD = hypothetical protein |
| | FLJ13855 |
| | /DB_XREF = gi: 12751494 /UG = Hs.168232 hypothetical protein FLJ13855 /FL = gb: NM_023079.1 |
| 203544_s_at | gb: NM_003473.1 /DEF = Homo sapiens signal transducing adaptor molecule (SH3 domain and ITAM motif) 1 (STAM), mRNA. /FEA = mRNA /GEN = STAM |
| | /PROD = signal transducing adaptor molecule (SH3 domainand ITAM motif) 1 /DB_XREF = gi: 4507248 /UG = Hs.153487 signal transducing adaptor molecule |
| | (SH3 domain and ITAM motif) 1 /FL = gb: U43899.1 gb: NM_003473.1 |
| 221580_s_at | gb: BC001972.1 /DEF = Homo sapiens, clone MGC: 5306, mRNA, complete cds. /FEA = mRNA /PROD = Unknown (protein for MGC: 5306) /DB_XREF = gi: 12805036 |
| | /UG = Hs.301732 hypothetical protein MGC5306 /FL = gb: BC001972.1 |
| 202629_at | Consensus includes gb: AV681579 /FEA = EST /DB_XREF = gi: 10283442 /DB_XREF = est: AV681579 /CLONE = GKBAFE05 /UG = Hs.84084 amyloid beta precursor |
| | protein (cytoplasmic tail)-binding protein 2 /FL = gb: AF017782.1 gb: NM_006380.1 |
| 205763_s_at | gb: NM_006773.2 /DEF = Homo sapiens DEADH (Asp-Glu-Ala-AspHis) box polypeptide 18 (Myc-regulated) (DDX18), mRNA. /FEA = mRNA /GEN = DDX18 |
| | /PROD = DEADH (Asp-Glu-Ala-AspHis) box polypeptide 18 /DB_XREF = gi: 13787205 /UG = Hs.100555 DEADH (Asp-Glu-Ala-AspHis) box polypeptide 18 (Myc- |
| | regulated) /FL = gb: NM_006773.2 |
| 212337_at | Consensus includes gb: AI687738 /FEA = EST /DB_XREF = gi: 4899032 /DB_XREF = est: tp93g08.x1 /CLONE = IMAGE: 2206910 /UG = Hs.699 peptidylprolyl isomerase |
| | B (cyclophilin B) |
| 212244_at | Consensus includes gb: AL050091.1 /DEF = Homo sapiens mRNA; cDNA DKFZp586F1918 (from clone DKFZp586F1918); partial cds. /FEA = mRNA |
| | /GEN = DKEZp586F1918 /PROD = hypothetical protein /DB_XREF = gi: 4884111 /UG = Hs.6283 DKFZP586F1918 protein |
| 222011_s_at | Consensus includes gb: BF224073 /FEA = EST /DB_XREF = gi: 11131299 /DB_XREF = est: 7q83e05.x1 /CLONE = IMAGE: 3704936 /UG = Hs.278544 acetyl-Coenzyme A |
| | acetyltransferase 2 (acetoacetyl Coenzyme A thiolase) |
| 204725_s_at | gb: NM_006153.1 /DEF = Homo sapiens NCK adaptor protein 1 (NCK1), mRNA. /FEA = mRNA /GEN = NCK1 /PROD = NCK adaptor protein 1 /DB_XREF = gi: 5453753 |
| | /UG = Hs.54589 NCK adaptor protein 1 /FL = gb: NM_006153.1 |
| 208860_s_at | gb: U09820.1 /DEF = Human helicase II (RAD54L) mRNA, complete cds. /FEA = mRNA /GEN = RAD54L /PROD = helicase II /DB_XREF = gi: 606832 /UG = Hs.96264 |
| | alpha thalassemiamental retardation syndrome X-linked (RAD54 (S. cerevisiae) homolog) /FL = gb: U09820.1 gb: NM_000489.1 gb: U12937.2 |
| 203224_at | Consensus includes gb: BF340123 /FEA = EST /DB_XREF = gi: 11286585 /DB_XREF = est: 602037283F1 /CLONE = IMAGE: 4185212 /UG = Hs.37558 hypothetical protein |
| | FLJ11149 /FL = gb: NM_018339.1 |
| 208619_at | gb: L40326.1 /DEF = Homo sapiens Hepatitis B virus X-associated protein 1 mRNA, complete cds. /FEA = mRNA /PROD = X-associated protein 1 |
| | /DB_XREF = gi: 695361 |
| | /UG = Hs.108327 damage-specific DNA binding protein 1 (127 kD) /FL = gb: U18299.1 gb: U32986.1 gb: NM_001923.2 gb: L40326.1 |
| 208765_s_at | Consensus includes gb: NM_005826.1 /DEF = Homo sapiens, heterogeneous nuclear ribonucleoprotein R (HNRPR), mRNA. /FEA = CDS /GEN = HNRPR |
| | /PROD = heterogeneous nuclear ribonucleoprotein R /DB_XREF = gi: 5031754 /UG = Hs.15265 heterogeneous nuclear ribonucleoprotein R |
| | /FL = gb: BC001449.1 gb: AF000364.1 gb: NM_005826.1 |
| 220832_at | gb: NM_016610.1 /DEF = Homo sapiens Toll-like receptor 8 (LOC51311), mRNA. /FEA = mRNA /GEN = LOC51311 /PROD = Toll-like receptor 8 |
| | /DB_XREF = gi: 7706147 |
| | /UG = Hs.272410 Toll-like receptor 8 /FL = gb: AF246971.1 gb: NM_016610.1 gb: AF245703.1 |
| 208398_s_at | gb: NM_004865.1 /DEF = Homo sapiens TBP-like 1 (TBPL1), mRNA. /FEA = mRNA /GEN = TBPL1 /PROD = TBP-like 1 /DB_XREF = gi: 4759233 /UG = Hs.13993 TBP-like |
| | 1 /FL = gb: AF130312.1 gb: NM_004865.1 |
| 205077_s_at | gb: NM_002643.1 /DEF = Homo sapiens phosphatidylinositol glycan, class F (PIGF), mRNA. /FEA = mRNA /GEN = PIGF /PROD = phosphatidylinositol glycan, |
| | class F /DB_XREF = gi: 4505796 /UG = Hs.166982 phosphatidylinositol glycan, class F /FL = gb: D13435.1 gb: NM_002643.1 |
| 210616_s_at | gb: AB020712.1 /DEF = Homo sapiens mRNA for KIAA0905 protein, complete cds. /FEA = mRNA /GEN = KIAA0905 /PROD = KIAA0905 protein /DB_XREF = gi: 4240298 |
| | /UG = Hs.70266 yeast Sec31p homolog /FL = gb: AB020712.1 |
| 211761_s_at | gb: BC005975.1 /DEF = Homo sapiens, calcyclin binding protein, clone MGC: 14660, mRNA, complete cds. /FEA = mRNA /PROD = calcyclin binding protein |
| | /DB_XREF = gi: 13543650 /FL = gb: BC005975.1 |
| 213164_at | Consensus includes gb: AI867198 /FEA = EST /DB_XREF = gi: 5540214 /DB_XREF = est: wa01c11.x1 /CLONE = IMAGE: 2296820 /UG = Hs.324787 solute carrier family |
| | 5 (inositol transporters), member 3 /FL = gb: NM_006933.1 |
| 219940_s_at | gb: NM_018386.1 /DEF = Homo sapiens hypothetical protein FLJ11305 (FLJ11305), mRNA. /FEA = mRNA /GEN = FLJ11305 /PROD = hypothetical protein |
| | FLJ11305 /DB_XREF = gi: 8922986 /UG = Hs.7049 hypothetical protein FLJ11305 /FL = gb: NM_018386.1 |
| 203800_s_at | Consensus includes gb: BG254653 /FEA = EST /DB_XREF = gi: 12764469 /DB_XREF = est: 602368621F1 /CLONE = IMAGE: 4476773 /UG = Hs.247324 hypothetical protein |
| | LOC63931 /FL = gb: NM_022100.1 |
| 213026_at | Consensus includes gb: BE965998 /FEA = EST /DB_XREF = gi: 11770950 /DB_XREF = est: 601659892R1 /CLONE = IMAGE: 3905710 /UG = Hs.264482 Apg12 (autophagy |
| | 12, S. cerevisiae)-like |
| 215165_x_at | Consensus includes gb: AL080099.1 /DEF = Homo sapiens mRNA; cDNA DKFZp564G1272 (from clone DKFZp564G1272); partial cds. /FEA = mRNA |
| | /GEN = DKFZp564G1272 /PROD = hypothetical protein /DB_XREF = gi: 5262522 /UG = Hs.2057 uridine monophosphate synthetase (orotate phosphoribosyl |
| | transferase and orotidine-5-decarboxylase) |
| 202864_s_at | gb: NM_003113.1 /DEF = Homo sapiens nuclear antigen Sp100 (SP100), mRNA. /FEA = mRNA /GEN = SP100 /PROD = nuclear antigen Sp100 /DB_XREF = gi: 4507164 |
| | /UG = Hs.77617 nuclear antigen Sp100 /FL = gb: M60618.1 gb: NM_003113.1 |
| 208407_s_at | gb: NM_001331.1 /DEF = Homo sapiens catenin (cadherin-associated protein), delta 1 (CTNND1), mRNA. /FEA = mRNA /GEN = CTNND1 /PROD = catenin |
| | (cadherin-associated protein), delta 1 /DB_XREF = gi: 10835009 /UG = Hs.166011 catenin (cadherin-associated protein), delta 1 /FL = gb: NM_001331.1 |
| | gb: AF062317.1 |
| 202419_at | gb: NM_002035.1 /DEF = Homo sapiens follicular lymphoma variant translocation 1 (FVT1), mRNA. /FEA = mRNA /GEN = FVT1 /PROD = follicular lymphoma |
| | variant translocation 1 /DB_XREF = gi: 4503816 /UG = Hs.74050 follicular lymphoma variant translocation 1 /FL = gb: NM_002035.1 |
| 201833_at | gb: NM_001527.1 /DEF = Homo sapiens histone deacetylase 2 (HDAC2), mRNA. /FEA = mRNA /GEN = HDAC2 /PROD = histone deacetylase 2 /DB_XREF = gi: 4557640 |
| | /UG = Hs.3352 histone deacetylase 2 /FL = gb: U31814.1 gb: NM_001527.1 |
| 217879_at | Consensus includes gb: AL566824 /FEA = EST /DB_XREF = gi: 12919571 /DB_XREF = est: AL566824 /CLONE = CS0DF025YN03 (3 prime) /UG = Hs.172405 call division |
| | cycle 27 /FL = gb: NM_001256.1 |

TABLE 5
Genes from Cluster Analysis 5

| Affymetrix | |
|---|---|
| internal | |
| designation | Description of the sequence in the GeneBank data base |
| 201466_s_at | gb: NM_002228.2 /DEF = Homo sapiens v-jun avian sarcoma virus 17 oncogene homolog (JUN), mRNA. /FEA = mRNA /GEN = JUN /PROD = v-jun avian sarcoma |
| | virus 17 oncogene homolog /DB_XREF = gi: 7710122 /UG = Hs.78465 v-jun avian sarcoma virus 17 oncogene homolog /FL = gb: BC002646.1 gb: NM_002228.2 |
| 202425_x_at | gb: NM_000944.1 /DEF = Homo sapiens protein phosphatase 3 (formerly 2B), catalytic subunit, alpha isoform (calcineurin A alpha) (PPP3CA), mRNA. |
| | /FEA = mRNA /GEN = PPP3CA /PROD = protein phosphatase 3 (formerly 2B), catalyticsubunit, alpha isoform (calcineurin A alpha) /DB_XREF = gi: 6715567 |
| | /UG = Hs.272458 protein phosphatase 3 (formerly 2B), catalytic subunit, alpha isoform (calcineurin A alpha) /FL = gb: J05480.1 gb: L14778.1 |
| | gb: NM_000944.1 gb: AL353950.1 |
| 218566_s_at | gb: NM_012124.1 /DEF = Homo sapiens chord domain-containing protein 1 (CHP1), mRNA. /FEA = mRNA /GEN = CHP1 /PROD = chord domain-containing protein 1 |
| | /DB_XREF = gi: 6912303 /UG = Hs.22857 cysteine and histidine-rich domain (CHORD)-containing, zinc-binding protein 1 /FL = gb: AF192466.1 |
| | gb: NM_012124.1 |
| 214349_at | Consensus includes gb: AV764378 /FEA = EST /DB_XREF = gi: 10922226 /DB_XREF = est: AV764378 /CLONE = MDSAOE03 /UG = Hs.163863 ESTs, Moderately similar to |
| | POL2 HUMAN RETROVIRUS-RELATED POL POLYPROTEIN H. sapiens |
| 204314_s_at | gb: NM_004379.1 /DEF = Homo sapiens cAMP responsive element binding protein 1 (CREB1), mRNA. /FEA = mRNA /GEN = CREB1 /PROD = cAMP responsive elemen |
| | binding protein 1 /DB_XREF = gi: 4758053 /UG = Hs.79194 cAMP responsive element binding protein 1 /FL = gb: M27691.1 gb: NM_004379.1 |
| 208753_s_at | gb: BC002387.1 /DEF = Homo sapiens, nucleosome assembly protein 1-like 1, clone MGC: 8688, mRNA, complete cds. /FEA = mRNA /PROD = nucleosome assembly |
| | protein 1-like 1 /DB_XREF = gi: 12803166 /UG = Hs.179662 nucleosome assembly protein 1-like 1 /FL = gb: BC002387.1 gb: AL162068.1 |
| 215452_x_at | Consensus includes gb: AL031133 /DEF = Human DNA sequence from clone 281H8 on chromosome 6q25.1-25.3. Contains up to four novel genes, one with |
| | similarity to KIAA0323 and worm C30F12.1 and another with Ubiquitin-Like protein gene SMT3 (the latter in an intron of a novel gene). Contains |
| | /FEA = mRNA_2 /DB_XREF = gi: 3676189 /UG = Hs.113293 Human DNA sequence from clone 281H8 on chromosome 6q25.1-25.3. Contains up to four novel |
| | genes, one with similarity to KIAA0323 and worm C30F12.1 and another with Ubiquitin-Like protein gene SMT3 (the latter in an intron of a |
| | novel gene). Contains ESTs, S |
| 204119_s_at | gb: U90339.1 /DEF = Human adenosine kinase short form mRNA, complete cds. /FEA = mRNA /PROD = adenosine kinase short form /DB_XREF = gi: 1906010 |
| | /UG = Hs.94382 adenosine kinase /FL = gb: U50196.1 gb: BC003568.1 gb: U90339.1 gb: NM_01123.1 |
| 201304_at | gb: NM_005000.2 /DEF = Homo sapiens NADH dehydrogenase (ubiquinone) 1 alpha subcomplex, 5 (13 kD, B13) (NDUFA5), nuclear gene encoding |
| | mitochondrial protein, mRNA. /FEA = mRNA /GEN = NDUFA5 /PROD = NADH dehydrogenase (ubiquinone) 1 alphasubcomplex, 5 /DB_XREF = gi: 13699821 |
| | /UG = Hs.83916 NADH dehydrogenase (ubiquinone) 1 alpha subcomplex, 5 (13 kD, B13) /FL = gb: BC000813.1 gb: NM_005000.2 gb: U53468.1 gb: U64028.1 |
| 218131_s_at | gb: NM_017660.1 /DEF = Homo sapiens hypothetical protein FLJ20085 (FLJ20085), mRNA. /FEA = mRNA /GEN = FLJ20085 /PROD = hypothetical protein FLJ20085 |
| | /DB_XREF = gi: 8923093 /UG = Hs.118964 hypothetical protein FLJ20085 /FL = gb: NM_017660.1 |
| 201788_at | gb: NM_007372.1 /DEF = Homo sapiens RNA helicase-related protein (RNAHP), mRNA. /FEA = mRNA /GEN = RNAHP /PROD = RNA helicase-related protein |
| | /DB_XREF = gi: 11321631 /UG = Hs.8765 RNA helicase-related protein /FL = gb: NM_007372.1 gb: AF083255.1 |
| 209780_at | gb: AL136883.1 /DEF = Homo sapiens mRNA; cDNA DKFZp434D166 (from clone DKFZp434D166); complete cds. /FEA = mRNA /GEN = DKFZp434D166 |
| | /PROD = hypothetical protein /DB_XREF = gi: 12053266 /UG = Hs.128653 hypothetical protein DKFZp564F013 /FL = gb: AL136883.1 |
| 219375_at | gb: NM_006090.1 /DEF = Homo sapiens cholineethanolaminephosphotransferase (CEPT1), mRNA. /FEA = mRNA /GEN = CEPT1 |
| | /PROD = cholineethanolaminephosphotransferase /DB_XREF = gi: 5174414 /UG = Hs.125031 cholineethanolaminephosphotransferase |
| | /FL = gb: AF068302.1 gb: NM_006090.1 |
| 209187_at | Consensus includes gb: AW516932 /FEA = EST /DB_XREF = gi: 7154941 /DB_XREF = est: xq04a05.x1 /CLONE = IMAGE: 2748848 /UG = Hs.16697 down-regulator of |
| | transcription 1, TBP-binding (negative cofactor 2) /FL = gb: BC002809.1 |
| 219279_at | gb: NM_017718.1 /DEF = Homo sapiens hypothetical protein FLJ20220 (FLJ20220), mRNA. /FEA = mRNA /GEN = FLJ20220 /PROD = hypothetical protein FLJ20220 |
| | /DB_XREF = gi: 8923209 /UG = Hs.21126 hypothetical protein FLJ20220 /FL = gb: NM_017718.1 |
| 206976_s_at | gb: NM_006644.1 /DEF = Homo sapiens heat shock 105 kD (HSP105B), mRNA. /FEA = mRNA /GEN = HSP105B /PROD = heat shock 105 kD /DB_XREF = gi: 5729878 |
| | /UG = Hs.36927 heat shock 105 kD /FL = gb: AB003333.1 gb: NM_006644.1 |
| 215596_s_at | Consensus includes gb: AL163248 /DEF = Homo sapiens chromosome 21 segment HS21C048 /FEA = mRNA_2 /DB_XREF = gi: 7717304 /UG = Hs.288773 zinc finger |
| | porotein 294 |
| 216511_s_at | Consensus includes gb: AJ270770 /DEF = Homo sapiens partial TCF-4 gene for T-cell transcription factor-4, exon 1 and joined CDS features |
| | /FEA = mRNA 1 /DB_XREF = gi: 9188625 /UG = Hs.283857 Homo sapiens partial TCF-4 gene for T-cell transcription factor-4, exon 1 and joined CDS features |
| 201889_at | gb: NM_014888.1 /DEF = Homo sapiens predicted osteoblast protein (GS3786), mRNA. /FEA = mRNA /GEN = GS3786 /PROD = predicted osteoblest protein |
| | /DB_XREF = gi: 7661713 /UG = Hs.29882 predicted osteoblast protein /FL = gb: D87120.1 gb: NM_014888.1 |
| 201435_s_at | Consensus includes gb: AW268640 /FEA = EST /DB_XREF = gi: 6655670 /DB_XREF = est: xv52a03.x1 /CLONE = IMAGE: 2816716 /UG = Hs.79306 eukaryotic translation |
| | initiation factor 4E /FL = gb: M15353.1 gb: NM_001968.1 |
| 212232_at | Consensus includes gb: AB023231.1 /DEF = Homo sapiens mRNA for KIAA1014 protein, partial cds. /FEA = mRNA /GEN = KIAA1014 /PROD = KIAA1014 protein |
| | /DB_XREF = gi: 4589677 /UG = Hs.6834 KIAA1014 protein |
| 214329_x_at | Consensus includes gb: AW474434 /FEA = EST /DB_XREF = gi: 7044540 /DB_XREF = est: xs95h07.x1 /CLONE = IMAGE: 2777437 /UG = Hs.301527 ESTs, Moderately |
| | similar to unknown H. sapiens |
| 212099_at | Consensus includes gb: AI263909 /FEA = EST /DB_XREF = gi: 3872112 /DB_XREF = est: qi08f09.x1 /CLONE = IMAGE: 1855913 /UG = Hs.204354 ras homolog gene |
| | family, member B /FL = gb: NM_004040.1 |
| 213618_at | Consensus includes gb: AB011152.1 /DEF = Homo sapiens mRNA for KIAA0580 protein, partial cds. /FEA = mRNA /GEN = KIAA0580 /PROD = KIAA0580 protein |
| | /DB_XREF = gi: 3043683 /UG = Hs.22572 KIAA0580 protein |
| 216899_s_at | Consensus includes gb: AC003999 /DEF = Human PAC clone RP5-1139P1 from 7p15-p21 /FEA = CDS /DB_XREF = gi: 2772566 /UG = Hs.52644 SKAP55 homologue |
| 201437_s_at | gb: NM_001968.1 /DEF = Homo sapiens eukaryotic translation initiation factor 4E (EIF4E), mRNA. /FEA = mRNA /GEN = EIF4E /PROD = eukeryotic translation |
| | initiation factor 4E /DB_XREF = gi: 4503534 /UG = Hs.79306 eukaryotic translation initiation factor 4E /FL = gb: M15353.1 gb: NM_001968.1 |
| 212122_at | Consensus includes gb: AW771590 /FEA = EST /DB_XREF = gi: 7703647 /DB_XREF = est: hn58g08.x1 /CLONE = IMAGE: 3032126 /UG = Hs.166982 phosphatidylinositol |
| | glycan, class F |
| 208666_s_at | Consensus includes gb: BE866412 /FEA = EST /DB_XREF = gi: 10315097 /DB_XREF = est: 601678647F1 /CLONE = IMAGE: 3961522 /UG = Hs.119222 suppression of |
| | tumorigenicity 13 (colon carcinoma) (Hsp70-interacting protein) /FL = gb: U17714.1 gb: AF116650.1 |
| 218252_at | gb: NM_018204.1 /DEF = Homo sapiens cytoskeleton associated protein 2 (CKAP2), mRNA. /FEA = mRNA /GEN = CKAP2 /PROD = cytoskeleton associated protein 2 |
| | /DB_XREF = gi: 8922641 /UG = Hs.24641 cytoskeleton associated protein 2 /FL = gb: AL136848.1 gb: NM_018204.1 |
| 216384_x_at | Consensus includes gb: AF257099 /DEF = Homo sapiens prothymosin alpha (PTMA) gene, complete cds /FEA = CDS /DB_XREF = gi: 8037944 /UG = Hs.283947 Homo |
| | sapiens prothymosin alpha (PTMA) gene, complete cds |
| 217100_s_at | Consensus includes gb: AK026451.1 /DEF = Homo sapiens cDNA: FLJ22798 fis, clone KAIA2617. /FEA = mRNA /DB_XREF = gi: 10439320 /UG = Hs.127287 KIAA0794 |
| | protein |
| 204313_s_at | Consensus includes gb: AA161486 /FEA = EST /DB_XREF = gi: 1735796 /DB_XREF = est: zq42d09.s1 /CLONE = IMAGE: 632369 /UG = Hs.79194 cAMP responsive element |
| | binding protein 1 /FL = gb: M27691.1 gb: NM_004379.1 |
| 214257_s_at | Consensus includes gb: AA890010 /FEA = EST /DB_XREF = gi: 3016889 /DB_XREF = est: aj89h08.s1 /CLONE = IMAGE: 1403679 /UG = Hs.50785 SEC22, vesicle trafficking |
| | protein (S. cerevisiae)-like 1 |
| 218595_s_at | gb: NM_018072.1 /DEF = Homo sapiens hypothetical protein FLJ10359 (FLJ10359), mRNA. /FEA = mRNA /GEN = FLJ10359 /PROD = hypothetical protein FLJ10359 |
| | /DB_XREF = gi: 8922377 /UG = Hs.285861 hypothetical protein FLJ10359 /FL = gb: NM_018072.1 |
| 213128_s_at | Consensus includes gb: AA527499 /FEA = EST /DB_XREF = gi: 2269568 /DB_XREF = est: ng41f07.s1 /CLONE = IMAGE: 937381 /UG = Hs.301667 Homo sapiens mRNA; cDN |
| | DKFZp566I043 (from clone DKFZp566I043) |
| 207719_x_at | gb: NM_014812.1 /DEF = Homo sapiens KIAA0470 gene product (KIAA0470), mRNA. /FEA = mRNA /GEN = KIAA0470 /PROD = KARP-1-binding protein |
| | /DB_XREF = gi: 7662141 /UG = Hs.25132 KIAA0470 gene product /FL = gb: AB022657.1 gb: NM_014812.1 |
| 200060_s_at | gb: BC001659.1 /DEF = Homo sapiens, RNA-binding protein S1, serine-rich domain, clone MGC: 1125, mRNA, complete cds. /FEA = mRNA |
| | /PROD = RNA-binding protein S1, serine-rich domain /DB_XREF = gi: 12804496 /UG = Hs.75104 RNA-binding protein S1, serine-rich domain |
| | /FL = gb: BC001659.1 gb: BC001838.1 |
| 200634_at | gb: NM-005022.1 /DEF = Homo sapiens profilin 1 (PFN1), mRNA. /FEA = mRNA /GEN = PFN1 /PROD = profilin 1 /DB_XREF = gi: 4826897 /UG = Hs.75721 profilin 1 |
| | /FL = gb: BC002475.1 gb: J03191.1 gb: NM_005022.1 |
| 202770_s_at | gb: NM_004354.1 /DEF = Homo sapiens cyclin G2 (CCNG2), mRNA. /FEA = mRNA /GEN = CCNG2 /PROD = cyclin G2 /DB_XREF = gi: 4757935 /UG = Hs.79069 cyclin G2 |
| | /FL = gb: U47414.1 gb: NM_004354.1 |
| 201008_s_at | Consensus includes gb: AA812232 /FEA = EST /DB_XREF = gi: 288I843 /DB_XREF = est: ob84h09.s1 /CLONE = IMAGE: 1338113 /UG = Hs.179526 upregulated by |
| | 1,25-dihydroxyvitamin D-3 /FL = gb: NM_006472.1 gb: S73591.1 |
| 218534_s_at | gb: NM_018046.1 /DEF = Homo sapiens hypothetical protein FLJ10253 (FLJ10283), mRNA. /FEA = mRNA /GEN = FLJ10283 /PROD = hypothetical protein FLJ10283 |
| | /DB_XREF = gi: 8922325 /UG = Hs.284216 hypothetical protein FLJ10283 /FL = gb: NM_018046.1 |
| 200993_at | Consensus includes gb: AA939270 /FEA = EST /DB_XREF = gi: 3099183 /DB_XREF = est: oq31b02.s1 /CLONE = IMAGE: 1587915 /UG = Hs.5151 RAN binding protein 7 |
| | /FL = gb: AF098799.1 gb: NM_006391.1 |
| 214102_at | Consensus includes gb: AK023737.1 /DEF = Homo sapiens cDNA FLJ13675 fis, clone PLACE1011875, highly similar to Homo sapiens mRNA for KIAA0580 |
| | protein. /FEA = mRNA /DB_XREF = gi: 10435758 /UG = Hs.287588 Homo sapiens cDNA FLJ13675 fis, clone PLACE1011875, highly similar to Homo sapiens |
| | mRNA for KIAA0580 protein |
| 213704_at | Consensus include gb: AA129753 /FEA = EST /DB_XREF = gi: 1690163 /DB_XREF = est: zl16a12.s1 /CLONE = IMAGE: 502078 /UG = Hs.78948 Rab geranylgeranyltransferase, |
| | beta subunit |
| 212367_at | Consensus includes gb: AI799061 /FEA = EST /DB_XREF = gi: 5364533 /DB_XREF = est: we98a10.x1 /CLONE = IMAGE: 2349114 /UG = Hs.6048 FEM-1 (C. elegans) |
| | homolog b /FL = gb: AF178632.1 gb: NM_015322.1 gb: AF204883.1 |
| 201873_s_at | gb: NM_002940.1 /DEF = Homo sapiens ATP-binding cassette, sub-family E (OABP), member 1 (ABCE1), mRNA. /FEA = mRNA /GEN = ABCE1 /PROD = ATP-binding |
| | cassette, sub-family E, member 1 /DB_XREF = gi: 4506558 /UG = Hs.12013 ATP-binding cassette, sub-family E (OABP), member 1 /FL = gb: NM_002940.1 |
| 218618_s_at | gb: NM_022763.1 /DEF = Homo sapiens hypothetical protein FLJ23399 (FLJ23399), mRNA. /FEA = mRNA /GEN = FLJ23399 /PROD = hypothetical protein FLJ23399 |
| | /DB_XREF = gi: 12232434 /UG = Hs.299883 hypothetical protein FLJ23399 /FL = gb: NM_022763.1 |
| 203689_s_at | Consensus includes gb: AI743037 /FEA = EST /DB_XREF = gi: 5111325 /DB_XREF = est: wg85d05.x1 /CLONE = IMAGE: 2371881 /UG = Hs.89764 fragile X mental |
| | retardation 1 /FL = gb: NM_002024.1 |
| 201074_at | Consensus includes gb: AA593983 /FEA = EST /DB_XREF = gi: 2409333 /DB_XRBF = est: nn16d01.s1 /CLONE = IMAGE: 1084033 /UG = Hs.172280 SWISNF related, matri |
| | associated, actin dependent regulator of chromatin, subfamily c, member 1 /FL = gb: U66615.1 gb: NM_003074.1 |
| 200778_s_at | Consensus includes gb: AI191427 /FEA = EST /DB_XREF = gi: 3742636 /DB_XREF = est: qe48g03.x1 /CLONE = IMAGE: 1742260 /UG = Hs.155595 neural precursor cell |
| | expressed, developmentally down-regulated 5 /FL = gb: D28540.1 gb: NM_004404.1 gb: D63878.1 |
| 202307_s_at | gb: NM_000593.2 /DEF = Homo sapiens ATP-binding cassette, sub-family B (MDRTAP), member 2 (ABCB2), mRNA. /FEA = mRNA /GEN = ABCB2 /PROD = ATP-bindin |
| | cassette, sub-family B, member 2 /DB_XREF = gi: 9665247 /UG = Hs.158164 ATP-binding cassette, sub-family B (MDRTAP), member 2 /FL = gb: L21204.1 |
| | gb: L21205.1 gb: L21206.1 gb: L21207.1 gb: L21208.1 gb: NM_000593.2 |
| 213145_at | Consensus includes gb: BF001666 /FEA = EST /DB_XREF = gi: 10701941 /DB_XREF = est: 7g91d12.x1 /CLONE = IMAGE: 3313847 /UG = Hs.12460 Homo sapiens clone |
| | 23870 mRNA sequence |
| 218588_s_at | gb: NM_018691.1 /DEF = Homo sapiens chromosome 5 open reading frame 3 (C5ORF3), mRNA. /FEA = mRNA /GEN = C5ORF3 /PROD = hypothetical protein |
| | /DB_XREF = gi: 8922068 /UG = Hs.166551 chromosome 5 open reading frame 3 /FL = gb: NM_018691.1 |
| 217834_s_at | gb: NM_006372.1 /DEF = Homo sapiens NS1-associated protein 1 (NSAP1), mRNA. /FEA = mRNA /GEN = NSAP1 /PROD = NS1-associated protein 1 |
| | /DB_XREF = gi: 5453805 /UG = Hs.155489 NS1-associated protein 1 /FL = gb: AF155568.1 gb: NM_006372.1 |
| 205038_at | Consensus includes gb: BG540504 /FEA = EST /DB_XREF = gi: 13532737 /DB_XREF = est: 602569230F1 /CLONE = IMAGE: 4693783 /UG = Hs.54452 zinc finger protein, |
| | subfamily 1A, 1 (Ikaros) /FL = gb: U40462.1 gb: NM_006060.1 |
| 203132_at | gb: NM_000321.1 /DEF = Homo sapiens retinoblastoma 1 (including osteosarcoma) (RB1), mRNA. /FEA = mRNA /GEN = RB1 /PROD = retinoblastoma 1 (including |
| | osteosarcoma) /DB_XREF = gi: 4506434 /UG = Hs.75770 retinoblastoma 1 (including osteosarcoma) /FL = gb: M33647.1 gb: M15400.1 gb: M28419.1 |
| | gb: NM_000321.1 |
| 211297_s_at | gb: L20320.1 /DEF = Human protein serinethreonine kinase stk1 mRNA, complete cds. /FEA = mRNA /PROD = protein serinethreonine kinase |
| | /DB_XREF = gi: 348242 /UG = Hs.184298 cyclin-dependent kinase 7 (homolog of Xenopus MO15 cdk-activating kinase) /FL = gb: L20320.1 |
| 212375_at | Consensus includes gb: AL563727 /FEA = EST /DB_XREF = gi: 12913403 /DB_XREF = est: AL563727 /CLONE = CS0DD007YL16 (3 prime) /UG = Hs.306094 trinucleotide |
| | repeat containing 12 |
| 208986_at | Consensus includes gb: AL559478 /FEA = EST /DB_XREF = gi: 12905019 /DB_XREF = est: AL559478 /CLONE = CS0DJ013YM17 (5 prime) /UG = Hs.21704 transcription |
| | factor 12 (HTF4, heliz-loop-helix transcription factors 4) /FL = gb: M80627.1 gb: M83233.1 gb: NM_003205.2 |
| 217956_s_at | gb: NM_021204.1 /DEF = Homo sapiens E-1 enzyme (MASA), mRNA. /FEA = mRNA /GEN = MASA /PROD = E-1 enzyme /DB_XREF = gi: 10864016 /UG = Hs.18442 E-1 enzyme /FL = |
| | gb: NM_021204.1 gb: AF113125.1 |
| 214870_x_at | Consensus includes gb: AC002045 /DEF = Human Chromosome 16 BAC clone CIT987SK-A-589H1 /FEA = mRNA_2 /DB_XREF = gi: 2951945 /UG = Hs.251928 nuclear |
| | pore complex interacting protein |
| 209852_x_at | gb: BC001423.1 /DEF = Homo sapiens, Similar to proteaseome (prosome, macropain) 28 subunit, 3, clone MGC: 1394, mRNA, complete cds. |
| | /FEA = mRNA /PROD = Similar to proteaseome (prosome, macropain) 28subunit, 3 /DB_XREF = gi: 12655138 /UG = Hs.152978 proteasome (prosome, macropain) |
| | activator subunit 3 (PA28 gamma; Ki) /FL = gb: BC001423.1 gb: BC002684.1 |
| 209063_x_at | Consensus includes gb: BF248165 /FEA = EST /DB_XREF = gi: 11164389 /DB_XREF = est: 601859364F1 /CLONE = IMAGE: 4069686 /UG = Hs.109643 polyadenylate |
| | binding protein-interacting protein 1 /FL = gb: AL136920.1 |
| 201177_s_at | gb: NM_005499.1 /DEF = Homo sapiens SUMO-1 activating enzyme subunit 2 (UBA2), mRNA. /FEA = mRNA /GEN = UBA2 /PROD = SUMO-1 activating enzyme |
| | subunit 2 /DB_XREF = gi: 4885648 /UG = Hs.4311 SUMO-1 activating enzyme subunit 2 /FL = gb: BC003153.1 gb: U35832.1 gb: AF090384.1 gb: AF079566.1 |
| | gb: AF110957.1 gb: NM_005499.1 gb: AL136905.1 |
| 212264_s_at | Consensus includes gb: BE645850 /FEA = EST /DB_XREF = gi: 9970161 /DB_XREF = est: 7e77c03.x1 /CLONE = IMAGE: 3288484 /UG = Hs.154978 KIAA0261 protein |
| 218878_s_at | gb: NM_012238.3 /DEF = Homo sapiens sirtuin (silent mating type information regulation 2, S. cerevisiae, homolog) 1 (SIRT1), mRNA. |
| | /FEA = mRNA /GEN = SIRT1 /PROD = sirtuin 1 /DB_XREF = gi: 13775598 /UG = Hs.31176 sirtuin (silent mating type information regulation 2, S. cerevisiae, |
| | homolog) 1 /FL = gb: NM_012238.3 gb: AF083106.2 |
| 202163_s_at | gb: NM_004779.1 /DEF = Homo sapiens CCR4-NOT transcription complex, subunit 8 (CNOT8), mRNA. /FEA = mRNA /GEN = CNOT8 /PROD = CCR4-NOT transcription |
| | complex, subunit 8 /DB_XREF = gi: 4758945 /UG = Hs.26703 CCR4-NOT transcription complex, subunit 8 /FL = gb: AF053318.1 gb: NM_004779.1 gb: AL122045.1 |
| | gb: AF180476.1 |
| 203584_at | gb: NM_014673.1 /DEF-Homo sapiens KIAA0103 gene product (KIAA0103), mRNA. /FEA = mRNA /GEN = KIAA0103 /PROD = KIAA0103 gene product |
| | /DB_XREF = gi: 7661909 /UG = Hs.154387 KIAA0103 gene product /FL = gb: D14659.1 gb: NM_014673.1 |
| 201901_s_at | Consensus includes gb: Z14077.1 /DEF = H. sapiens mRNA for YY1NF-E1 protein. /FEA = mRNA /PROD-YY1 NF-E1 /DB_XREF = gi: 38010 /UG = Hs.97496 YY1 |
| | transcription factor /FL = gb: M77698.1 gb: M76541.1 gb: NM_003403.2 |
| 213743_at | Consensus includes gb: BE674119 /FEA = EST /DB_XREF = gi: 10034660 /DB_XREF = est: 7d75b03.x1 /CLONE = IMAGE: 3278765 /UG = Hs.155478 cyclin T2 |
| 202883_s_at | Consensus includes gb: T79584 /FEA = EST /DB_XREF = gi: 698093 /DB_XREF = est: yd71e11.s1 /CLONE = IMAGE: 113660 /UG = Hs.108705 protein phosphatase 2 |
| | (formerly 2A), regulatory subunit A (PR 65), beta isoform /FL = gb: NM_002716.1 gb: AF163473.1 gb: M65254.1 gb: AF087438.1 |
| 202069_s_at | Consensus includes gb: AI826060 /FEA = EST /DB_XREF = gi: 5446731 /DB_XREF = est: wk28a12.x1 /CLONE = IMAGE: 2413630 /UG = Hs.250616 isocitrate |
| | dehydrogenase 3 (NAD+) alpha /FL = gb: NM_005530.1 gb: U07681.1 |
| 222303_at | Consensus includes gb: AV700891 /FEA = EST /DB_XREF = gi: 10302862 /DB_XREF = est: AV700891 /CLONE = GKCBQD03 /UG = Hs.292477 ESTs |
| 203102_s_at | gb: NM_002408.2 /DEF = Homo sapiens mannosyl (alpha-1,6-)-glycoprotein beta-1,2-N-acetylglucosaminyltransferase (MGAT2), mRNA. /FEA = mRNA |
| | /GEN = MGAT2 /PROD = alpha-1,6-mannosyl-glycoproteinbeta-1,2-N-acetylglucosaminyltransferase /DB_XREF = gi: 6031183 /UG = Hs.172195 mannosyl |
| | (alpha-1,6-)-glycoprotein beta-1,2-N-acetylglucosaminyltransferase /FL = gb: NM_002408.2 |
| 212982_at | Consensus includes gb: AI621223 /FEA = EST /DB_XREF = gi: 4630349 /DB_XREF = est: ts77a09.x1 /CLONE = IMAGE: 2237272 /UG = Hs.4014 KIAA0946 protein; |
| | Huntingtin interacting protein H |
| 200050_at | gb: NM_007145.1 /DEF = Homo sapiens zinc finger protein 146 (ZNF146), mRNA. /FEA = mRNA /GEN = ZNF146 /PROD = zinc finger protein 146 |
| | /DB_XREF = gi: 6005965 /UG = Hs.301819 zinc finger protein 146 /FL = gb: BC005154.1 gb: NM_007145.1 |
| 202430_s_at | gb: NM_021105.1 /DEF = Homo sapiens phospholipid scramblase 1 (PLSCR1), mRNA. /FEA = mRNA /GEN = PLSCR1 /PROD = phospholipid scramsblase 1 |
| | /DB_XREF = gi: 10863876 /UG = Hs.198282 phospholipid scramblase 1 /FL = gb: NM_021105.1 gb: AB006746.1 gb: AF098642.1 |
| 218757_s_at | gb: NM_023010.1 /DEF = Homo sapiens similar to yeast Upf3, variant B (UPF3B), mRNA. /FEA = mRNA /GEN = UPF3B /PROD = similar to yeast Upf3, |
| | variant B /DB_XREF = gi: 12711673 /UG = Hs.103832 similar to yeast Upf3, variant B /FL = gb: AY013251.1 gb: NM_023010.1 |
| 214030_at | Consensus includes gb: BE501352 /FEA = EST /DB_XREF = gi: 9703760 /DB_XREF = est: 7a41e05.x1 /CLONE = IMAGE: 3221312 /UG = Hs.23294 ESTs, Weakly similar |
| | to T15138 hypothetical protein T28F2.4 —Caenorhabditis elegans C. elegans |
| 218093_s_at | gb: NM_017664.1 /DEF = Homo sapiens hypothetical protein FLJ20093 (FLJ20093), mRNA. /FEA = mRNA /GEN = FLJ20093 /PROD = hypothetical protein FLJ20093 |
| | /DB_XREF = gi: 8923103 /UG = Hs.172572 hypothetical protein FLJ20093 /FL = gb: NM_017664.1 |
| 203486_s_at | Consensus includes gb: BF195973 /FEA = EST /DB_XREF = gi: 11083411 /DB_XREF = est: 7o88c12.x1 /CLONE = IMAGE: 3643391 /UG = Hs.102708 DKFZP434A043 |
| | protein /FL = gb: NM_015396.1 |
| 219303_at | gb: NM_024546.1 /DEF = Homo sapiens hypothetical protein FLJ13449 (FLJ13449), mRNA. /FEA = mRNA /GEN = FLJ13449 /PROD = hypothetical protein |
| | FLJ13449 /DB_XREF = gi: 13375708 /UG = Hs.10711 hypothetical protein FLJ13449 /FL = gb: AL136651.1 gb: NM_024546.1 |
| 202173_s_at | gb: NM_007146.1 /DEF = Homo sapiens zinc finger protein 161 (ZNF161), mRNA. /FEA = mRNA /GEN = ZNF161 /PROD = zinc finger protein 161 |
| | /DB_XREF = gi: 6005967 /UG = Hs.6557 zinc finger protein 161 /FL = gb: D28118.1 gb: NM_007146.1 |
| 216903_s_at | Consensus includes gb: AK022697.1 /DEF = Homo sapiens cDNA FLJ12635 fis, clone NT2RM4001865, highly similar to Homo sapiens mRNA for atopy |
| | related autoantigen CALC. /FEA = mRNA /DB_XREF = gi: 10434244 /UG = Hs.61628 calcium binding atopy-related autoantigen 1 |
| 205842_s_at | gb: AF001362.1 /DEF-Homo sapiens Jak2 kinase (JAK2) mRNA, complete cds. /FEA = mRNA /GEN = JAK2 /PROD-Jak2 kinase /DB_XREF = gi: 3236321 |
| | /UG = Hs.115541 Janus kinase 2 (a protein tyrosine kinase) /FL = gb: NM_004972.2 gb: AF005216.1 gb: AF058925.1 gb: AF001362.1 |
| 212702_s_at | Consensus includes gb: N45111 /FEA = EST /DB_XREF = gi: 1186277 /DB_XREF = est: yz12f12.s1 /CLONE = IMAGE: 282863 /UG = Hs.330988 Homo sapiens, Similar to |
| | Bicaudal D (Drosophila) homolog 1, clone IMAGE: 3622452, mRNA, partial cds |
| 201664_at | gb: AL136877.1 /DEF = Homo sapiens mRNA; cDNA DKFZp434F205 (from clone DKFZp434F205); complete cds. /FEA = mRNA /GEN = DKFZp434E205 |
| | /PROD = hypothetical protein /DB_XREF = gi: 6807670 /UG = Hs.50758 SMC4 (structural maintenance of chromosomes 4, yeast)-like 1 /FL = gb: AB019987.1 |
| | gb: NM_005496.1 gb: AL136877.1 |
| 202060_at | gb: NM_014633.1 /DEF = Homo sapiens KIAA0155 gene product (KIAA0155), mRNA. /FEA = mRNA /GEN = KIAA0155 /PROD = KIAA0155 gene product |
| | /DB_XREF = gi: 7661949 /UG = Hs.173288 KIAA0155 gene product /FL = gb: NM_014633.1 gb: D63875.1 |
| 203177_x_at | gb: NM_003201.1 /DEF-Homo sapiens transcription factor 6-like 1 (mitochondrial transcription factor 1-like) (TCF6L1), mRNA. |
| | /FEA = mRNA /GEN-TCF6L1 /PROD = transcription factor 6-like 1 (mitochondrialtranscription factor 1-like) /DB_XREF = gi: 4507400 |
| | /UG = Hs.75133 transcription factor 6-like 1 (mitochondrial transcription factor 1-like) /FL = gb: M62810.1 gb: NM_003201.1 |
| 209748_at | gb: AB029006.1 /DEF = Homo sapiens mRNA for KIAA1083 protein, complete cds. /FEA = mRNA /GEN = KIAA1083 /PROD = KIAA1083 protein /DB_XREF = gi: 5689502 |
| | /UG = Hs.26334 spastic paraplegia 4 (autosomal dominant; spastin) /FL = gb: AB029006.1 |
| 200660_at | gb: NM_005620.1 /DEF = Homo sapiens S100 calcium-binding protein A11 (calgizzarin) (S100A11), mRNA. /FEA = mRNA /GEN = S100A11 /PROD = S100 |
| | calcium-binding protein A11 /DB_XREF = gi: 5032056 /UG = Hs.256290 S100 calcium-binding protein A11 (calgizzarin) /FL = gb: D49355.1 |
| | gb: BC001410.1 gb: D50374.1 gb: NM_005620.1 gb: D38583.1 |
| 222119_s_at | Consensus includes gb: AL117620.1 /DEF = Homo sapiens mRNA; cDNA DKFZp564K2364 (from clone DKFZp564K2364); partial cds. /FEA = mRNA |
| | /GEN = DKFZp564K2364 /PROD = hypothetical protein /DB_XREF = gi: 5912200 /UG = Hs.284289 vitiligo-associated protein VIT-1 |
| 208934_s_at | gb: AF342815.1 /DEF = Homo sapiens colorectal carcinoma-derived galectin-8 variant I mRNA, complete cds. /FEA = mRNA /PROD = colorectal |
| | carcinoma-derived galectin-8 variantI /DB_XREF = gi: 13249298 /UG = Hs.4082 lectin, galactoside-binding, soluble, 8 (galectin 8) |
| | /FL = gb: AF342815.1 gb: L78132.1 gb: AF074000.1 gb: NM_006499.1 |
| 211698_at | gb: AF349444.1 /DEF = Homo sapiens Rb- and p300-binding protein EID-1 mRNA, complete cds. /FEA = mRNA /PROD = Rb- and p300-binding protein EID-1 |
| | /DB_XREF = gi: 13549113 /FL = gb: AF349444.1 |
| 212053_at | Consensus includes gb: AK025504.1 /DEF = Homo sapiens cDNA: FLJ21851 fis, clone HEP01962. /FEA = mRNA /DB_XREF = gi: 10438041 /UG = Hs.170218 KIAA0251 |
| | protein |
| 209174_s_at | gb: BC000978.2 /DEF = Homo sapiens, hypothetical protein FLJ20259, clone MGC: 5396, mRNA, complete cds. /FEA = mRNA /PROD = hypothetical protein |
| | FLJ20259 /DB_XREF = gi: 12803027 /UG = Hs.9956 hypothetical protein FLJ20259 /FL = gb: BC000978.2 gb: NM_017730.1 |
| 203476_at | gb: NM_006670.1 /DEF = Homo sapiens 5T4 oncofetal trophoblast glycoprotein (5T4), mRNA. /FEA = mRNA /GEN = 5T4 /PROD = 5T4 oncofetal trophoblast |
| | glycoprotein /DB_XREF = gi: 5729717 /UG = Hs.82128 5T4 oncofetal trophoblast glycoprotein /FL = gb: NM_006670.1 |
| 203007_x_at | gb: AF077198.1 /DEF = Homo sapiens lysophospholipase mRNA, complete cds. /FEA = mRNA /PROD = lysophospholipase /DB_XREF = gi: 4679009 /UG = Hs.12540 |
| | lysophospholipase I /FL = gb: AF081281.1 gb: AF077198.1 gb: NM_006330.1 gb: AF291053.1 |
| 212721_at | Consensus includes gb: AI810380 /FEA = EST /DB_XREF = gi: 5396946 /DB_XREF = est: wb87h10.x1 /CLONE = IMAGE: 2312707 /UG = Hs.47274 Homo sapiens mRNA; cDN |
| | DKFZp564B176 (from clone DKFZp564B176) |
| 218461_at | gb: NM_016301.1 /DEF = Homo sapiens protein x 0004 (LOC51184), mRNA. /FEA = mRNA /GEN = LOC51184 /PROD = protein x 0004 /DB_XREF = gi: 9994188 |
| | /UG = Hs.284164 |
| | protein x 0004 /FL = gb: AF117229.1 gb: NM_016301.1 |
| 214218_s_at | Consensus includes gb: AV699347 /FEA = EST /DB_XREF = gi: 10301318 /DB_XREF = est: AV699347 /CLONE = GKCBPH07 /UG = Hs.83623 nuclear receptor subfamily 1 |
| | group I, member 3 |
| 218178_s_at | gb: NM_020412.1 /DEF = Homo sapiens CHMP1.5 protein (CHMP1.5), mRNA. /FEA = mRNA /GEN = CHMP1.5 /PROD = CHMP1.5 protein /DB_XREF = gi: 9966900 |
| | /UG = Hs.42733 |
| | CHMP1.5 protein /FL = gb: AF281064.1 gb: NM_020412.1 |
| 217851_s_at | gb: NM_016045.1 /DEF = Homo sapiens CGI-107 protein (LOC51012), mRNA. /FEA = mRNA /GEN = LOC51012 /PROD = CGI-107 protein /DB_XREF = gi: 7705609 |
| | /UG = Hs.3945 CGI-107 protein /FL = gb: AF151865.1 gb: NM_016045.1 |
| 202329_at | gb: NM_004383.1 /DEF = Homo sapiens c-src tyrosine kinase (CSK), mRNA. /FEA = mRNA /GEN = CSK /PROD = c-src tyrosine kinase /DB_XREF = gi: 4758077 |
| | /UG = Hs.77793 c-src tyrosine kinase /FL = gb: NM_004383.1 |
| 203302_at | gb: NM_000788.1 /DEF = Homo sapiens deoxycytidine kinase (DCK), mRNA. /FEA = mRNA /GEN = DCK /PROD = deoxycytidine kinase /DB_XREF = gi: 4503268 |
| | /UG = Hs.709 deoxycytidine kinase /FL = gb: M60527.1 gb: NM_000788.1 |
| 201949_x_at | Consensus includes gb: AL572341 /FEA = EST /DB_XREF = gi: 12930514 /DB_XREF = est: AL572341 /CLONE = CS0DI007YC13 (3 prime) /UG = Hs.76368 capping protei |
| | (actin filament) muscle Z-line, beta /FL = gb: NM_004930.1 gb: U03271.1 |
| 218096_at | gb: NM_018361.1 /DEF = Homo sapiens hypothetical protein FLJ11210 (FLJ11210), mRNA. /FEA = mRNA /GEN = FLJ11210 /PROD = hypothetical protein |
| | FLJ11210 /DB_XREF = gi: 8922941 /UG = Hs.27842 hypothetical protein FLJ11210 /FL = gb: NM_018361.1 |
| 203255_at | gb: NM_018693.1 /DEF = Homo sapiens vitiligo-associated protein VIT-1 (VIT1), mRNA. /FEA = mRNA /GEN = VIT1 /PROD = vitiligo-associated protein VIT-1 |
| | /DB_XREF = gi: 10048403 /UG = Hs.284289 vitiligo-associated protein VIT-1 /FL = gb: NM_018693.1 gb: AF264714.1 |
| 203855_at | gb: NM_014969.1 /DEF = Homo sapiens KIAA0893 protein (KIAA0893), mRNA. /FEA = mRNA /GEN = KIAA0893 /PROD = KIAA0893 protein /DB_XREF = gi: 7662363 |
| | /UG = Hs.3830 KIAA0893 protein /FL = gb: AB020700.1 gb: NM_014969.1 |
| 218846_at | gb: NM_004830.1 /DEF = Homo sapiens cofactor required for Sp1 transcriptional activation, subunit 3 (130 kD) (CRSP3), mRNA. /FEA = mRNA /GEN = CRSP |
| | /PROD = cofactor required for Sp1 transcriptionalactivation, subunit 3 (130 kD) /DB_XREF = gi: 7019352 /UG = Hs.29679 cofactor required for Sp1 |
| | transcriptional activation, subunit 3 (130 kD) /FL = gb: AF135022.1 gb: AF105332.1 gb: NM_004830.1 |
| 208988_at | Consensus includes gb: BE675843 /FEA = EST /DB_XREF = gi: 10036384 /DB_XREF = est: 7f17b04.x1 /CLONE = IMAGE: 3294895 /UG = Hs.219614 f-box and leucine- |
| | rich repeat protein 11 /FL = gb: AF179221.1 |
| 216202_s_at | Consensus includes gb: U15555.1 /DEF = Human serine palmitoyltransferase (LCB2) mRNA, partial cds. /FEA = mRNA /GEN = LCB2 /PROD = serine |
| | palmitoyltransferase /DB_XREF = gi: 1001944 /UG = Hs.59403 serine palmitoyltransferase, long chain base subunit 2 |
| 216954_x_at | Consensus includes gb: S77356.1 /DEF = Homo sapiens oligomycin sensitivity conferral protein oscp-like protein mRNA, partial cds. /FEA = mRNA |
| | /PROD = oligomycin sensitivity conferral proteinoscp-like protein /DB_XREF = gi: 944964 /UG = Hs.76572 ATP synthase, H+ transporting, mitochondrial |
| | F1 complex, 0 subunit (oligomycin sensitivity conferring protein) |
| 212753_at | Consensus includes gb: AI692203 /FEA = EST /DB_XREF = gi: 4969543 /DB_XREF = est: wd37h12.x1 /CLONE = IMAGE: 2330375 /UG = Hs.8834 ring finger protein 3 |
| 207691_x_at | gb: NM_001776.1 /DEF = Homo sapiens ectonucleoside triphosphate diphosphohydrolase 1 (ENTPD1), mRNA. /FEA = mRNA /GEN = ENTPD1 /PROD = ectonucleosid |
| | triphosphate diphosphohydrolase1 /DB_XREF = gi: 4502666 /UG = Hs.205353 ectonucleoside triphosphate diphosphohydrolase 1 /FL = gb: NM_001776.1 |
| 200967_at | gb: NM_000942.1 /DEF = Homo sapiens peptidylprolyl isomerase B (cyclophilin B) (PPIB), mRNA. /FEA = mRNA /GEN = PPIB /PROD = peptidylprolyl |
| | isomerase B (cyclophilin B) /DB_XREF = gi: 4758949 /UG = Hs.699 peptidylprolyl isomerase B (cyclophilin B) /FL = gb: BC001125.1 gb: M60857.1 |
| | gb: M63573.1 gb: NM_000942.1 |
| 201988_s_at | Consensus includes gb: BF438056 /FEA = EST /DB_XREF = gi: 11450573 /DB_XREF = est: 7q66e01.x1 /CLONE = IMAGE: 3703369 /UG = Hs.13313 cAMP responsive element |
| | binding protein-like 2 /FL = gb: AF039081.1 gb: NM_001310.1 |
| 202142_at | gb: BC003090.1 /DEF = Homo sapiens, COP9 homolog, clone MGC: 1297, mRNA, complete cds. /FEA = mRNA /PROD = COP9 homolog /DB_XREF = gi: 13111846 |
| | /UG = Hs.75193 COP9 homolog /FL = gb: BC003090.1 gb: U51205.1 gb: NM_006710.1 |
| 218238_at | gb: NM_012341.1 /DEF = Homo sapiens GTP-binding protein (NGB), mRNA. /FEA = mRNA /GEN = NGB /PROD = GTP-binding protein /DB_XREF = gi: 6912531 |
| | /UG = Hs.215766 GTP-binding protein /FL = gb: AF325353.1 gb: AF120334.1 gb: NM_012341.1 |
| 205552_s_at | gb: NM_002534.1 /DEF = Homo sapiens 2,5-oligoadenylate synthetase 1 (40-46 kD) (OAS1), transcript variant E16, mRNA. /FEA = mRNA /GEN = OAS1 |
| | /PROD = 2,5-oligoadenylate synthetase 1, isoform E16 /DB_XREF = gi: 8051622 /UG = Hs.82396 2,5-oligoadenylate synthetase 1 (40-46 kD) |
| | /FL = gb: BC000562.1 |
| | gb: D00068.1 gb: NM_002534.1 |
| 202541_at | Consensus includes gb: BF589679 /FEA = EST /DB_XREF = gi: 11682003 /DB_XREF = est: naa08b05.x1 /CLONE = IMAGE: 3253977 /UG = Hs.146401 small inducible |
| | cytokine subfamily ε, member 1 (endothelial monocyte-activating) /FL = gb: NM_004757.1 gb: U10117.1 |
| 221699_s_at | gb: AF334103.1 /DEF = Homo sapiens nucleolar protein GU2 mRNA, complete cds. /FEA = mRNA /PROD = nucleolar protein GU2 /DB_XREF = gi: 13540303 |
| | /FL = gb: AF334103.1 |
| 202090_s_at | gb: NM_006830.1 /DEF = Homo sapiens ubiquinol-cytochrome c reductase (6.4 kD), subunit (UQCR), mRNA. /FEA = mRNA /GEN = UQCR |
| | /PROD = ubiquinol-cytochrome c reductase (6.4 kD)subunit /DB_XREF = gi: 5803216 /UG = Hs.8372 ubiquinol-cytochrome c reductase (6.4 kD) subunit |
| | /FL = gb: D55636.1 gb: NM_006830.1 |
| 202382_s_at | gb: NM_005471.1 /DEF = Homo sapiens glucosamine-6-phosphate isomerase (GNPI), mRNA. /FEA = mRNA /GEN = GNPI /PROD = glucosamine-6-phosphate |
| | isomerase /DB_XREF = gi: 13027377 /UG = Hs.278500 glucosamine-6-phosphate isomerase /FL = gb: NM_005471.1 gb: AF029914.1 gb: AF048826.1 gb: D31766.1 |
| 220408_x_at | gb: NM_017569.1 /DEF = Homo sapiens transcription factor (p38 interacting protein) (P38IP), mRNA. /FEA = mRNA /GEN = P38IP /PROD = transcription |
| | factor (p38 interacting protein) /DB_XREF = gi: 8923734 /UG = Hs.171185 transcription factor (p38 interacting protein) /FL = gb: AF093250.1 |
| | gb: NM_017569.1 |
| 208264_s_at | gb: NM_003758.1 /DEF = Homo sapiens eukaryotic translation initiation factor 3, subunit 1 (alpha, 35 kD) (EIF3S1), mRNA. /FEA = mRNA /GEN = EIF3S1 |
| | /PROD = eukaryotic translation initiation factor 3, subunit 1 (alpha, 35 kD) /DB_XREF = gi: 4503510 /UG = Hs.173987 eukaryotic translation initiation |
| | factor 3, subunit 1 (alpha, 35 kD) /FL = gb: U97670.1 gb: NM_003758.1 |
| 202872_at | Consensus includes gb: AW024925 /FEA = EST /DB_XREF = gi: 5878455 /DB_XREF = est: wu92g09.x1 /CLONE = IMAGE: 2527552 /UG = Hs.86905 ATPase, H+ transporting, |
| | lysosomal (vacuolar proton pump) 42 kD /FL = gb: NM_001695.1 |
| 214835_s_at | Consensus includes gb: AF131748.1 /DEF = Homo sapiens clone 25191 GTP-specific succinyl-CoA synthetase beta subunit (SCS) mRNA sequence, |
| | partial cds. /FEA = mRNA /PROD = GTP-specific succinyl-CoA synthetase betasubunit /DB_XREF = gi: 4406563 /UG = Hs.247309 succinate-CoA ligase, |
| | GDP-forming, beta subunit |
| 205263_at | gb: AF082283.1 /DEF = Homo sapiens CARD-containing apoptotic signaling protein (BCL10) mRNA, complete cds. /FEA = mRNA /GEN = BCL10 |
| | /PROD = CARD-containing apoptotic signaling protein /DB_XREF = gi: 4092066 /UG = Hs.193516 B-cell CLLlymphoma 10 /FL = gb: AF082283.1 |
| | gb: AF057700.1 gb: AF100338.1 gb: NM_003921.1 gb: AF127386.1 gb: AF134395.1 gb: AF105066.1 |
| 218512_at | gb: NM_018256.1 /DEF = Homo sapiens hypothetical protein FLJ10881 (FLJ10881), mRNA. /FEA = mRNA /GEN = FLJ10881 /PROD = hypothetical protein |
| | FLJ10881 /DB_XREF = gi: 8922736 /UG = Hs.73291 hypothetical protein FLJ10881 /FL = gb: AF242546.1 gb: NM_018256.1 |
| 210283_x_at | gb: BC005295.1 /DEF = Homo sapiens, Similar to polyadenylate binding protein-interacting protein 1, clone MGC: 12360, mRNA, complete cds. |
| | /FEA = mRNA /PROD = Similar to polyadenylate bindingprotein-interacting protein 1 /DB_XREF = gi: 13529010 /UG = Hs.109643 polyadenylate binding |
| | protein-interacting protein 1 /FL = gb: BC005295.1 |
| 202724_s_at | gb: NM_002015.2 /DEF = Homo sapiens forkhead box O1A (rhabdomyosarcoma) (FOXO1A), mRNA. /FEA = mRNA /GEN = FOXO1A /PROD = forkhead box O1A |
| | /DB_XREF = gi: 9257221 /UG = Hs.170133 forkhead box O1A (rhabdomyosarcoma) /FL = gb: AF032885.1 gb: U02310.1 gb: NM_002015.2 |
| 212058_at | Consensus includes gb: AI184562 /FEA = EST /DB_XREF = gi: 3735200 /DB_XREF = est: qd60b05.x1 /CLONE = IMAGE: 1733841 /UG = Hs.7976 KIAA0332 protein |
| 212006_at | Consenes includes gb: AU149908 /FEA = EST /DB_XREF = gi: 11011429 /DB_XREF = est: AU149908 /CLONE = NT2RP2000896 /UG = Hs.77495 UBX |
| | domain-containing 1 |
| 222040_at | Consensus includes gb: AI144007 /FEA = EST /DB_XREF = gi: 3665816 /DB_XREF = est: qb33c06.x1 /CLONE = IMAGE: 1698058 /UG = Hs.249495 |
| | heterogeneous nuclear ribonucleoprotein A1 |
| 202373_s_at | gb: AF255648.1 /DEF = Homo sapiens rGAP-iso mRNA, complete cds. /FEA = mRNA /PROD = rGAP-iso /DB_XREF = gi: 12005820 /UG = Hs.197289 rab3 |
| | GTPase-activating protein, non-catalytic subunit (150 kD) /FL = gb: AF255648.1 gb: AF004828.1 gb: NM_012414.1 |
| 209301_at | gb: M36532.1 /DEF = Human carbonic anhydrase II mRNA, complete cds. /FEA = mRNA /GEN = CA2 /DB_XREF = gi: 179194 /UG = Hs.155097 carbonic anhydrase II |
| | /FL = gb: J03037.1 gb: M36532.1 gb: NM_000067.1 |
| 213264_at | Consensus includes gb: AW025150 /FEA = EST /DB_XREF = gi: 5878680 /DB_XREF = est: wu94e06.x1 /CLONE = IMAGE: 2527714 /UG = Hs.211601 mitogen-activated |
| | protein kinase kinase kinase 12 |
| 202863_at | gb: NM_003113.1 /DEF = Homo sapiens nuclear antigen Sp100 (SP100), mRNA. /FEA = mRNA /GEN = SP100 /PROD = nuclear antigen Sp100 /DB_XREF = gi: 4507164 |
| | /UG = Hs.77617 nuclear antigen Sp100 /FL = gb: M60618.1 gb: NM_003113.1 |
| 217679_x_at | Consensus includes gb: AI683552 /FEA = EST /DB_XREF = gi: 4893734 /DB_XREF = est: tx67h02.x1 /CLONE = IMAGE: 2274675 /UG = Hs.201605 ESTs, Moderately |
| | similar to ALU8 HUMAN ALU SUBFAMILY SX SEQUENCE CONTAMINATION WARNING ENTRY H. sapiens |
| 212802_s_at | Consensus includes gb: AK023841.1 /DEF = Homo sapiens cDNA FLJ13779 fis, clone PLACE4000445, highly similar to Homo sapiens mRNA; cDNA |
| | DKFZp434C212 (from clone DKFZp434C212). /FEA = mRNA /DB_XREF = gi: 10435900 /UG = Hs.172069 DKFZP434C212 protein |
| 210346_s_at | gb: AF212224.1 /DEF = Homo sapiens CLK4 mRNA, complete cds. /FEA = mRNA /PROD = CLK4 /DB_XREF = gi: 9437514 /UG = Hs.295231 Homo sapiens CLK4 mRNA, |
| | complete cds /FL = gb: AF212224.1 |
| 208848_at | gb: M30471.1 /DEF = Human class III alcohol dehydrogenase (ADH5) chi subunit mRNA, complete cds. /FEA = mRNA /GEN = ADH5 /PROD = alcohol |
| | dehydrogenase 3 |
| | /DB_XREF = gi: 178133 /UG = Hs.78989 alcohol dehydrogenase 5 (class III), chi polypeptide /FL = gb: NM_000671.2 gb: M29872.1 gb: M30471.1 |
| 221978_at | Consensus includes gb: BE138825 /FEA = EST /DB_XREF = gi: 8601325 /DB_XREF = est: xw96a07.x1 /CLONE = IMAGE: 2835828 /UG = Hs.110309 major histocompatibility |
| | complex, class I, F |
| 217526_at | Consensus includes gb: AI478300 /FEA = EST /DB_XREF = gi: 4371526 /DB_XREF = est: tm39e01.x1 /CLONE = IMAGE: 2160504 /UG = Hs.192789 ESTs, Weakly similar |
| | to ALU6 HUMAN ALU SUBFAMILY SP SEQUENCE CONTAMINATION WARNING ENTRY H. sapiens |
| 218322_s_at | gb: NM_016234.2 /DEF = Homo sapiens long-chain fatty acid coenzyme A ligase 5 (FACL5), mRNA. /FEA = mRNA /GEN = FACL5 /PROD = long-chain fatty acid |
| | coenzyme A ligase 5 /DB_XREF = gi: 12669912 /UG = Hs.11638 long-chain fatty acid coenzyme A ligase 5 /FL = gb: NM_016234.2 gb: AB033899.1 |
| 212613_at | Consensus includes gb: AI991252 /FEA = EST /DB_XREF = gi: 5838157 /DB_XREF = est: wu41e09.x1 /CLONE = IMAGE: 2522632 /UG = Hs.87497 butyrophilin, |
| | subfamily 3, member A2 |
| 204566_at | gb: NM_003620.1 /DEF = Homo sapiens protein phosphatase 1D magnesium-dependent, delta isoform (PPM1D), mRNA. /FEA = mRNA /GEN = PPMID |
| | /PROD = protein phosphatase 1D magnesium-dependent, delta isoform /DB_XREF = gi: 4505996 /UG = Hs.100980 protein phosphatase 1D magnesium-dependent, |
| | delta isoform /FL = gb: U78305.1 gb: NM_003620.1 |

TABLE 6
Genes from Cluster Analysis 6

| Affymetrix | |
|---|---|
| internal | |
| designation | Description of the sequence in the GeneBank data base |
| 202018_s_at | gb: NM_002343.1 /DEF = Homo sapiens lactotransferrin (LTF), mRNA. /FEA = mRNA /GEN = LTF /PROD = lactotransferrin /DB_XREF = gi: 4505042 |
| | /UG = Hs.105938 lactotransferrin /FL = gb: AF332168.1 gb: M93150.1 gb: M83202.1 gb: NM_002343.1 |
| 216379_x_at | Consensus includes gb: AK000168.1 /DEF = Homo sapiens cDNA FLJ20161 fis, clone COL09252, highly similar to L33930 |
| | Homo sapiens CD24 signal transducer mRNA. /FEA = mRNA /DB_XREF = gi: 7020079 /UG = Hs.332045 Homo sapiens cDNA FLJ20161 fis, |
| | clone COL09252, highly similar to L33930 Homo sapiens CD24 signal transducer mRNA |
| 209771_x_at | Consensus includes gb: AA761181 /FEA = EST /DB_XREF = gi: 2810111 /DB_XREF = est: nz09g03.s1 /CLONE = IMAGE: 1287316 |
| | /UG = Hs.286124 CD24 antigen (small cell lung carcinoma cluster 4 antigen) /FL = gb: X69397.1 |
| 206676_at | gb: M33326.1 /DEF = Human nonspecific cross-reacting antigen (NCA) mRNA, complete cds. |
| | /FEA = mRNA /GEN = NCA /PROD = non-specific cross reacting antigen /B_XREF = gi: 189101 |
| | /UG = Hs.41 carcinoembryonic antigen-related cell adhesion molecule 8 /FL = gb: M33326.1 gb: NM_001816.1 |
| 210254_at | gb: L35848.1 /DEF = Homo sapiens IgE receptor beta chain (HTm4) mRNA, complete cds. /FEA = mRNA /GEN = HTm4 /PROD = IgE receptor beta |
| | subunit /DB_XREF = gi: 561638 /UG = Hs.99960 membrane-spanning 4-domains, subfamily A, member 3 (hematopoietic cell-specific) |
| | /FL = gb: NM_006138.1 gb: L35848.1 |
| 203887_s_at | gb: NM_000361.1 /DEF = Homo sapiens thrombomodulin (THBD), mRNA. /FEA = mRNA /GEN = THBD /PROD = thrombomodulin /DB_XREF = gi: 4507482 |
| | /UG = Hs.2030 thrombomodulin /FL = gb: M16552.1 gb: NM_000361.1 |
| 212768_s_at | Consensus includes gb: AL390736 /DEF = Human DNA sequence from clone RP11-209J19 on chromosome 13 Contains ESTs, STSs and GSSs. |
| | Contains the gene for the GW112 protein with two isoforms (GW112 and KIAA4294) /FEA = mRNA /DB_XREF = gi: 11182238 /UG = Hs.273321 |
| | differentially expressed in hematopoietic lineages |
| 202887_s_at | gb: NM_019058.1 /DEF = Homo sapiens hypothetical protein (FLJ20500), mRNA. /FEA = mRNA /GEN = FLJ20500 /PROD = hypothetical protein |
| | /DB_XREF = gi: 9506686 /UG = Hs.111244 hypothetical protein /FL = gb: AL136668.1 gb: NM_019058.1 |
| 200916_at | gb: NM_003564.1 /DEF = Homo sapiens transgelin 2 (TAGLN2), mRNA. /FEA = mRNA /GEN = TAGLN2 /PROD = transgelin 2 /DB_XREF = gi: 4507356 |
| | /UG = Hs.75725 transgelin 2 /FL = gb: D21261.1 gb: NM_003564.1 |
| 206157_at | gb: NM_002852.1 /DEF = Homo sapiens pentaxin-related gene, rapidly induced by IL-1 beta (PTX3), mRNA. /FEA = mRNA /GEN = PTX3 |
| | /PROD = pentaxin-related gene, rapidly induced by IL-1beta /DB_XREF = gi: 4506332 /UG = Hs.2050 pentaxin-related gene, rapidly induced |
| | by IL-1 beta /FL = gb: M31166.1 gb: NM_002852.1 |
| 209369_at | gb: M63310.1 /DEF = Human 1,2-cyclic-inositol-phosphate phosphodiesterase (ANX3) mRNA, complete cds. /FEA = mRNA /GEN = ANX3 |
| | /PROD = 1,2-cyclic-inositol-phosphate phosphodiesterase /DB_XREF = gi: 178696 /UG = Hs.1378 annexin A3 /FL = gb: BC000871.1 |
| | gb: M63310.1 gb: M20560.1 gb: NM_005139.1 |
| 202497_x_at | Consensus includes gb: AI631159 /FEA = EST /DB_XREF = gi: 4682489 /DB_XREF = est: ts93d05.x1 /CLONE = IMAGE: 2238825 /UG = Hs.7594 solute |
| | carrier family 2 (facilitated glucose transporter), member 3 /FL = gb: M20681.l gb: NM_006931.1 |
| 205513_at | gb: NM_001062.1 /DEF = Homo sapiens transcobalamin I (vitamin B12 binding protein, R binder family) (TCN1), mRNA. /FEA = mRNA |
| | /GEN = TCN1 /PROD = transcobalamin I (vitamin B12 binding protein, Rbinder family) /DB_XREF = gi: 4507406 /UG = Hs.2012 transcobalamin I |
| | (vitamin B12 binding protein, R binder family) /FL = gb: J05068.1 gb: NM_001062.1 |
| 206697_s_at | gb: NM_005143.1 /DEF = Homo sapiens haptoglobin (HP), mRNA. /FEA = mRNA /GEN = HP /PROD = haptoglobin /DB_XREF = gi: 4826761 /UG = Hs.75990 |
| | haptoglobin /FL = gb: K00422.1 gb: L29394.1 gb: NM_005143.1 |
| 202146_at | Consensus includes gb: AA747426 /FEA = EST /DB_XREF = gi: 2787384 /DB_XREF = est: nx88e08.s1 /CLONE = IMAGE: 1269350 /UG = Hs.7879 interferon- |
| | related developmental regulator 1 /FL = gb: BC001272.1 gb: NM_001550.1 |
| 266_s_at | L33930 /FEATURE = /DEFINITION = HUMCD24B Homo sapiens CD24 signal transducer mRNA, complete cds and 3 region |
| 211657_at | gb: M18728.1 /DEF = Human nonspecific crossreacting antigen mRNA, complete cds. /FEA = mRNA /GEN = NCA; NCA; NCA /PROD = non-specific cross |
| | reacting antigen /DB_XREF = gi: 189084 /FL = gb: M18728.1 |
| 205214_at | gb: NM_004226.1 /DEF = Homo sapiens serinethreonine kinase 17b (apoptosis-inducing) (STK17B), mRNA. /FEA = mRNA /GEN = STK17B |
| | /PROD = serinethreonine kinase 17b(apoptosis-inducing) /DB_XREF = gi: 4758193 /UG = Hs.120996 serinethreonine kinase 17b |
| | (apoptosis-inducing) /FL = gb: AB011421.1 gb: NM_004226.1 |
| 207574_s_at | gb: NM_015675.1 /DEF = Homo sapiens growth arrest and DNA-damage-inducible, beta (GADD45B), mRNA. /FEA = mRNA /GEN = GADD45B |
| | /PROD = DKFZP566B133 protein /DB_XREF = gi: 9945331 /UG = Hs.110571 growth arrest and DNA-damage-inducible, beta /FL = gb: AF090950.1 |
| | gb: NM_015675.1 |
| 206343_s_at | gb: NM_013959.1 /DEF = Homo sapiens neuregulin 1 (NRG1), transcript variant SMDF, mRNA. /FEA = mRNA /GEN = NRG1 |
| | /PROD = neuregulin 1 isoform SMDF //DB_XREF = gi: 7669517 /UG = HS.172816 neuregulin 1 /FL = gb: NM_013959.1 gb: L41827.1 |
| 214146_s_at | Consensus includes gb: R64130 /FEA = EST /DB_XREF = gi: 836009 /DB_XREF = est: yi18h03.s1 /CLONE = IMAGE: 139637 |
| | /UG = Hs.2164 pro-platelet basic protein (includes platelet basic protein, beta-thromboglobulin, |
| | connective tissue-activating peptide III, neutrophil-activating peptide-2) |
| 215716_s_at | Consensus includes gb: L14561 /DEF = Homo sapiens plasma membrane calcium ATPase isoform 1 (ATP2B1) gene, alternative |
| | splice products, partial cds /FEA = mRNA 2 /DB_XREF = gi: 4165324 /UG = Hs.78546 ATPase, Ca++ transporting, plasma membrane 1 |
| 201179_s_at | gb: J03005.1 /DEF = Human alternative guanine nucleotide-binding regulatory protein (G) alpha-inhibitory-subunit mRNA, |
| | complete cds. /FEA = mRNA /GEN = GNAI1 /DB_XREF = gi: 183183 /UG = Hs.73799 guanine nucleotide binding protein (G protein), |
| | alpha inhibiting activity polypeptide 3 /FL = gb: J03005.1 gb: J03198.1 gb: M27543.1 gb: J03238.1 gb: NM_006496.1 |
| 201626_at | Consensus includes gb: BG292233 /FEA = EST /DB_XREF = gi: 13050848 /DB_XREF = est: 602386668F1 |
| | /CLONE = IMAGE: 4515521 /UG = Hs.56205 insulin induced gene 1 /FL = gb: NM_005542.1 |
| 209728_at | gb: BC005312.1 /DEF = Homo sapiens, clone MGC: 12387, mRNA, complete cds. /FEA = mRNA /PROD = Unknown (protein for MGC: 12387) |
| | /DB_XREF = gi: 13529055 /UG = Hs.318720 Homo sapiens, clone MGC: 12387, mRNA, complete cds /FL = gb: BC005312.1 gb: M16942.1 |
| 212665_at | Consensus includes gb: AL556438 /FEA = EST /DB_XREF = gi: 12899113 /DB_XREF = est: AL556438 /CLONE = CS0DK004YI18 (3 prime) |
| | /UG = Hs.12813 DKFZP434J214 protein |
| 200665_s_at | gb: NM_003118.1 /DEF = Homo sapiens secreted protein, acidic, cysteine-rich (osteonectin) (SPARC), mRNA. /FEA = mRNA |
| | /GEN = SPARC /PROD = secreted protein, acidic, cysteine-rich(osteonectin) /DB_XREF = gi: 4507170 /UG = Hs.111779 secreted protein, acidic, |
| | cysteine-rich (osteonectin) /FL = gb: BC004974.1 gb: J03040.1 gb: NM_003118.1 |
| 201407_s_at | Consensus includes gb: AI186712 /FEA = EST /DB_XREF = gi: 3737350 /DB_XREF = est: qe82f01.x1 /CLONE = IMAGE: 1745497 /UG = Hs.21537 protein |
| | phosphatase 1, catalytic subunit, beta isoform /FL = gb: NM_002709.1 gb: AF092905.1 |
| 203505_at | gb: AF285167.1 /DEF = Homo sapiens ATP-binding cassette transporter 1 (ABCA1) mRNA, complete cds. /FEA = mRNA |
| | /GEN = ABCA1 /PROD = ATP-binding cassette transporter 1 /DB_XREF = gi: 9755158 /UG = Hs.211562 ATP-binding cassette, |
| | sub-family A (ABC1), member 1 /FL = gb: AF165281.1 gb: NM_005502.1 gb: AF285167.1 |
| 221802_s_at | Consensus includes gb: AU157109 /FEA = EST /DB_XREF = gi: 11018630 /DB_XREF = est: AU157109 /CLONE = PLACE1006159 |
| | /UG = Hs.23740 KIAA1598 protein |
| 201844_s_at | Consensus includes gb: W84482 /FEA = EST /DB_XREF = gi: 1395613 /DB_XREF = est: zd89h07.s1 /CLONE = IMAGE: 356701 |
| | /UG = Hs.7910 RING1 and YY1 binding protein /FL = gb: AF179286.1 gb: AB029551.1 gb: NM_012234.1 |
| 206390_x_at | gb: NM_002619.1 /DEF = Homo sapiens platelet factor 4 (PF4), mRNA. /FEA = mRNA /GEN = PF4 /PROD = platelet factor 4 |
| | /DB_XREF = gi: 4505732 /UG = Hs.81564 platelet factor 4 /FL = gb: M25897.1 gb: NM_002619.1 |
| 212569_at | Consensus includes gb: AV699744 /FEA = EST /DB_XREF = gi: 10301715 /DB_XREF = est: AV699744 /CLONE = GKCEDF05 /UG = Hs.8118 KIAA0650 protein |
| 211960_s_at | Consensus includes gb: BG261416 /FEA = EST /DB_XREF = gi: 12771232 /DB_XREF = est: 602373192F1 |
| | /CLONE = IMAGE: 4484422 /UG = Hs.237955 hypothetical protein PR02706 |
| 202422_s_at | gb: NM_022977.1 /DEF = Homo sapiens fatty-acid-Coenzyme A ligase, long-chain 4 (FACL4), transcript variant 2, mRNA. |
| | /FEA = mRNA /GEN = FACL4 /PROD = long-chain fatty-acid-Coenzyme A ligase 4, isoform 2 /DB_XREF = gi: 12669908 |
| | /UG = Hs.81452 fatty-acid-Coenzyme A ligase, long-chain 4 /FL = gb: NM_022977.1 |
| 221958_s_at | Consensus includes gb: AA775681 /FEA = EST /DB_XREF = gi: 2835015 /DB_XREF = est: zf31a02.s1 /CLONE = IMAGE: 378506 |
| | /UG = Hs.250746 hypothetical protein FLJ23091 |
| 204621_s_at | Consensus includes gb: AI935096 /FEA = EST /DB_XREF = gi: 5673966 /DB_XREF = est: wp13e10.x1 /CLONE = IMAGE: 2464746 |
| | /UG = Hs.82120 nuclear receptor subfamily 4, group A, member 2 /FL = gb: NM_006186.1 |
| 201409_s_at | gb: NM_002709.1 /DEF = Homo sapiens protein phosphatase 1, catalytic subunit, beta isoform (PPP1CB), mRNA. |
| | /FEA = mRNA /GEN = PPP1CB /PROD = protein phosphatase 1, catalytic subunit, betaisoform /DB_XREF = gi: 4506004 |
| | /UG = Hs.21537 protein phosphatase 1, catalytic subunit, beta isoform /FL = gb: NM_002709.1 gb: AF092905.1 |
| 201662_s_at | gb: D89053.1 /DEF = Homo sapiens mRNA for Acyl-CoA synthetase 3, complete cds. /FEA = mRNA /PROD = Acyl-CoA synthetase 3 |
| | /DB_XREF = gi: 4165017 /UG = Hs.268012 fatty-acid-Coenzyme A ligase, long-chain 3 /FL = gb: NM_004457.2 gb: D89053.1 gb: AF116690.1 |
| 220987_s_at | gb: NM_030952.1 /DEF = Homo sapiens hypothetical protein DKFZp434J037 (DKFZP434J037), mRNA. /FEA = mRNA |
| | /GEN = DKFZP434J037 /PROD = hypothetical protein DKFZp434J037 /DB_XREF = gi: 13569921 /FL = gb: NM_030952.1 |
| 219607_s_at | gb: NM_024021.1 /DEF = Homo sapiens membrane-spanning 4-domains, subfamily A, member 4 (MS4A4), mRNA. |
| | /FEA = mRNA /GEN = MS4A4 /PROD = membrane-spanning 4-domains, subfamily A, member4 /DB_XREF = gi: 13430865 |
| | /UG = Hs.325960 membrane-spanning 4-domains, subfamily A, member 4 /FL = gb: AB013102.1 b: NM_024021.1 gb: AF068288.1 gb: NM_016650.1 |
| 201565_s_at | gb: NM_002166.1 /DEF = Homo sapiens inhibitor of DNA binding 2, dominant negative helix-loop-helix protein (ID2), mRNA. /FEA = mRNA |
| | /GEN = ID2 /PROD = inhibitor of DNA binding 2, dominant negativehelix-loop-helix protein /DB_XREF = gi: 4504570 |
| | /UG = Hs.180919 inhibitor of DNA binding 2, dominant negative helix-loop-helix protein /FL = gb: M97796.1 gb: NM_002166.1 gb: D13891.1 |
| 213327_s_at | Consensus includes gb: AI820101 /FEA = EST /DB_XREF = gi: 5439180 /DB_XREF = est: wg56d12.x1 |
| | /CLONE = IMAGE: 2369111 /UG = Hs.42400 ubiquitin specific protease 12 |
| 203603_s_at | gb: NM_014795.1 /DEF = Homo sapiens zinc finger homeobox 1B (ZFHX1B), mRNA. /FEA = mRNA /GEN = ZFHX1B /PROD = zinc finger homeobox 1B |
| | /DB_XREF = gi: 7662183 /UG = Hs.34871 zinc finger homeobox 1B /FL = gb: AB011141.1 gb: NM_014795.1 |
| 217963_s_at | gb: NM_014380.1 /DEF = Homo sapiens p75NTR-associated cell death executor; ovarian granulosa cell protein (13 kD) (DXS6984E), mRNA. |
| | /FEA = mRNA /GEN = DXS6984E /PROD = p75NTR-associated cell death executor; ovariangranulosa cell protein (13 kD) /DB_XREF = gi: 7657043 |
| | /UG = Hs.17775 p75NTR-associated cell death executor; ovarian granulosa cell protein (13 kD) /FL = gb: NM_014380.1 gb: AF187064.1 |
| 206710_s_at | gb: NM_012307.1 /DEF = Homo sapiens differentially expressed in adenocarcinoma of the lung (KIAA0987), mRNA. |
| | /FEA = mRNA /GEN = KIAA0987 /PROD = differentially expressed in adenocarcinoma ofthe lung /DB_XREF = gi: 6912469 |
| | /UG = Hs.103839 erythrocyte membrane protein band 4.1-like 3 /FLFL = gb: AF069072.1 gb: NM_012307.1 |
| 204342_at | gb: NM_013386.1 /DEF = Homo sapiens hypothetical protein (DKFZp586G0123), mRNA. /FEA = mRNA /GEN = DKFZp586G0123 |
| | /PROD = hypothetical protein /DB_XREF = gi: 9558726 /UG = Hs.24713 hypothetical protein /FL = gb: AL050209.1 gb: NM_013386.1 |
| 204393_s_at | gb: NM_001099.2 /DEF = Homo sapiens acid phosphatase, prostate (ACPP), mRNA. /FEA = mRNA /GEN = ACPP |
| | /PROD = prostatic acid phosphatase precursor /DB_XREF = gi: 6382063 /UG = Hs.1852 acid phosphatase, prostate |
| | /FL = gb: M24902.1 gb: M34840.1 gb: NM_001099.2 |
| 200719_at | Consensus includes gb: BE964043 /FEA = EST /DB_XREF = gi: 11767371 /DB_XREF = est: 601657616R1 |
| | /CLONE = IMAGE: 3875955 /UG = Hs.171626 transcription elongation factor B (SIII), polypeptide 1-like /FL = gb: NM_003197.2 |
| 202731_at | gb: NM_014456.1 /DEF = Homo sapiens programmed cell death 4 (PDCD4), mRNA. /FEA = mRNA /GEN = PDCD4 /PROD = programmed cell death 4 |
| | /DB_XREF = gi: 7657448 /UG = Hs.296251 programmed cell death 4 /FL = gb: U96628.1 gb: NM_014456.1 |
| 204094_s_at | gb: NM_014779.1 /DEF = Homo sapiens KIAA0669 gene product (KIAA0669), mRNA. /FEA = mRNA /GEN = KIAA0669 /PROD = KIAA0669 gene product |
| | /DB_XREF = gi: 7662235 /UG = Hs.52526 KIAA0669 gene product /FL = gb: AB014569.1 gb: NM_014779.1 |
| 208690_s_at | gb: BC000915.1 /DEF = Homo sapiens, Similar to LIM protein, clone MGC: 5344, mRNA, complete cds. |
| | /FEA = mRNA /PROD = Similar to LIM protein /DB_XREF = gi: 12654194 /UG = Hs.75907 PDZ and LIM domain 1 (elfin) /FL = gb: BC000915.1 |
| 208979_at | gb: AF128458.1 /DEF = Homo sapiens nuclear receptor coactivacor RAP250 mRNA, complete cds. /FEA = mRNA |
| | /PROD = nuclear receptor coactivator RAP250 /DB_XREF = gi: 7140573 /UG = Hs.159613 thyroid hormone receptor binding protein |
| | /FL = gb: AF177388.1 gb: AF208227.1 gb: AF128458.1 gb: NM_014071.1 gb: AF171667.1 gb: AF245115.1 |
| 201772_at | gb: NM_015878.1 /DEF = Homo sapiens antizyme inhibitor (LOC51582), mRNA. /FEA = mRNA /GEN = LOC51582 /PROD = antizyme inhibitor |
| | /DB_XREF = gi: 7706219 /UG = Hs.223014 antizyme inhibitor /FL = gb: D88674.1 gb: NM_015878.1 |
| 201345_s_at | gb: NM_003339.1 /DEF = Homo sapiens ubiquitin-conjugating enzyme E2D 2 (homologous to yeast UBC45) (UBE2D2), mRNA. |
| | /FEA = mRNA /GEN = UBE2D2 /PROD = ubiquitin-conjugating enzyme E2D 2 (homologousto yeast UBC45) |
| | /DB_XREF = gi: 4507774 /UG = Hs.108332 ubiquitin-conjugating enzyme E2D 2 (homologous to yeast (UBC45) |
| | /FL = gb: U39317.1 gb: NM_003339.1 |
| 221060_s_at | gb: NM_003266.1 /DEF = Homo sapiens toll-like receptor 4 (TLR4), mRNA. /FEA = mRNA /GEN = TLR4 |
| | /PROD = toll-like receptor 4 /DB_XREF = gi: 4507532 /UG = Hs.159239 toll-like receptor 4 /FL = gb: U88880.1 gb: NM_003266.1 |
| 208878_s_at | gb: AF092132.1 /DEF = Homo sapiens PAK2 mRNA, complete cds. /FEA = mRNA /PROD = PAK2 /DB_XREF = gi: 5138913 |
| | /UG = Hs.284275 Homo sapiens PAK2 mRNA, 7complete cds /FL = gb: AF092132.1 |
| 212628_at | Consensus includes gb: BG292065 /FEA = EST /DB_XREF = gi: 13050507 /DB_XREF = est: 602386350F1 /CLONE = IMAGE: 4515036 |
| | /UG = Hs.69171 protein kinase C-like 2 |
| 212928_at | Consensus includes gb: AL050331 /DEF = Human DNA sequence from clone 486I3 on chromosome 6q22.1-22.3. |
| | Contains the part of a gene for a novel protein, the gene for KIAA0721 (NAP (Nucleosome Assembly Protein) |
| | domain containg protein), the TSPYL gene for TSPY-like (testis specific protei . . . /FEA = mRNA_1 |
| | /DB_XREF = gi: 5668655 /UG = Hs.284141 KIAA0721 protein /FL = gb: NM_021648.1 |
| 221568_s_at | gb: AF090900.1 /DEF = Homo sapiens clone HQ0189 PRO0189 mRNA, complete cds. /FEA = mRNA /PROD = PRO0189 |
| | /DB_XREF = gi: 6690176 /UG = Hs.91393 Homo sapiens cDNA: FLJ21887 fis, clone HEP03135, |
| | highly similar to AF090900 Homo sapiens clone HQ0189 PRO0189 mRNA /FL = gb: AF090900.1 |
| 201408_at | Consensus includes gb: W67887 /FEA = EST /DB_XREF = gi: 1376776 /DB_XREF = est: zd38c11.s1 /CLONE = IMAGE: 342932 |
| | /UG = Hs.21537 protein phosphatase 1, catalytic subunit, beta isoform /FL = gb: NM_002709.1 gb: AF092905.1 |
| 202270_at | gb: NM_002053.1 /DEF = Homo sapiens guanylate binding protein 1, interferon-inducible, 67 kD (GBP1), mRNA. |
| | /FEA = mRNA /GEN = GBP1 /PROD = guanylate binding protein 1, interferon-inducible, 67 kD /DB_XREF = gi: 4503938 |
| | UG = Hs.62661 guanylate binding protein 1, interferon-inducible, 67 kD /FL = gb: BC002666.1 gb: M55542.1 gb: NM_002053.1 |
| 202606_s_at | gb: NM_012290.1 /DEF = Homo sapiens tousled-like kinase 1 (TLK1), mRNA. /FEA = mRNA /GEN = TLK1 /PROD = tousled-like kinase 1 |
| | /DB_XREF = gi: 6912719 /UG = Hs.18895 tousled-like kinase 1 /FL = gb: AB004885.1 gb: NM_012290.1 gb: AF246219.1 |
| 216037_x_at | Consensus includes gb: AA664011 /FEA = EST /DB_XREF = gi: 2618002 /DB_XREF = est: ac03f10.s1 /CLONE = IMAGE: 855403 |
| | /UG = Hs.173638 transcription factor 7-like 2 (T-cell specific, HMG-box) |
| 221561_at | gb: L21934.2 /DEF = Homo sapiens acyl-coenzyme A: cholesterol acyltransferase mRNA, complete cds. |
| | /FEA = mRNA /PROD = acyl-coenzyme A: cholesterol acyltransferase /DB_XREF = gi: 4878021 |
| | /UG = Hs.14553 sterol O-acyltransferase (acyl-Coenzyme A: cholesterol acyltransferase) 1 /FL = gb: NM_003101.1 gb: L21934.2 |
| 203680_at | gb: NM_002736.1 /DEF = Homo sapiens protein kinase, cAMP-dependent, regulatory, type II, beta (PRKAR2B), mRNA. |
| | /FEA = mRNA /GEN = PRKAR2B /PROD = protein kinase, cAMP-dependent, regulatory, typeII, beta /DB_XREF = gi: 4506064 |
| | /UG = Hs.77439 protein kinase, cAMP-dependent, regulatory, type II, beta /FL = gb: M31158.1 gb: NM_002736.1 |
| 215111_s_at | Consensus includes gb: AK027071.1 /DEF = Homo sapiens cDNA: FLJ23418 fis, clone HEP21245, |
| | highly similar to HSU35048 Human TSC-22 protein mRNA. /FEA = mRNA /DB_XREF = gi: 10440100 |
| | /UG = Hs.114360 transforming growth factor beta-stimulated protein TSC-22 |
| 202211_at | gb: BC005122.1 /DEF = Homo sapiens, ADP-ribosylation factor GTPase activating protein 1, |
| | clone MGC: 10272, mRNA, complete cds. /FEA = mRNA |
| | /PROD = ADP-ribosylation factor GTPase activatingprotein 1 /DB_XREF = gi: 13477296 /UG = Hs.13014 ADP-ribosylation factor GTPase activating |
| | protein 1 /FL = gb: BC005122.1 gb: AF111847.1 gb: NM_014570.1 |
| 221918_at | Consensus includes gb: AI742210 /FEA = EST /DB_XREF = gi: 5110498 /DB_XREF = est: wg39c02.x1 /CLONE = IMAGE: 2367458 /UG = Hs.183302 ESTs |
| 213251_at | Consensus includes gb: AV712064 /FEA = EST /DB_XREF = gi: 10731370 /DB_XREF = est: AV712064 /CLONE = DCAAUD05 /UG = Hs.9456 SWISNF related, matrix |
| | associated, |
| | actin dependent regulator of chromatin, subfamily a, member 5 |
| 209186_at | gb: M23114.1 /DEF = Homo sapiens calcium-ATPase (HK1) mRNA, complete cds. /FEA = mRNA /GEN = HK1 /DB_XREF = gi: 184100 /UG = Hs.1526 ATPase, Ca++ |
| | transporting, cardiac muscle, slow twitch 2 /FL = gb: M23114.1 |
| 210996_s_at | gb: U43430.1 /DEF = Human epsilon isoform 14-3-3 protein mRNA, complete cds. /FEA = mRNA /PROD = 14-3-3 protein /DB_XREF = gi: 4096984 /UG = Hs.79474 |
| | tyrosine 3-monooxygenasetryptophan 5-monooxygenase activation protein, epsilon polypeptide /FL = gb: U43399.1 gb: U43430.1 |
| 213624_at | Consensus includes gb: AA873600 /FEA = EST /DB_XREF = gi: 2969722 /DB_XREF = est: ob12a02.s1 /CLONE = IMAGE: 1323434 /UG = Hs.42945 acid sphingomyelinase- |
| | like phosphodiesterase |
| 204634_at | gb: NM_003157.1 /DEF = Homo sapiens serinethreonine kinase 2 (STK2), mRNA. /FEA = mRNA /GEN = STK2 /PROD = serinethreonine kinase 2 |
| | /DB_XREF = gi: 4507276 |
| | /UG = Hs.1087 serinethreonine kinase 2 /FL = gb: L20321.1 gb: NM_003157.1 |
| 212515_s_at | Consensus includes gb: BG492602 /FEA = EST /DB_XREF = gi: 13454114 /DB_XREF = est: 602536568F1 /CLONE = IMAGE: 4655296 /UG = Hs.147916 DEADH |
| | (Asp-Glu-Ala-AspHis) box polypeptide 3 |
| 218170_at | gb: NM_016048.1 /DEF = Homo sapiens CGI-111 protein (LOC51015), mRNA. /FEA = mRNA /GEN = LOC51015 /PROD = CGI-111 protein /DB_XREF = gi: 7705613 |
| | /UG = Hs.11085 CGI-111 protein /FL = gb: AF151869.1 gb: NM_016048.1 |
| 210293_s_at | gb: BC005032.1 /DEF = Homo sapiens, Sec23 (S. cerevisiae) homolog B, clone MGC: 12666, mRNA, complete cds. /FEA = mRNA /PROD = Sec23 (S. cerevisiae) |
| | homolog B /DB_XREF = gi: 13477148 /UG = Hs.173497 Sec23 (S. cerevisiae) homolog B /FL = gb: BC005032.1 |
| 221493_at | gb: AL136629.1 /DEF = Homo sapiens mRNA; cDNA DKFZp564D152 (from clone DKFZp564D152); complete cds. /FEA = mRNA /GEN = DKFZp564D152 |
| | /PROD = hypothetical protein /DB_XREF = gi: 12052783 /UG = Hs.278479 TSPY-like /FL = gb: AL136629.1 |
| 213506_at | Consensus includes gb: BE965369 /FEA = EST /DB_XREF = gi: 11769659 /DB_XREF = est: 601659282R1 /CLONE = IMAGE: 3895653 /UG = Hs.168102 Human proteinase activated |
| | receptor-2 mRNA, 3UTR |
| 201016_at | Consensus includes gb: BE542684 /FEA = EST /DB_XREF = gi: 9771329 /DB_XREF = est: 601067180F1 /CLONE = IMAGE: 3453571 /UG = Hs.4310 eukaryotic translation |
| | initiation factor 1A /FL = gb: BC000793.1 gb: L18960.1 gb: NM_001412.1 |
| 203414_at | gb: NM_012329.1 /DEF = Homo sapiens monocyte to macrophage differentiation-associated (MMD), mRNA. /FEA = mRNA /GEN = MMD /PROD = monocyte to |
| | macrophagedifferentiation-associated, precursor /DB_XREF = gi: 6912507 /UG = Hs.79889 monocyte to macrophage differentiation-associated |
| | /FL = gb: NM_012329.1 |
| 213510_x_at | Consensus includes gb: AW194543 /FEA = EST /DB_XREF = gi: 6473381 /DB_XREF = est: xb27c02.x1 /CLONE = IMAGE: 2577506 /UG = Hs.234573 Homo sapiens mRNA |
| | for |
| | TL132 |
| 218539_at | gb: NM_017943.1 /DEF = Homo sapiens hypothetical protein FLJ20725 (FLJ20725), mRNA. /FEA = mRNA /GEN = FLJ20725 /PROD = hypothetical protein |
| | FLJ20725 |
| | /DB_XREF = gi: 8923650 /UG = Hs.15467 hypothetical protein FLJ20725 /FL = gb: NM_017943.1 |
| 218172_s_at | gb: NM_018630.1 /DEF = Homo sapiens hypothetical protein PRO2577 (PRO2577), mRNA. /FEA = mRNA /GEN = PRO2577 /PROD = hypothetical protein PRO2577 |
| | /DB_XREF = gi: 8924181 /UG = Hs.241576 hypothetical protein PRO2577 /FL = gb: AF116708.1 gb: NM_018630.1 |
| 202538_s_at | gb: NM_014043.1 /DEF = Homo sapiens DKFZP564O123 protein (DKFZP564O123), mRNA. /FEA = mRNA /GEN = DKFZP564O123 /PROD = DKFZP564O123 protein |
| | /DB_XREF = gi: 7661633 /UG = Hs.11449 DKFZP564O123 protein /FL = gb: AF151842.1 gb: AL080122.1 gb: NM_014043.1 |
| 221504_s_at | gb: AF112204.1 /DEF = Homo sapiens Vacuolar proton pump subunit SFD alpha isoform mRNA, complete cds. /FEA = mRNA /PROD = Vacuolar proton pump |
| | subunit |
| | SFD alpha isoform /DB_XREF = gi: 6563195 /UG = Hs.19575 CGI-11 protein /FL = gb: AF298777.1 gb: AF132945.1 gb: AF112204.1 gb: NM_015941.1 |
| 212213_x_at | Consensus includes gb: AB011139.1 /DEF = Homo sapiens mRNA for KIAA0567 protein, partial cds. /FEA = mRNA /GEN = KIAA0567 /PROD = KIAA0567 protein |
| | /DB_XREF = gi: 3043657 /UG = Hs.147946 optic atrophy 1 (autosomal dominant) |
| 216652_s_at | Consensus includes gb: AL137673.1 /DEF = Homo sapiens mRNA; cDNA DKFZp434H0872 (from clone DKFZp434H0872). /FEA = mRNA /DB_XREF = gi: 6807841 |
| | /UG = Hs.306454 Homo sapiens mRNA; cDNA DKFZp434H0872 (from clone DKFZp434H0872) |
| 221472_at | Consensus includes gb: Z97053 /DEF = Human DNA sequence from clone RP1-179M20 on chromosome 20 Contains a 3 end of a novel gene similar to |
| | cellular |
| | retinaldehyde-binding protein, the TDE1 gene (Tumour differentially expressed 1), the PKIG gene encoding protein kinase (cAMP-dependent . . . |
| | /FEA = mRNA 2 /DB_XREF = gi: 9650676 /UG = Hs.272168 tumor differentially expressed 1 /FL = gb: U49188.1 gb: AF112227.1 gb: AF153979.1 gb: NM_006811.1 |
| 212526_at | Consensus includes gb: AK002207.1 /DEF = Homo sapiens cDNA FLJ11345 fis, clone PLACE1010877, highly similar to Homo sapiens mRNA for KIAA0610 |
| | protein. /FEA = mRNA /DB_XREF = gi: 7023938 /UG = Hs.118087 KIAA0610 protein |
| 200624_s_at | Consensus includes gb: AA577695 /FEA = EST /DB_XREF = gi: 2355879 /DB_XREF = est: nn22h02.s1 /CLONE = IMAGE: 1084659 /UG = Hs.78825 matrin 3 |
| | /FL = gb: NM_018834.1 gb: AB018266.1 |
| 201091_s_at | Consensus includes gb: BE748755 /FEA = EST /DB_XREF = gi: 10162747 /DB_XREF = est: 601571933T1 /CLONE = IMAGE: 3838737 /UG = Hs.278554 heterochromatin- |
| | like |
| | protein 1 /FL = gb: AF136630.1 gb: NM_016587.1 |
| 217863_at | Consensus includes gb: AI348378 /FEA = EST /DB_XREF = gi: 4085584 /DB_XREF = est: qo20h06.x1 /CLONE = IMAGE: 1909115 /UG = Hs.75251 DEADH (Asp-Glu-Ala- |
| | AspHis) |
| | box binding protein 1 /FL = gb: AF077951.1 gb: AF167160.1 gb: NM_016166.1 |
| 202804_at | Consensus includes gb: AI539710 /FEA = EST /DB_XREF = gi: 4453845 /DB_XREF = est: tp77b05.x1 /CLONE = IMAGE: 2205297 /UG = Hs.89433 ATP-binding cassette, |
| | sub-family C (CFTRMRP), member 1 /FL = gb: L05628.1 gb: NM_004996.2 |
| 214198_s_at | Consensus includes gb: AU150824 /FEA = EST /DB_XREF = gi: 11012345 /DB_XREF = est: AU150824 /CLONE = NT2RP2003689 /UG = Hs.2491 DiGeorge syndrome |
| | critical |
| | region gene 2 |
| 217976_s_at | gb: NM_016141.1 /DEF = Homo sapiens dynein light chain-A (LOC51143), mRNA. /FEA = mRNA /GEN = LOC51143 /PROD = dynein light chain-A |
| | /DB_XREF = gi: 7705852 |
| | /UG = Hs.266483 dynein light chain-A /FL = gb: AF078849.1 gb: NM_016141.1 |
| 202318_s_at | gb: AF306508.1 /DEF = Homo sapiens SUMO-1 specific protease FKSG6 mRNA, complete cds. /FEA = mRNA /PROD = SUMO-1 specific protease FKSG6 |
| | /DB_XREF = gi: 11096243 /UG = Hs.27197 SUMO-1-specific protease /FL = gb: AF307849.1 gb: AF305608.1 gb: AF196304.1 gb: NM_015571.1 |
| 208673_s_at | gb: AF107405.1 /DEF = Homo sapiens pre-mRNA splicing factor (SFRS3) mRNA, complete cds. /FEA = mRNA /GEN = SFRS3 /PROD = pre-mRNA splicing factor |
| | /DB_XREF = gi: 5531903 /UG = Hs.167460 splicing factor, arginineserine-rich 3 /FL = gb: BC000914.1 gb: AF107405.1 |
| 218268_at | gb: NM_022771.1 /DEF = Homo sapiens hypothetical protein FLJ12085 (FLJ12085), mRNA. /FEA = mRNA /GEN = FLJ12085 /PROD = hypothetical protein |
| | FLJ12085 |
| | /DB_XREF = gi: 12232450 /UG = Hs.48827 hypothetical protein FLJ12085 /FL = gb: NM_022771.1 |
| 209250_at | gb: BC000951.2 /DEF = Homo sapiens, degenerative spermatocyte (homolog Drosophila; lipid desaturase), clone MGC: 5079, mRNA, complete cds. |
| | /FEA = mRNA /PROD = degenerative spermatocyte (homolog Drosophila; lipid desaturase) /DB_XREF = gi: 12803018 /UG = Hs.185973 degenerative spermatocyte |
| | (homolog Drosophila; lipid desaturase) /FL = gb: BC000961.2 |
| 218698_at | gb: NM_015957.1 /DEF = Homo sapiens CGI-29 protein (LOC51074), mRNA. /FEA = mRNA /GEN = LOC51074 /PROD = CGI-29 protein /DB_XREF = gi: 7705723 |
| | /UG = Hs.104058 |
| | CGI-29 protein /FL = gb: AF132963.1 gb: NM_015957.1 |

1. An array consisting of oligo- or poly-nucleotide probes applied and immobilized on a surface of a solid substrate, characterized in that sequences of a selection, or all, of the selective monocyte macrophage genes in Tables 1 to 6 are fixed on said surface.

2. The array according to claim 1, characterized in that additional further genes are used, wherein said additional genes are known to be expressed in a cell and to constitute part of the basic genotype of the cell.

3. The array according to claim 1, further characterized in that complementary RNA is bonded on the surface of the array with the aforementioned genes for inverse detection of the sequences in Tables 1 to 6.

4. The array according to claim 1, characterized in that the genes, their partial and oligomer sequences are selected genes of rheumatoid arthritis or other chronic inflammatory diseases, relevant for the disease and side effects, before and after anti-TNF therapy.

5. The array according to claim 1, characterized in that the genes, their partial and oligomer sequences are genes of the monocyte/macrophage cell system, which are regulated in a manner specific to the disease.

6. The array according to claim 1, further characterized in that alleles, derivatives and/or splicing variants of the gene or partial-gene sequences and oligomer sequences are present on the surface.

7. The array according to claim 1, characterized in that it contains gene sequences on the surface, which present a partial sequence identify of at least 80% in the protein-coding mRNA segments.

8. The array according to claim 1, further characterized in that the surface of the substrate is coated with reactive groups, metal compounds or alloys.

9. The array according to claim 1, further characterized in that the genes or gene sequences are applied in the form of RNA by cDNA spotting techniques, immobilizing techniques and techniques with oligomer synthesis or in an enantiomorphic form.

10-18. (canceled)

19. A method for detecting individual genes wherein said method utilizes a gene or gene sequence from Tables 1 to 6.

20. Use of the genes or gene sequences according to Tables 1 to 6, characterized in that they are provided with labeling or a reporter function.

21. A method for reverse detection of total RNA or messenger RNA bonded to a solid phase in an RNA array, operating on up to 500 tissue and/or blood samples wherein said method utilizes a gene or gene sequence from Tables 1 to 6.

22. A method for the diagnosis or monitoring of a disease condition, including, when desired, monitoring of treatment of the disease, wherein said method comprises contacting a sample with an array consisting of oligo- or poly-nucleotide probes applied and immobilized on a surface of a solid substrate, characterized in that sequences of a selection, or all, of the monocyte macrophage genes in Tables 1 to 6 are fixed on said surface.

23. The method according to the claim 22 wherein said method utilizes probes labeled for identification with fluorescence dye, an enzyme, protein or radioactive marker and permitting amplification.

24. The method according to claim 22, wherein said method utilizes probe and wherein signals are amplified via coupled alkaline phosphatase, peroxidase, biotin digoxigenin, protein molecules, metal chelates of beads.

25. The method according to claim 22, wherein said method utilizes probes and wherein streptavidin, metal chelates, beads or antibodies are employed for additional amplification of the signals.

26. The method according to claim 22, for inverse detection of total RNA or messenger RNA fixed to the solid phase.

27. The method according to claim 22, for measurement of the monocyte/macrophage activation or the inflammatory activity in the blood or in the cell tissue.

28. The method according to claim 22, for fine diagnosis as well as for early detection of inflammatory diseases and rheumatoid arthritis.

29. The method according to claim 22, for follow-up of side effects in anti-TNF therapy in cases of inflammatory diseases and rheumatoid arthritis.

30. The method according to claim 22, for monitoring the therapy and for establishment of a prognosis in cases of inflammatory diseases and rheumatoid arthritis.

31. The method according to claim 22, for the identification of pharmaceutical targets in cases of inflammatory diseases and rheumatoid arthritis.


SEQUENCE LISTING (1 sequence)

SEQ ID NO: 1 - protein, 5 aa, Homo sapiens
DEADH